(19) United States
(12) Patent Application Publication
(10) Pub. No.: US 2006/0275282 A1
(43) Pub. Date: Dec. 7, 2006

(54) Antibodies and Fc fusion proteins with altered immunogenicity

(75) Inventors: Gregory L. Moore, Pasadena, CA (US); Shannon Alicia Marshall, San Francisco, CA (US)

Correspondence Address: Robin M. Silva, Esq.;Dorsey & Whitney LLP, Intellectual Property Department, 555 California Street, Suite 1000, San Francisco, CA, 94104-1513, US

(73) Assignee: Xencor, Inc.

(21) Appl. No.: 11/332,619

(22) Filed: Jan. 12, 2006

Related U.S. Application Data
(60) Provisional application No. 60/643,313, filed on Jan. 12, 2005.
(60) Provisional application No. 60/652,958, filed on Feb. 14, 2005.
(60) Provisional application No. 60/654,636, filed on Feb. 17, 2005.

(51) Int. Cl. A61K 39/395; C07K 16/18

(52) U.S. Cl. 424/130.1; 530/387.1

(57) ABSTRACT

Variant antibodies and Fc fusion proteins with reduced immunogenicity are described. In particular, the variants of antibodies and Fc fusion proteins have reduced ability to bind one or more human class II MHC molecules are described.

This application claims benefit under 35 U.S.C. §119(e) to U.S. Ser. No. 60/643,313, filed Jan. 12, 2005, U.S. Ser. No. 60/652,958, filed Feb. 14, 2005, and U.S. Ser. No. 60/654,636, filed Feb. 17, 2005, which are expressly incorporated by reference in their entirety.

### FIELD OF THE INVENTION

The present invention relates to variant antibodies and Fc fusion proteins with reduced immunogenicity. In particular, variants of antibodies and Fc fusion proteins with reduced ability to bind one or more human class II MHC molecules are described.

### SEQUENCE LISTING

Pursuant to 37 C.F.R. §1.77(b)(4), reference is made to a Sequence Listing submitted on a compact disc as required by 37 C.F.R. §1.52(e)(5), which is incorporated by reference herein in its entirety. Sequences 1-1939 are provided on the compact discs, for which three are being filed herewith as Computer Readable Format Copy, Copy 1 and Copy 2.

### BACKGROUND OF THE INVENTION

Antibody Therapeutics

Monoclonal antibodies are used therapeutically for the treatment of a variety of conditions including cancer, inflammation, and cardiovascular disease. A number of features of antibodies, such as specificity for target, ability to mediate immune effector mechanisms, and long half-life in serum, make antibodies powerful therapeutics. There are currently over ten antibody products on the market and hundreds in development. In addition to antibodies, a related class of proteins that is finding an expanding role in research and therapy is the Fc fusion (Chamow et al., 1996, Trends Biotechnol 14:52-60; Ashkenazi et al., 1997, Curr Opin Immunol 9:195-200).

An Fc fusion is a protein wherein one or more polypeptides is operably linked to Fc. An Fc fusion combines the Fc region of an antibody, and thus its favorable effector functions and pharmacokinetics, with the target-binding region of a receptor, ligand, or some other protein or protein domain. The role of the latter is to mediate target recognition, and thus it is functionally analogous to the antibody variable region. Because of the structural and functional overlap of Fc fusions with antibodies, the discussion on antibodies in the present invention extends directly to Fc fusions.

Despite such widespread use, antibodies and Fc fusion proteins are not fully optimized for clinical use. One limitation is that some antibodies and Fc fusions, including antibodies with fully human sequence content, elicit unwanted immune responses.

Immunogenicity

Immunogenicity is a major barrier to the development and utilization of protein therapeutics, including antibodies and Fc fusion proteins. Several factors can contribute to protein immunogenicity, including but not limited to the protein sequence, the route and frequency of administration, and the patient population. Although immune responses are typically most severe for non-human proteins, such as murine antibodies, even therapeutics with mostly or entirely human sequence content may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis. Unwanted immune responses may reduce the efficacy of antibody and Fc fusion protein therapeutics by directly interfering with antigen recognition, altering interactions with effector molecules, or perturbing the serum half-life or tissue distribution of the therapeutic.

Unwanted immune responses have been observed for a number of antibodies and Fc fusion proteins. Murine antibodies including Oncoscint® (anti-TAG) and OKT3® (anti-CD3) elicited immune responses in a majority of patients. Immune responses affecting at least 5% of patients have been reported for Fc fusions and chimeric, humanized, and fully human antibodies, including Reopro® (chimeric anti-GPIIb/IIIa), Remicade® (infliximab, chimeric anti-TNF), Zenapax® (humanized anti-IL2R), (lenercept IgG-p55 TNFR fusion) and Enbrel® (etanercept, IgG1-p75 TNFR fusion) (Koren et al. (2002) Curr. Pharm. Biotechnol. 3: 349-360; Porter (2001) J. Pharm. Sci. 90: 1-11).

Because of the clinical success of monoclonal antibodies, immunogenicity reduction of these proteins has been an intense area of investigation. Antibodies are a unique system for the development of immunogenicity reduction methods because of the large number of highly conserved antibody sequences and the wealth of high-resolution structural information. A number of strategies for reducing antibody immunogenicity have been developed, such as chimerization and humanization. The central aim of all of these approaches has been the reduction of nonhuman, and correspondingly immunogenic content, while maintaining affinity for the antigen. For a description of the concepts of chimeric and humanized antibodies see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see, e.g., U.S. Pat. No. 4,816,567). Humanized antibodies comprise a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). Methods for humanizing non-human antibodies are well known in the art, and can be essentially performed following the method of Winter and co-workers (Jones et al., 1986, Nature 321:522-525; Riechmann et al.,1988, Nature 332:323-329; Verhoeyen et al., 1988, Science, 239:1534-1536). Additional examples of humanized murine monoclonal antibodies are also known in the art, for example antibodies binding human protein C (O'Connor et al., 1998, Protein Eng 11:321-8), interleukin 2 receptor (Queen et al., 1989, Proc Natl Acad Sci, USA 86:10029-33), and human epidermal growth factor receptor 2 (Carter et al., 1992, Proc Natl Acad Sci USA 89:4285-9). A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

Despite the significant clinical application of antibodies engineered using these methods, these methods remain nonrobust with regard to their ability to reduce immunogenicity. A number of humanized antibodies have elicited substantial immune reaction in clinical studies, with incidences of immune response as high as 63% of patients (Ritter et al., 2001, Cancer Research 61: 6851-6859).

Several methods have been developed to modulate the immunogenicity of proteins. In some cases, PEGylation has been observed to reduce the fraction of patients who raise neutralizing antibodies by sterically blocking access to antibody agretopes (see, e.g., Hershfield et al. 1991 PNAS 88:7185-7189; Bailon. et al. 2001 Bioconjug. Chem. 12: 195-202; He et al. 1999 Life Sci. 65: 355-368). Methods that improve the solution properties of a protein therapeutic may also reduce immunogenicity, as aggregates have been observed to be more immunogenic than soluble proteins.

A more general approach to immunogenicity reduction involves mutagenesis targeted at the agretopes and epitopes in the protein sequence and structure that are most responsible for stimulating the immune system. Such agretopes and epitopes may be present even in fully human sequences. Some success has been achieved by randomly replacing solvent-exposed residues to lower binding affinity to panels of known neutralizing antibodies (see, e.g., Laroche et al. Blood 96: 1425-1432 (2000)). Due to the incredible diversity of the antibody repertoire, mutations that lower affinity to known antibodies will typically lead to production of an another set of antibodies rather than abrogation of immunogenicity. However, in some cases it may be possible to decrease surface antigenicity by replacing hydrophobic and charged residues on the protein surface with polar neutral residues (see Meyer et al. Protein Sci. 10: 491-503 (2001)).

An alternate approach is to disrupt T-cell activation. Removal of MHC-binding agretopes offers a much more tractable approach to immunogenicity reduction, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see, e.g., WO 98/52976, WO 02/079232, and WO 00/3317.

While mutations in MHC-binding agretopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

There remains a need for novel antibodies and Fc fusion proteins having reduced immunogenicity. Variants of antibodies and Fc fusion proteins with reduced immunogenicity could find use in the treatment of a number of antibody and Fc fusion protein responsive conditions.

### SUMMARY OF THE INVENTION

The present invention provides novel antibodies and Fc fusion proteins having reduced immunogenicity as compared to parent antibodies and Fc fusion proteins. In an additional aspect, the present invention is directed to methods for engineering or designing less immunogenic antibodies and Fc fusion proteins for therapeutic use.

An aspect of the present invention are antibodies and Fc fusion proteins that show decreased binding affinity for one or more class II MHC alleles relative to a parent antibody or Fc fusion protein and which significantly maintain the activity of the parent antibody or Fc fusion protein.

In a further aspect, the invention provides recombinant nucleic acids encoding the variant antibodies and Fc fusion proteins, expression vectors, and host cells.

In an additional aspect, the invention provides methods of producing a variant antibody or Fc fusion protein comprising culturing the host cells of the invention under conditions suitable for expression of the variant antibody or Fc fusion protein.

In a further aspect, the invention provides pharmaceutical compositions comprising a variant antibody or Fc fusion protein or nucleic acid encoding an antibody or Fc fusion protein of the invention and a pharmaceutical carrier.

In a further aspect, the invention provides methods for preventing or treating antibody or Fc fusion protein responsive disorders comprising administering a variant antibody or Fc fusion protein or nucleic acid encoding an antibody or Fc fusion protein of the invention to a patient.

In an additional aspect, the invention provides methods for screening the class II MHC haplotypes of potential patients in order to identify individuals who are particularly likely to raise an immune response to given antibody or Fc fusion protein therapeutics.

The present invention provides variant antibodies and Fc fusion proteins comprising amino acid sequences with at least one amino acid insertion, deletion, or substitution compared to the parent antibody or Fc fusion protein.

In one aspect, the present invention includes a non-naturally occurring protein comprising a variant Fc region having the formula:

-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(l151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-;

wherein X(118) is A; X(119) is S; X(120) is T; X(121) is K; X(122) is G; X(123) is P; X(124) is S; X(125) is V; X(126) is F; X(127) is P; X(128) is L; X(129) is A; X(130) is P; X(131) is S; X(132) is S; X(133) is K; X(134) is S; X(135) is T; X(136) is S; X(137) is G; X(138) is G; X(139) is T; X(140) is A; X(141) is A; X(142) is L; X(143) is G; X(144) is C; X(145) is L; X(146) is V; X(147) is K; X(148) is D; X(149) is Y; X(150) is F; X(151) is P; X(152) is E; X(153) is P; X(154) is V; X(155) is T; X(156) is V; X(157) is S; X(158) is W; X(159) is N; X(160) is S; X(161) is G; X(162) is A; X(163) is L; X(164) is T; X(165) is S; X(166) is G; X(167) is V; X(168) is H; X(169) is T; X(170) is F; X(171) is P; X(172) is A; X(173) is V; X(174) is L; X(175) is Q; X(176) is S; X(177) is S; X(178) is G; X(179) is L; X(180) is Y; X(181) is S; X(182) is L; X(183) is S; X(184) is S; X(185) is V; X(186) is V; X(187) is T; X(188) is V; X(189) is P; X(190) is S; X(191) is S; X(192) is S; X(193) is L; X(194) is G; X(195) is T; X(196) is Q; X(197) is T; X(198) is Y; X(199) is I; X(200) is C; X(201) is N; X(202) is V; X(203) is N; X(204) is H; X(205) is K; X(206) is P; X(207) is S; X(208) is N; X(209) is T; X(210) is K; X(211) is V; X(212) is D; X(213) is K; X(214) is K; X(215) is V; X(216) is E; X(217) is P; X(218) is K; X(219) is S; X(220) is C; X(221) is D; X(222) is K; X(223) is T; X(224) is H; X(225) is T; X(226) is C; X(227) is P; X(228) is P; X(229) is C; X(230) is P; X(231) is A; X(232) is P; X(233) is E; X(234) is L; X(235) is L; X(236) is G; X(237) is G; X(238) is P; X(239) is S; X(240) is V; X(241) is F; X(242) is L; X(243) is F; X(244) is P; X(245) is P; X(246) is K; X(247) is P; X(248) is K; X(249) is D; X(250) is T; X(251) is selected from the group consisting of L, D, E, H, and T; X(252) is selected from the group consisting of M, D, E, and H; X(253) is selected from the group consisting of I, D, E, F, H, K, L, N, P, Q, R, S, T, V, W, and Y; X(254) is selected from the group consisting of S, E, K, N, P, Q, R, V, and W; X(255) is R; X(256) is selected from the group consisting of T, I, L, M, P, S, V, W, and Y; X(257) is P; X(258) is E; X(259) is selected from the group consisting of V and T; X(260) is T; X(261) is C; X(262) is V; X(263) is V; X(264) is V; X(265) is D; X(266) is V; X(267) is S; X(268) is H; X(269) is E; X(270) is D; X(271) is P; X(272) is E; X(273) is V; X(274) is K; X(275) is F; X(276) is N; X(277) is W; X(278) is selected from the group consisting of Y, D and E; X(279) is selected from the group consisting of V, A, Q, and T; X(280) is D; X(281) is G; X(282) is selected from the group consisting of V, F, I, L, P, Q, W, and Y; X(283) is E; X(284) is V; X(285) is selected from the group consisting of H, E, P, and T; X(286) is N; X(287) is A; X(288) is K; X(289) is T; X(290) is K; X(291) is P; X(292) is R; X(293) is E; X(294) is E; X(295) is Q; X(296) is Y; X(297) is N; X(298) is S; X(299) is T; X(300) is Y, X(301) is selected from the group consisting of R, G, K, and Q; X(302) is selected from the group consisting of D, E, H, K, Q, S, and T; X(303) is selected from the group consisting of V, N, P, Q, R, and S; X(304) is S; X(305) is selected from the group consisting of V, G, P, and T; X(306) is selected from the group consisting of L, F, H, I, N, T, V, and Y; X(307) is T; X(308) is selected from the group consisting of V, A, N, P, and S; X(309) is selected from the group consisting of L, F, G, I, M, N, Q, S, T, V, W, and Y; X(310) is H; X(311) is selected from the group consisting of Q, D, E, G, P, T, and W; X(312) is D; X(313) is W; X(314) is L; X(315) is N; X(316) is G; X(317) is K; X(318) is E; X(319) is Y; X(320) is K; X(321) is C; X(322) is K; X(323) is V; X(324) is S; X(325) is N; X(326) is K; X(327) is A; X(328) is L; X(329) is P; X(330) is A; X(331) is P; X(332) is l; X(333) is E; X(334) is K; X(335) is T; X(336) is l; X(337) is S; X(338) is K; X(339) is A; X(340) is K; X(341) is G; X(342) is Q; X(343) is P; X(344) is R; X(345) is E; X(346) is P; X(347) is Q; X(348) is V; X(349) is Y; X(350) is T; X(351) is L; X(352) is P; X(353) is P; X(354) is S; X(355) is R; X(356) is D; X(357) is E; X(358) is L; X(359) is T; X(360) is K; X(361) is N; X(362) is Q; X(363) is V; X(364) is S; X(365) is L; X(366) is T; X(367) is C; X(368) is L; X(369) is V; X(370) is K; X(371) is G; X(372) is F; X(373) is Y; X(374) is P; X(375) is S; X(376) is D; X(377) is I; X(378) is A; X(379) is V; X(380) is E; X(381) is W; X(382) is E; X(383) is S; X(384) is N; X(385) is G; X(386) is Q; X(387) is P; X(388) is E; X(389) is N; X(390) is N; X(391) is Y; X(392) is K; X(393) is T; X(394) is T; X(395) is P; X(396) is P; X(397) is V; X(398) is L; X(399) is D; X(400) is S; X(401) is D; X(402) is G; X(403) is S; X(404) is selected from the group consisting of F, H, I, L, M, N, Q, T, and V; X(405) is selected from the group consisting of F and W; X(406) is selected from the group consisting of L, A, D, E, G, K, N, Q, S, T, and V; X(407) is selected from the group consisting of Y and H; X(408) is S; X(409) is K; X(410) is selected from the group consisting of L, I and Q; X(411) is T; X(412) is selected from the group consisting of V and P; X(413) is D; X(414) is K; X(415) is S; X(416) is R; X(417) is W; X(418) is Q; X(419) is Q; X(420) is G; X(421) is N; X(422) is V; X(423) is F; X(424) is S; X(425) is C; X(426) is S; X(427) is V; X(428) is M; X(429) is H; X(430) is E; X(431) is A; X(432) is selected from the group consisting of L, E and K; X(433) is selected from the group consisting of H, D, G, P, S, T, and W; X(434) is selected from the group consisting of N, D, E, G, H, S, T, and W; X(435) is selected from the group consisting of H, G, K, M, N, P, S, T, and V; X(436) is Y; X(437) is selected from the group consisting of T, D, E, G, H, K, N, Q, and S; X(438) is selected from the group consisting of Q, G, P, S, and T; and, X(439) is K; X(440) is selected from the group consisting of S, D, E, G, H, K, N, P, Q, R, and T, X(441) is L; X(442) is S; X(443) is L; X(444) is S; X(445) is P; X(446) is G; X(447) is K. The non-naturally occurring protein has a reduced immunogenicity as compared with SEQ ID NO:1.

In another aspect, the present invention is directed to a non-naturally occurring protein comprising a variant Fc region comprising at least one amino acid modification of a naturally occurring protein sequence selected from the group consisting of SEQ ID NO:1, the modification at a position selected from the group consisting of positions 251, 252, 253, 254, 256, 259, 278, 279, 282, 285, 301, 302, 303, 305, 306, 308, 309, 311, 404, 405, 406, 407, 410, 412, 432, 433, 434, 435, 437, 438, and 440, wherein the modification at position 251 is selected from the group consisting of D, E, H, and T; wherein the modification at position 252 is selected from the group consisting of D, E, and H; wherein the modification at position 253 is selected from the group consisting of D, E, F, H, K, L, N, P, Q, R, S, T, V, W, and Y; wherein the modification at position 254 is selected from the group consisting of E, K, N, P, Q, R, V, and W; wherein the modification at position 256 is selected from the group consisting of I, L, M, P, S, V, W, and Y; wherein the modification at position 259 is T; wherein the modification at position 278 is selected from the group consisting of D and E; wherein the modification at position 279 is selected from the group consisting of A, Q, and T; wherein the modification at position 282 is selected from the group consisting of F, I, L, P, Q, W, and Y; wherein the modification at position 285 is selected from the group consisting of P and T; wherein the modification at position 301 is selected from the group consisting of G, K, and Q; wherein the modification at position 302 is selected from the group consisting of A, D, E, H, K, Q, S, and T; wherein the modification at position 303 is selected from the group consisting of N, P, Q, R, and S; wherein the modification at position 305 is selected from the group consisting of G, P, and T; wherein the modification at position 306 is selected from the group consisting of F, H, I, N, T, V, and Y; wherein the modification at position 308 is selected from the group consisting of A, N, P, and S; wherein the modification at position 309 is selected from the group consisting of F, G, I, M, N, Q, S, T, V, W, and Y; wherein the modification at position 311 is selected from the group consisting of D, E, G, P, T, and W; wherein the modification at position 404 is selected from the group consisting of H, I, L, M, N, Q, T, and V; wherein the modification at position 405 is W; wherein the modification at position 406 is selected from the group consisting of A, D, E, G, K, N, Q, S, T, and V; wherein the modification at position 407 is H; wherein the modification at position 410 is selected from the group consisting of I and Q; wherein the modification at position 412 is P; wherein the modification at position 432 is selected from the group consisting of E and K; wherein the modification at position 433 is selected from the group consisting of D, G, P, S, T, and W; wherein the modification at position 434 is selected from the group consisting of D, E, G, H, S, T, and W; wherein the modification at position 435 is selected from the group consisting of G, K, M, N, P, S, T, and V; wherein the modification at position 437 is selected from the group consisting of D, E, G, H, K, N, Q, and S; wherein the modification at position 438 is selected from the group consisting of G, P, S, and T; and, wherein the modification at position 440 is selected from the group consisting of D, E, G, H, K, N, P, Q, R, and T. The non-naturally occurring protein has reduced immunogenicity as compared with a protein comprising a naturally-occurring Fc region of SEQ ID NO:1.

In another variation, the variant protein the modification is made to an amino acid in one of the following agretopes: Agretope 1 (125-133), Agretope 2 (126-134), Agretope 3 (128-136), Agretope 4 (145-153), Agretope 5 (149-157), Agretope 6 (167-175), Agretope 7 (174-182), Agretope 8 (179-187), Agretope 9 (180-188), Agretope 10 (182-190), Agretope 11a (185-193), Agretope 12 (202-210), Agretope 15 (240-248), Agretope 16 (251-259), Agretope 17a (262-270), Agretope 18 (277-285), Agretope 19a (300-308), Agretope 20a (302-310), Agretope 21a (303-311), Agretope 23 (369-377), Agretope 24a (404-412), Agretope 25a (406-414), Agretope 27a (422-430), and Agretope 28a (432-440). Alternatively, the variant protein the modification is made to an amino acid in one of the following agretopes: Agretope 16 (251-259), Agretope 17a (262-270), Agretope 18 (277-285), Agretope 19a (300-308), Agretope 20a (302-310), Agretope 21 a (303-311), Agretope 23 (369-377), Agretope 24a (404-412), Agretope 25a (406-414), Agretope 27a (422-430), and Agretope 28a (432-440).

In a further variation, the modification at position 251 is selected from the group consisting of D, E, H, and T; wherein the modification at position 252 is D; the modification at position 253 is selected from the group consisting of D and E; the modification at position 256 is selected from the group consisting of M, W, and Y; the modification at position 278 is D; the modification at position 282 is selected from the group consisting of F, L, Q, W, and Y; the modification at position 303 is selected from the group consisting of N, P, Q, R, and S; the modification at position 311 is D; the modification at position 404 is selected from the group consisting of H, N, Q, and T; the modification at position 432 is selected from the group consisting of E and K; and, the modification at position 437 is E.

In another variation, the variant protein the modification is made to an amino acid in an agretope Agretope 4 (145-153), Agretope 5 (149-157), Agretope 6 (167-175), Agretope 7 (174-182), Agretope 8 (179-187), Agretope 9 (180-188), Agretope 10 (182-190), Agretope 11b (185-193), Agretope 15 (240-248), Agretope 16 (251-259), Agretope 17a (262-270), Agretope 18 (277-285), Agretope 19b (300-308), Agretope 20b (302-310), Agretope 21b (303-311), Agretope 22a (348-356), Agretope 23 (369-377), Agretope 24a (404-412), Agretope 25a (406-414), Agretope 27a (422-430), and Agretope 28a (432-440).

In another aspect, the present invention is directed to a non-naturally occurring protein comprising a variant Fc region having the formula:

-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-; X(118) is A; X(119) is S; X(120) is T; X(121) is K; X(122) is G; X(123) is P; X(124) is S; X(125) is V; X(126) is F; X(127) is P; X(128) is L; X(129) is A; X(130) is P; X(131) is C; X(132) is S; X(133) is R; X(134) is S; X(135) is T; X(136) is S; X(137) is E; X(138) is S; X(139) is T; X(140) is A; X(141) is A; X(142) is L; X(143) is G; X(144) is C; X(145) is L; X(146) is V; X(147) is K; X(148) is D; X(149) is Y; X(150) is F; X(151) is P; X(152) is E; X(153) is P; X(154) is V; X(155) is T; X(156) is V; X(157) is S; X(158) is W; X(159) is N; X(160) is S; X(161) is G; X(162) is A; X(163) is L; X(164) is T; X(165) is S; X(166) is G; X(167) is V; X(168) is H; X(169) is T; X(170) is F; X(171) is P; X(172) is A; X(173) is V; X(174) is L; X(175) is Q; X(176) is S; X(177) is S; X(178) is G; X(179) is L; X(180) is Y; X(181) is S; X(182) is L; X(183) is S; X(184) is S; X(185) is V; X(186) is V; X(187) is T; X(188) is V; X(189) is P; X(190) is S; X(191) is S; X(192) is N; X(193) is F; X(194) is G; X(195) is T; X(196) is Q; X(197) is T; X(198) is Y; X(199) is T; X(200) is C; X(201) is N; X(202) is V; X(203) is D; X(204) is H; X(205) is K; X(206) is P; X(207) is S; X(208) is N; X(209) is T; X(210) is K; X(211) is V; X(212) is D; X(213) is K; X(214) is T; X(215) is V; X(216) is E; X(217) is R; X(218) is K; X(219) is C; X(220) is C; X(221) is a bond; X(222) is V; X(223) is a bond; X(224) is E; X(225) is a bond; X(226) is C; X(227) is P; X(228) is P; X(229) is C; X(230) is P; X(231) is A; X(232) is P; X(233) is P; X(234) is V; X(235) is A; X(236) is a bond; X(237) is G; X(238) is P; X(239) is S; X(240) is V; X(241) is F; X(242) is L; X(243) is F; X(244) is P; X(245) is P; X(246) is K; X(247) is P; X(248) is K; X(249) is D; X(250) is T; X(251) is selected from the group consisting of L, D, E, H, and T; X(252) is selected from the group consisting of M, D, E, and H; X(253) is selected from the group consisting of I, D, E, F, H, K, L, N, P, Q, R, S, T, V, W, and Y; X(254) is selected from the group consisting of S, E, K, N, P, Q, R, V, and W; X(255) is R; X(256) is selected from the group consisting of T, I, L, M, P, S, V, W, and Y; X(257) is P; X(258) is E; X(259) is selected from the group consisting of V and T; X(260) is T; X(261) is C; X(262) is V; X(263) is V; X(264) is V; X(265) is D; X(266) is V; X(267) is S; X(268) is H; X(269) is E; X(270) is D; X(271) is P; X(272) is E; X(273) is V; X(274) is Q; X(275) is F; X(276) is N; X(277) is W; X(278) is selected from the group consisting of Y, D and E; X(279) is selected from the group consisting of V, A, Q, and T; X(280) is D; X(281) is G; X(282) is selected from the group consisting of V, F, I, L, Q, and W; X(283) is E; X(284) is V; X(285) is selected from the group consisting of H, P, and T; X(286) is N; X(287) is A; X(288) is K; X(289) is T; X(290) is K; X(291) is P; X(292) is R; X(293) is E; X(294) is E; X(295) is Q; X(296) is F; X(297) is N; X(298) is S; X(299) is T; X(300) is F; X(301) is selected from the group consisting of R, G, K, and Q; X(302) is selected from the group consisting of V, A, D, E, H, K, P, Q, S, and T; X(303) is selected from the group consisting of V, N, P, and Q; X(304) is S; X(305) is selected from the group consisting of V, G and P; X(306) is selected from the group consisting of L, I, N, T, V, and Y; X(307) is T; X(308) is V; X(309) is V; X(310) is H; X(311) is Q; X(312) is D; X(313) is W; X(314) is L; X(315) is N; X(316) is G; X(317) is K; X(318) is E; X(319) is Y; X(320) is K; X(321) is C; X(322) is K; X(323) is V; X(324) is S; X(325) is N; X(326) is K; X(327) is G; X(328) is L; X(329) is P; X(330) is A; X(331) is P; X(332) is I; X(333) is E; X(334) is K; X(335) is T; X(336) is l; X(337) is S; X(338) is K; X(339) is T; X(340) is K; X(341) is G; X(342) is Q; X(343) is P; X(344) is R; X(345) is E; X(346) is P; X(347) is Q; X(348) is V; X(349) is Y; X(350) is T; X(351) is L; X(352) is P; X(353) is P; X(354) is S; X(355) is R; X(356) is E; X(357) is E; X(358) is M; X(359) is T; X(360) is K; X(361) is N; X(362) is Q; X(363) is V; X(364) is S; X(365) is L; X(366) is T; X(367) is C; X(368) is L; X(369) is V; X(370) is K; X(371) is G; X(372) is F; X(373) is Y; X(374) is P; X(375) is S; X(376) is D; X(377) is I; X(378) is A; X(379) is V; X(380) is E; X(381) is W; X(382) is E; X(383) is S; X(384) is N; X(385) is G; X(386) is Q; X(387) is P; X(388) is E; X(389) is N; X(390) is N; X(391) is Y; X(392) is K; X(393) is T; X(394) is T; X(395) is P; X(396) is P; X(397) is M; X(398) is L; X(399) is D; X(400) is S; X(401) is D; X(402) is G; X(403) is S; X(404) is selected from the group consisting of F, H, L, M, and N; X(405) is selected from the group consisting of F, D, E, T, and W; X(406) is selected from the group consisting of L, A, D, E, G, K, N, Q, S, T, and V; X(407) is selected from the group consisting of Y and H; X(408) is S; X(409) is K; X(410) is selected from the group consisting of L, I, K, and Q; X(411) is T; X(412) is selected from the group consisting of V and P; X(413) is D; X(414) is K; X(415) is S; X(416) is R; X(417) is W; X(418) is Q; X(419) is Q; X(420) is G; X(421) is N; X(422) is V; X(423) is F; X(424) is S; X(425) is C; X(426) is S; X(427) is V; X(428) is M; X(429) is H; X(430) is E; X(431) is A; X(432) is selected from the group consisting of L, E and K; X(433) is selected from the group consisting of H, D, G, P, S, T, and W; X(434) is selected from the group consisting of N, D, E, G, H, S, T, and W; X(435) is selected from the group consisting of H, G, K, M, N, P, S, T, and V; X(436) is Y; X(437) is selected from the group consisting of T, D, E, G, H, K, N, Q, and S; X(438) is selected from the group consisting of Q, G, P, S, and T; and X(439) is K; X(440) is selected from the group consisting of S, D, E, G, H, K, N, P, Q, R, and T; X(441) is L; X(442) is S; X(443) is L; X(444) is S; X(445) is P; X(446) is G; and X(447) is K. The non-naturally occurring protein has reduced immunogenicity as compared with a protein comprising SEQ ID NO:2.

In a further aspect, the present invention is directed to a non-naturally occurring protein comprising a variant Fc region comprising at least one amino acid modification of SEQ ID NO:2, the modification at a position selected from the group consisting of positions 251, 252, 253, 254, 256, 259, 278, 279, 282, 285, 301, 302, 303, 305, 306, 404, 405, 406, 407, 410, 412, 432, 433, 434, 435, 437, 438, and 440. The modification at position 251 is selected from the group consisting of D, E, H, and T; the modification at position 252 is selected from the group consisting of D, E, and H; the modification at position 253 is selected from the group consisting of D, E, F, H, K, L, N, P, Q, R, S, T, V, W, and Y; the modification at position 254 is selected from the group consisting of E, K, N, P, Q, R, V, and W; the modification at position 256 is selected from the group consisting of I, L, M, P, S, V, W, and Y; the modification at position 259 is T; the modification at position 278 is selected from the group consisting of D and E; the modification at position 279 is selected from the group consisting of A, Q, and T; the modification at position 282 is selected from the group consisting of F, I, L, Q, and W; the modification at position 285 is selected from the group consisting of P and T; the modification at position 301 is selected from the group consisting of G, K, and Q; the modification at position 302 is selected from the group consisting of A, D, E, H, K, P, Q, S, and T; the modification at position 303 is selected from the group consisting of N, P, and Q; the modification at position 305 is selected from the group consisting of G and P; the modification at position 306 is selected from the group consisting of I, N, T, V, and Y; the modification at position 404 is selected from the group consisting of H, L, M, and N; the modification at position 405 is selected from the group consisting of D, E, T, and W; the modification at position 406 is selected from the group consisting of A, D, E, G, K, N, Q, S, T, and V; the modification at position 407 is H; the modification at position 410 is selected from the group consisting of I, K, and Q; the modification at position 412 is P; the modification at position 432 is selected from the group consisting of E and K; the modification at position 433 is selected from the group consisting of D, G, P, S, T, and W; the modification at position 434 is selected from the group consisting of D, E, G, H, S, T, and W; the modification at position 435 is selected from the group consisting of G, K, M, N, P, S, T, and V; the modification at position 437 is selected from the group consisting of D, E, G, H, K, N, Q, and S; the modification at position 438 is selected from the group consisting of G, P, S, and T; and, the modification at position 440 is selected from the group consisting of D, E, G, H, K, N, P, Q, R, and T, the non-naturally occurring protein having reduced immunogenicity as compared with a protein comprising a naturally-occurring Fc region.

In a further variation, at least one modification is made to the group consisting of positions 251, 252, 253, 254, 256, 259, 278, 279, 282, 285, 302, 303, 305, 306, 404, 405, 406, 407, 410, 412, 432, 433, 434, 435, 437, 438, and 440. The modification at position 251 is selected from the group consisting of D, E, H, and T; the modification at position 252 is D; the modification at position 253 is selected from the group consisting of D and E; the modification at position 256 is selected from the group consisting of M, W, and Y; the modification at position 278 is D; the modification at position 282 is selected from the group consisting of F, L, Q, and W; the modification at position 404 is selected from the group consisting of H and N; the modification at position 432 is selected from the group consisting of E and K; and, the modification at position 437 is E.

In a further aspect, the present invention is directed to a non-naturally occurring protein comprising a variant Fc region having the formula:

-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-.

In this aspect, X(118) is A; X(119) is S; X(120) is T; X(121) is K; X(122) is G; X(123) is P; X(124) is S; X(125) is V; X(126) is F; X(127) is P; X(128) is L; X(129) is A; X(130) is P; X(131) is C; X(132) is S; X(133) is R; X(134) is S; X(135) is T; X(136) is S; X(137) is G; X(138) is G; X(139) is T; X(140) is A; X(141) is A; X(142) is L; X(143) is G; X(144) is C; X(145) is L; X(146) is V; X(147) is K; X(148) is D; X(149) is Y; X(150) is F; X(151) is P; X(152) is E; X(153) is P; X(154) is V; X(155) is T; X(156) is V; X(157) is S; X(158) is W; X(159) is N; X(160) is S; X(161) is G; X(162) is A; X(163) is L; X(164) is T; X(165) is S; X(166) is G; X(167) is V; X(168) is H; X(169) is T; X(170) is F; X(171) is P; X(172) is A; X(173) is V; X(174) is L; X(175) is Q; X(176) is S; X(177) is S; X(178) is G; X(179) is L; X(180) is Y; X(181) is S; X(182) is L; X(183) is S; X(184) is S; X(185) is V; X(186) is V; X(187) is T; X(188) is V; X(189) is P; X(190) is S; X(191) is S; X(192) is S; X(193) is L; X(194) is G; X(195) is T; X(196) is Q; X(197) is T; X(198) is Y; X(199) is T; X(200) is C; X(201) is N; X(202) is V; X(203) is N; X(204) is H; X(205) is K; X(206) is P; X(207) is S; X(208) is N; X(209) is T; X(210) is K; X(211) is V; X(212) is D; X(213) is K; X(214) is R; X(215) is V; X(216) is E; X(217) is L; X(218) is K; X(219) is T; X(220) is P; X(221) is the sequence LGD; X(222) is T; X(223) is T; X(224) is H; X(225) is T; X(226) is C; X(227) is P; X(228) is the sequence RCPEPKSCDTPPPCPRCPEPKSCDTPPPCPRCPEPKSCDTPPPCPR; X(229) is C; X(230) is P; X(231) is A; X(232) is P; X(233) is E; X(234) is L; X(235) is L; X(236) is G; X(237) is G; X(238) is P; X(239) is S; X(240) is V; X(241) is F; X(242) is L; X(243) is F; X(244) is P; X(245) is P; X(246) is K; X(247) is P; X(248) is K; X(249) is D; X(250) is T; X(251) is selected from the group consisting of L, D, E, H, and T; X(252) is selected from the group consisting of M, D, E, and H; X(253) is selected from the group consisting of I, D, E, F, H, K, L, N, P, Q, R, S, T, V, W, and Y; X(254) is selected from the group consisting of S, E, K, N, P, Q, R, V, and W; X(255) is R; X(256) is selected from the group consisting of T, I, L, M, P, S, V, W, and Y; X(257) is P; X(258) is E; X(259) is selected from the group consisting of V and T; X(260) is T; X(261) is C; X(262) is V; X(263) is V; X(264) is V; X(265) is D; X(266) is V; X(267) is S; X(268) is H; X(269) is E; X(270) is D; X(271) is P; X(272) is E; X(273) is V; X(274) is Q; X(275) is F; X(276) is K; X(277) is W; X(278) is selected from the group consisting of Y, D, E, and S; X(279) is selected from the group consisting of V, A, Q, and T; X(280) is D; X(281) is G; X(282) is selected from the group consisting of V, F, G, I, L, P, Q, W, and Y; X(283) is selected from the group consisting of E and W; X(284) is V; X(285) is selected from the group consisting of H, E, P, and T; X(286) is N; X(287) is A; X(288) is K; X(289) is T; X(290) is K; X(291) is P; X(292) is R; X(293) is E; X(294) is E; X(295) is Q; X(296) is Y; X(297) is N; X(298) is S; X(299) is T; X(300) is selected from the group consisting of F, A, D, E, G, H, K, M, N, P, Q, R, S, T, and V; X(301) is selected from the group consisting of R, D, E, G, H, K, and Q; X(302) is selected from the group consisting of V, A, D, E, H, K, P, Q, S, and T; X(303) is selected from the group consisting of V, D, E, N, P, Q, and S; X(304) is S; X(305) is selected from the group consisting of V, G, P, and T; X(306) is selected from the group consisting of L, F, H, I, N, T, V, and Y; X(307) is T; X(308) is selected from the group consisting of V, A, N, P, and S; X(309) is selected from the group consisting of L, F, G, I, M, N, Q, S, T, V, W, and Y; X(310) is H; X(311) is selected from the group consisting of Q, D, E, G, P, T, and W; X(312) is D; X(313) is W; X(314) is L; X(315) is N; X(316) is G; X(317) is K; X(318) is E; X(319) is Y; X(320) is K; X(321) is C; X(322) is K; X(323) is V; X(324) is S; X(325) is N; X(326) is K; X(327) is A; X(328) is L; X(329) is P; X(330) is A; X(331) is P; X(332) is l; X(333) is E; X(334) is K; X(335) is T; X(336) is l; X(337) is S; X(338) is K; X(339) is T; X(340) is K; X(341) is G; X(342) is Q; X(343) is P; X(344) is R; X(345) is E; X(346) is P; X(347) is Q; X(348) is V; X(349) is Y; X(350) is T; X(351) is L; X(352) is P; X(353) is P; X(354) is S; X(355) is R; X(356) is E; X(357) is E; X(358) is M; X(359) is T; X(360) is K; X(361) is N; X(362) is Q; X(363) is V; X(364) is S; X(365) is L; X(366) is T; X(367) is C; X(368) is L; X(369) is V; X(370) is K; X(371) is G; X(372) is F; X(373) is Y; X(374) is P; X(375) is S; X(376) is D; X(377) is I; X(378) is A; X(379) is V; X(380) is E; X(381) is W; X(382) is E; X(383) is S; X(384) is S; X(385) is G; X(386) is Q; X(387) is P; X(388) is E; X(389) is N; X(390) is N; X(391) is Y; X(392) is N; X(393) is T; X(394) is T; X(395) is P; X(396) is P; X(397) is M; X(398) is L; X(399) is D; X(400) is S; X(401) is D; X(402) is G; X(403) is S; X(404) is F; X(405) is F; X(406) is L; X(407) is Y; X(408) is S; X(409) is K; X(410) is L; X(411) is T; X(412) is V; X(413) is D; X(414) is K; X(415) is S; X(416) is R; X(417) is W; X(418) is Q; X(419) is Q; X(420) is G; X(421) is N; X(422) is I; X(423) is F; X(424) is S; X(425) is C; X(426) is S; X(427) is V; X(428) is M; X(429) is H; X(430) is E; X(431) is A; X(432) is L; X(433) is H; X(434) is N; X(435) is R; X(436) is F;-X(437) is T; X(438) is Q; X(439) is K; X(440) is S; X(441) is L; X(442) is S; X(443) is L; X(444) is S; X(445) is P; X(446) is G; and X(447) is K. The non-naturally occurring protein having reduced immunogenicity as compared with a protein comprising SEQ ID NO:3.

In a further aspect, the non-naturally occurring protein comprising a variant Fc region comprising at least one amino acid modification of a naturally occurring protein sequence comprising SEQ ID NO:3. The modification is at a position selected from the group consisting of positions 251, 252, 253, 254, 256, 259, 278, 279, 282, 283, 285, 300, 301, 302, 303, 305, 306, 308, 309, and 311. The modification at position 251 is selected from the group consisting of D, E, H, and T; the modification at position 252 is selected from the group consisting of D, E, and H; the modification at position 253 is selected from the group consisting of D, E, F, H, K, L, N, P, Q, R, S, T, V, W, and Y; the modification at position 254 is selected from the group consisting of E, K, N, P, Q, R, V, and W; the modification at position 256 is selected from the group consisting of I, L, M, P, S, V, W, and Y; the modification at position 259 is T; the modification at position 278 is selected from the group consisting of D, E, and S; the modification at position 279 is selected from the group consisting of A, Q, and T; the modification at position 282 is selected from the group consisting of F, G, I, L, P, Q, W, and Y; the modification at position 283 is W; the modification at position 285 is selected from the group consisting of E, P, and T; the modification at position 300 is selected from the group consisting of A, D, E, G, H, K, M, N, P, Q, R, S, T, and V; the modification at position 301 is selected from the group consisting of D, E, G, H, K, and Q; the modification at position 302 is selected from the group consisting of A, D, E, H, K, P, Q, S, and T; the modification at position 303 is selected from the group consisting of D, E, N, P, Q, and S; the modification at position 305 is selected from the group consisting of G, P, and T; the modification at position 306 is selected from the group consisting of F, H, I, N, T, V, and Y; the modification at position 308 is selected from the group consisting of A, N, P, and S; the modification at position 309 is selected from the group consisting of F, G, I, M, N, Q, S, T, V, W, and Y; and, the modification at position 311 is selected from the group consisting of D, E, G, P, T, and W.

In a further variation, at least one modification is made to the group consisting of positions 251, 252, 253, 254, 256, 259, 278, 279, 282, 283, 285, 302, 303, 305, 306, 308, 309, and 311; and, the modification at position 251 is selected from the group consisting of D, E, H, and T; the modification at position 252 is D; the modification at position 253 is selected from the group consisting of D and E; the modification at position 256 is selected from the group consisting of M, W, and Y; the modification at position 278 is D; the modification at position 282 is selected from the group consisting of F, L, Q, and W; the modification at position 303 is selected from the group consisting of N, P, Q, and S; and, the modification at position 311 is D.

In a still further variation, the modification is made to an amino acid in the group consisting of Agretope 4 (145-153), Agretope 5 (149-157), Agretope 6 (167-175), Agretope 7 (174-182), Agretope 8 (179-187), Agretope 9 (180-188), Agretope 10 (182-190), Agretope 11a (185-193), Agretope 12 (202-210), Agretope 13 (215-223), Agretope 15 (240-248), Agretope 16 (251-259), Agretope 17a (262-270), Agretope 18 (277-285), Agretope 19b (300-308), Agretope 20a (302-310), Agretope 21a (303-311), Agretope 22a (348-356), Agretope 23 (369-377), Agretope 27b (422-430), and Agretope 28b (432-440). In another variation, the modification is made to an amino acid in the group consisting of Agretope 16 (251-259), Agretope 17a (262-270), Agretope 18 (277-285), Agretope 19b (300-308), Agretope 20a (302-310), Agretope 21 a (303-311), Agretope 22a (348-356), Agretope 23 (369-377), Agretope 27b (422-430), and Agretope 28b (432-440).

In another aspect, the present invention is directed to a non-naturally occurring protein comprising a variant Fc region having the formula:

-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-.

X(118) is A; X(119) is S; X(120) is T; X(121) is K; X(122) is G; X(123) is P; X(124) is S; X(125) is V; X(126) is F; X(127) is P; X(128) is L; X(129) is A; X(130) is P; X(131) is C; X(132) is S; X(133) is R; X(134) is S; X(135) is T; X(136) is S; X(137) is E; X(138) is S; X(139) is T; X(140) is A; X(141) is A; X(142) is L; X(143) is G; X(144) is C; X(145) is L; X(146) is V; X(147) is K; X(148) is D; X(149) is Y; X(150) is F; X(151) is P; X(152) is E; X(153) is P; X(154) is V; X(155) is T; X(156) is V; X(157) is S; X(158) is W; X(159) is N; X(160) is S; X(161) is G; X(162) is A; X(163) is L; X(164) is T; X(165) is S; X(166) is G; X(167) is V; X(168) is H; X(169) is T; X(170) is F; X(171) is P; X(172) is A; X(173) is V; X(174) is L; X(175) is Q; X(176) is S; X(177) is S; X(178) is G; X(179) is L; X(180) is Y; X(181) is S; X(182) is L; X(183) is S; X(184) is S; X(185) is V; X(186) is V; X(187) is T; X(188) is V; X(189) is P; X(190) is S; X(191) is S; X(192) is S; X(193) is L; X(194) is G; X(195) is T; X(196) is K; X(197) is T; X(198) is Y; X(199) is T; X(200) is C; X(201) is N; X(202) is V; X(203) is D; X(204) is H; X(205) is K; X(206) is P; X(207) is S; X(208) is N; X(209) is T; X(210) is K; X(211) is V; X(212) is D; X(213) is K; X(214) is R; X(215) is V; X(216) is E; X(217) is S; X(218) is K; X(219) is Y; X(220) is G; X(221) is a bond; X(222) is a bond; X(223) is a bond; X(224) is P; X(225) is P; X(226) is C; X(227) is P; X(228) is S; X(229) is C; X(230) is P; X(231) is A; X(232) is P; X(233) is E; X(234) is F; X(235) is L; X(236) is G; X(237) is G; X(238) is P; X(239) is S; X(240) is V; X(241) is F; X(242) is L; X(243) is F; X(244) is P; X(245) is P; X(246) is K; X(247) is P; X(248) is K; X(249) is D; X(250) is T; X(251) is selected from the group consisting of L, D, E, H, and T; X(252) is selected from the group consisting of M, D, E, and H; X(253) is selected from the group consisting of I, D, E, F, H, K, L, N, P, Q, R, S, T, V, W, and Y; X(254) is selected from the group consisting of S, E, K, N, P, Q, R, V, and W; X(255) is R; X(256) is selected from the group consisting of T, I, L, M, P, S, V, W, and Y; X(257) is P; X(258) is E; X(259) is selected from the group consisting of V and T; X(260) is T; X(261) is C; X(262) is V; X(263) is V; X(264) is V; X(265) is D; X(266) is V; X(267) is S; X(268) is Q; X(269) is E; X(270) is D; X(271) is P; X(272) is E; X(273) is V; X(274) is Q; X(275) is F; X(276) is N; X(277) is W; X(278) is selected from the group consisting of Y, D and E; X(279) is selected from the group consisting of V, A, Q, T, and W; X(280) is D; X(281) is G; X(282) is selected from the group consisting of V, F, G, I, L, Q, and W; X(283) is E; X(284) is V; X(285) is selected from the group consisting of H, P, and T; X(286) is N; X(287) is A; X(288) is K; X(289) is T; X(290) is K; X(291) is P; X(292) is R; X(293) is E; X(294) is E; X(295) is Q; X(296) is F; X(297) is N; X(298) is S; X(299) is T; X(300) is Y; X(301) is selected from the group consisting of R, G, K, and Q; X(302) is selected from the group consisting of V, A, E, H, K, Q, S, and T; X(303) is selected from the group consisting of V, N, P, Q, R, and S; X(304) is S; X(305) is selected from the group consisting of V, G, P, and T; X(306) is selected from the group consisting of L, F, H, I, N, T, V, and Y; X(308) is selected from the group consisting of V, A, N, P, and S; X(309) is selected from the group consisting of L, F, G, I, M, N, Q, S, T, V, W, and Y; X(310) is H; X(311) is selected from the group consisting of Q, D, E, G, P, T, and W; X(312) is D; X(313) is W; X(314) is L; X(315) is N; X(316) is G; X(317) is K; X(318) is E; X(319) is Y; X(320) is K; X(321) is C; X(322) is K; X(323) is V; X(324) is S; X(325) is N; X(326) is K; X(327) is G; X(328) is L; X(329) is P; X(330) is S; X(331) is S; X(332) is l; X(333) is E; X(334) is K; X(335) is T; X(336) is I; X(337) is S; X(338) is K; X(339) is A; X(340) is K; X(341) is G; X(342) is Q; X(343) is P; X(344) is R; X(345) is E; X(346) is P; X(347) is Q; X(348) is V; X(349) is Y; X(350) is T; X(351) is L; X(352) is P; X(353) is P; X(354) is S; X(355) is Q; X(356) is E; X(357) is E; X(358) is M; X(359) is T; X(360) is K; X(361) is N; X(362) is Q; X(363) is V; X(364) is S; X(365) is L; X(366) is T; X(367) is C; X(368) is L; X(369) is V; X(370) is K; X(371) is G; X(372) is F; X(373) is Y; X(374) is P; X(375) is S; X(376) is D; X(377) is l; X(378) is A; X(379) is V; X(380) is E; X(381) is W; X(382) is E; X(383) is S; X(384) is N; X(385) is G; X(386) is Q; X(387) is P; X(388) is E; X(389) is N; X(390) is N; X(391) is Y; X(392) is K; X(393) is T; X(394) is T; X(395) is P; X(396) is P; X(397) is V; X(398) is L; X(399) is D; X(400) is S; X(401) is D; X(402) is G; X(403) is S; X(404) is selected from the group consisting of F, H, I, L, M, N, Q, T, and V; X(405) is selected from the group consisting of F and W; X(406) is selected from the group consisting of L, A, D, E, G, K, N, Q, S, T, and V; X(407) is selected from the group consisting of Y and M; X(408) is S; X(409) is selected from the group consisting of R, G, Q, and S; X(410) is selected from the group consisting of L, F, Q, and Y; X(411) is T; X(412) is selected from the group consisting of V and P; X(413) is D; X(414) is K; X(415) is S; X(416) is R; X(417) is W; X(418) is Q; X(419) is E; X(420) is G; X(421) is N; X(422) is V; X(423) is F; X(424) is S; X(425) is C; X(426) is S; X(427) is V; X(428) is M; X(429) is H; X(430) is E; X(431) is A; X(432) is selected from the group consisting of L, E and K; X(433) is selected from the group consisting of H, D, G, P, S, T, and W; X(434) is selected from the group consisting of N, D, E, G, H, S, T, and W; X(435) is selected from the group consisting of H, G, K, M, N, P, S, T, and V; X(436) is Y; X(437) is selected from the group consisting of T, D, E, G, H, K, N, Q, and S; X(438) is selected from the group consisting of Q, G, P, S, and T; (439) is K; X(440) is selected from the group consisting of S, D, E, G, H, K, N, P, Q, R, and T; X(441) is L; X(442) is S; X(443) is L; X(444) is S; X(445) is L; X(446) is G; and X(447) is K. The non-naturally occurring protein has reduced immunogenicity as compared with a protein comprising SEQ ID NO:4.

In another aspect, the present invention is directed to a non-naturally occurring protein comprising a variant Fc region comprising at least one amino acid modification of SEQ ID NO:4. At least one modification is made to the group consisting of positions 251, 252, 253, 254, 256, 259, 278, 279, 282, 283, 285, 300, 301, 302, 303, 305, 306, 308, 309, 311, 404, 405, 406, 407, 409, 410, 412, 432, 433, 434, 435, 437, 438, and 440; and, wherein the modification at position 251 is selected from the group consisting of D, E, H, and T; wherein the modification at position 252 is selected from the group consisting of D, E, and H; wherein the modification at position 253 is selected from the group consisting of D, E, F, H, K, L, N, P, Q, R, S, T, V, W, and Y; wherein the modification at position 254 is selected from the group consisting of E, K, N, P, Q, R, V, and W; wherein the modification at position 256 is selected from the group consisting of I, L, M, P, S, V, W, and Y; wherein the modification at position 259 is T; wherein the modification at position 278 is selected from the group consisting of D and E; wherein the modification at position 279 is selected from the group consisting of A, Q, T, and W; wherein the modification at position 282 is selected from the group consisting of F, G, I, L, P, Q, W, and Y; wherein the modification at position 283 is G; wherein the modification at position 285 is selected from the group consisting of E, P, and T; wherein the modification at position 300 is selected from the group consisting of A, D, E, G, H, K, M, N, P, Q, R, S, T, and V; wherein the modification at position 301 is selected from the group consisting of D, E, G, K, and Q; wherein the modification at position 302 is selected from the group consisting of A, E, H, K, Q, S, and T; wherein the modification at position 303 is selected from the group consisting of D, E, N, P, Q, R, and S; wherein the modification at position 305 is selected from the group consisting of G, P, and T; wherein the modification at position 306 is selected from the group consisting of F, H, I, N, T, V, and Y; wherein the modification at position 308 is selected from the group consisting of A, N, P, and S; wherein the modification at position 309 is selected from the group consisting of F, G, I, M, N, Q, S, T, V, W, and Y; wherein the modification at position 311 is selected from the group consisting of D, E, G, P, T, and W; wherein the modification at position 404 is selected from the group consisting of H, I, L, M, N, Q, T, and V; wherein the modification at position 405 is W; wherein the modification at position 406 is selected from the group consisting of A, D, E, G, K, N, Q, S, T, and V; wherein the modification at position 407 is M; wherein the modification at position 409 is selected from the group consisting of G, Q, and S; wherein the modification at position 410 is selected from the group consisting of F, Q, and Y; wherein the modification at position 412 is P; wherein the modification at position 432 is selected from the group consisting of E and K; wherein the modification at position 433 is selected from the group consisting of D, G, P, S, T, and W; wherein the modification at position 434 is selected from the group consisting of D, E, G, H, S, T, and W; wherein the modification at position 435 is selected from the group consisting of G, K, M, N, P, S, T, and V; wherein the modification at position 437 is selected from the group consisting of D, E, G, H, K, N, Q, and S; wherein the modification at position 438 is selected from the group consisting of G, P, S, and T; and, wherein the modification at position 440 is selected from the group consisting of D, E, G, H, K, N, P, Q, R, and T. The non-naturally occurring protein having reduced immunogenicity as compared with a protein comprising SEQ ID NO:4.

In one variation, the modification is made to the group consisting of positions 251, 252, 253, 254, 256, 259, 278, 279, 282, 283, 285, 300, 301, 302, 303, 305, 306, 308, 309, 311, 404, 405, 406, 407, 409, 410, 412, 432, 433, 434, 435, 437, 438, and 440; and, wherein the modification at position 251 is selected from the group consisting of D, E, H, and T; wherein the modification at position 252 is D; wherein the modification at position 253 is selected from the group consisting of D and E; wherein the modification at position 256 is selected from the group consisting of M, W, and Y; wherein the modification at position 278 is D; wherein the modification at position 282 is selected from the group consisting of F, G, L, Q, W, and Y; wherein the modification at position 300 is selected from the group consisting of A, D, E, G, H, K, N, P, Q, R, S, and T; wherein the modification at position 301 is D; wherein the modification at position 303 is selected from the group consisting of D, E, N, P, Q, R, and S; wherein the modification at position 311 is D; wherein the modification at position 404 is selected from the group consisting of H, N, Q, and T; wherein the modification at position 432 is selected from the group consisting of E and K; and, wherein the modification at position 437 is E.

In a further variation, at least one modification is made to an amino acid in Agretope 16 (251-259), Agretope 17b (262-270), Agretope 18 (277-285), Agretope 19a (300-308), Agretope 20a (302-310), Agretope 21a (303-311), Agretope 22b (348-356), Agretope 23 (369-377), Agretope 24b (404-412), Agretope 25b (406-414), Agretope 26 (407-415), Agretope 27a (422-430), or Agretope 28a(432-440), Alternatively, at least one modification is made to an amino acid in Agretope 4 (145-153), Agretope 5 (149-157), Agretope 6 (167-175), Agretope 7 (174-182), Agretope 8 (179-187), Agretope 9 (180-188), Agretope 10 (182-190), Agretope 11a (185-193), Agretope 14 (234-242), Agretope 15 (240-248), Agretope 16 (251-259), Agretope 17b (262-270), Agretope 18 (277-285), Agretope 19a (300-308), Agretope 20a (302-310), Agretope 21a (303-311), Agretope 22b (348-356), Agretope 23 (369-377), Agretope 24b (404-412), Agretope 25b (406-414), Agretope 26 (407-415), Agretope 27a (422-430), and Agretope 28a (432-440).

### BRIEF DESCRIPTION OF THE FIGURES

FIG. 1. Antibody structure and function. Shown is a model of a full length human IgG1 antibody, modeled using a humanized Fab structure from pdb accession code 1CE1 (James et al., 1999, J Mol Biol 289:293-301, entirely incorporated by reference) and a human IgG1 Fc structure from pdb accession code 1DN2 (DeLano et al., 2000, Science 287:1279-1283, entirely incorporated by reference). The flexible hinge that links the Fab and Fc regions is not shown. IgG1 is a homodimer of heterodimers, made up of two light chains and two heavy chains. The Ig domains that comprise the antibody are labeled, and include V_{L} and C_{L} for the light chain, and V_{H}, Cgamma1(Cγ1), Cgamma2 (Cγ2), and Cgamma3 (Cγ3) for the heavy chain. The Fc region is labeled. Binding sites for relevant proteins are labeled, including the antigen binding site in the variable region, and the binding sites for FcγRs, FcRn, C1q, and proteins A and G in the Fc region.

FIG. 2 shows amino acid sequences of various antibodies, Fc fusions, and fragments and variants thereof.

FIG. 3 shows a method for engineering less immunogenic antibodies and Fc fusion proteins.

FIG. 4 shows a schematic representation of a method for in vitro testing of the immunogenicity of antibodies and Fc fusion proteins and peptides derived from antibodies and Fc fusion proteins with IVV technology.

FIG. 5 shows conservative mutations (BLOSUM62 score>=0).

FIG. 6 shows MHC agretopes in the IgG constant regions (SEQ ID NO:1-4).

FIG. 7 shows allele binding specificity of predicted agretopes in the IgG constant regions (SEQ ID NO:1-4).

FIG. 8 shows the IScore of MHC binding agretopes in antibody germline heavy chain variable region (VH, SEQ. ID. NO. 5-31).

FIG. 9 shows the IScore of MHC binding agretopes in antibody germline heavy chain variable region (VH, SEQ. ID. NO. 32-58).

FIG. 10 shows the IScore of MHC binding agretopes in antibody germline kappa light chain variable region (VH, SEQ. ID. NO. 59-84).

FIG. 11 shows the IScore of MHC binding agretopes in antibody germline kappa light chain variable region (VH, SEQ. ID. NO. 85-104).

FIG. 12 shows the IScore of MHC binding agretopes in antibody germline lambda light chain variable region (VH, SEQ. ID. NO. 105-129).

FIG. 13 shows the IScore of MHC binding agretopes in antibody germline lambda light chain variable region (VH, SEQ. ID. NO. 130-144).

FIG. 14 shows the B(wt), I(alt), and and B(alt) scores of agretope 5 (IgG1,2,3,4 constant region residues 149-157).

FIG. 15 shows the B(wt), I(alt), and and B(alt) scores of agretope 16 (IgG1,2,3,4 constant region residues 251-259).

FIG. 16 shows the B(wt), I(alt), and and B(alt) scores of agretope 18 (IgG1,2,3,4 constant region residues 277-285).

FIG. 17 shows the B(wt), I(alt), and and B(alt) scores of agretope 19a (IgG1,4 constant region residues 300-308).

FIG. 18 shows the B(wt), I(alt), and and B(alt) scores of agretope 19b (IgG2,3 constant region residues 300-308).

FIG. 19 shows the B(wt), I(alt), and and B(alt) scores of agretope 21a (IgG1,3,4 constant region residues 303-311).

FIG. 20 shows the B(wt), I(alt), and and B(alt) scores of agretope 24a (IgG1,2 constant region residues 404-412).

FIG. 21 shows the B(wt), I(alt), and and B(alt) scores of agretope 24b (IgG4 constant region residues 404-412).

FIG. 22 shows the B(wt), I(alt), and and B(alt) scores of agretope 28a (IgG1,2,4 constant region residues 432-440).

FIG. 23 shows suitable less immunogenic variants of agretope 16 (IgG1,2,3,4 constant region residues 251-259).

FIG. 24 shows suitable less immunogenic variants of agretope 18 (IgG1,2,3,4 constant region residues 277-285).

FIG. 25 shows suitable less immunogenic variants of agretope 19a (IgG1,4 constant region residues 300-308).

FIG. 26 shows suitable less immunogenic variants of agretope 19b (IgG2,3 constant region residues 300-308).

FIG. 27 shows suitable less immunogenic variants of agretope 21a (IgG1,3,4 constant region residues 303-311).

FIG. 28 shows suitable less immunogenic variants of agretope 24a (IgG1,2 constant region residues 404-412).

FIG. 29 shows less immunogenic variants of agretope 24b (IgG4 constant region residues 404-412).

FIG. 30 shows less immunogenic variants of agretope 28a (IgG1,2,4 constant region residues 432-440).

FIG. 31 shows MHC agretopes in Fc variants with significantly decreased IScore at one or more agretopes.

FIG. 32 shows MHC agretopes in Fc variants with significantly increased IScore at one or more agretopes.

FIG. 33 shows IScore of MHC agretopes in especially preferred Fc variants versus the parent human IgG1 sequence (SEQ ID NO:1).

FIG. 34 shows ingle amino acid changes in human heavy chain variable domain germline sequences that preserve fully human sequence content and reduce IScore for at least one predicted agretope.

FIG. 35 shows single amino acid changes in human light chain variable domain germline sequences that preserve fully human sequence content and reduce IScore for at least one predicted agretope.

### DETAILED DESCRIPTION OF THE INVENTION

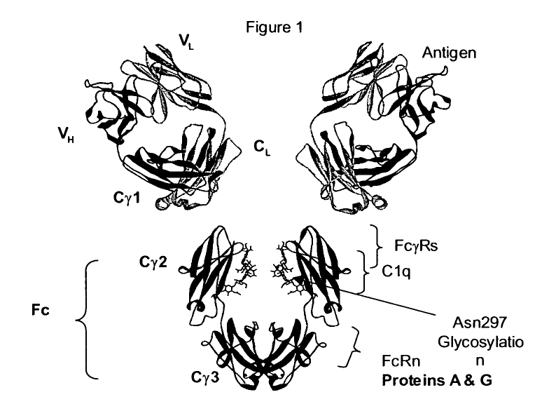

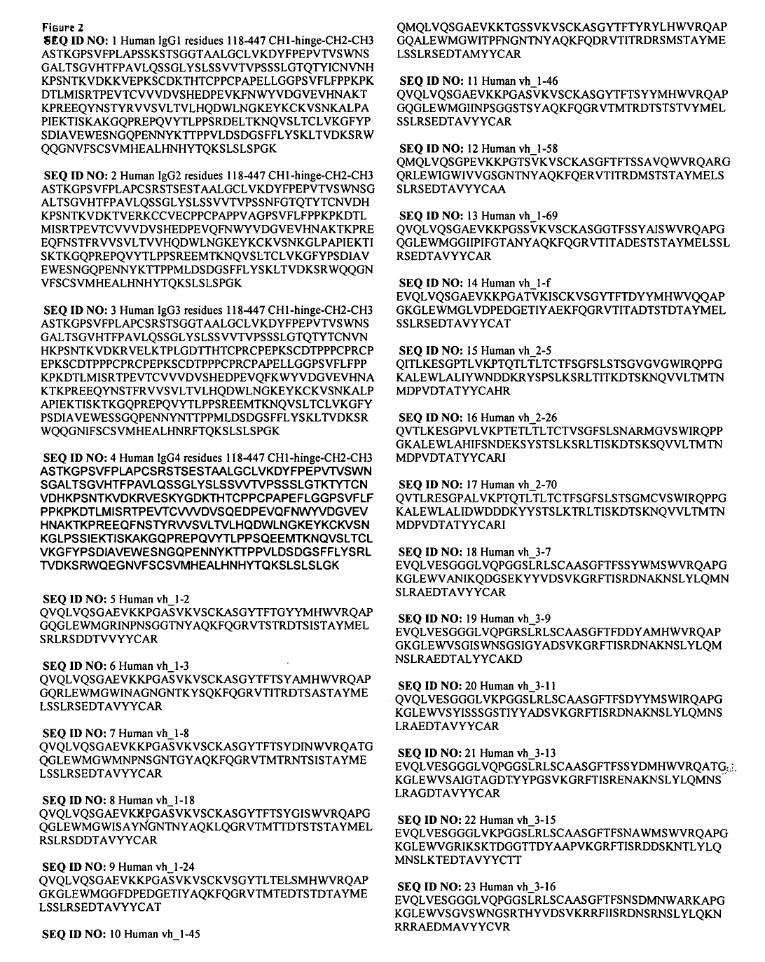

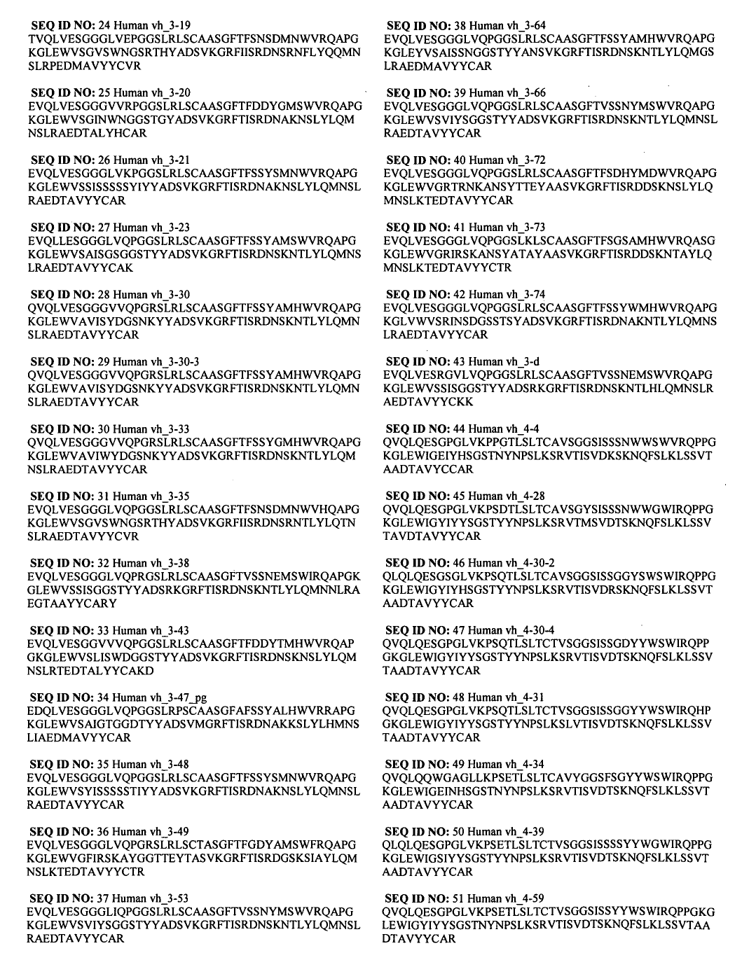

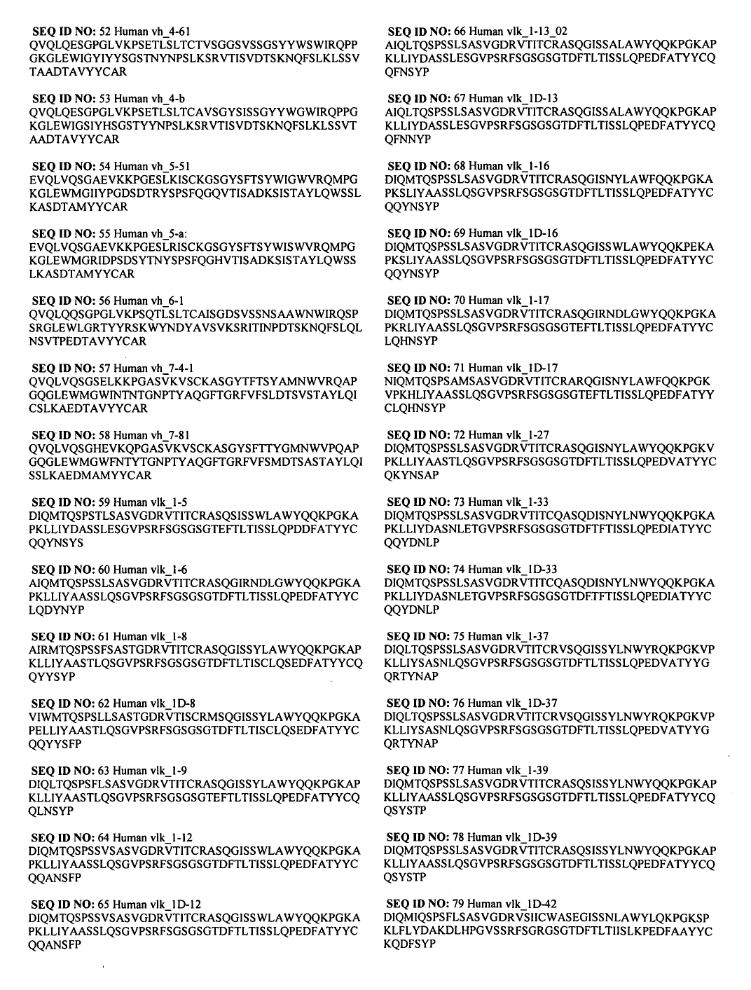

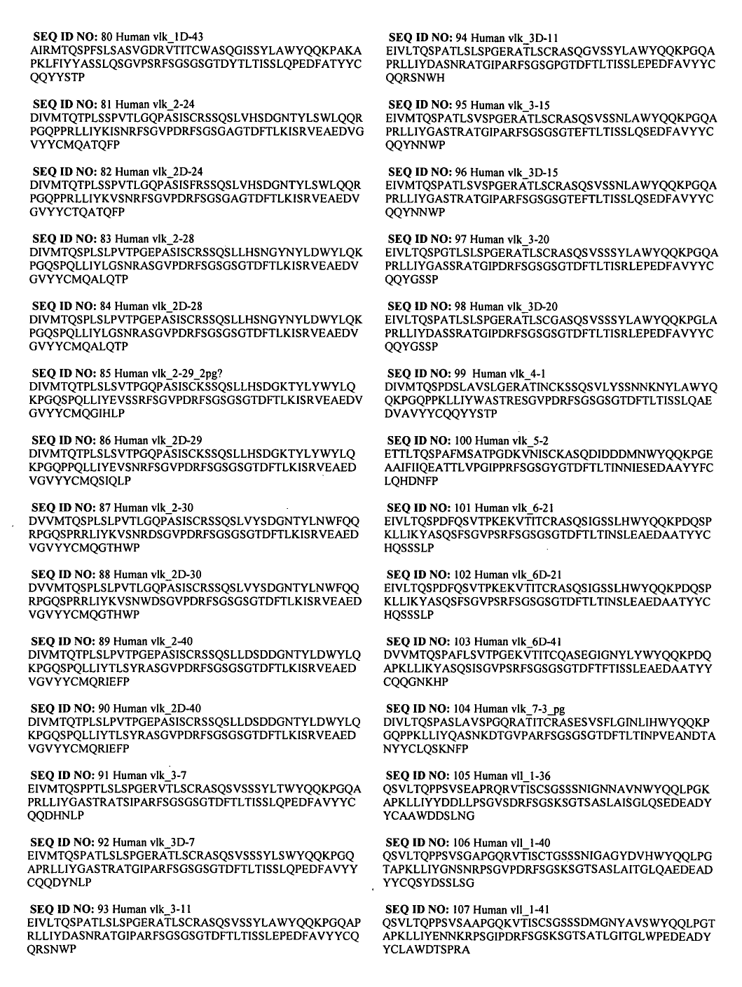

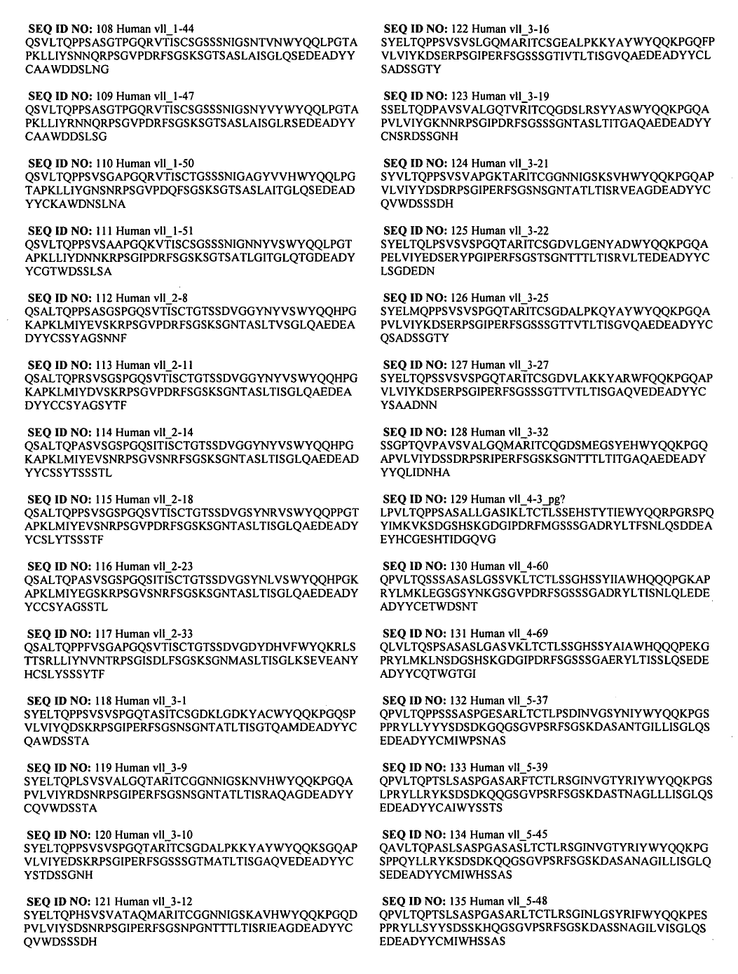

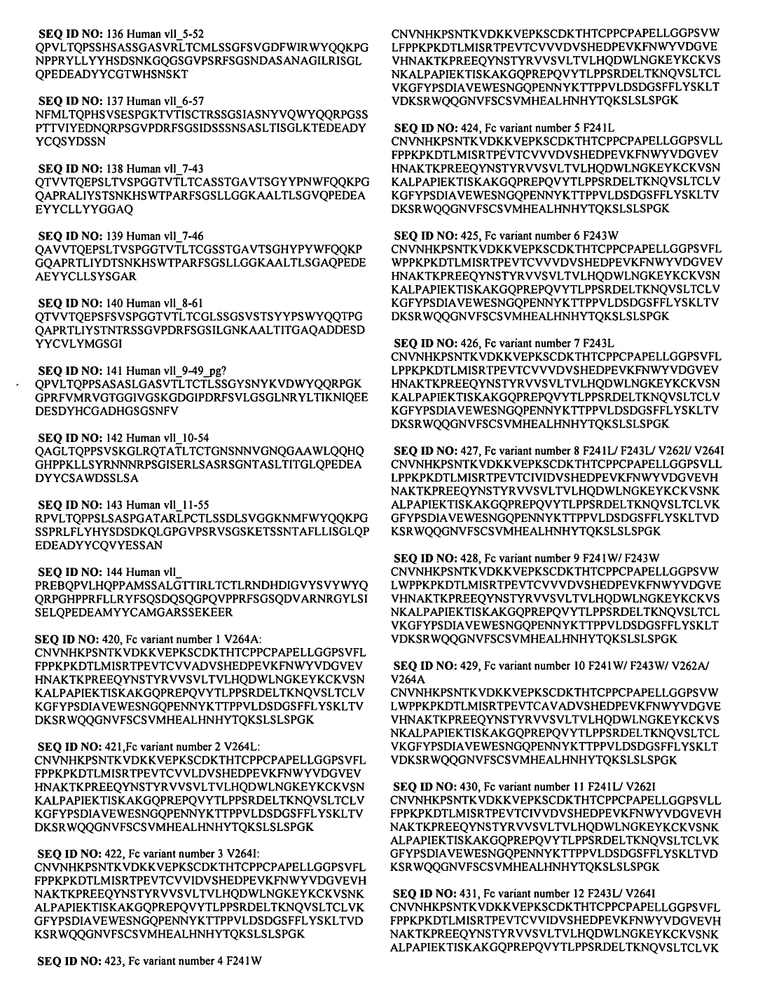

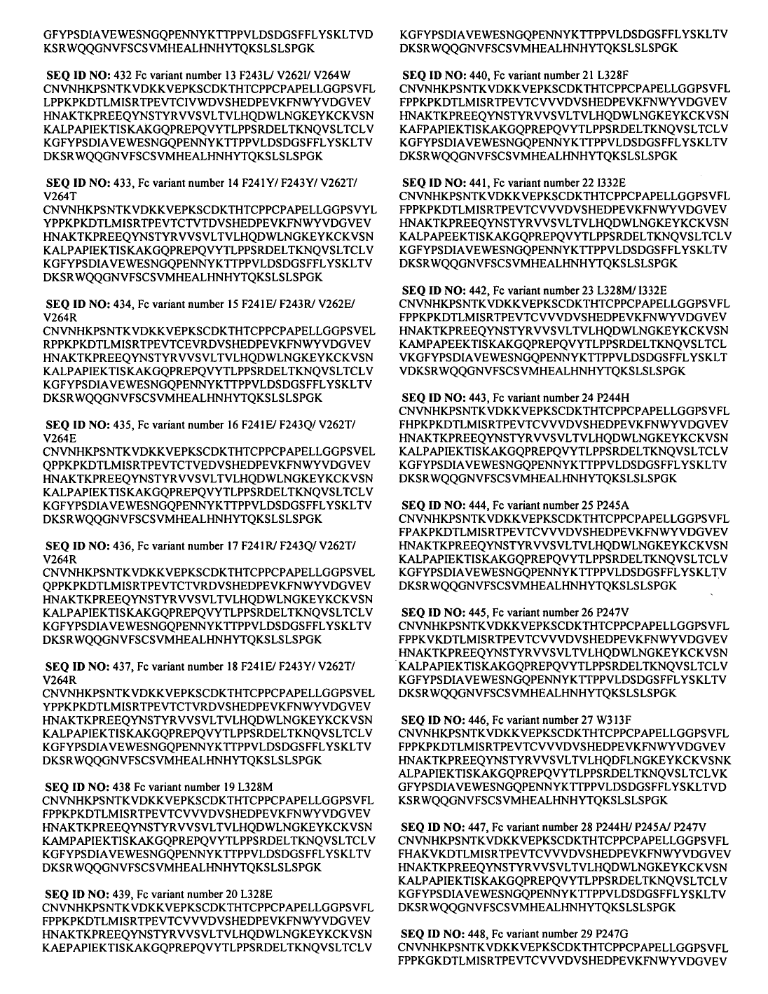

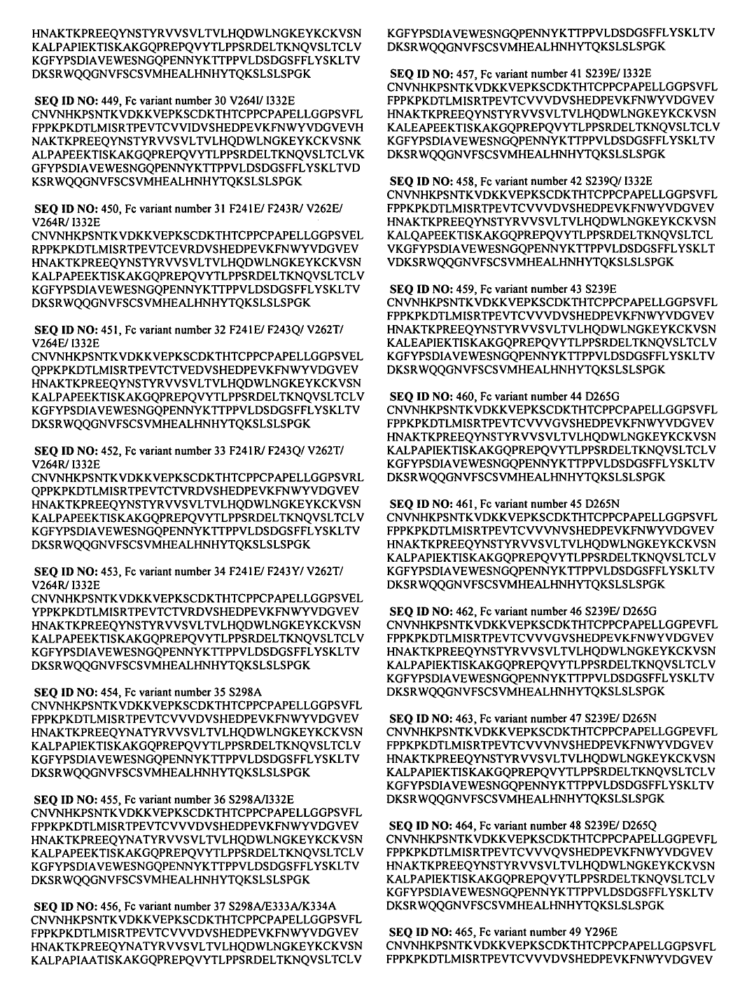

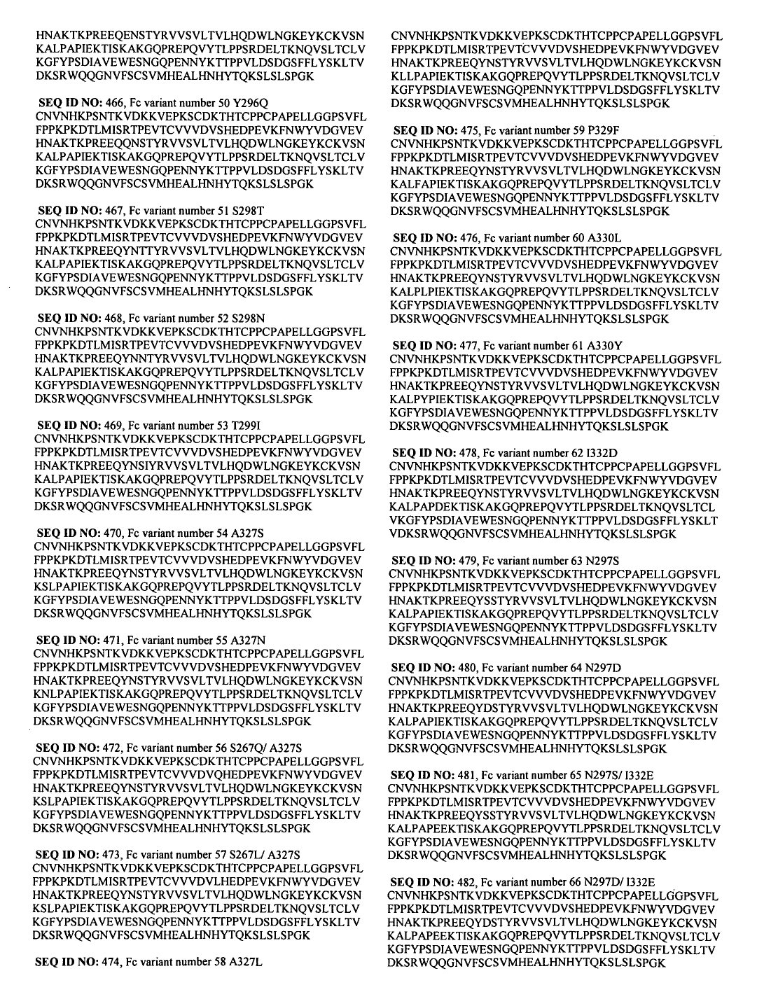

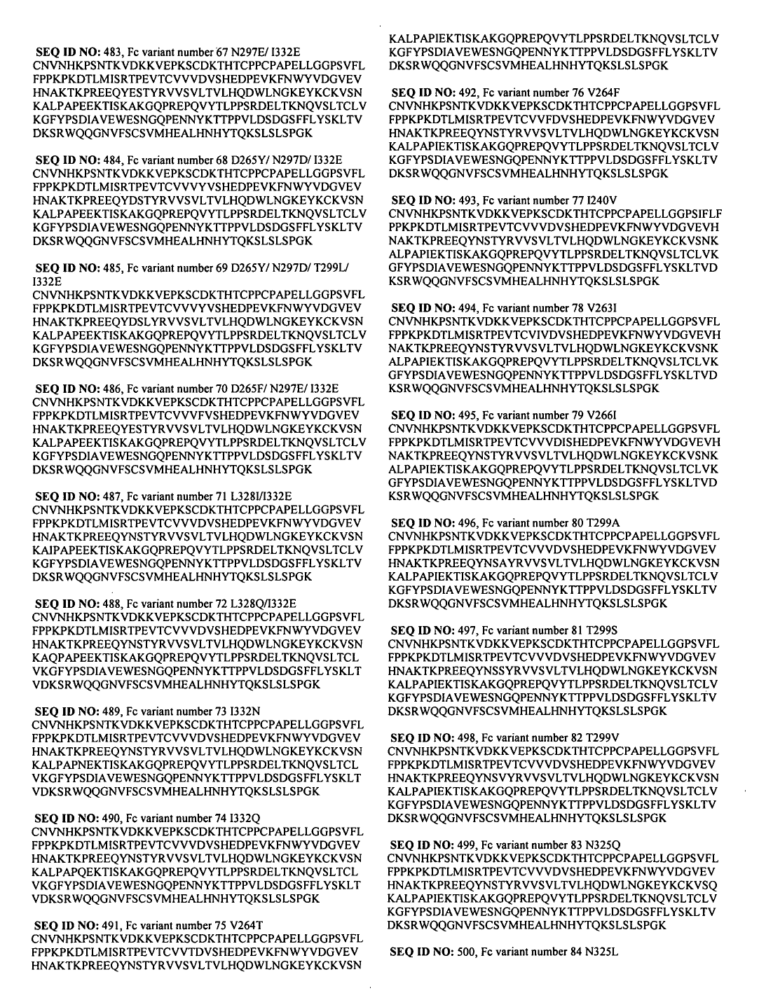

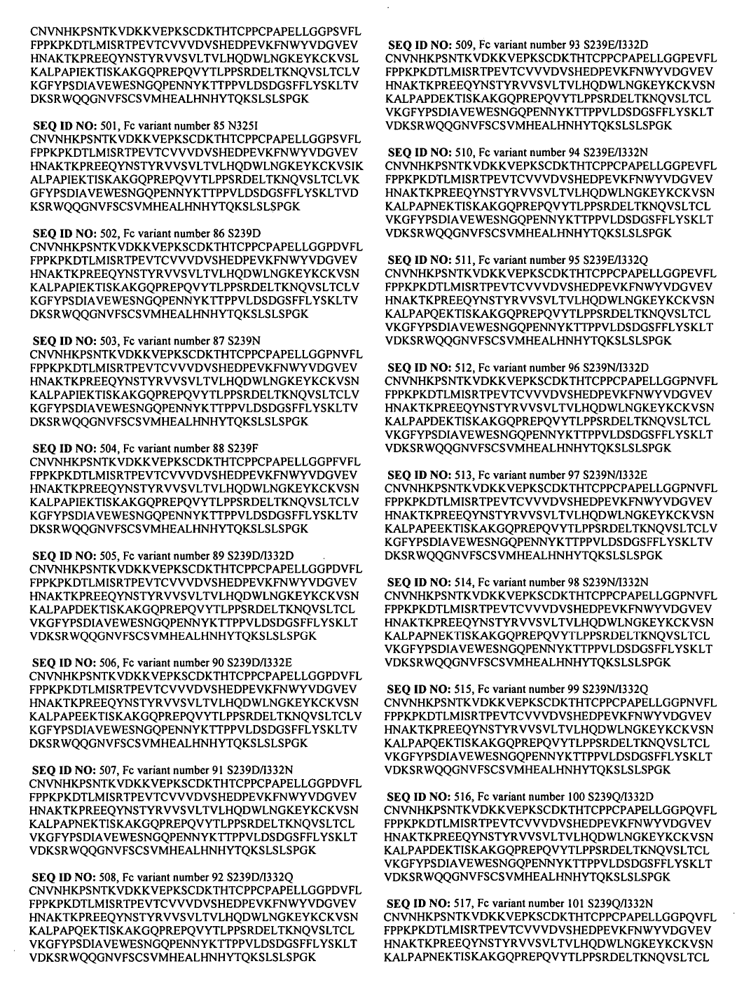

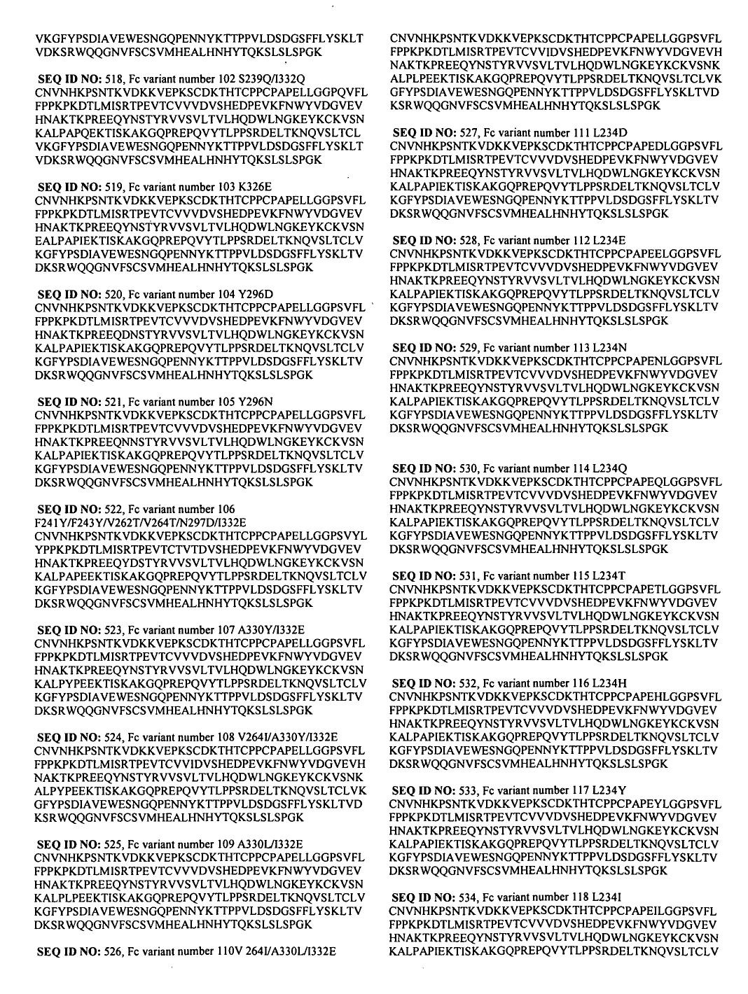

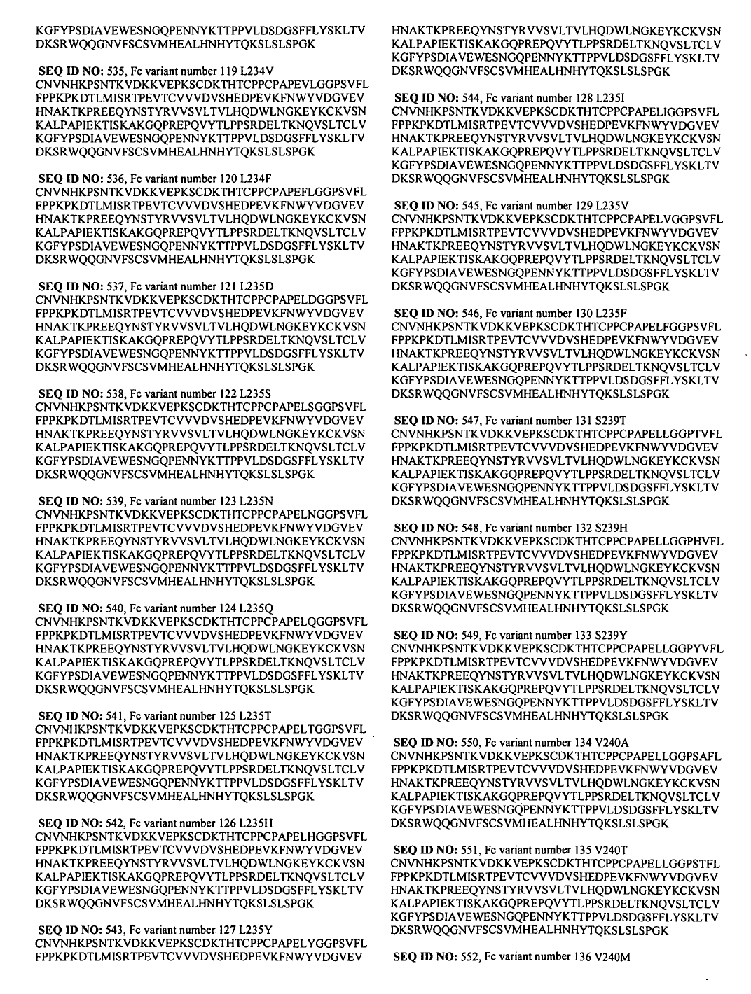

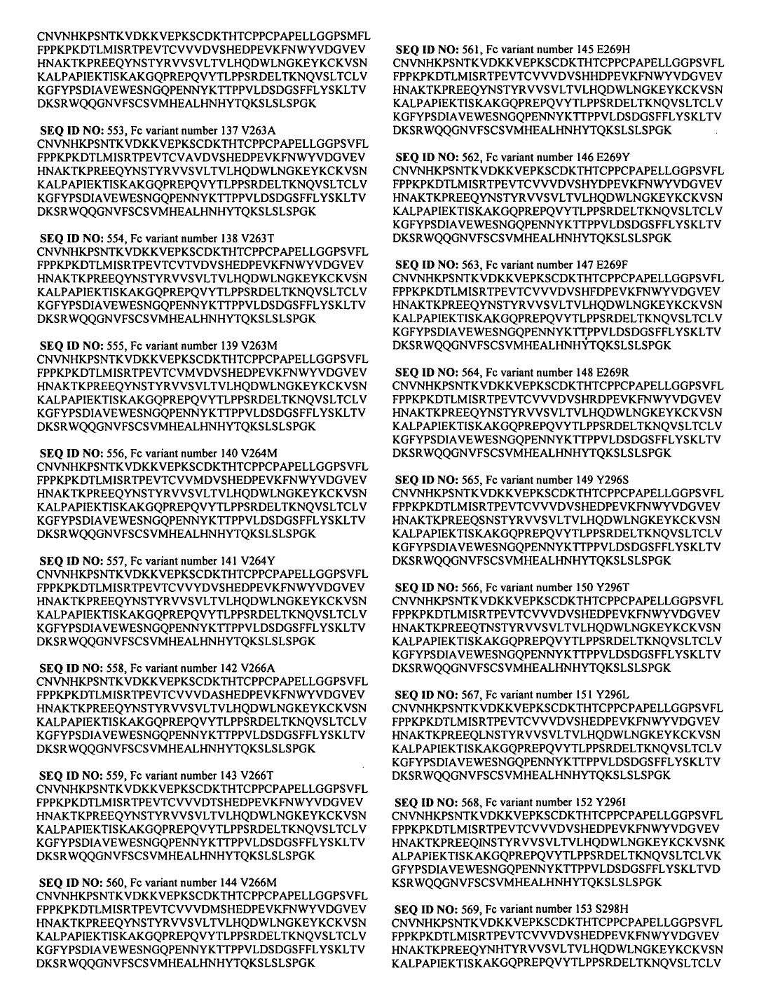

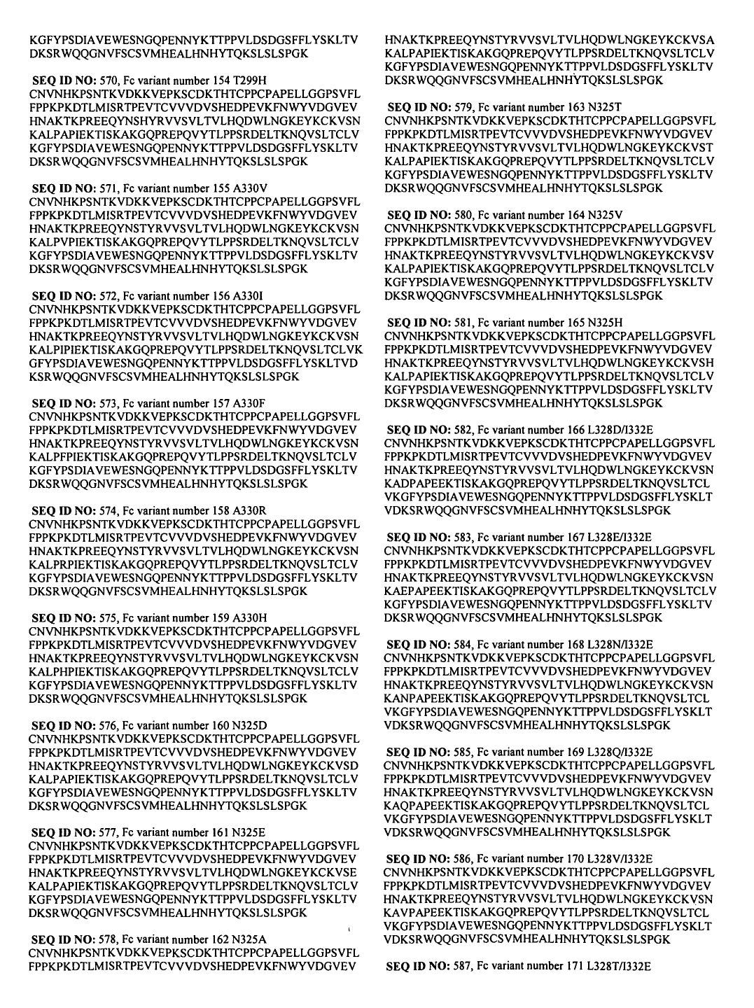

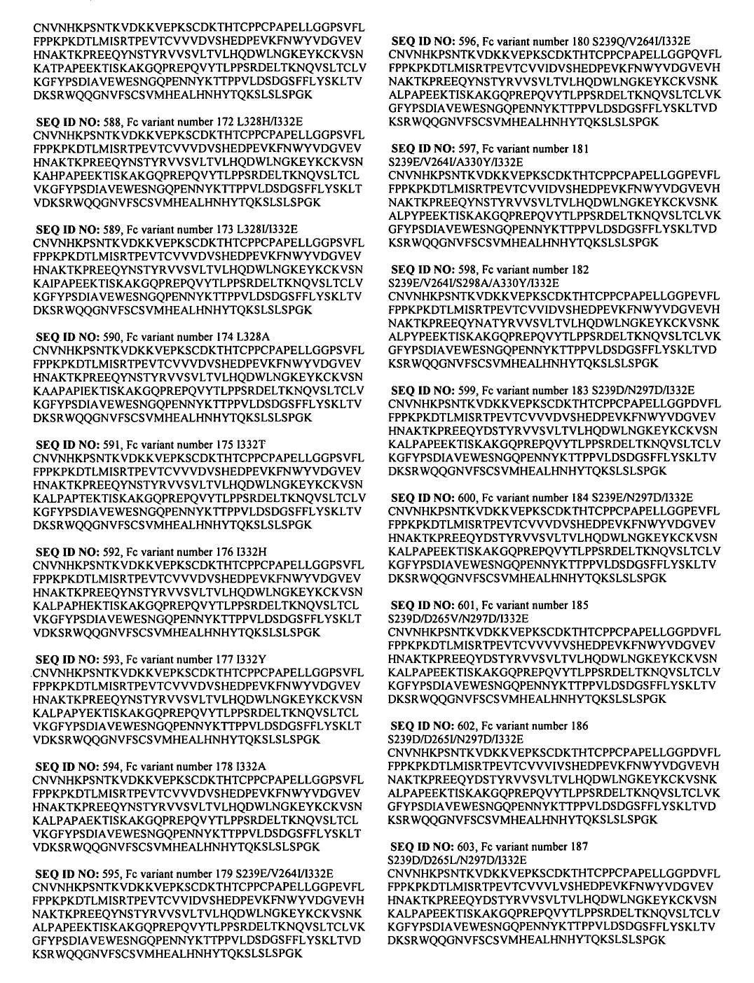

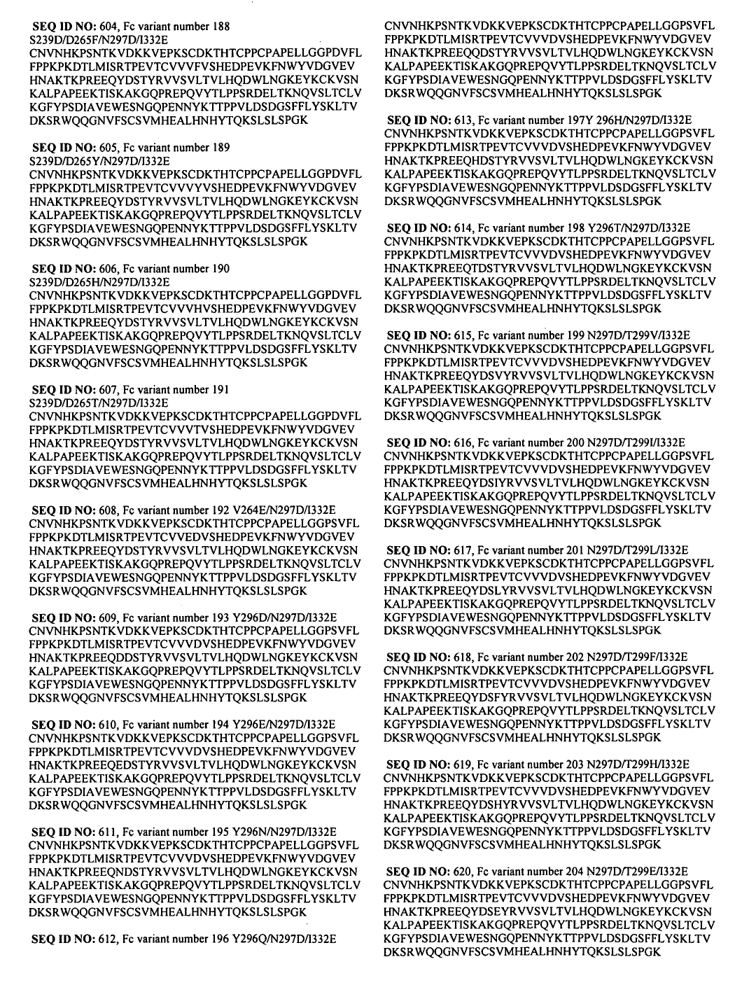

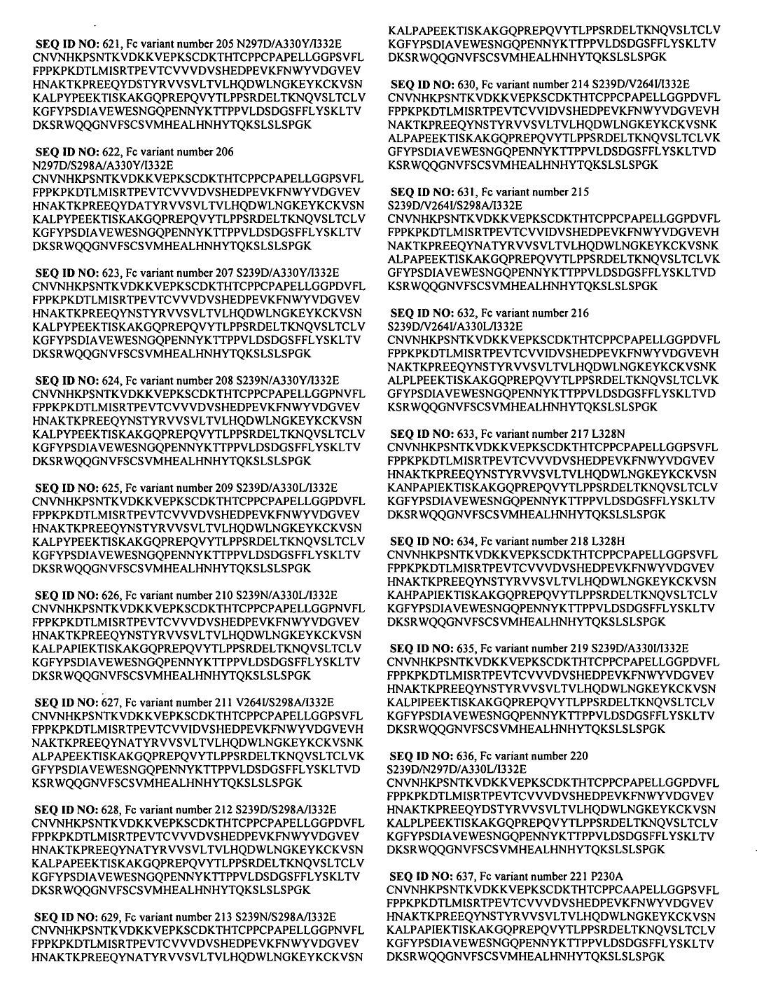

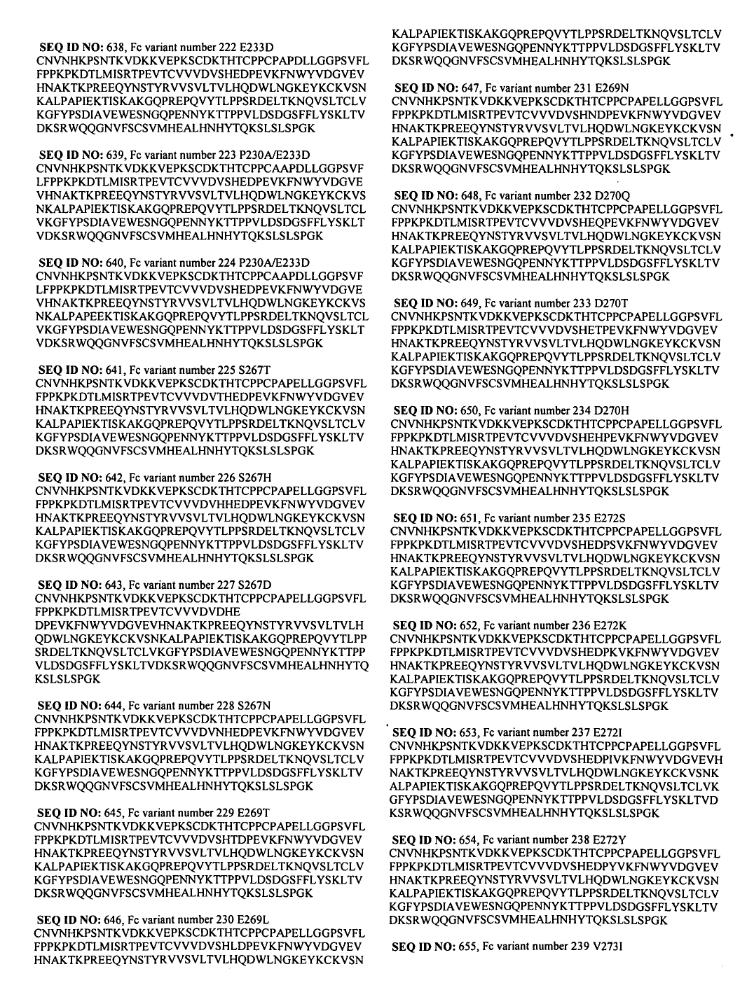

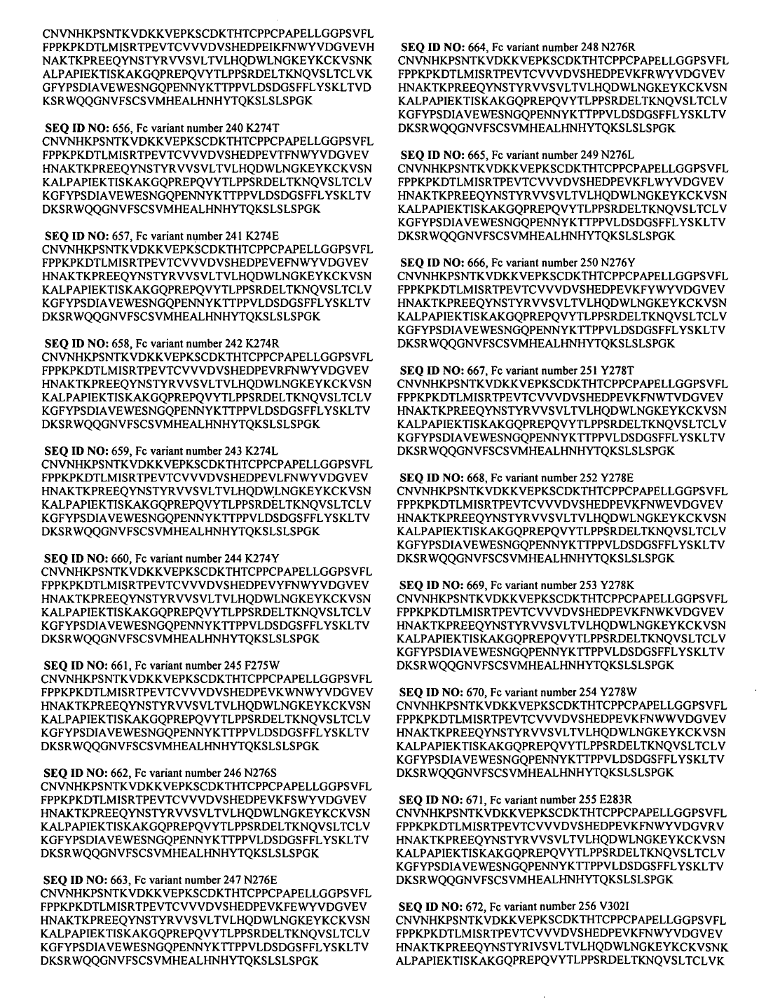

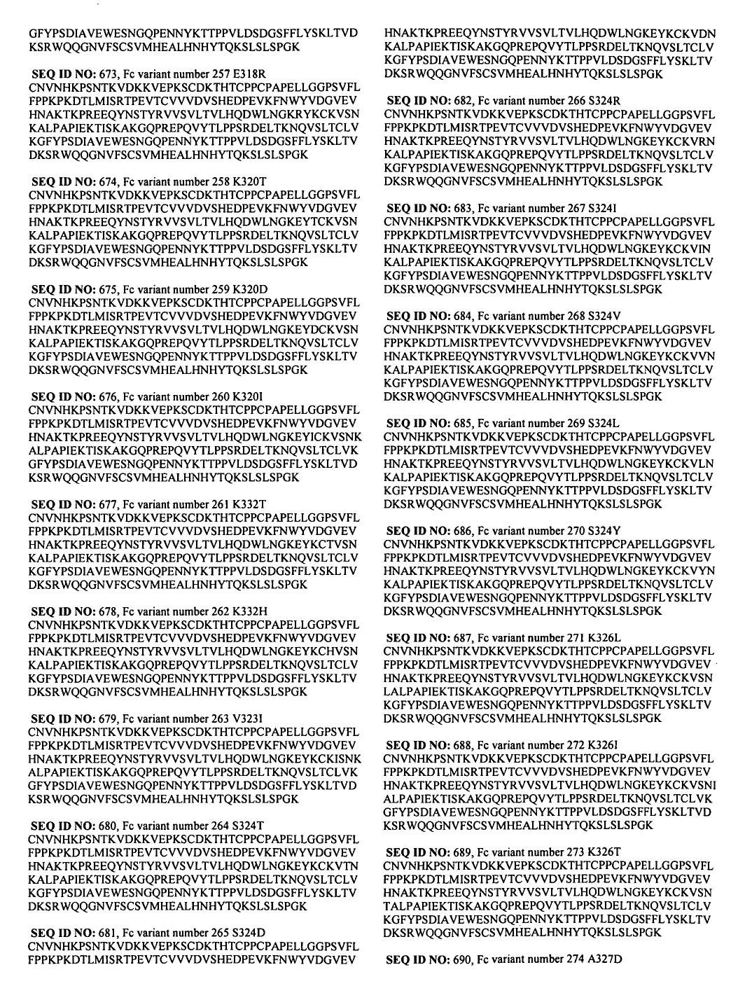

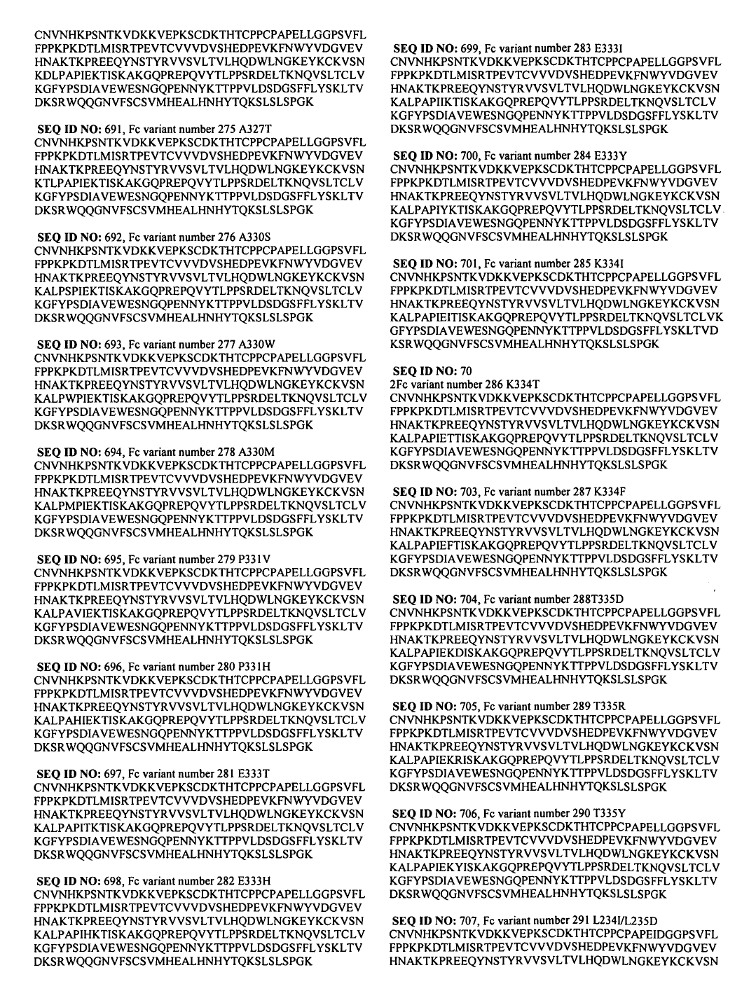

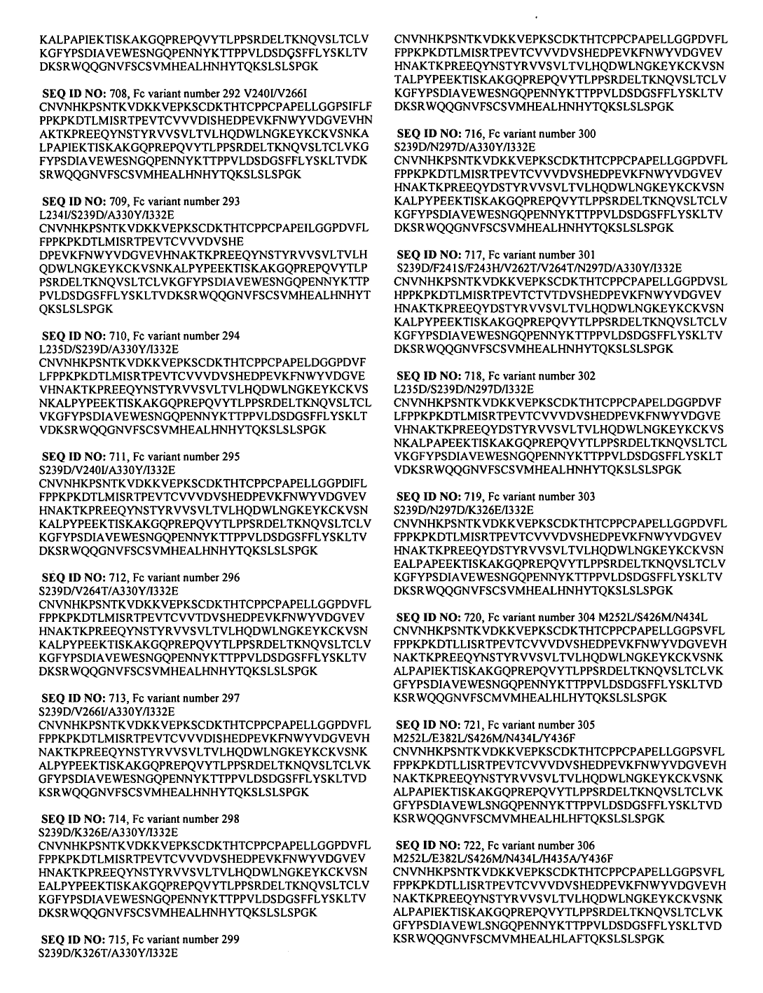

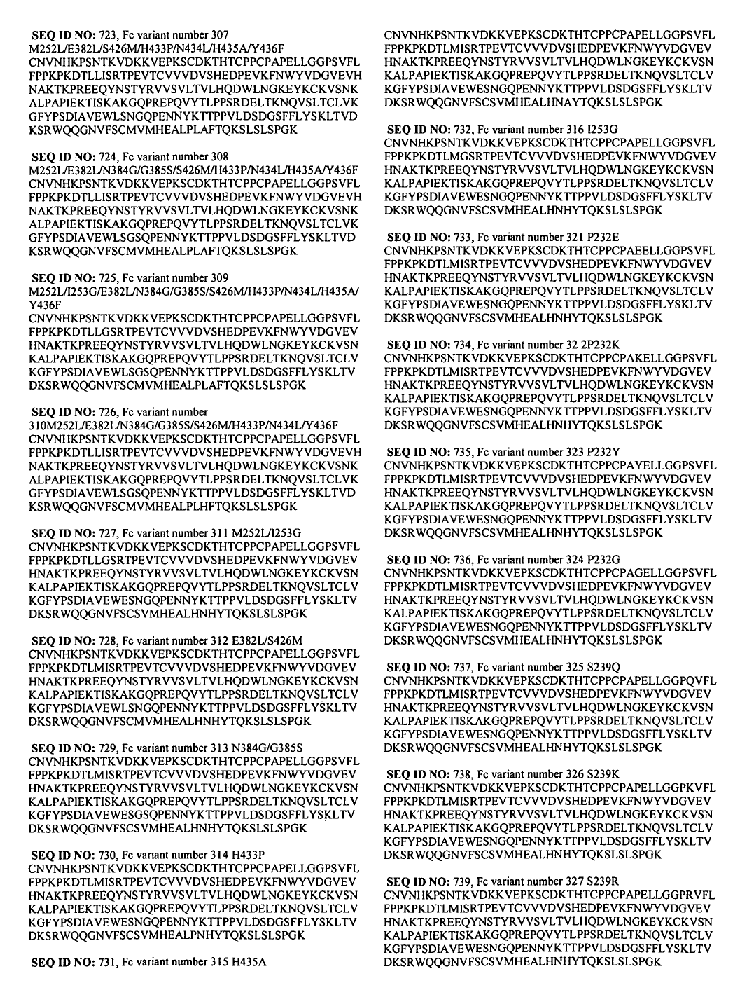

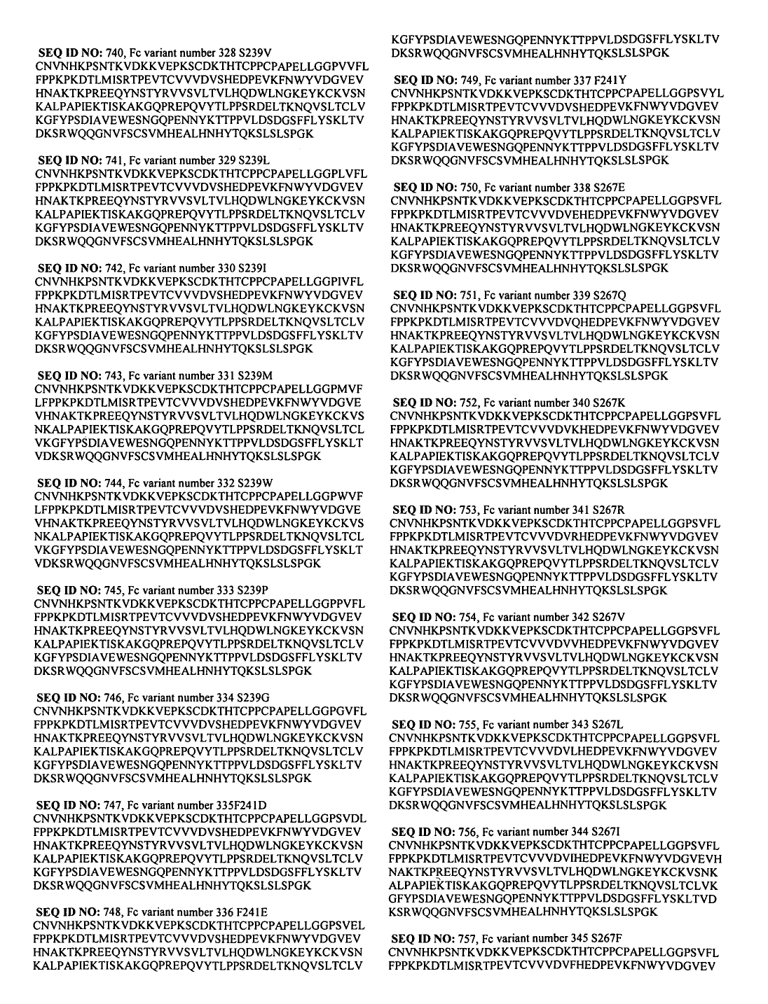

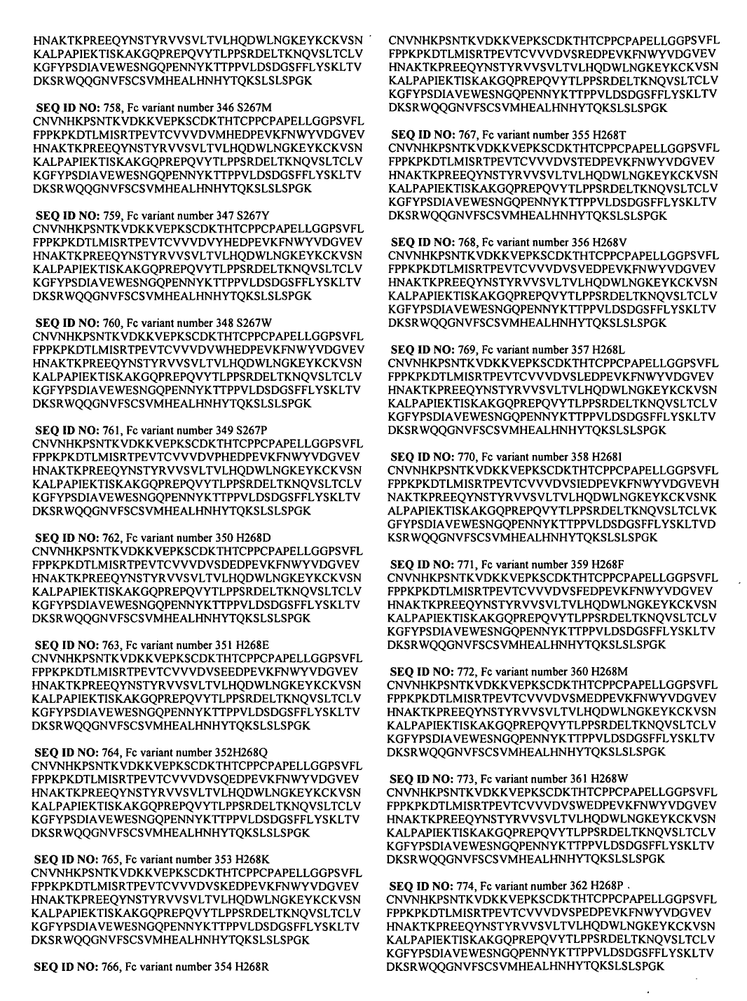

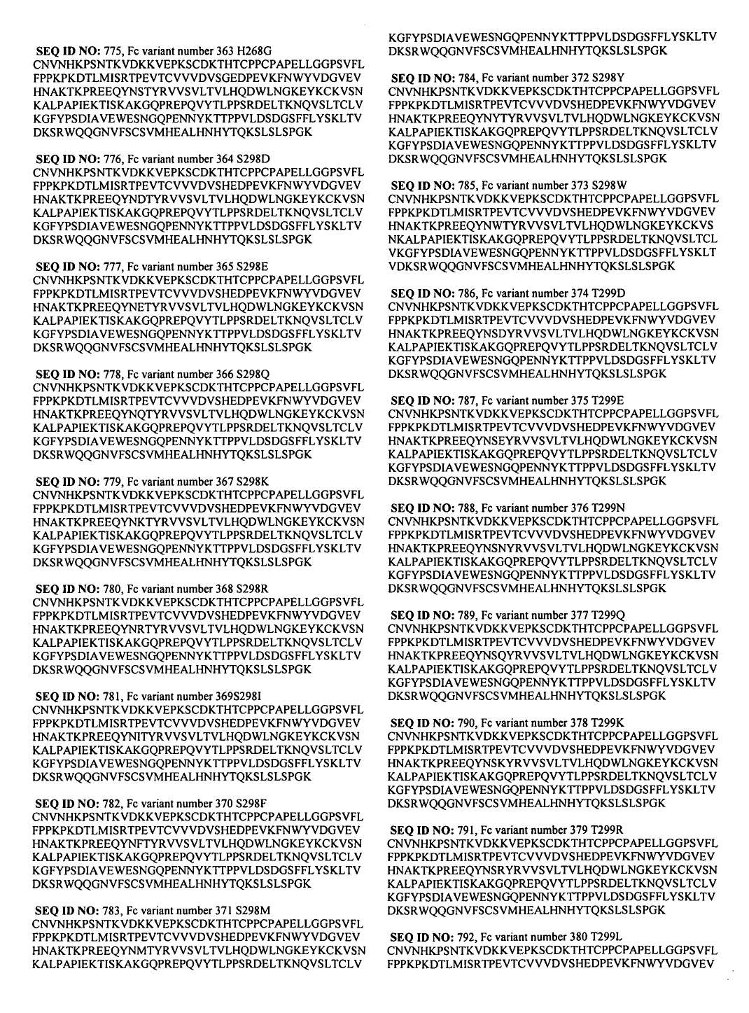

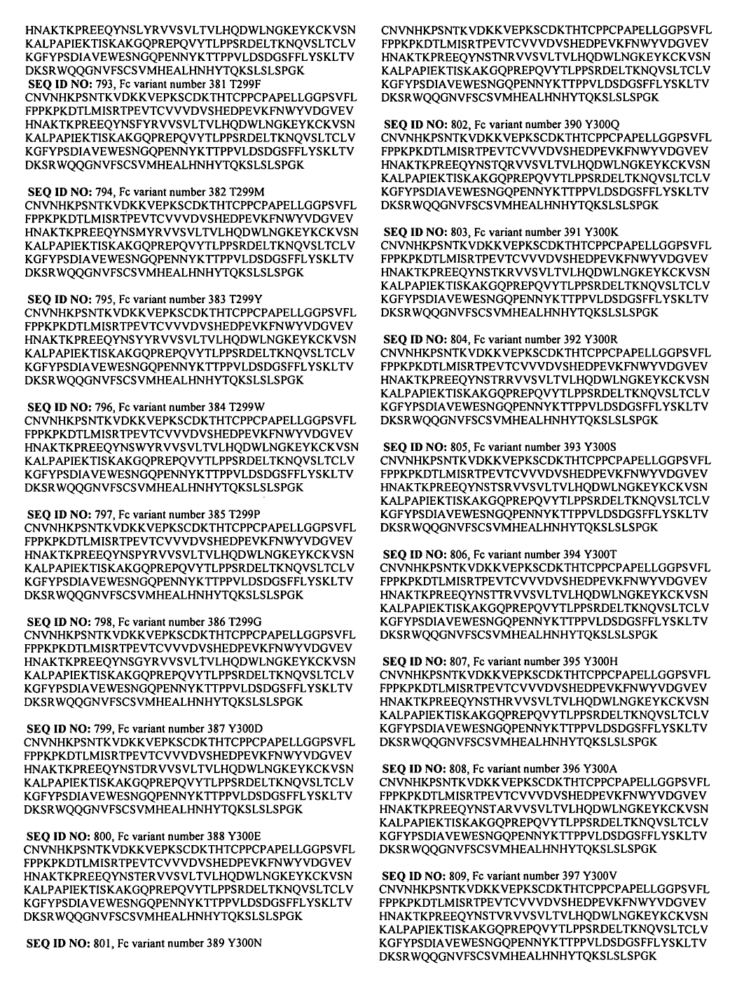

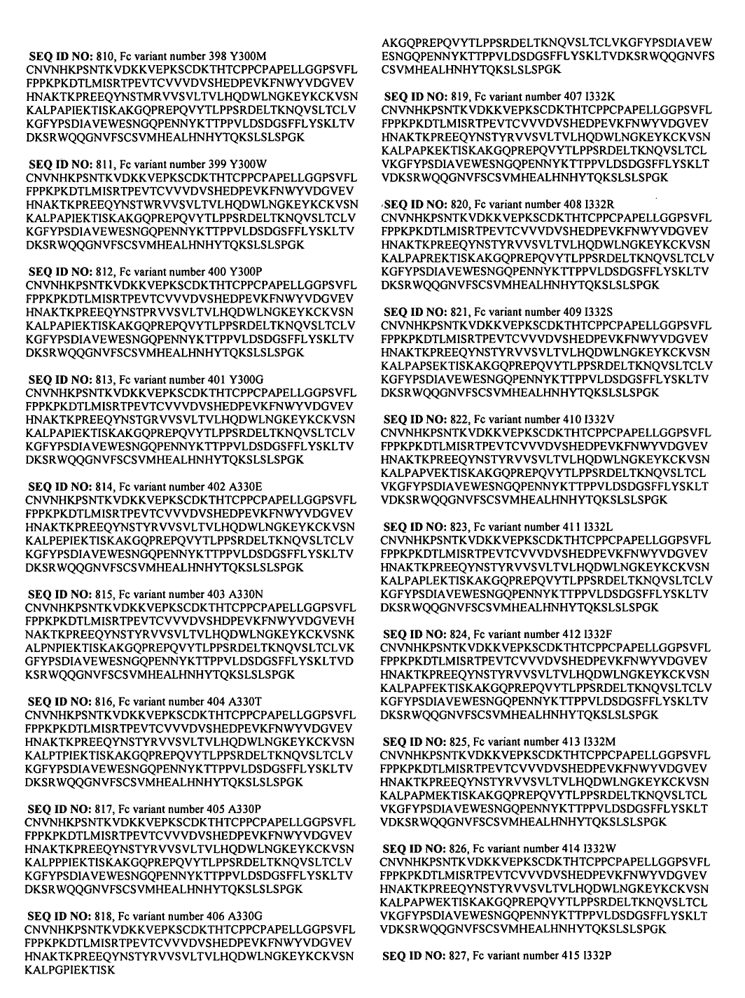

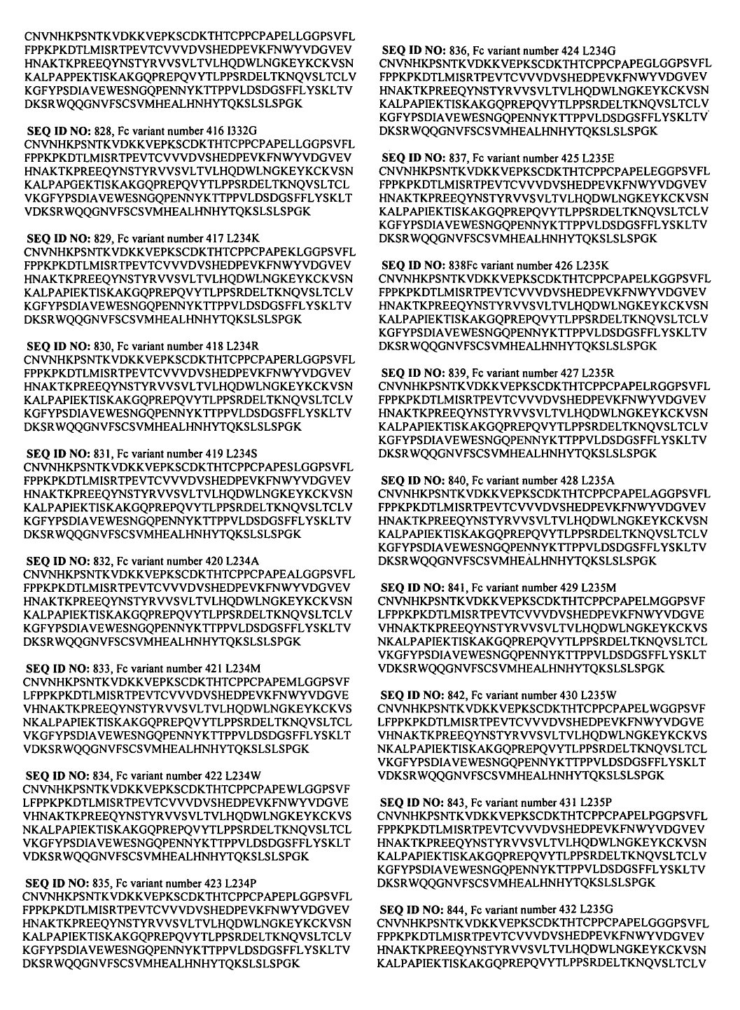

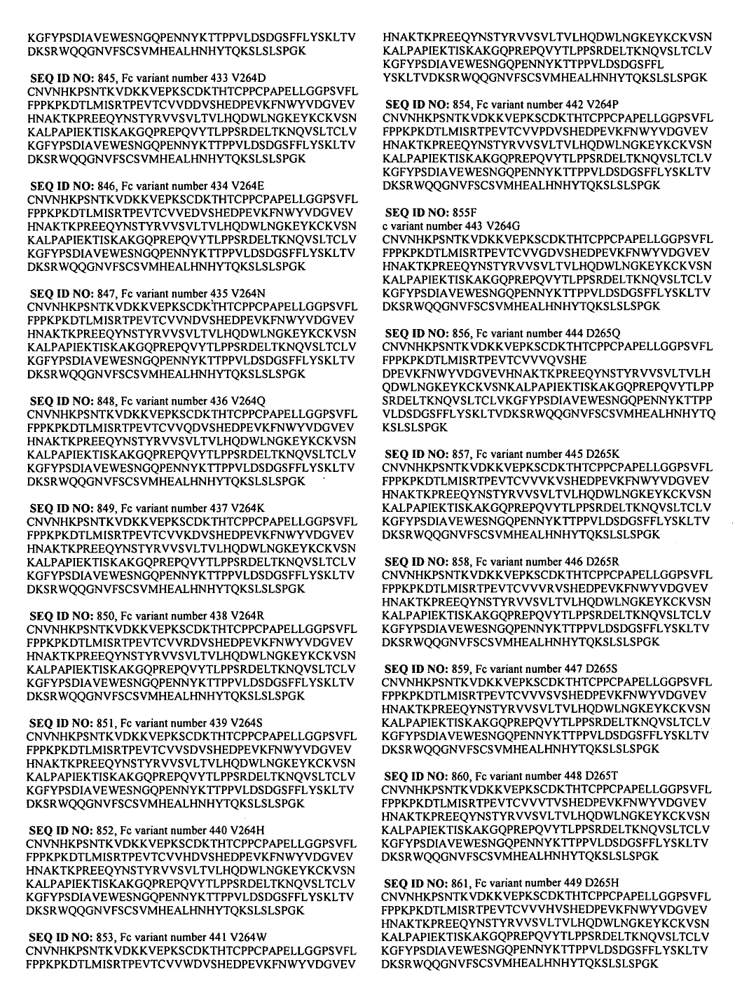

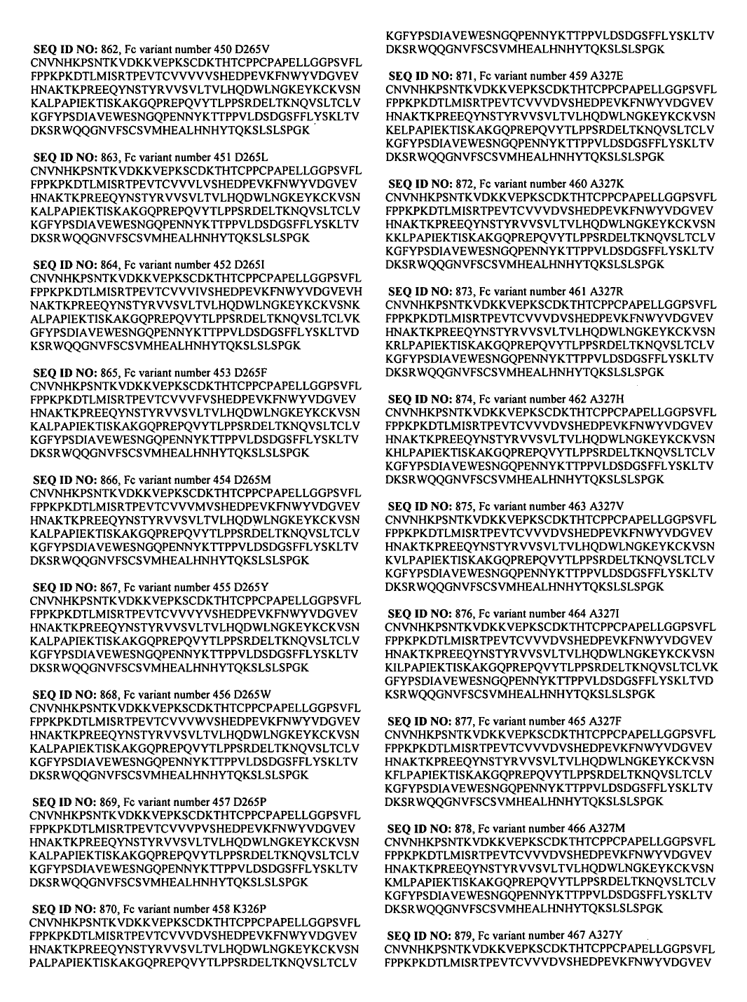

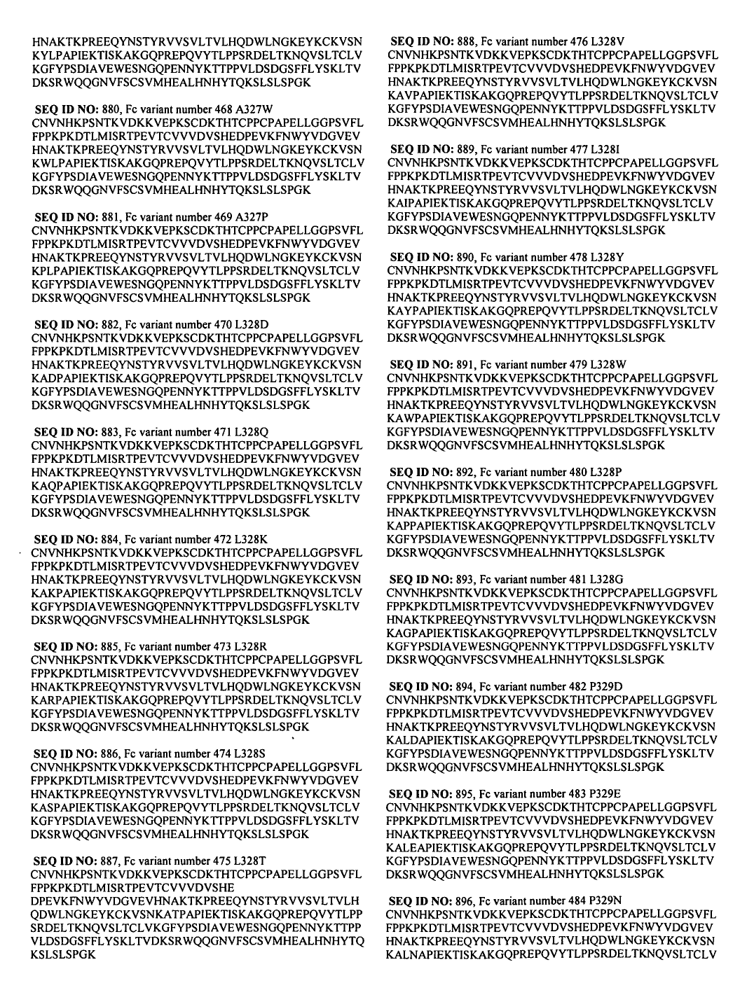

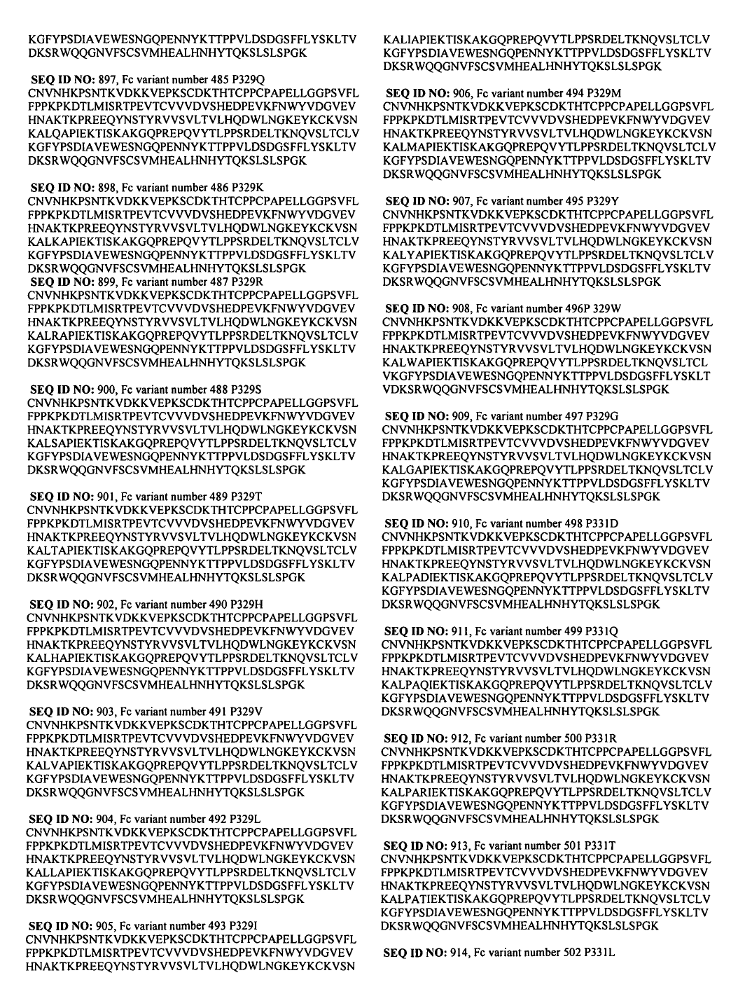

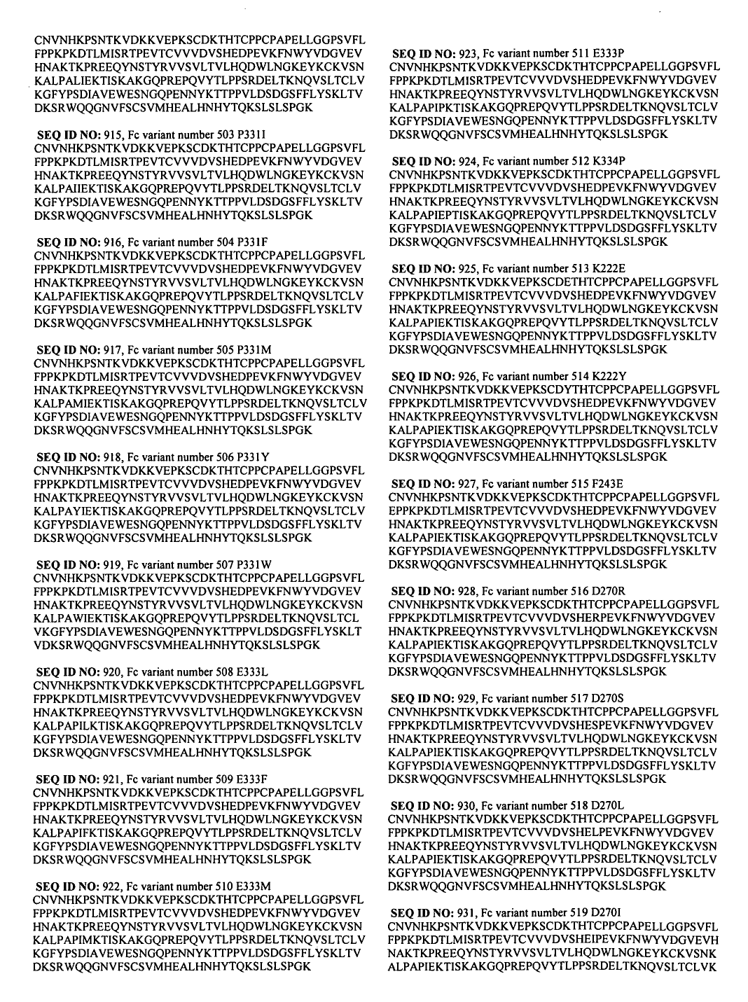

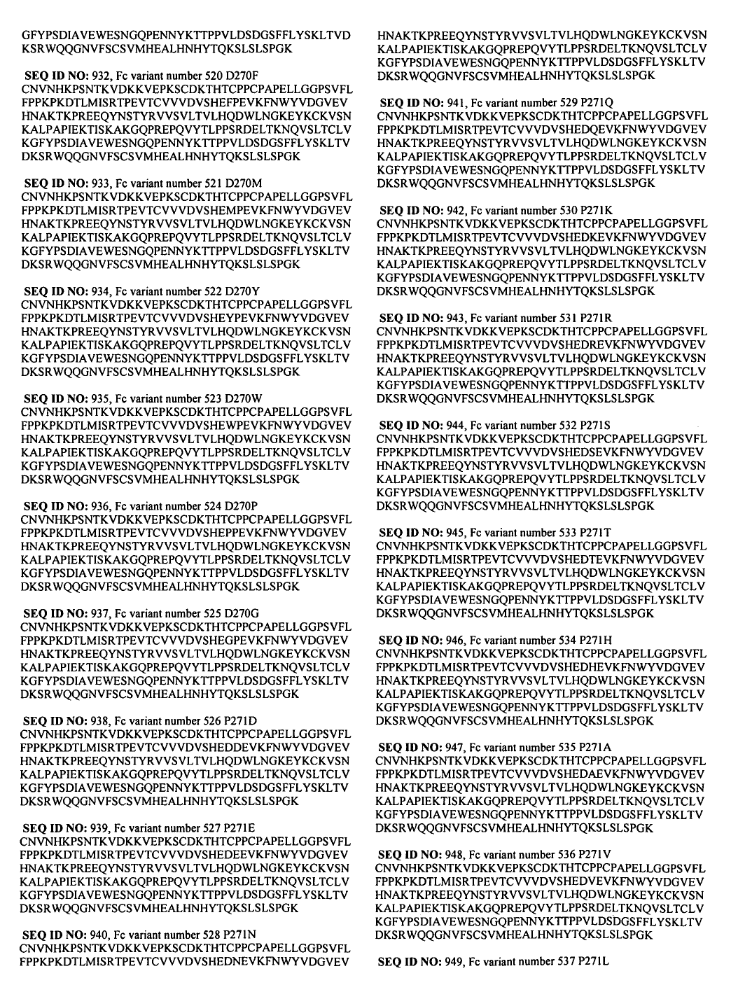

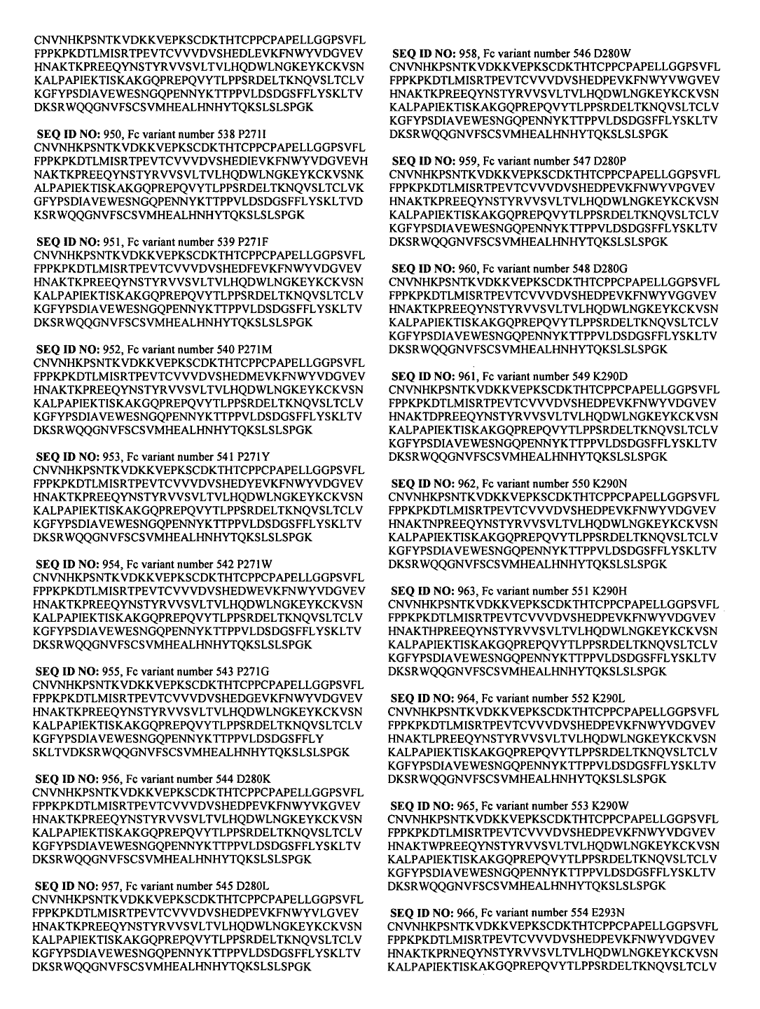

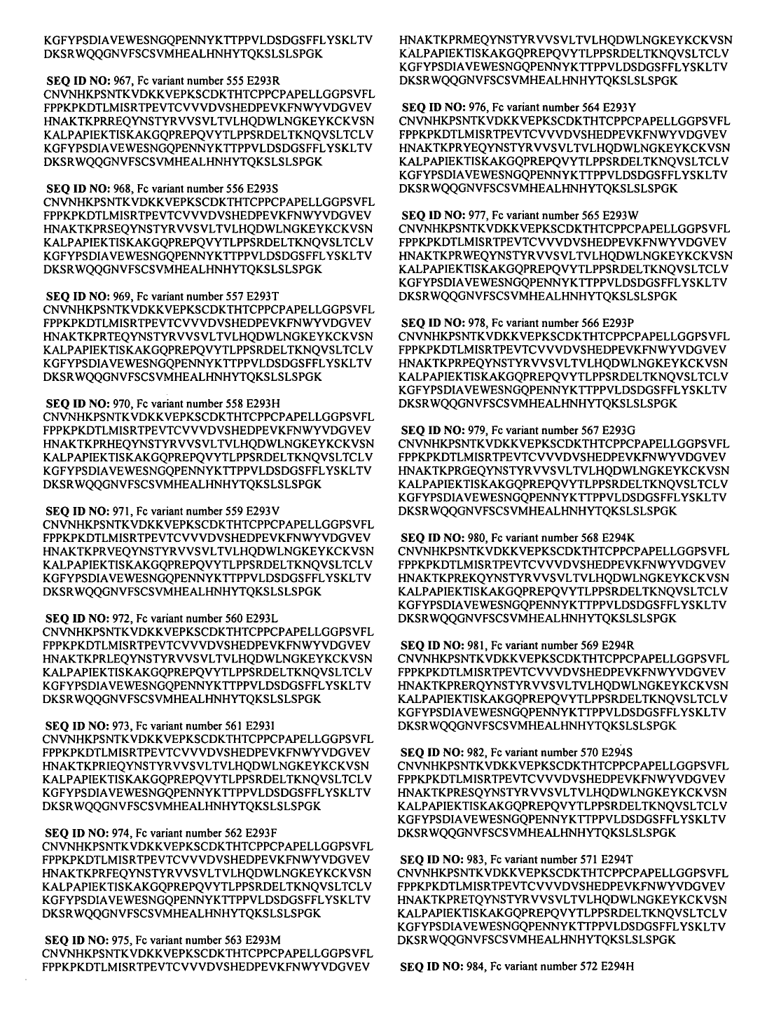

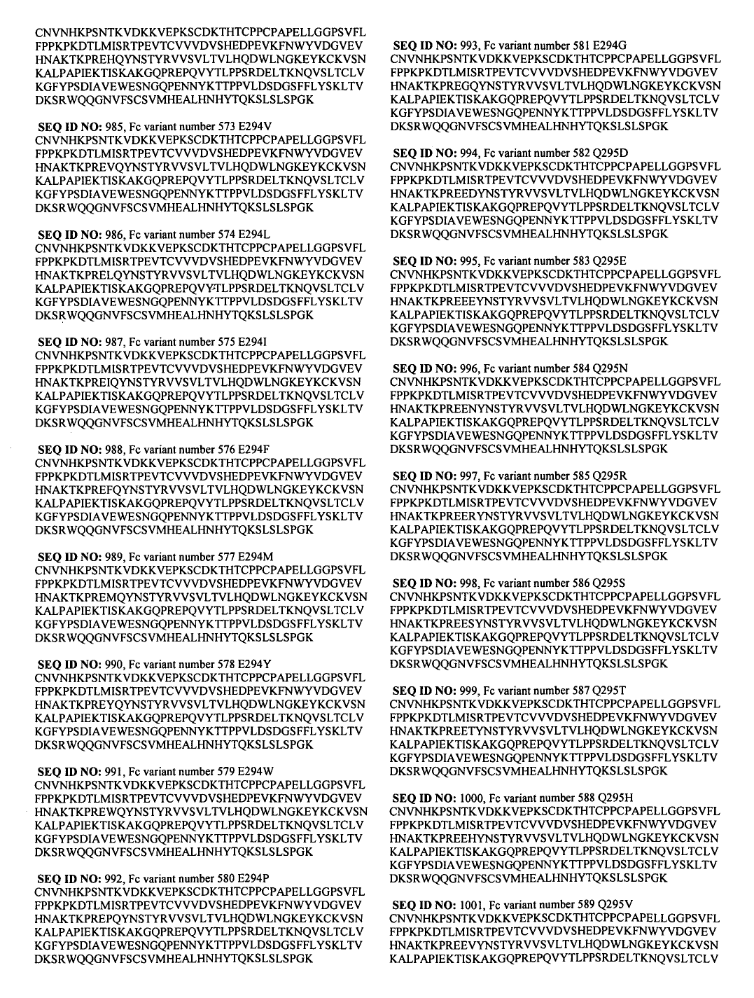

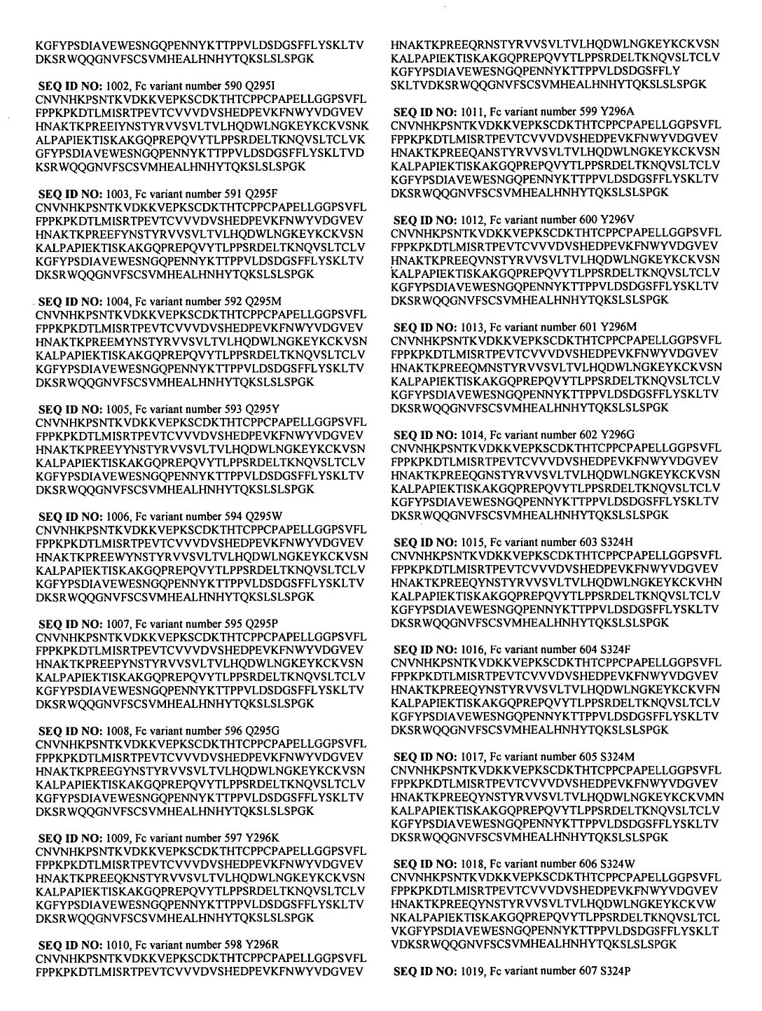

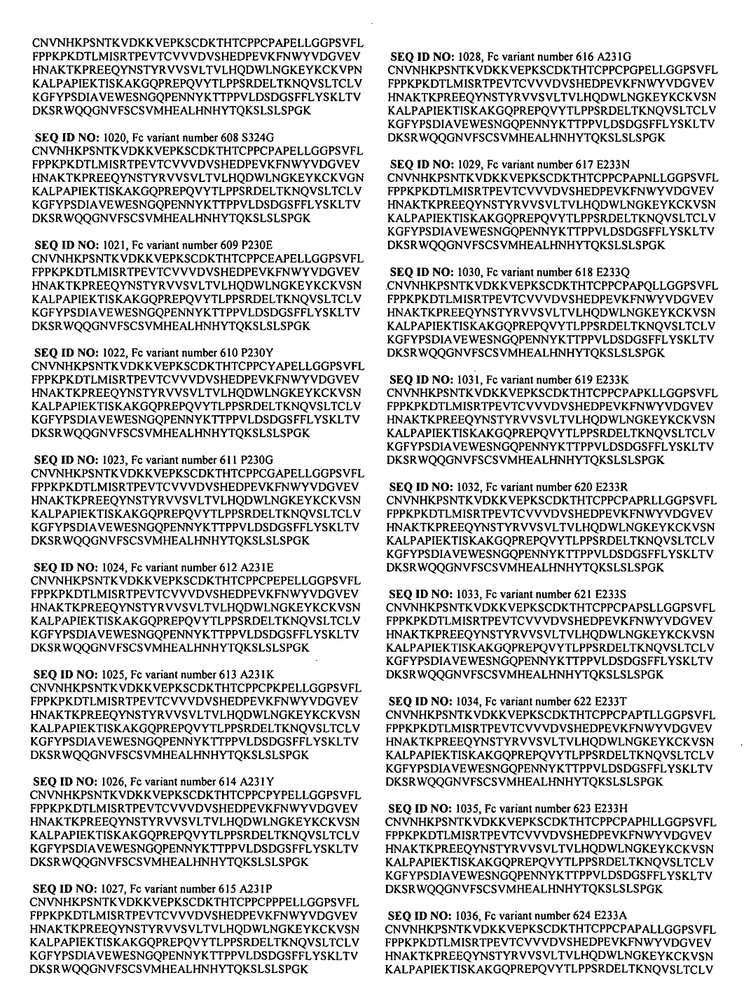

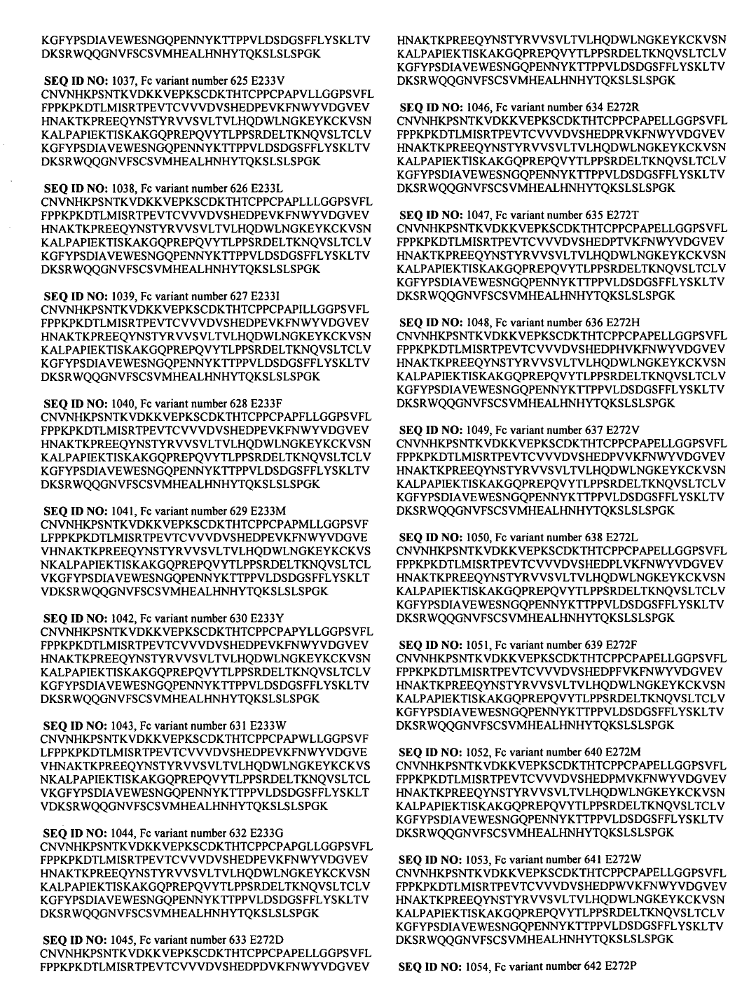

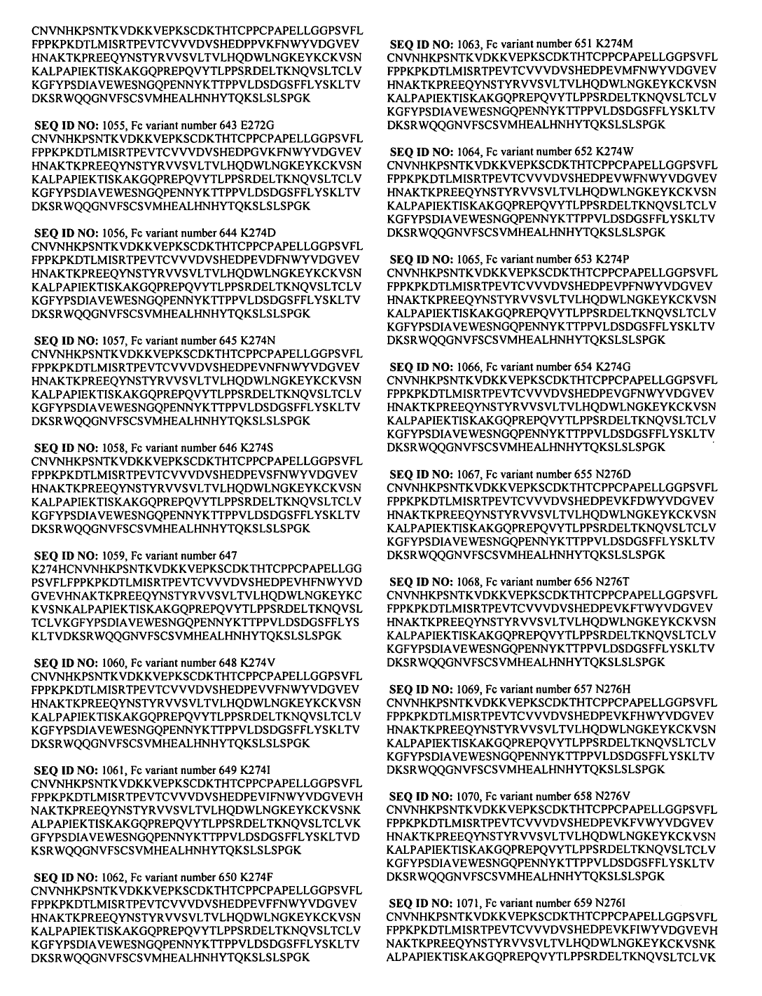

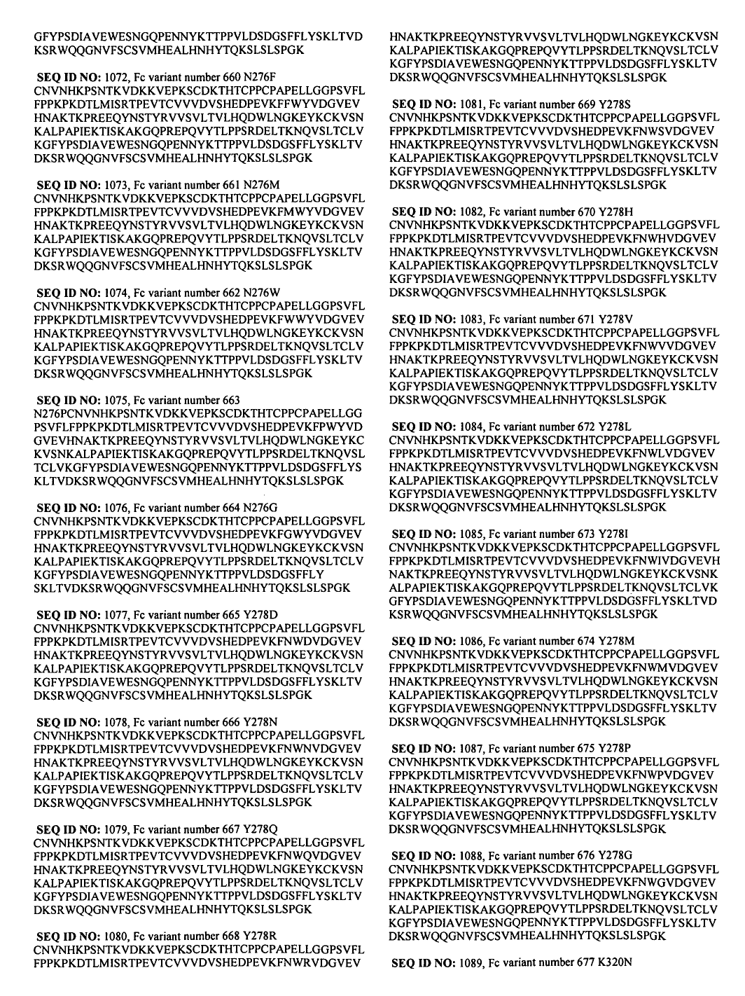

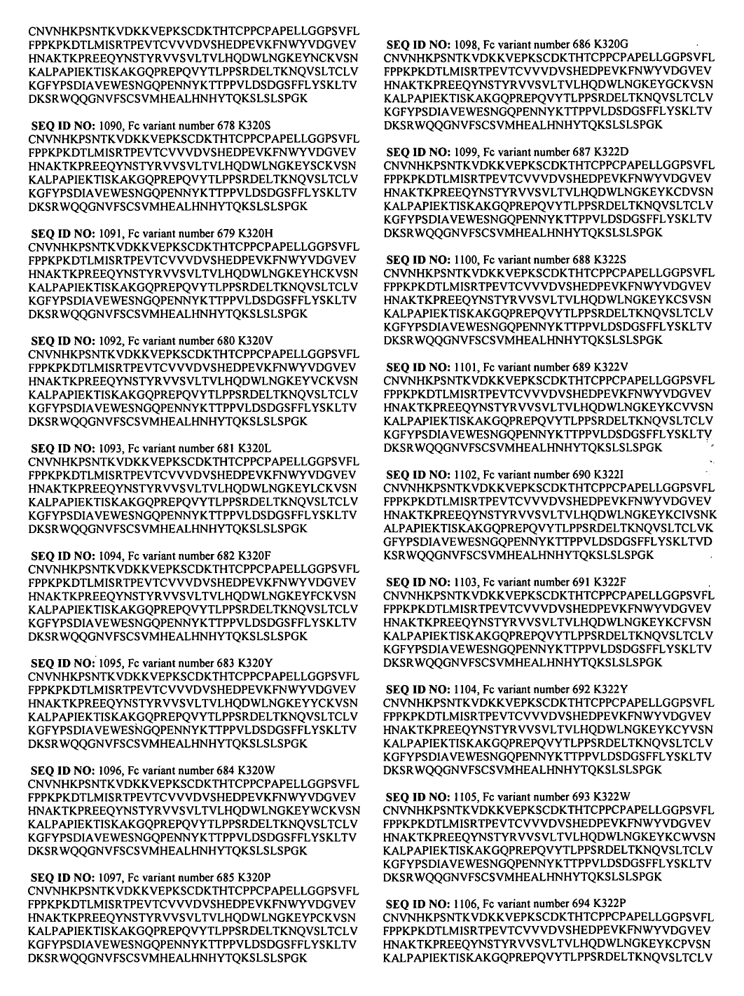

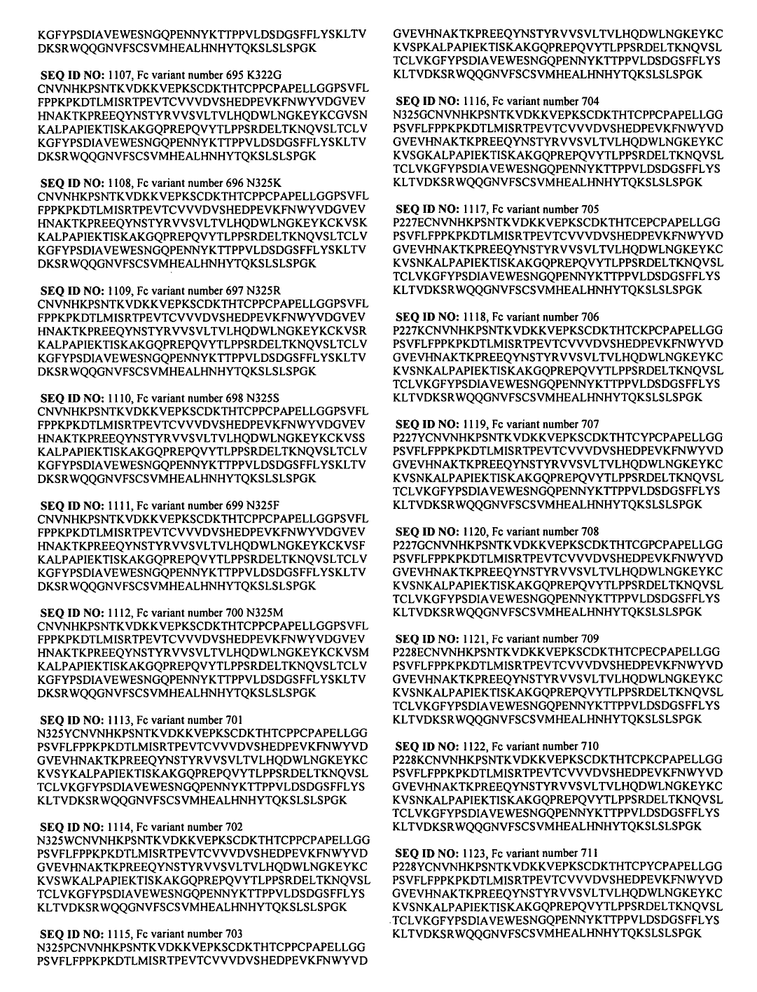

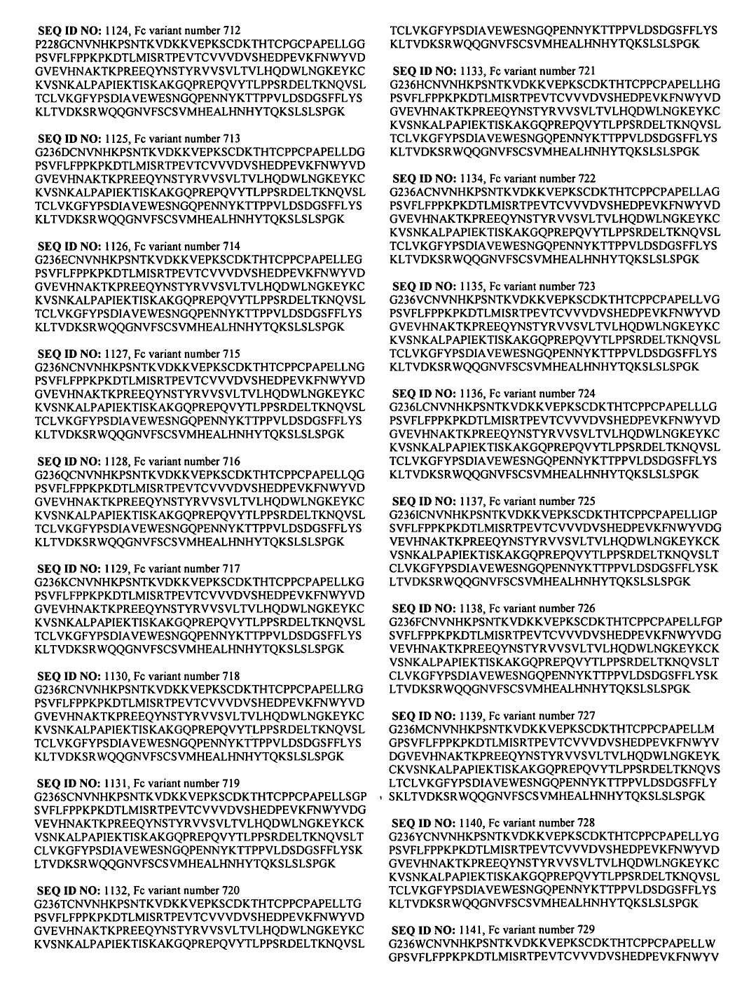

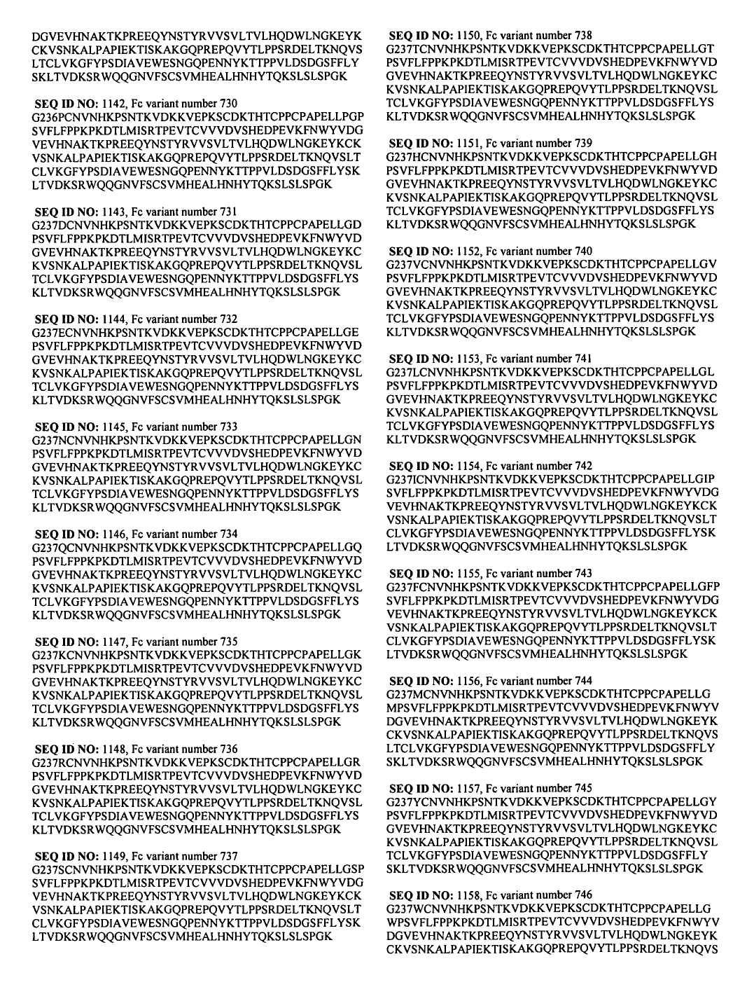

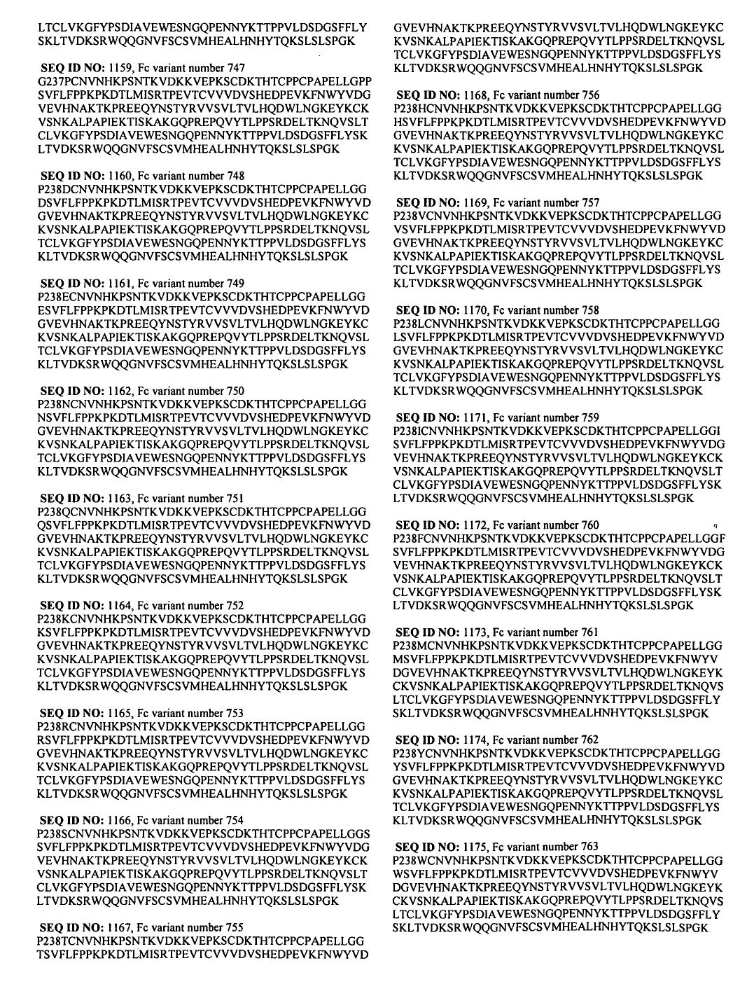

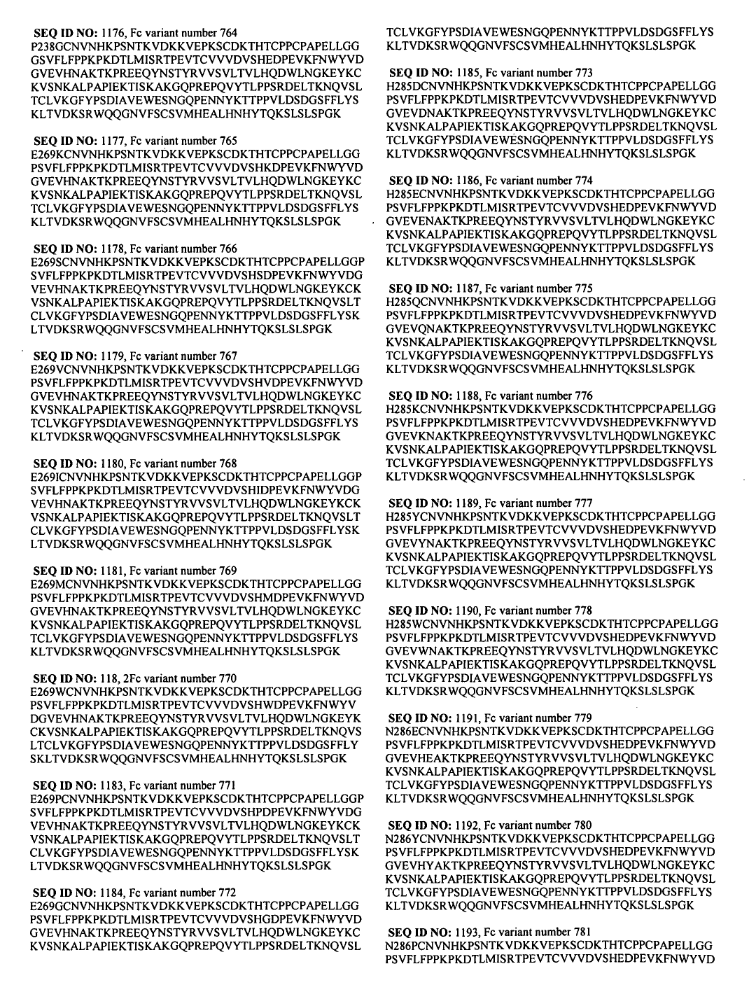

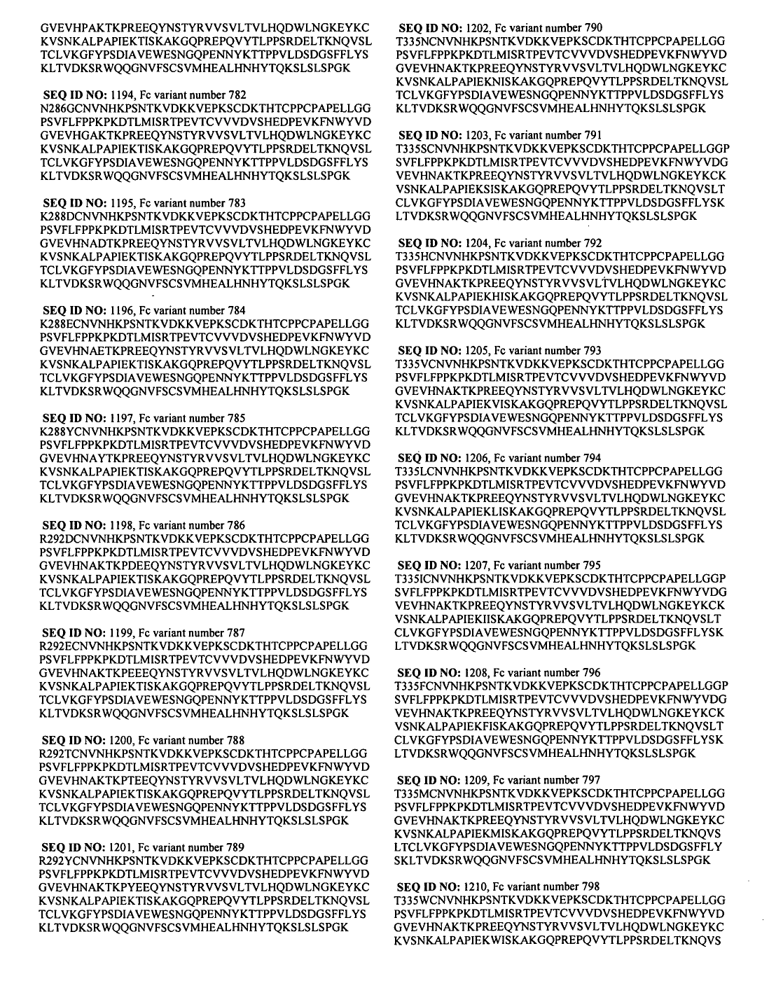

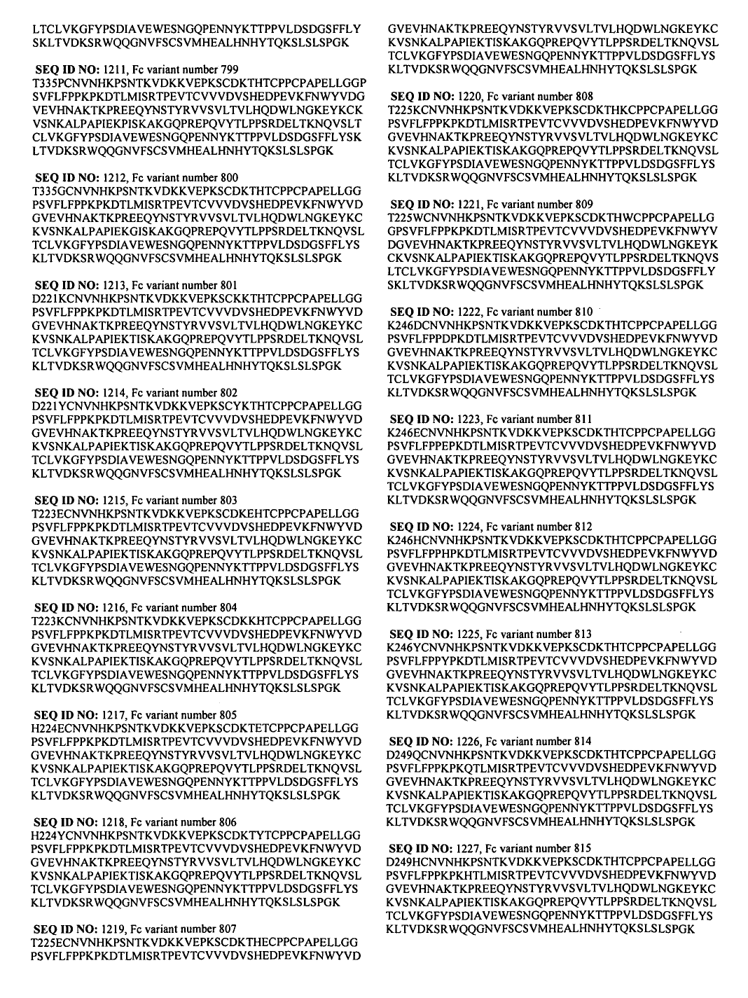

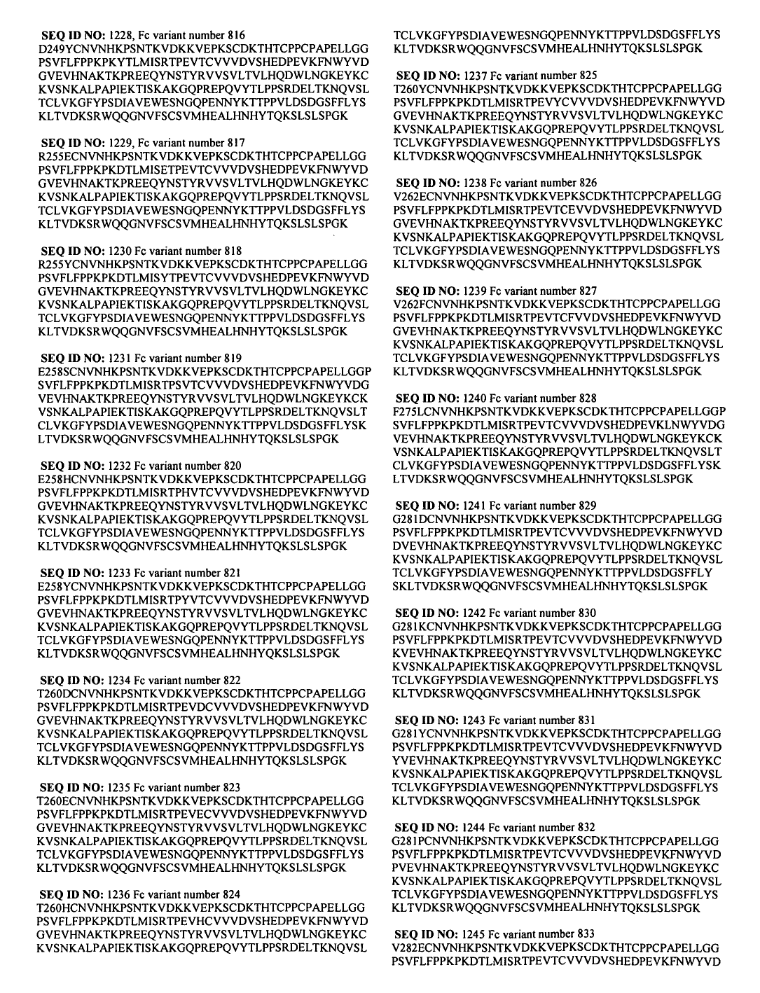

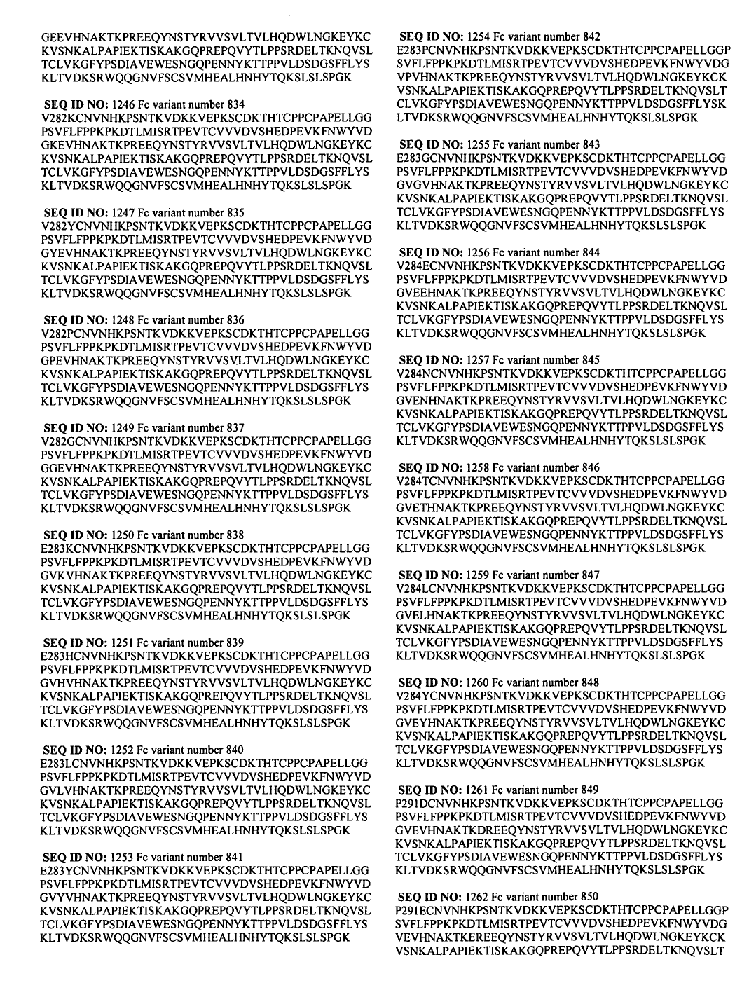

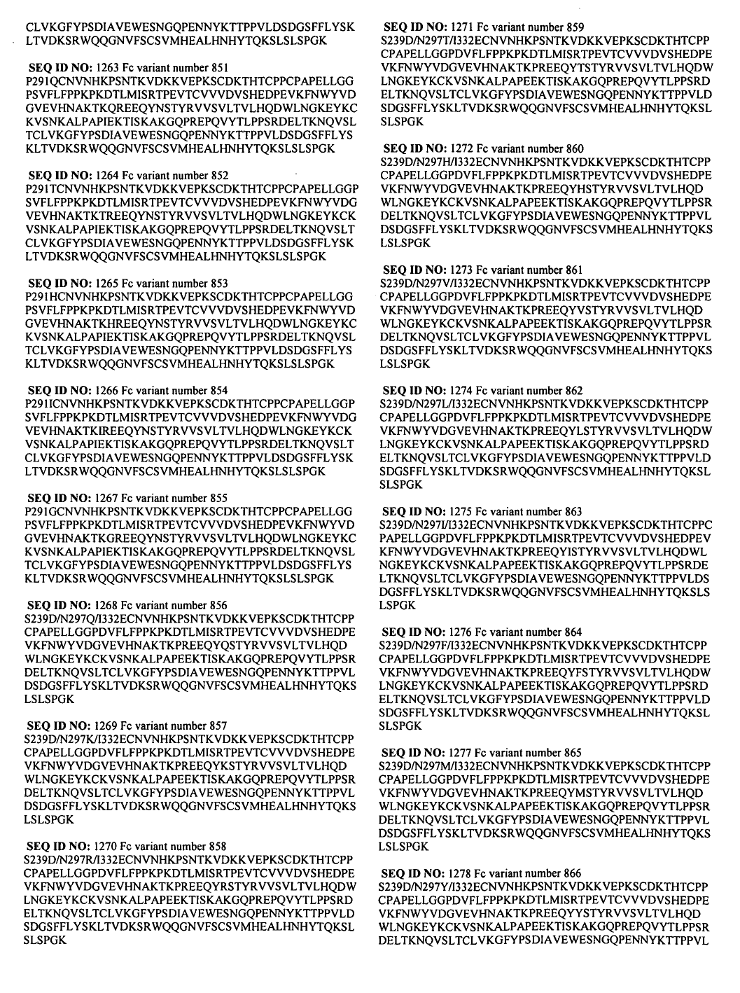

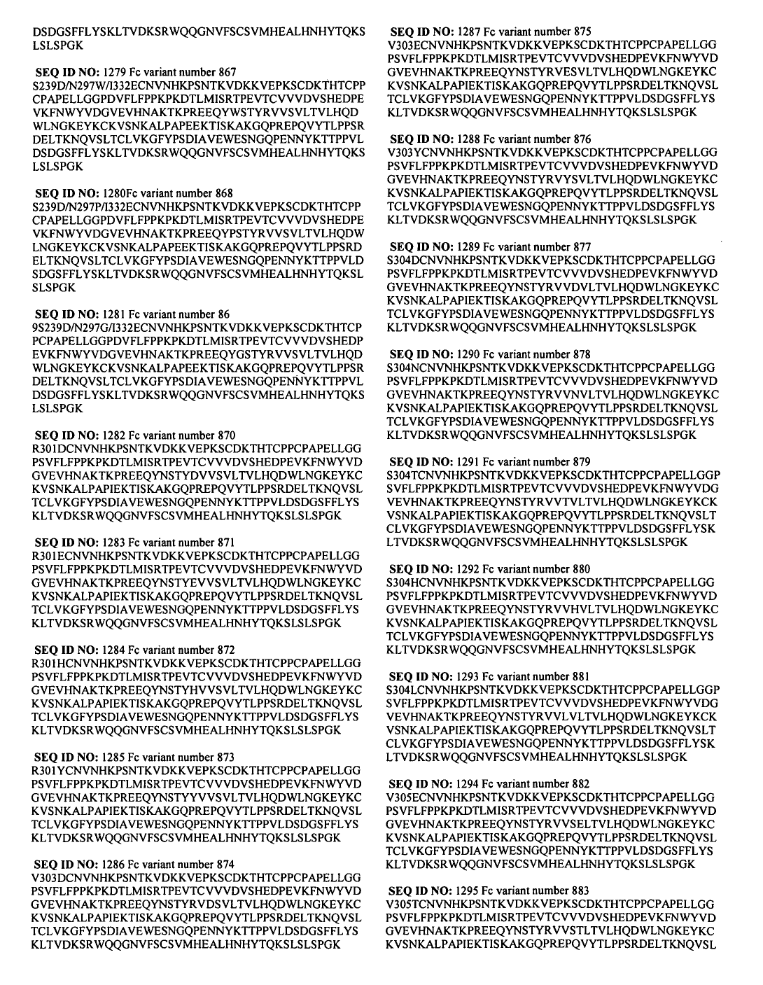

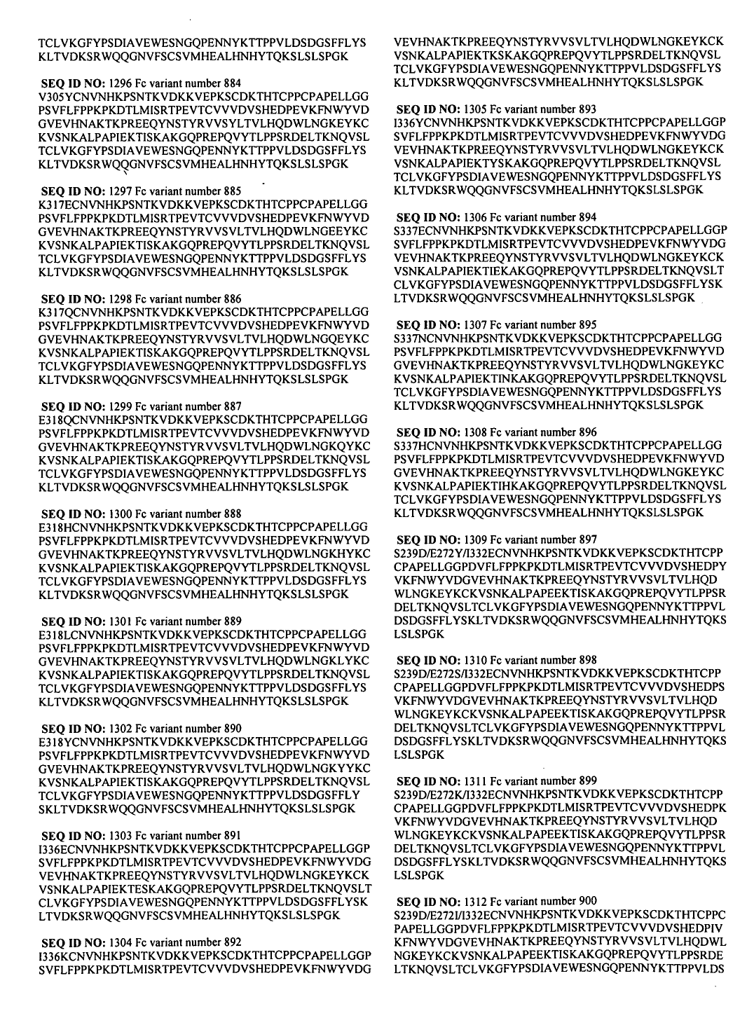

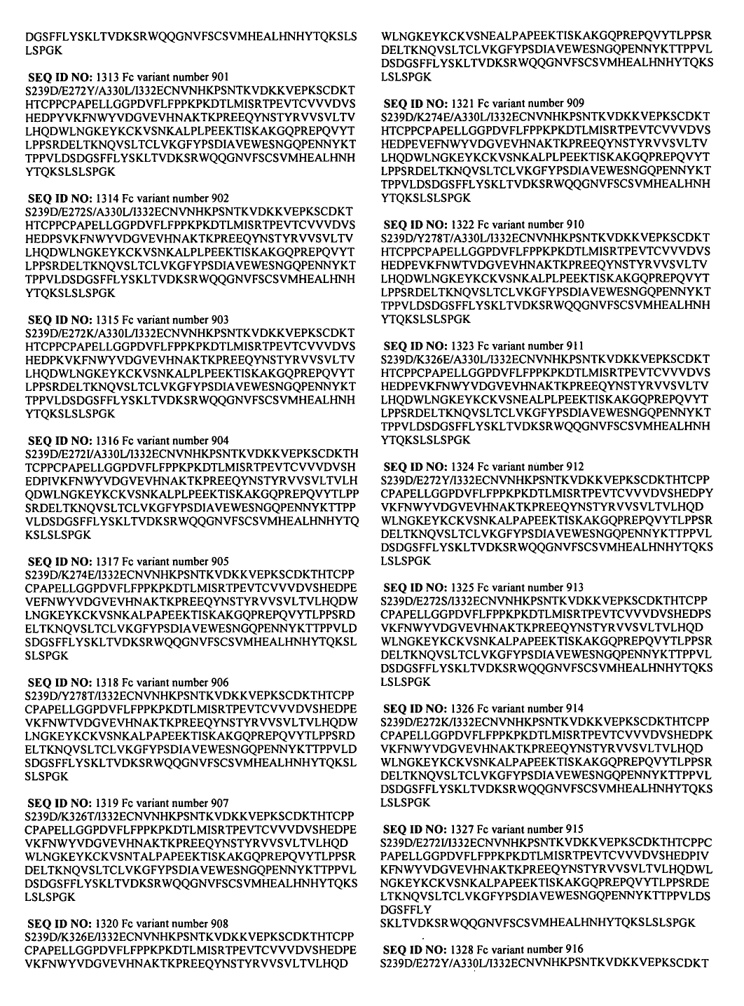

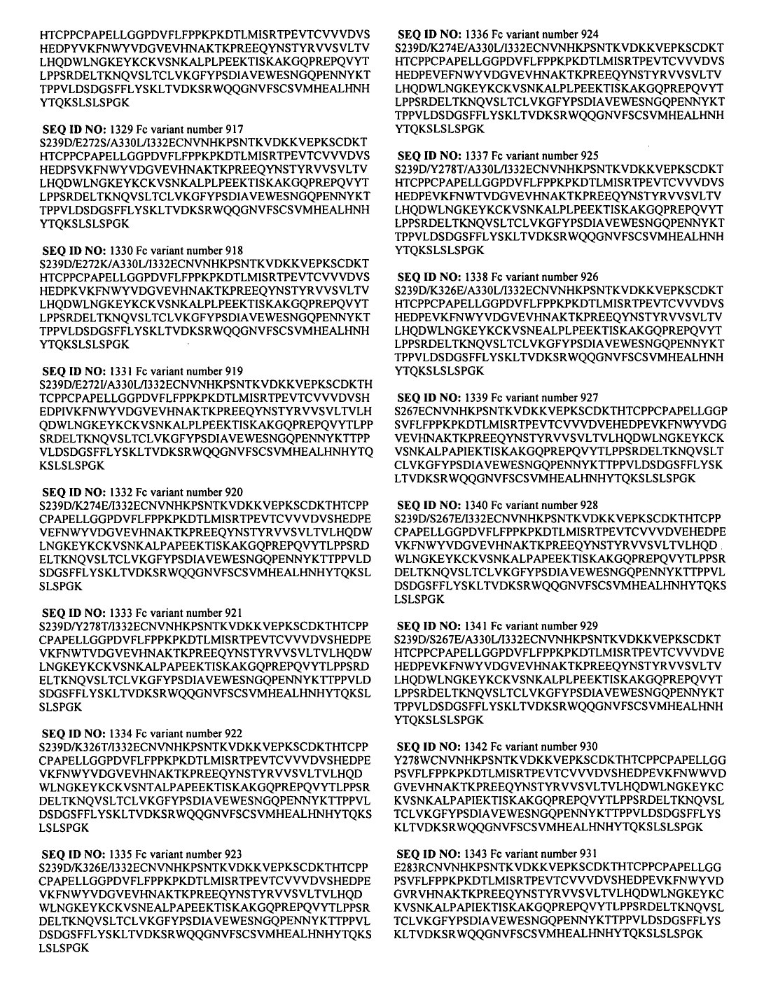

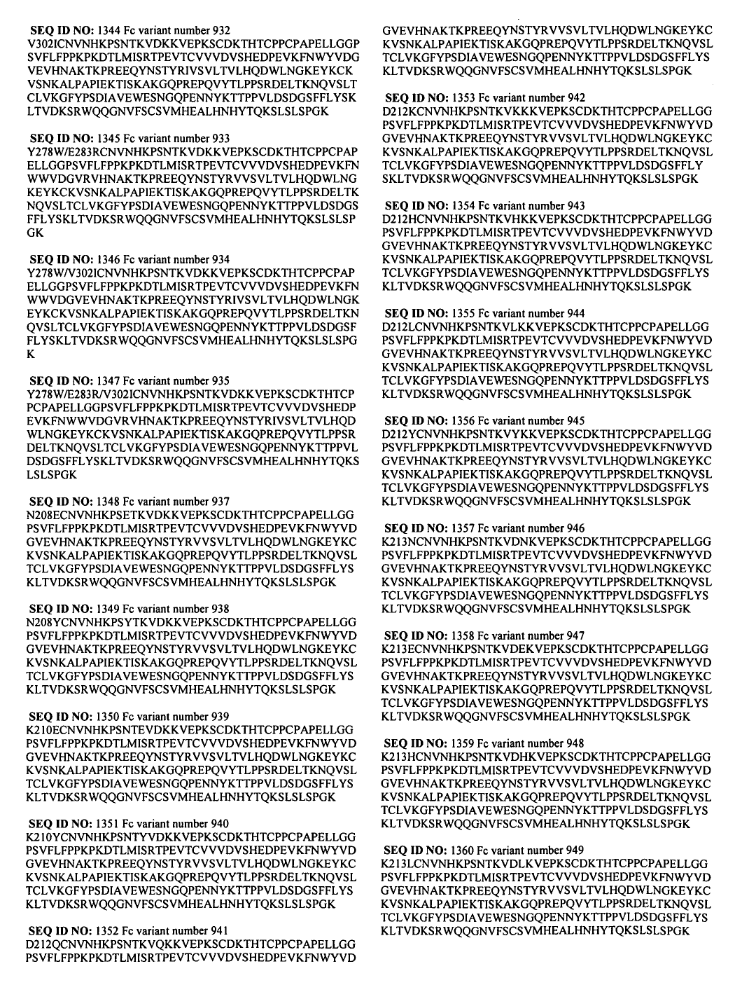

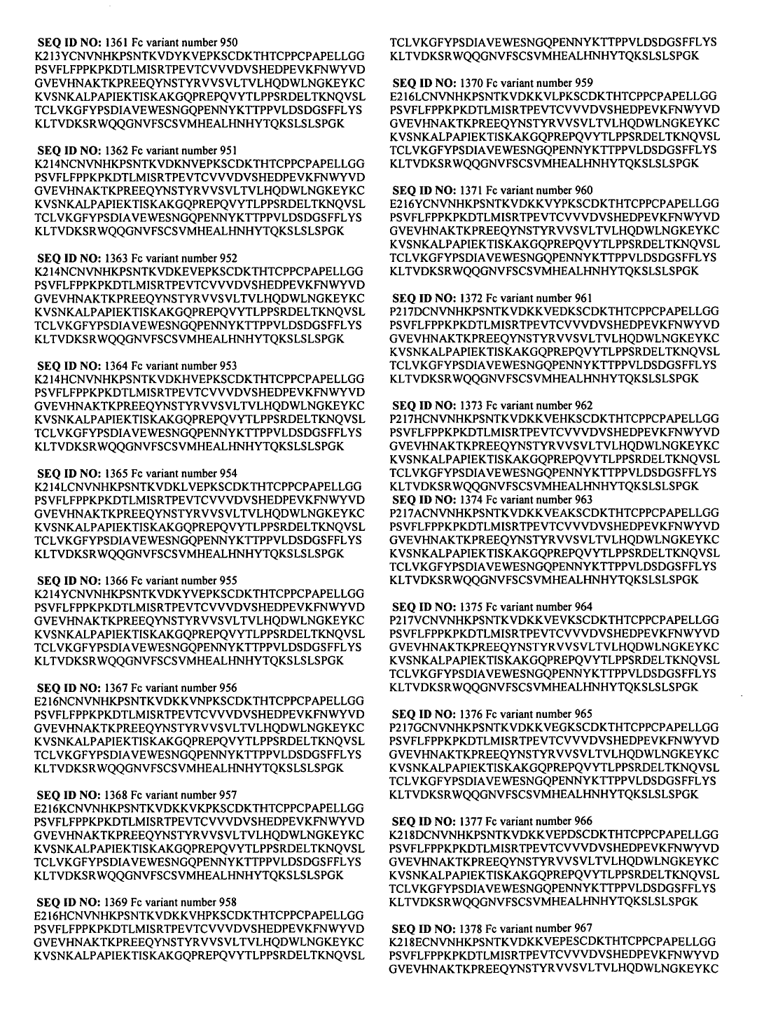

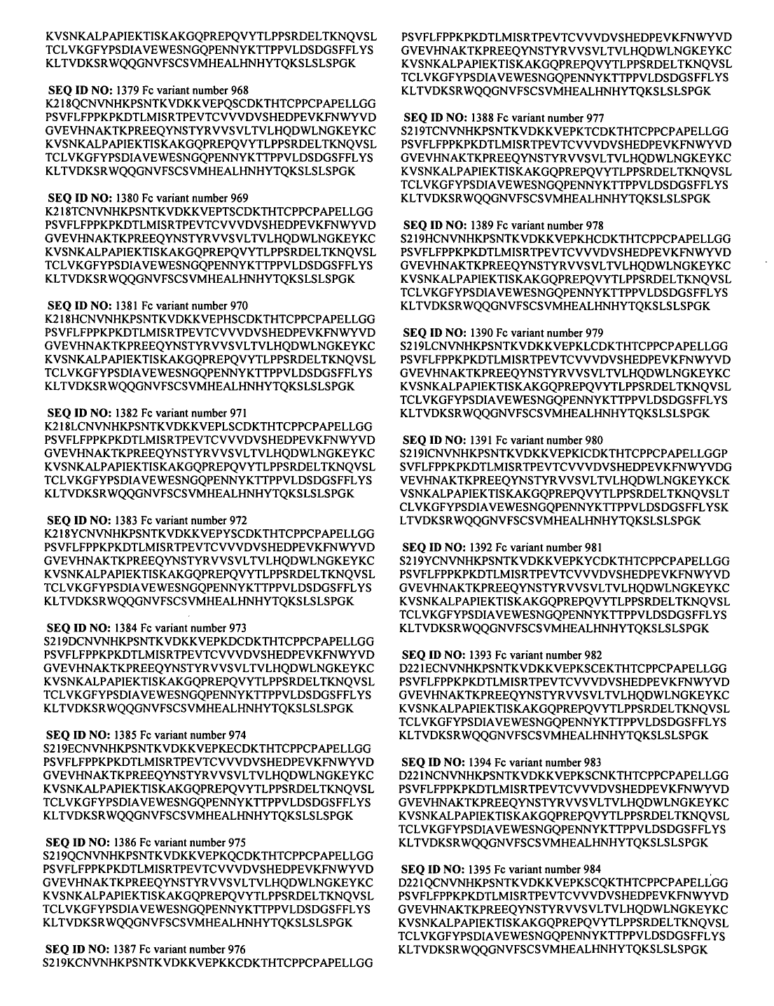

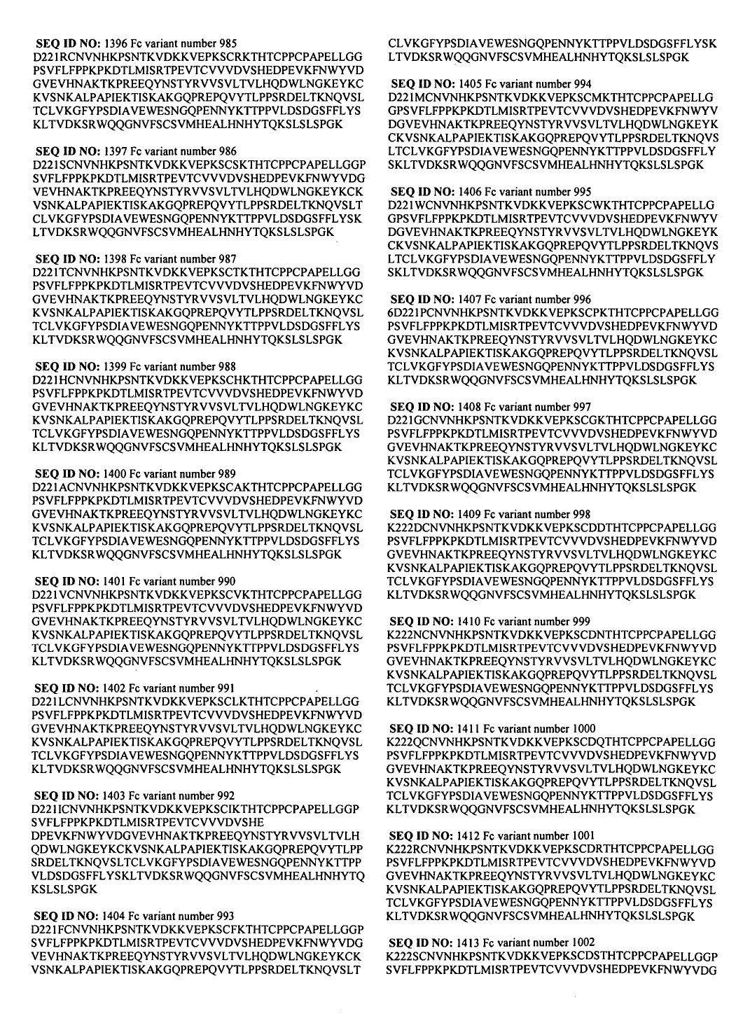

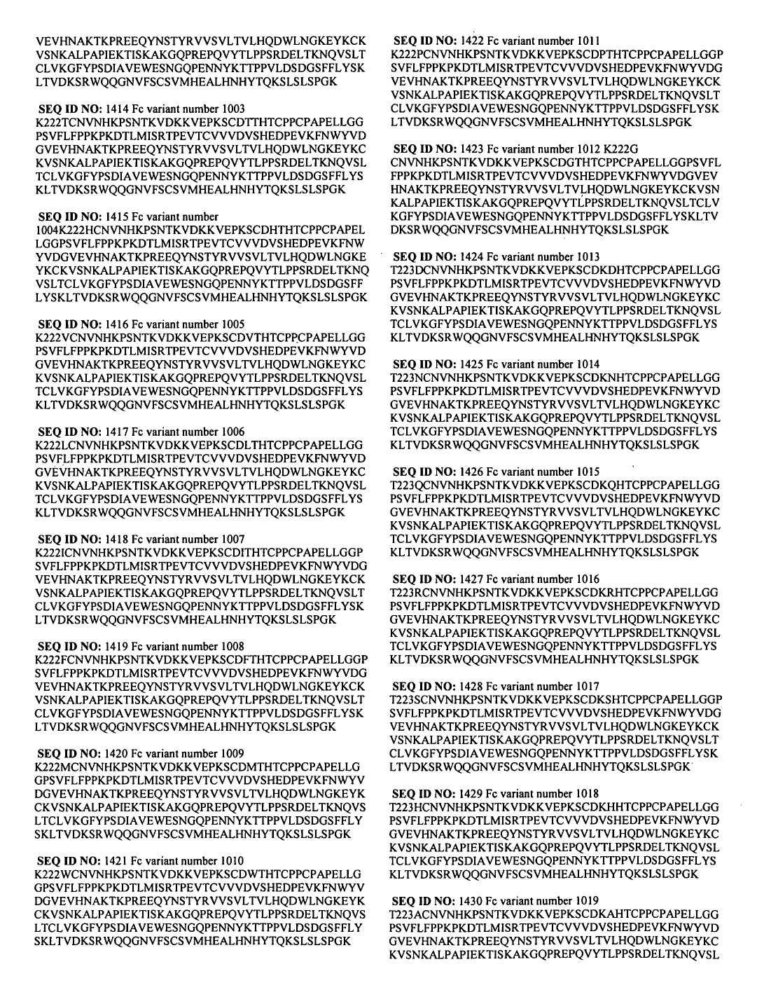

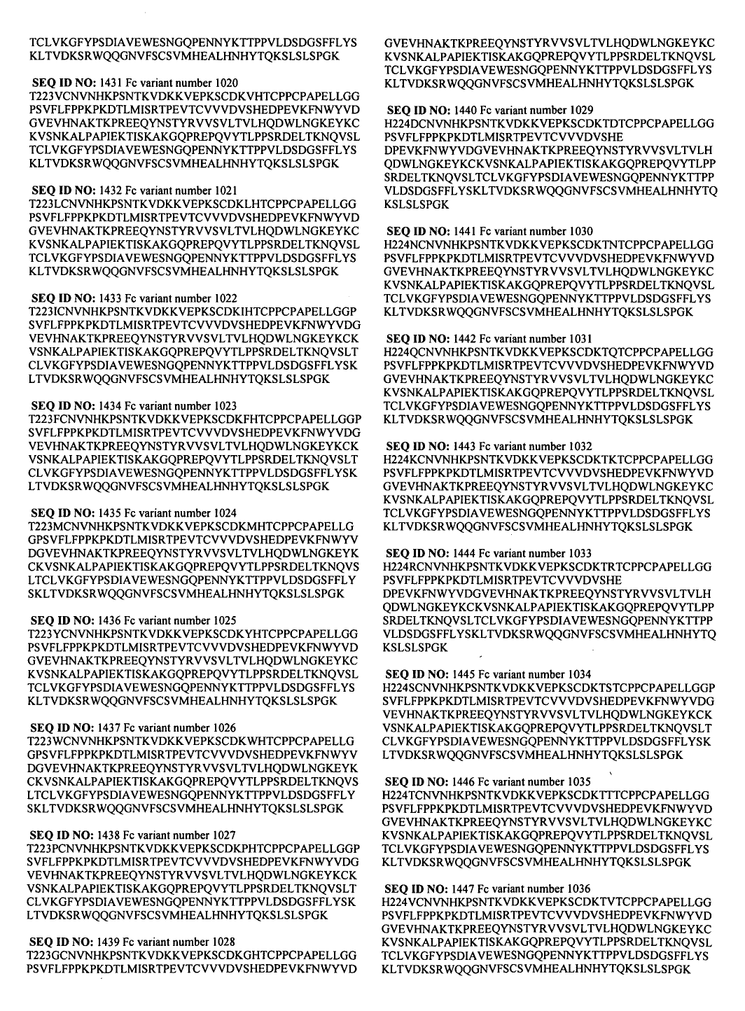

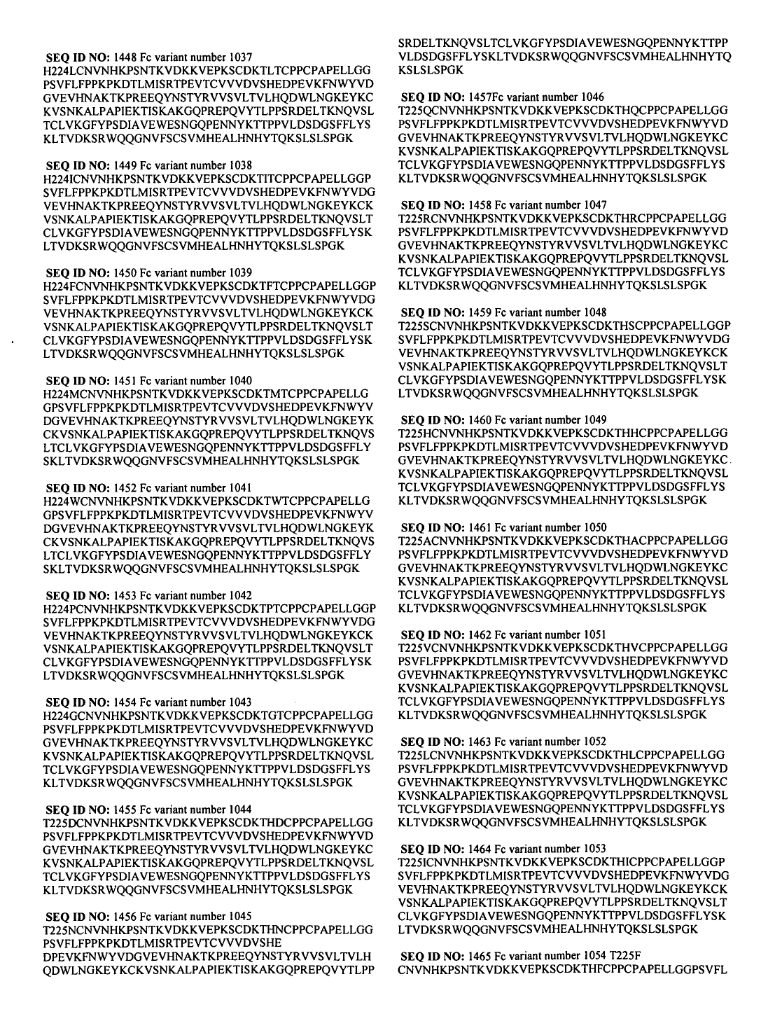

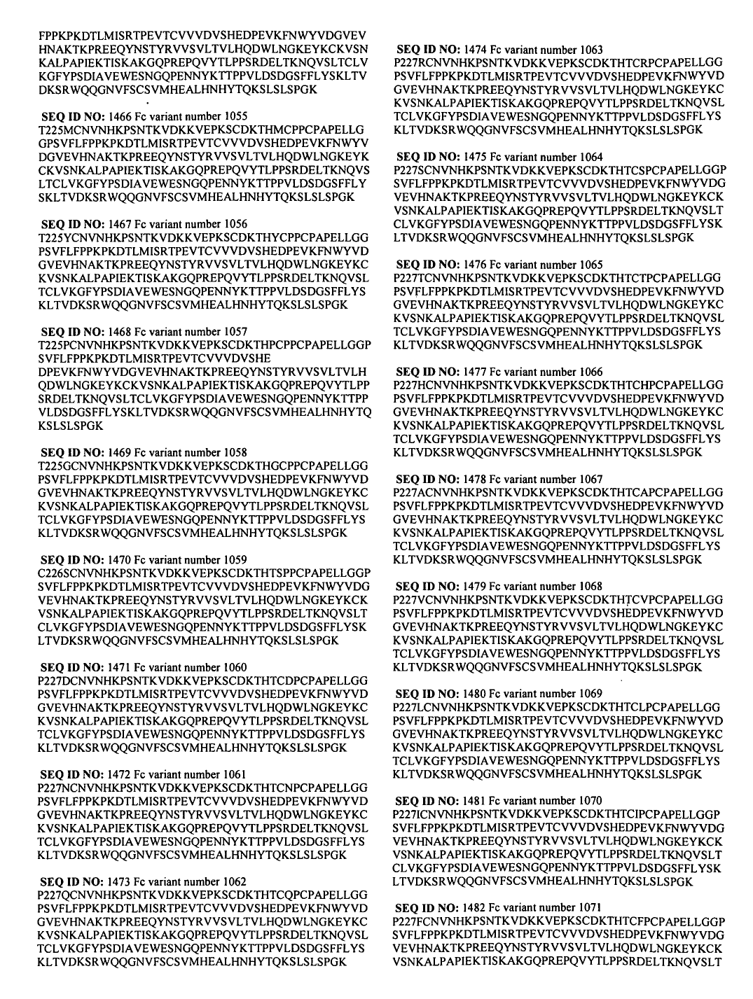

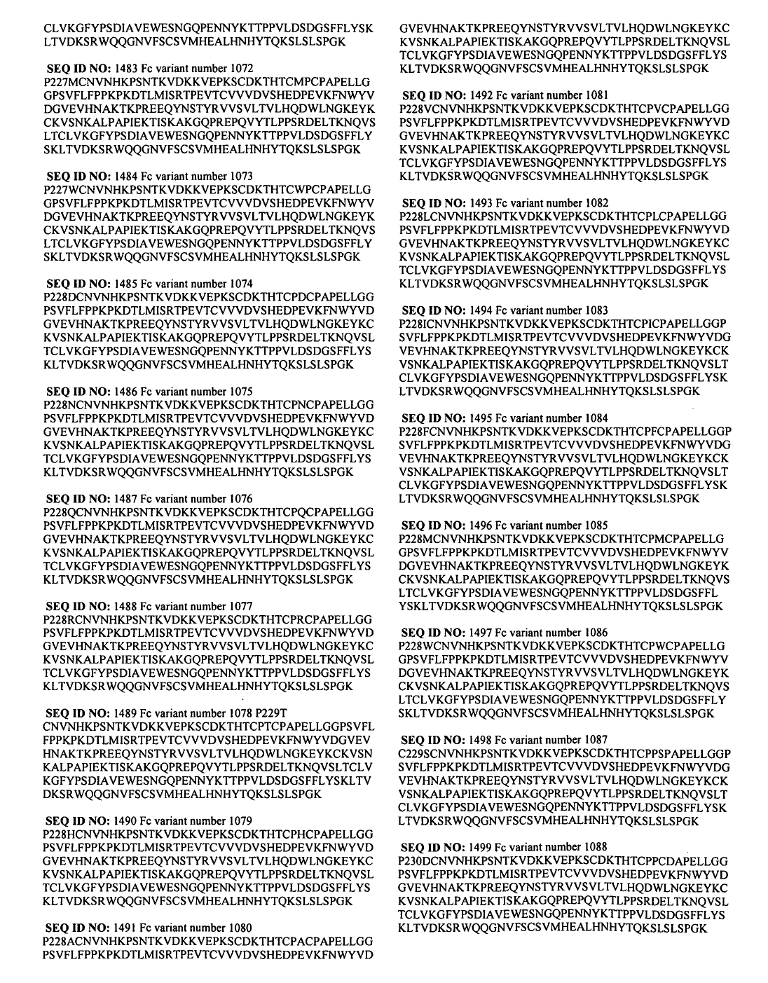

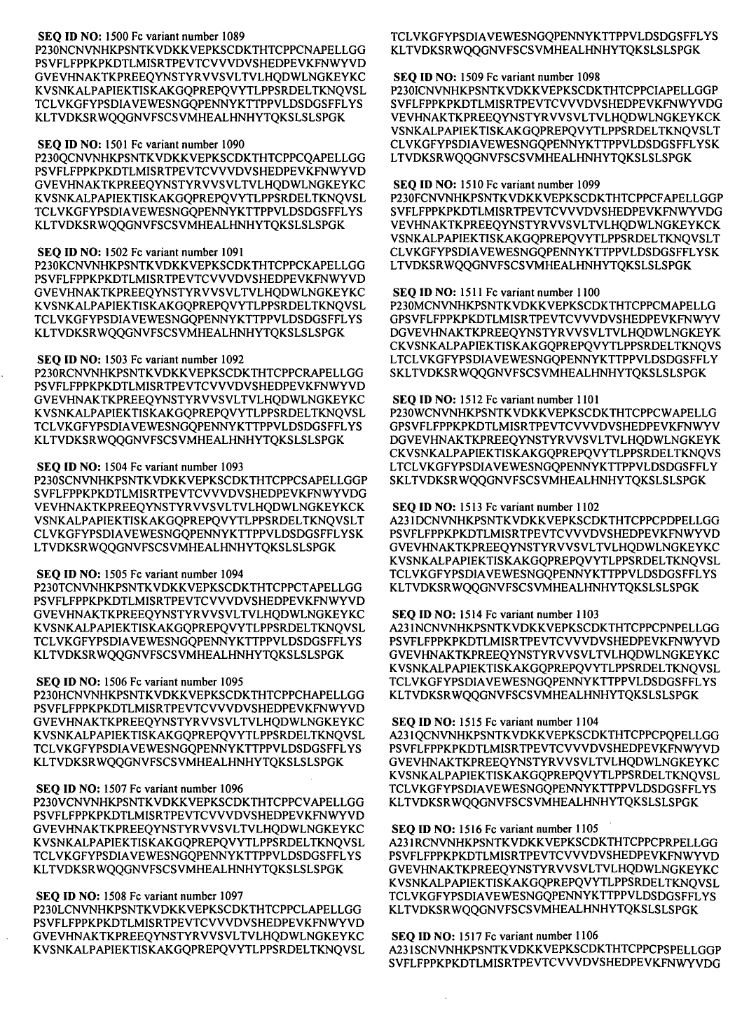

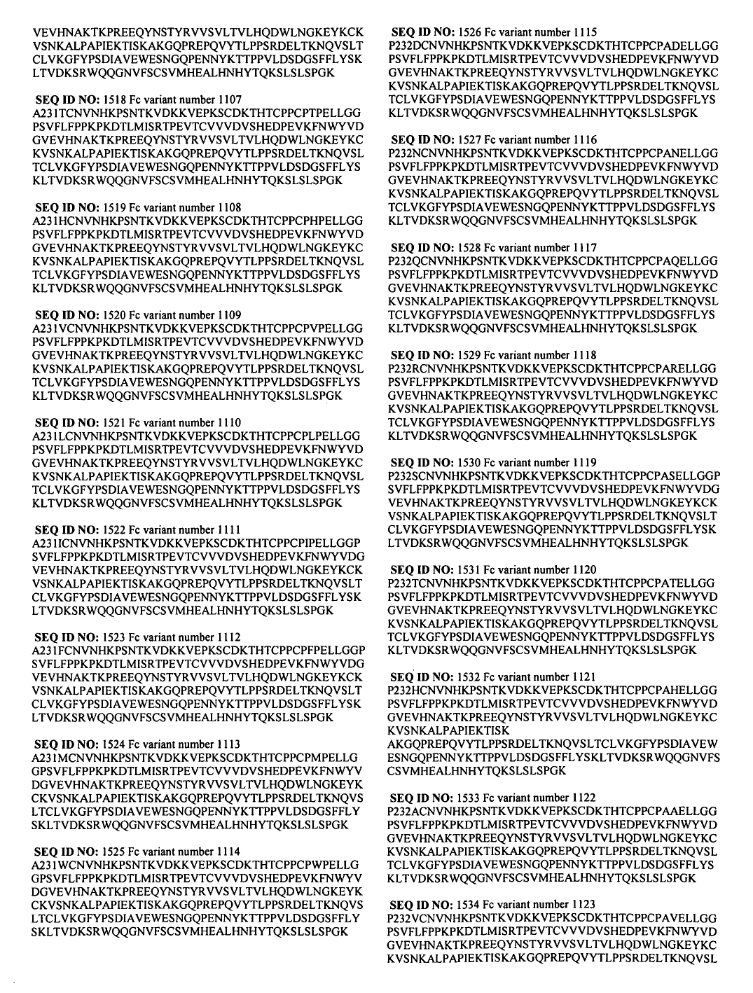

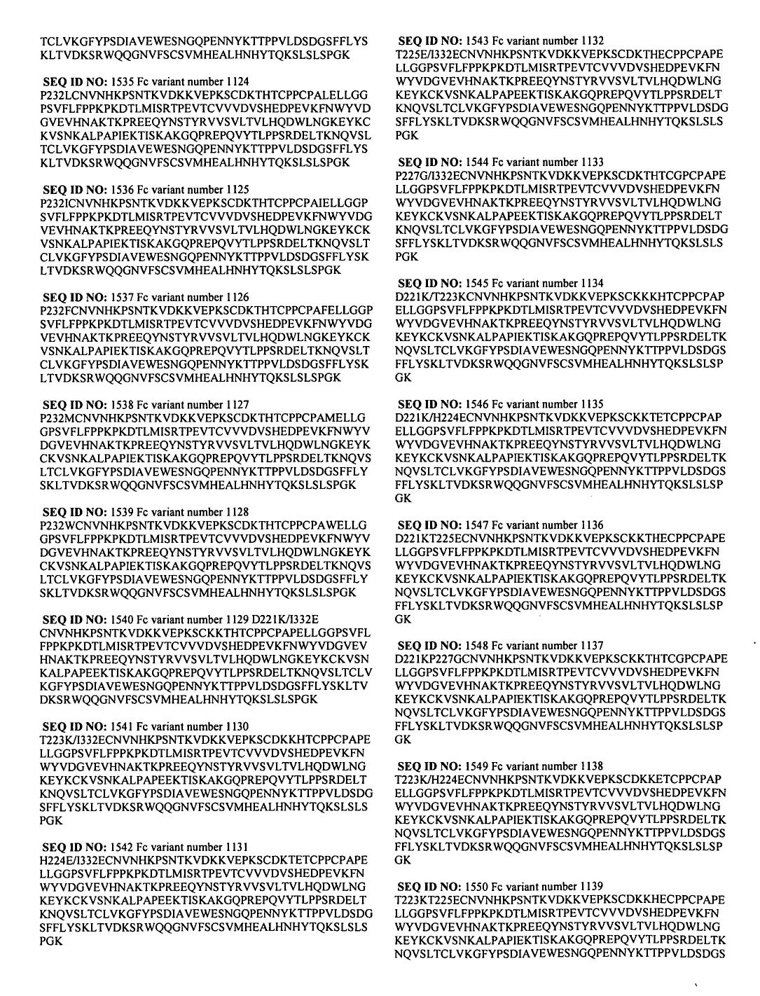

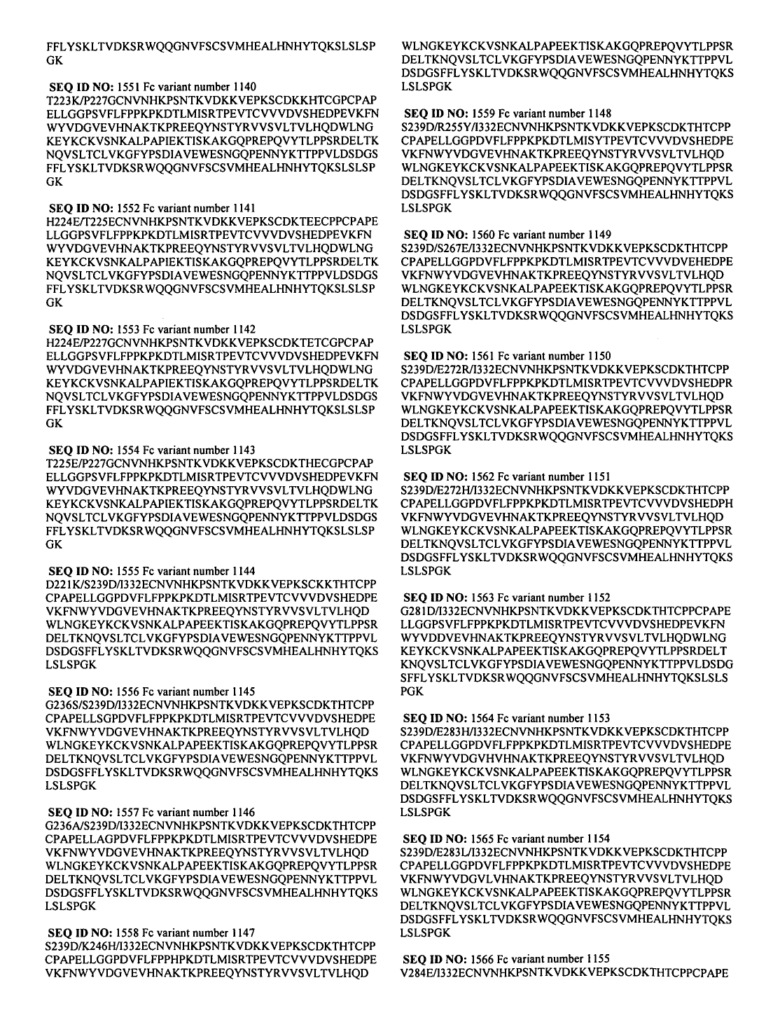

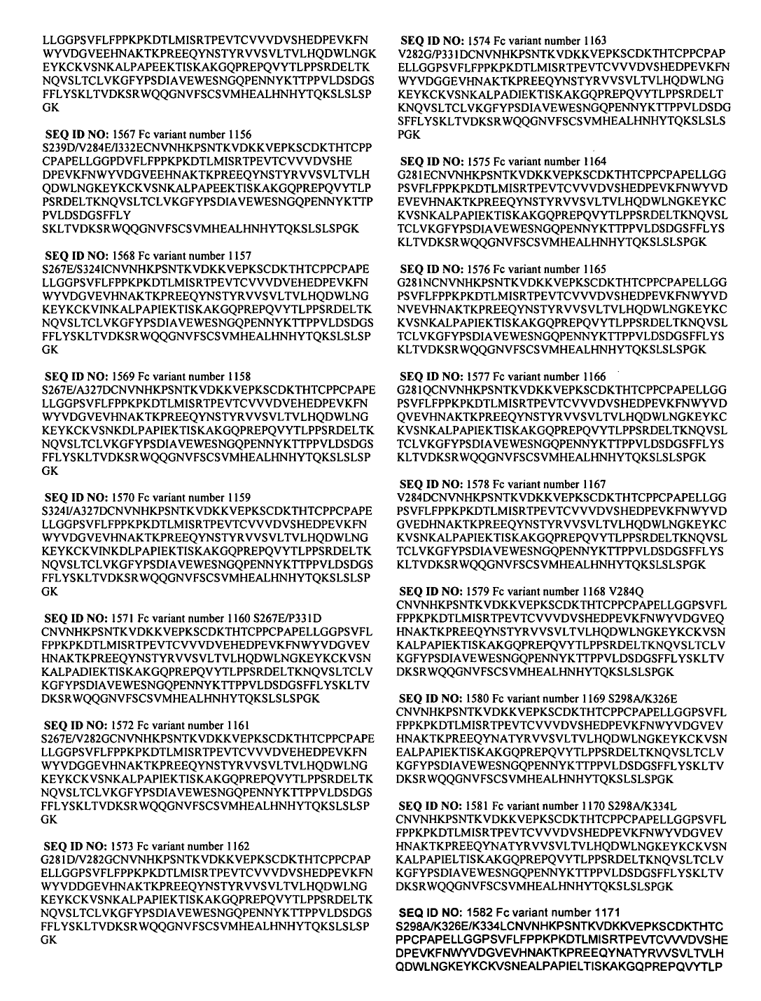

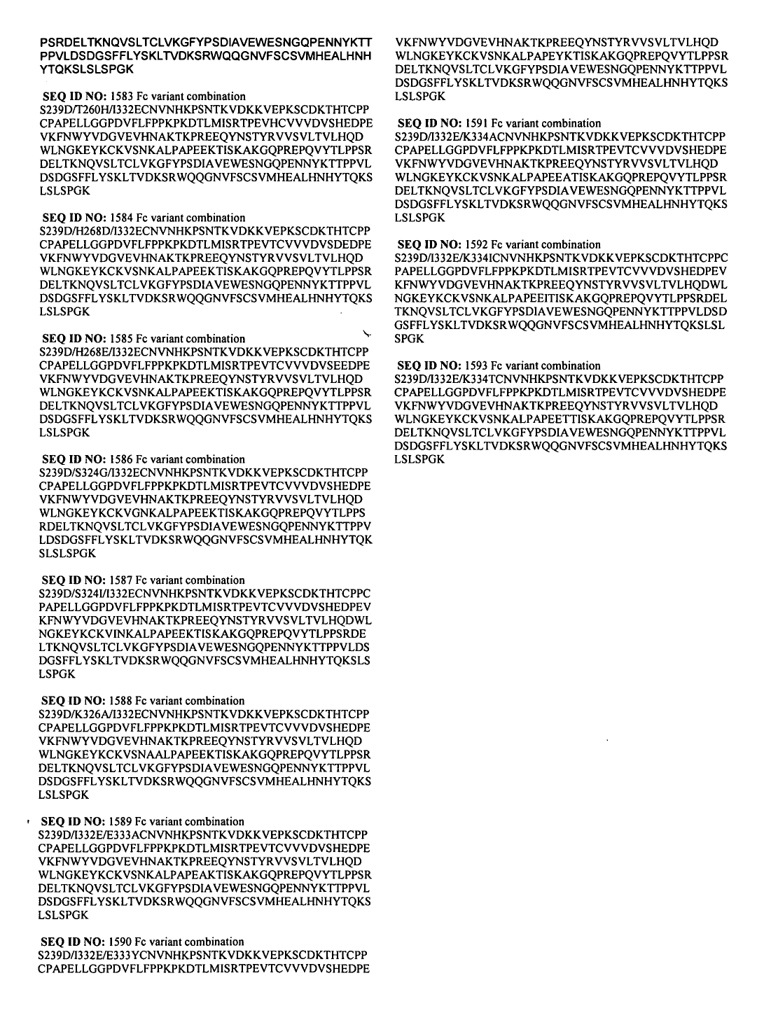

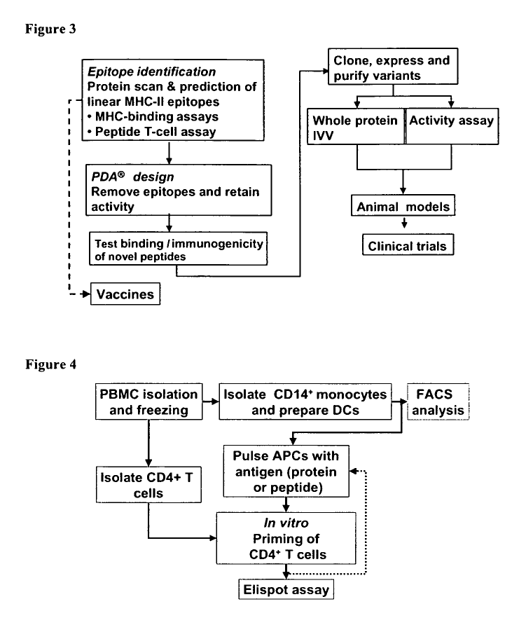

By “nine-mer peptide frame” and grammatical equivalents herein is meant a linear sequence of nine amino acids that is located in a protein of interest. nine-mer frames may be analyzed for their propensity to bind one or more class II MHC alleles. By “antigen” and grammatical equivalents is meant a molecule or molecules that are recognized by an antibody or Fc fusion. Examples of suitable antigens include, but are not limited to, 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33, (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3,-4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DCTRAIL R2 TNFRH2), TNFRST23 (DCTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors. By “allele” and grammatical equivalents herein is meant an alternative form of a gene. Specifically, in the context of class II MHC molecules, alleles comprise all naturally occurring sequence variants of DRA, DRB1, DRB3/4/5, DQA1, DQB1, DPA1, and DPB1 molecules. By “antibody or Fc fusion protein responsive disorders or conditions” and grammatical equivalents herein is meant diseases, disorders, and conditions that can benefit from treatment with an antibody or Fc fusion protein. Examples of antibody or Fc fusion protein-responsive disorders include, but are not limited to, autoimmune diseases, cancer, inflammatory disorders, infectious diseases, and additional conditions including but not limited to heart conditions such as congestive heart failure (CHF), myocarditis and other conditions of the myocardium; skin conditions such as rosecea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological conditions. By “autoimmune diseases” herein include allogenic islet graft rejection, alopecia areata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erthematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. More particular examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (eg. nasopharyngeal cancer, salivary gland carcinoma, and esophagael cancer), lung (eg. small-cell lung cancer, non-small cell lung cancer, adenocarcinoma of the lung and squamous carcinoma of the lung), digestive system (eg. gastric or stomach cancer including gastrointestinal cancer, cancer of the bile duct or biliary tract, colon cancer, rectal cancer, colorectal cancer, and anal carcinoma), reproductive system (eg. testicular, penile, or prostate cancer, uterine, vaginal, vulval, cervical, ovarian, and endometrial cancer), skin (eg. melanoma, basal cell carcinoma, squamous cell cancer, actinic keratosis), liver (eg. liver cancer, hepatic carcinoma, hepatocellular cancer, and hepatoma), bone (eg. osteoclastoma, and osteolytic bone cancers) additional tissues and organs (eg. pancreatic cancer, bladder cancer, kidney or renal cancer, thyroid cancer, breast cancer, cancer of the peritoneum, and Kaposi's sarcoma), and tumors of the vascular system (eg. angiosarcoma and hemagiopericytoma). By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, as illustrated in FIG. 1, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below. By “Fc polypeptide” as used herein is meant a polypeptide that comprises all or part of an Fc region. Fc polypeptides include antibodies, Fc fusions, isolated Fc molecules, and Fc fragments. By “Fc fusion” as used herein is meant a protein wherein one or more polypeptides or small molecules is operably linked to an Fc region or a derivative thereof. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200). An Fc fusion combines the Fc region of an immunoglobulin with a fusion partner, which in general can be any protein or small molecule. The role of the non-Fc part of an Fc fusion, i.e. the fusion partner, may be to mediate target binding, and thus it is functionally analogous to the variable regions of an antibody. The fusion partner may also play a role as a chemoattractant. Virtually any protein or small molecule may be linked to Fc to generate an Fc fusion. Protein fusion partners may include, but are not limited to, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target Such targets may be any molecule, preferrably an extracellular receptor, that is implicated in disease. Specific examples of particular drugs that may serve as Fc fusion partners can be found in L. S. Goodman et al., Eds., Goodman and Gilman's The Pharmacological Basis of Therapeutics (McGraw-Hill, New York, ed. 9, 1996). A variety of linkers, defined and described below, may be used to covalently link Fc to a fusion partner to generate an Fc fusion. By “germline” as used herein is meant the set of sequences that compose the natural genetic repertoire of a protein, and its associated alleles. By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, a variant protein can be said to have “reduced immunogenicity” if it elicits neutralizing or non-neutralizing antibodies in lower titer or in fewer patients than the wild type protein. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A variant protein also can be said to have “reduced immunogenicity” if it shows decreased binding to one or more MHC alleles or if it induces T-cell activation in a decreased fraction of patients relative to the parent protein. In a preferred embodiment, the probability of T-cell activation is decreased by at least 5%, with at least 50% or 90% decreases being especially preferred. By “inflammatory disorders” herein include acute respiratory distress syndrome (ARDS), acute septic arthritis, allergic encephalomyelitis, allergic rhinitis, allergic vasculitis, allergy, asthma, atherosclerosis, chronic inflammation due to chronic bacterial or viral infectionis, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy. By “infectious diseases” herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infections diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibria cholerae, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium marneffei, and the like. Furthermore, infectious diseases may be caused by protozoa and parasites such as chlamydia, kokzidioa, leishmania, malaria, rickettsia, trypanosoma, and the like. By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. By “matrix method” and grammatical equivalents thereof herein is meant a method for calculating peptide-MHC affinity in which a matrix is used that contains a score for each possible residue at each position in the peptide, interacting with a given MHC allele. The binding score for a given peptide-MHC interaction is obtained by summing the matrix values for the amino acids observed at each position in the peptide. By “MHC-binding agretopes” and grammatical equivalents herein is meant peptides that are capable of binding to one or more class II MHC alleles with appropriate affinity to enable the formation of MHC-peptide-T-cell receptor complexes and subsequent T-cell activation. MHC-binding agretopes are linear peptide sequences that comprise at least approximately 9 residues. By “parent protein” as used herein is meant a protein that is subsequently modified to generate a variant protein. Said parent protein may be a wild-type or naturally occurring protein from any organism, including but not limited to humans, mice, rats, rabbits, camels, llamas, dromedaries, monkeys, preferably mammals and most preferably humans and mice and rats. Said parent protein may also be a variant or engineered protein, including but not limited to a chimeric antibody, a humanized antibody, or an antibody or Fc fusion obtained using a display technology. “Parent protein” may refer to the protein itself, compositions that comprise the parent protein, or any amino acid sequence that encodes it. Accordingly, “parent protein” as used herein is meant an antibody or Fc fusion protein that is modified to generate a variant antibody or Fc fusion protein. By “patient” herein is meant both humans and other animals, particularly mammals, and organisms. Thus the methods are applicable to both human therapy and veterinary applications. In the preferred embodiment the patient is a mammal, and in the most preferred embodiment the patient is human. By “position” as used herein is meant a location in the sequence of a protein. Positions may be numbered sequentially, or according to an established format, for example the EU index (Kabat et al., 1991, Sequences of Proteins of Immunological Interest, 5th Ed., United States Public Health Svice, National Institutes of Health, Bethesda). For example, position 297 is a position in the human antibody IgG1. Corresponding positions are determined as outlined above, generally through alignment with other parent sequences. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized. By “treatment” herein is meant to include therapeutic treatment, as well as prophylactic, or suppressive measures for the disease or disorder. Thus, for example, successful administration of a variant antibodies and Fc fusion proteins protein prior to onset of the disease may result in treatment of the disease. As another example, successful administration of a variant antibodies and Fc fusion proteins protein after clinical manifestation of the disease to combat the symptoms of the disease comprises “treatment” of the disease. “Treatment” also encompasses administration of a variant antibodies and Fc fusion proteins protein after the appearance of the disease in order to eradicate the disease. Successful administration of an agent after onset and after clinical symptoms have developed, with possible abatement of clinical symptoms and perhaps amelioration of the disease, further comprises “treatment” of the disease. Those “in need of treatment” include mammals already having the disease or disorder, as well as those prone to having the disease or disorder, including those in which the disease or disorder is to be prevented. By “variant antibody and Fc fusion protein nucleic acids” and grammatical equivalents herein are meant nucleic acids that encode a variant antibody or Fc fusion protein. Due to the degeneracy of the genetic code, an extremely large number of nucleic acids may be made, all of which encode a variant antibody or Fc fusion protein of the present invention, by simply modifying the sequence of one or more codons in a way which does not change the amino acid sequence of the variant antibody or Fc fusion protein. By “variant antibodies and Fc fusion proteins” and grammatical equivalents thereof herein are meant non-naturally occurring antibodies and Fc fusion proteins which differ from the wild type or parent antibody or Fc fusion protein by at least 1 amino acid insertion, deletion, or substitution. Antibody and Fc fusion protein variants are characterized by the predetermined nature of the variation, a feature that sets them apart from naturally occurring allelic or interspecies variation of antibody and Fc protein sequences. The variant antibodies and Fc fusion proteins may contain insertions, deletions, and/or substitutions at the N-terminus, C-terminus, or internally. In a preferred embodiment, variant antibodies and Fc fusion proteins have at least 1 residue that differs from the naturally occurring antibody or Fc fusion protein sequence, with at least 2, 3, 4, or 5 different residues being more preferred. Variant antibodies and Fc fusion proteins may contain further modifications, for instance mutations that alter stability or solubility or which enable or prevent posttranslational modifications such as PEGylation or glycosylation. Variant antibodies and Fc fusion proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild type sequence is SEQ_ID NO:1.

Antibody Sequence and Structure

Antibodies are immunological proteins that bind a specific antigen. In most mammals, including humans and mice, antibodies are constructed from paired heavy and light polypeptide chains. Each chain is made up of individual immunoglobulin (Ig) domains, and thus the generic term immunoglobulin is used for such proteins. Each chain is made up of two distinct regions, referred to as the variable and constant regions. The light and heavy chain variable regions show significant sequence diversity between antibodies, and are responsible for binding the target antigen. The constant regions show less sequence diversity, and are responsible for binding a number of natural proteins to elicit important biochemical events. In humans, there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region. FIG. 1 shows an IgG1 antibody, used here as an example to describe the general structural features of immunoglobulins. IgG antibodies are tetrameric proteins composed of two heavy chains and two light chains. The IgG heavy chain is composed of four immunoglobulin domains linked from N- to C-terminus in the order V_{H}-Cγ1-Cγ2-Cγ3, referring to the heavy chain variable domain, constant gamma 1 domain, constant gamma 2 domain, and constant gamma 3 domain respectively. The IgG light chain is composed of two immunoglobulin domains linked from N- to C-terminus in the order V_{L}-C_{L}, referring to the light chain variable domain and the light chain constant domain respectively.

In certain variations, antibodies are substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes respectively. Exemplary antibodies include full length antibodies and antibody fragments, and may refer to a natural antibody from any organism, an engineered antibody, or an antibody generated recombinantly for experimental, therapeutic, or other purposes as further defined below. Exemplary antibodies also include antibody fragments, as are known in the art, such as Fab, Fab′, F(ab′)₂, Fv, scFv, or other antigen-binding subsequences of antibodies, either produced by the modification of whole antibodies or those synthesized de novo using recombinant DNA technologies. Particularly preferred are full length antibodies that comprise Fc variants as described herein. Antibodies also include monoclonal and polyclonal antibodies. Antibodies can be antagonists, agonists, neutralizing, inhibitory, or stimulatory.

The variable region of an antibody contains the antigen binding determinants of the molecule, and thus determines the specificity of an antibody for its target antigen. The variable region is so named because it is the most distinct in sequence from other antibodies within the same class. The majority of sequence variability occurs in the complementarity determining regions (CDRs). There are 6 CDRs total, three each per heavy and light chain, designated V_{H} CDR1, V_{H} CDR2, V_{H} CDR3, V_{L} CDR1, V_{L} CDR2, and V_{L} CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens. A number of high-resolution structures are available for a variety of variable region fragments from different organisms, some unbound and some in complex with antigen. The sequence and structural features of antibody variable regions are well characterized (Morea et al., 1997, Biophys Chem 68:9-16; Morea et al., 2000, Methods 20:267-279), and the conserved features of antibodies have enabled the development of a wealth of antibody engineering techniques (Maynard et al., 2000, Annu Rev Biomed Eng 2:339-376). For example, it is possible to graft the CDRs from one antibody, for example a murine antibody, onto the framework region of another antibody, for example a human antibody. This process, referred to in the art as “humanization”, enables generation of less immunogenic antibody therapeutics from nonhuman antibodies. Fragments comprising the variable region can exist in the absence of other regions of the antibody, including for example the antigen binding fragment (Fab) comprising V_{H}-Cγ1 and V_{H}-C_{L}, the variable fragment (Fv) comprising V_{H} and V_{L}, the single chain variable fragment (scFv) comprising V_{H} and V_{L} linked together in the same chain, as well as a variety of other variable region fragments (Little et al., 2000, Immunol Today 21:364-370).

Identification of MHC-Binding Agretopes in Antibodies and Fc Fusion Proteins

MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naïve T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in antibodies and Fc fusion proteins.

Sequence-based information can be used to determine a binding score for a given peptide-MHC interaction (see, e.g., Mallios, Bioinformatics 15: 432-439 (1999); Mallios, Bioinformatics 17: p 942-948 (2001); Sturniolo et al. Nature Biotech. 17: 555-561(1999)). It is possible to use structure-based methods in which a given peptide is computationally placed in the peptide-binding groove of a given MHC molecule and the interaction energy is determined (for example, see WO 98/59244 and WO 02/069232). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see, e.g., WO 02/77187).

In a preferred embodiment, MHC-binding propensity scores are calculated for each 9-residue frame along the antibodies and Fc fusion proteins sequence using a matrix method (see Sturniolo et al., supra; Marshall et al., J. Immunol. 154: 5927-5933 (1995), and Hammer et al., J. Exp. Med. 180: 2353-2358 (1994)). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each nine-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding agretopes are identified as the nine-mer frames that bind to several class II MHC alleles. In an especially preferred embodiment, MHC-binding agretopes are predicted to bind at least 10 alleles at 5% threshold and/or at least 5 alleles at 1% threshold. Such nine-mer frames may be especially likely to elicit an immune response in many members of the human population.

In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003).

In a preferred embodiment, MHC binding agretopes are predicted for MHC heterodimers comprising highly prevalent MHC alleles. Class II MHC alleles that are present in at least 10% of the US population include but are not limited to: DPA1*0103, DPA1*0201, DPB1*0201, DPB1*0401, DPB1*0402, DQA1*0101, DQA1*0102, DQA1*0201, DQA1*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA1*0104, DPA1*0302, DPA1*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA1*0103, DQA1*0104, DQA1*0301, DQA1*0302, DQA1*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database (.ebi.ac.uk/imgt/hla/).

MHC binding agretopes may also be predicted for MHC heterodimers comprising less prevalent alleles. Information about MHC alleles in humans and other species can be obtained, for example, from the IMGT/HLA sequence database (.ebi.ac.uk/imgt/hla/).

In an especially preferred embodiment, an immunogenicity score is determined for each peptide, wherein said score depends on the fraction of the population with one or more MHC alleles that are hit at multiple thresholds. For example, the equation
IScore=N(W₁P₁+W₃P₃+W₅P₅)
may be used, where P₁ is the percent of the population hit at 1%, P₃ is the percent of the population hit at 3%, P₅ is the percent of the population hit at 5%, each W is a weighting factor, and N is a normalization factor. In a preferred embodiment, W₁=10, W₃=5, W₅=2, and N is selected so that possible scores range from 0 to 100. In this embodiment, agretopes with IScore greater than or equal to 10 are preferred and agretopes with IScore greater than or equal to 25 are especially preferred.

In an additional preferred embodiment, MHC-binding agretopes are identified as the nine-mer frames that are located among “nested” agretopes, or overlapping 9-residue frames that are each predicted to bind a significant number of alleles. Such sequences may be especially likely to elicit an immune response.

Preferred MHC-binding agretopes are those agretopes that are predicted to bind, at a 3% threshold, to MHC alleles that are present in at least 5% of the population. Preferred MHC-binding agretopes in the constant regions of human IgG1, IgG2, IgG3, and IgG4 include, but are not limited to, agretope 5 (residues 149-157, IgG1, IgG2, IgG3, and IgG4), agretope 7 (residues 174-182, IgG1, IgG2, IgG3, and IgG4), agretope 8 (residues 179-187, IgG1, IgG2, IgG3, and IgG4), agretope 9 (residues 180-188, IgG1, IgG2, IgG3, and IgG4), agretope 10 (residues 182-190, IgG1, IgG2, IgG3, and IgG4), agretope 11a (residues 185-193, IgG1, IgG3, and IgG4), agretope 11b (residues 185-193, IgG2), agretope 13 (residues 215-221, IgG3), agretope 14 (residues 234-242, IgG4), agretope 16 (residues 251-259, IgG1, IgG2, IgG3, and IgG4), agretope 17b (residues 262-270, IgG4), agretope 18 (residues 277-285, IgG1, IgG2, IgG3, and IgG4), agretope 19a (residues 300-308, IgG1 and IgG4), agretope 19b (residues 300-308, IgG2 and IgG3), agretope 20a (residues 302-310, IgG1, IgG3, and IgG4), agretope 20b (residues 302-310, IgG2), agretope 21a (residues 303-311, IgG1, IgG3, and IgG4), agretope 23 (residues 369-377, IgG1, IgG2, IgG3, and IgG4), agretope 24a (residues 404-412, IgG1 and IgG2), agretope 24b (residues 404-412, IgG4), and agretope 28a (residues 432-440, IgG1, IgG2, and IgG4).

Especially preferred MHC-binding agretopes are those agretopes that are predicted to bind, at a 1% threshold, to MHC alleles that are present in at least 10% of the population. Especially preferred MHC-binding agretopes in the constant regions of human IgG1, IgG2, IgG3, and IgG4 include, but are not limited to, agretope 5 (residues 149-157, IgG1, IgG2, IgG3, and IgG4), agretope 16 (residues 251-259, IgG1, IgG2, IgG3, and IgG4), agretope 18 (residues 277-285, IgG1, IgG2, IgG3, and IgG4), agretope 19a (residues 300-308, IgG1 and IgG4), agretope 19b (residues 300-308, IgG2 and IgG3), agretope 21a (residues 303-311, IgG1, IgG3, and IgG4), agretope 24a (residues 404-412, IgG1 and IgG2), agretope 24b (residues 404-412, IgG4), and agretope 28a (residues 432-440, IgG1, IgG2, and IgG4).

Additional especially preferred MHC-binding agretopes are those agretopes whose sequences partially overlap with additional MHC-binding agretopes. Sets of overlapping MHC-binding agretopes in the constant regions of human IgG1, IgG2, IgG3, and IgG4 include, but are not limited to, residues 174-193 (IgG1, IgG2, IgG3, and IgG4), residues 300-310 (IgG2), and residues 300-311 (IgG1, IgG3, and IgG4).

Alternate preferred MHC-binding agretopes are those agretopes that have IScore greater than or equal to 10 in the constant regions of human IgG1, IgG2, IgG3, and IgG4 include, but are not limited to, agretope 5 (residues 149-157, IgG1, IgG2, IgG3, and IgG4), agretope 7 (residues 174-182, IgG1, IgG2, IgG3, and IgG4), agretope 9 (residues 180-188, IgG1, IgG2, IgG3, and IgG4), agretope 11a (residues 185-193, IgG1, IgG3, and IgG4), agretope 14 (residues 234-242, IgG4), agretope 16 (residues 251-259, IgG1, IgG2, IgG3, and IgG4), agretope 17b (residues 262-270, IgG4), agretope 18 (residues 277-285, IgG1, IgG2, IgG3, and IgG4), agretope 19a (residues 300-308, IgG1 and IgG4), agretope 19b (residues 300-308, IgG2 and IgG3), agretope 20a (residues 302-310, IgG1, IgG3, and IgG4), agretope 20b (residues 302-310, IgG2), agretope 21a (residues 303-311, IgG1, IgG3, and IgG4), agretope 23 (residues 369-377, IgG1, IgG2, IgG3, and IgG4), agretope 24a (residues 404-412, IgG1 and IgG2), agretope 24b (residues 404-412, IgG4), and agretope 28a (residues 432-440, IgG1, IgG2, and IgG4).

Alternate especially preferred MHC-binding agretopes are those agretopes that have IScore greater than or equal to 25. Preferred MHC-binding agretopes in the constant regions of human IgG1, IgG2, IgG3, and IgG4 include, but are not limited to, agretope 16 (residues 251-259, IgG1, IgG2, IgG3, and IgG4), agretope 19a (residues 300-308, IgG1 and IgG4), agretope 19b (residues 300-308, IgG2 and IgG3), agretope 24a (residues 404-412, IgG1 and IgG2), agretope 24b (residues 404-412, IgG4), and agretope 28a (residues 432-440, IgG1, IgG2, and IgG4).

Confirmation of MHC-Binding Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naive T cells and antigen presenting cells from matched donors can be stimulated with a peptide containing an agretope of interest, and T-cell activation can be monitored. It is also possible to first stimulate T cells with the whole protein of interest, and then re-stimulate with peptides derived from the whole protein. If sera are available from patients who have raised an immune response to antibodies and Fc fusion proteins, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Design of Active, Less-Immunogenic Variants

In a preferred embodiment, the above-determined MHC-binding agretopes are replaced with alternate amino acid sequences to generate active variant antibodies and Fc fusion proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more nine-mer agretope identified above, one or more possible alternate nine-mer sequences are analyzed for immunogenicity as well as structural and functional compatibility. The preferred alternate nine-mer sequences are then defined as those sequences that have low predicted immunogenicity and a high probability of being structured and active. It is possible to consider only the subset of nine-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Maximizing Tolerized Human Sequence Content

To date, the most successful efforts in reducing antibody immunogenicity have been methods such as chimerization and humanization that replace non-human sequence content with human sequence content. However, even fully human antibodies may be immunogenic. This may arise from agretopes located partially or fully in the variable domains, especially the CDRs, as novel sequence diversity may be sampled in these regions. In addition, it is possible that some germline variable domain sequences are not fully tolerized. This may be especially likely for germline sequences that are rarely present in productively rearranged antibodies. Similarly, in Fc fusions, immunogenicity may arise from agretopes located partially or fully in the linker region or at the junction between the Fc and the fusion partner.

In a preferred embodiment, antibodies and Fc fusion proteins are prepared to maximize abundantly expressed human sequence content. For example, it has been reported that VH 1-3, VH 3-23, VLK A27, VLK 2-1, and VLL 14-7 are frequently found in productively recombined human antibodies (de Wildt et al. 1999 J. Mol. Biol. 285: 895-901; Ignatovich et al. 1999 J. Mol. Biol. 294: 457-465). Accordingly, agretopes in these sequences may be fully tolerized. Engineered antibodies, including antibodies using a consensus sequence for the variable domain frameworks, may be designed such that the only predicted agretopes are those also present in abundance in the endogenous antibody repertoire.

Substitution Matrices

In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see, e.g., Bowie et al. Science 247: 1306-1310 (1990), Bowie and Sauer Proc. Nat. Acad. Sci. USA 86: 2152-2156 (1989), and Reidhaar-Olson and Sauer Proteins 7: 306-316 (1990)). However, non-conservative mutations can destabilize protein structure and reduce activity (see, e.g., Lim et al. Biochem. 31: 4324-4333 (1992)). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997)). The use of substitution matrices to design peptides with improved properties has been disclosed; see Adenot et al. J. Mol. Graph. Model. 17: 292-309 (1999).

Substitution matrices include, but are not limited to, the BLOSUM matrices (Henikoff and Henikoff, Proc. Nat. Acad. Sci. USA 89: 10917 (1992), the PAM matrices, the Dayhoff matrix, and the like. For a review of substitution matrices, see, e.g., Henikoff Curr. Opin. Struct. Biol. 6: 353-360 (1996). It is also possible to construct a substitution matrix based on an alignment of a given protein of interest and its homologs; see, e.g., Henikoff and Henikoff Comput. Appl. Biosci. 12:135-143 (1996).

In a preferred embodiment, each of the substitution mutations that are considered has a BLOSUM62 score of zero or higher. According to this metric, preferred substitutions include, but are not limited to:

In addition, it is preferred that the total BLOSUM62 score of an alternate sequence for a nine residue MHC-binding agretope is decreased only modestly when compared to the BLOSUM62 score of the wild type nine residue agretope. In a preferred embodiment, the score of the variant nine mer is at least 50% of the wild type score, with at least 67%, 80% or 90% being especially preferred.

Alternatively, alternate sequences can be selected that minimize the absolute reduction in BLOSUM score; for example it is preferred that the score decrease for each nine-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine Substitutions

In an alternate embodiment, one or more alanine substitutions may be made, regardless of whether an alanine substitution is conservative or non-conservative. As is known in the art, incorporation of sufficient alanine substitutions may be used to disrupt intermolecular interactions.

Residues Critical for the Structure or Function of Antibodies or Fc Fusion Proteins

In a preferred embodiment, variant nine-mers are selected such that residues that have been or can be identified as especially critical for maintaining the structure or function of antibodies and Fc fusion proteins retain their wild type identity. In alternate embodiments, it may be desirable to produce variant antibodies and Fc fusion proteins that do not retain wild type activity. In such cases, residues that have been identified as critical for function may be specifically targeted for modification.

The variable region of an antibody contains the antigen binding determinants of the molecule, and thus determines the specificity of an antibody for its target antigen. The most important determinants are the CDRs. For Fc fusions, key functional residues will depend on the fusion partner chosen and may be determined by standard methods known in the art.

The Fc region of an antibody or Fc fusion protein interacts with a number of Fc receptors and ligands, imparting an array of important functional capabilities referred to as effector functions. One key class of molecules are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIb-NA2) (Jefferis et al., 2002, Immunol Lett 82:57-65). All FcγRs bind the same region on Fc, at the N-terminal end of the Cγ2 domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K)(Sondermann et al., 2000, Nature 406:267-273.) (pdb accession codes 1IIS and 1IIX)(Radaev et al., 2001, J Biol Chem 276:16469-16477), as well as has the structure of the human IgE Fc/FcεRIα complex (pdb accession code 1 F6A)(Garman et al., 2000, Nature 406:259-266). Antibody or Fc fusion protein residues that mediate (either directly or indirectly) binding to Fc gamma receptors include, but are not limited to, positions 230, 233, 234, 235, 236, 237, 239, 240, 241, 243, 244, 245, 247, 262, 263, 264, 265, 266, 267, 269, 270, 272, 273, 274, 275, 276, 278, 283, 296, 297, 298, 299, 302, 313, 318, 323, 324, 325, 326, 327, 328, 329, 330, 332, and 333.

An overlapping but separate site on Fc serves as the interface for the complement protein C1q. In the same way that Fc/FcγR binding mediates ADCC, Fc/C1q binding mediates complement dependent cytotoxicity (CDC). C1q forms a complex with the serine proteases C1r and C1s to form the C1 complex. C1q is capable of binding six antibodies, although binding to two IgGs is sufficient to activate the complement cascade. There is currently no structure available for the Fc/C1q complex; however, mutagenesis studies have mapped the binding site on human IgG for C1q to a region involving residues D270, K322, K326, P329, and P331, and E333 (Idusogie et al., 2000, J Immunol 164:4178-4184; Idusogie et al., 2001, J Immunol 166:2571-2575). Additional residues that may mediate C1q binding include, but are not limited to, L234 and L235.

A site on Fc between the Cγ2 and Cγ3 domains mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481) provide insight into the interaction of Fc with these proteins. Residues that may mediate FcRn binding include, but are not limited to, K248, D249, T250, L251, M252, I253, S254, R255, T256, P257, N286, K288, T307, L309, H310, Q311, L314, D315, E430, H433, N434, H435, and Y436.

A key feature of the Fc region is the conserved N-linked glycosylation that occurs at N297. This carbohydrate, or oligosaccharide as it is sometimes referred, plays a critical structural and functional role for the antibody, and is one of the principle reasons that antibodies must be produced using mammalian expression systems. While not wanting to be limited to one theory, it is believed that the structural purpose of this carbohydrate may be to stabilize or solubilize Fc, determine a specific angle or level of flexibility between the Cγ3 and Cγ2 domains, keep the two Cγ2 domains from aggregating with one another across the central axis, or a combination of these. Efficient Fc binding to FcγR and C1q requires this modification, and alterations in the composition of the N297 carbohydrate or its elimination affect binding to these proteins (Umaña et al., 1999, Nat Biotechnol 17:176-180; Davies et al., 2001, Biotechnol Bioeng 74:288-294; Mimura et al., 2001, J Biol Chem 276:45539-45547.; Radaev et al., 2001, J Biol Chem 276:16478-16483; Shields et al., 2001, J Biol Chem 276:6591-6604; Shields et al., 2002, J Biol Chem 277:26733-26740; Simmons et al., 2002, J Immunol Methods 263:133-147). Yet the carbohydrate makes little if any specific contact with FcγRs (Radaev et al., 2001, J Biol Chem 276:16469-16477), indicating that the functional role of the N297 carbohydrate in mediating Fc/FcγR binding may be via the structural role it plays in determining the Fc conformation. This is supported by a collection of crystal structures of four different Fc glycoforms, which show that the composition of the oligosaccharide impacts the conformation of Cγ2 and as a result the Fc/FcγR interface (Krapp et al., 2003, J Mol Biol 325:979-989).

Protein Design Methods

Protein design methods and MHC agretope identification methods may be used together to identify stable, active, and minimally immunogenic protein sequences (see WO03/006154). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in FIG. 5 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

A wide variety of methods is known for generating and evaluating sequences. These include, but are not limited to, sequence profiling (Bowie and Eisenberg, Science 253(5016): 164-70, (1991)), residue pair potentials (Jones, Protein Science 3: 567-574, (1994)), and rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)).

Protein Design Automation® (PDA®) Technology

In an especially preferred embodiment, rational design of improved CD40L variants is achieved by using Protein Design Automation® (PDA®) technology. (See U.S. Pat. Nos. 6,188,965; 6,269,312; 6,403,312; 6,708,120; 6,801,861; 6,804,611; 6,972,356; WO98/47089 and U.S. Ser. Nos. 09/652,699; 09/866,511; 09/990,769; 09/812,034; 09/877,695; 10/057,552; 10/071,859; 10/888,748; 09/782,004; 09/927,790; 10/218,102; 10/218,102; 10/666,311; 10/666,307; and 60/602,546, filed Aug. 17, 2004, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

PDA® utilizes three-dimensional structural information. In a most preferred embodiment, the structure of antibodies and Fc fusion proteins is determined using X-ray crystallography or NMR methods, which are well known in the art. Numerous high resolution structures of antibodies and Fc fusions have been determined, both in isolation and bound to various antigens and effector molecules. Relevant structures include but are not limited to PDB ascession codes 1CE1, 1FVE, and 1L7I (humanized Fab); 1DN2 (human IgG1 Fc); 1E4K, 1IIS and 1IIX (human Fc bound to the extracellular domain of human FcγRIIIb; and 1F6A (human IgE Fc/FcεRIα complex).

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

Combining Immunogenicity Reduction Strategies

In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. Nos. 09/903,378; 10/039,170; 10/339,788; 10/638,995; and 10/754,296.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity. See for example, U.S. Ser. Nos. 09/903,378; 10/039,170; 10/339,788; 10/638,995; and 10/754,296.

In a further preferred embodiment, an antibody remastering approach is used to mimimize the presence of non-human linear and tertiary epitopes (see, e.g., U.S. Ser. No. 11/004,590. filed Dec. 3, 2004, Ser. No. 10/______, filed Dec. 6, 2004, and entitled Methods Of Generating Variant Proteins With Increased Host String Content And Compositions Thereof).

In an additional preferred embodiment, a variant protein with reduced binding affinity for one or more class II MHC alleles is further modified by derivatization with PEG or another molecule. As is known in the art, PEG may sterically interfere with antibody binding or improve protein solubility, thereby reducing immunogenicity. In an especially preferred embodiment, rational PEGylation methods are used. See for example, U.S. Ser. Nos. 10/811,492 and 10/820,466.

In a preferred embodiment, PDA® technology and matrix method calculations are used to remove more than one MHC-binding agretope from a protein of interest.

Optimization of Additional Factors

The antibodies and Fc fusion proteins of the invention may be further modified to confer additional desired properties. For example, modifications may be made to provide altered or optimized effector functions, including but not limited to ADCC, ADCP, and CDC, optimized pharmacokinetics including serum half-life and bioavailability, improved affinity or specificity for the target antigen, enhanced stability and solubility (including resistance to proteolysis, deamidation, oxidation, methylation, and hydroxylation), increased expression yield, and the like. Additional modifications include modifications that remove or reduce the ability of heavy chains to form inter-chain disulfide linkages, modifications that alter oligomerization state, and substitutions that enable site-specific covalent modification.

Generating the Variants

Variant antibodies and Fc fusion proteins of the invention and nucleic acids encoding them may be produced using a number of methods known in the art.

Preparing Nucleic Acids

In one embodiment of the present invention, the library sequences are used to create nucleic acids that encode the member sequences, and that may then be cloned into host cells, expressed and assayed, if desired. Thus, nucleic acids, and particularly DNA, may be made that encode each member protein sequence. These practices are carried out using well-known procedures. For example, a variety of methods that may find use in the present invention are described in Molecular Cloning—A Laboratory Manual, 3^{rd} Ed. (Maniatis, Cold Spring Harbor Laboratory Press, New York, 2001), and Current Protocols in Molecular Biology (John Wiley & Sons).

As will be appreciated by those skilled in the art, the generation of exact sequences for a library comprising a large number of sequences is potentially expensive and time consuming. Accordingly, there are a variety of techniques that may be used to efficiently generate libraries of the present invention. Such methods that may find use in the present invention are described or referenced in U.S. Pat. No. 6,403,312; U.S. Ser. No. 09/782,004; U.S. Ser. No. 09/927,790; U.S. Ser. No. 10/218,102; PCT WO 01/40091; and PCT WO 02/25588. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode the variant antibodies or Fc fusion proteins.

Expression Vectors

The nucleic acids that encode the antibodies or Fc fusion protein variants of the present invention may be incorporated into an expression vector in order to express the protein. A variety of expression vectors may be utilized for protein expression. Expression vectors may comprise self-replicating extra-chromosomal vectors or vectors which integrate into a host genome. Expression vectors are constructed to be compatible with the host cell type. Thus, expression vectors that find use in the present invention include but are not limited to those which enable protein expression in mammalian cells, bacteria, insect cells, yeast, and in in vitro systems. As is known in the art, a variety of expression vectors are available, commercially or otherwise, that may find use in the present invention for expressing Fc variant proteins.

Expression vectors typically comprise a protein operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the antibody or Fc fusion protein variant, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

Tags and Fusions that Facilitate Protein Production

Antibody or Fc fusion protein variants may be operably linked to a fusion partner to enable targeting of the expressed protein, purification, screening, display, and the like. Fusion partners may be linked to the antibody or Fc fusion protein variant sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS. (SEQ ID NO: 1938)

A fusion partner may be a targeting or signal sequence that directs the antibody or Fc fusion protein to a desired cellular location or to the extracellular media. As is known in the art, certain signaling sequences may target a protein to be either secreted into the growth media, or into the periplasmic space, located between the inner and outer membrane of the cell.

A fusion partner may also be a sequence that encodes a peptide or protein that enables purification and/or screening. Such fusion partners include but are not limited to polyhistidine tags (His-tags) (for example H₆ and H₁₀ or other tags for use with Immobilized Metal Affinity Chromatography (IMAC) systems (e.g. Ni⁺² affinity columns)), GST fusions, MBP fusions, Strep-tag, the BSP biotinylation target sequence of the bacterial enzyme BirA, and epitope tags which are targeted by antibodies (for example c-myc tags, flag-tags, and the like). As will be appreciated by those skilled in the art, such tags may be useful for purification, for screening, or both. For example, an antibody or Fc fusion protein variant may be purified using a His-tag by immobilizing it to a Ni⁺² affinity column, and then after purification the same His-tag may be used to immobilize the antibody to a Ni⁺² coated plate to perform an ELISA or other binding assay (as described below).

A fusion partner may enable the use of a selection method to screen antibody or Fc fusion protein variants (see below). Fusion partners that enable a variety of selection methods are well-known in the art, and all of these find use in the present invention. For example, by fusing the members of an Fc variant library to the gene III protein, phage display can be employed (Kay et al., Phage display of peptides and proteins: a laboratory manual, Academic Press, San Diego, Calif., 1996; Lowman et al., 1991, Biochemistry 30:10832-10838; Smith, 1985, Science 228:1315-1317).

Fusion partners may enable antibody or Fc fusion protein variants to be labeled. Alternatively, a fusion partner may bind to a specific sequence on the expression vector, enabling the fusion partner and associated antibody or Fc fusion protein variant to be linked covalently or noncovalently with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466 describe such a fusion partner and technique that may find use in the present invention.

Transfection

The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

Expression

The Fc variants of the present invention may be produced by culturing a host cell transformed with nucleic acid, preferably an expression vector, containing nucleic acid encoding the Fc variants, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC® cell line catalog, available from the American Type Culture Collection.

In a preferred embodiment, the Fc variants are expressed in mammalian expression systems, including systems in which the expression constructs are introduced into the mammalian cells using virus such as retrovirus or adenovirus. Any mammalian cells may be used, with human, mouse, rat, hamster, and primate cells being particularly preferred. Suitable cells also include known research cells, including but not limited to Jurkat T cells, NIH3T3, CHO, BHK, COS, HEK293, PER C.6, HeLa, Sp2/0, NS0 cells and variants thereof. In an alternately preferred embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, Fc variants are produced in insect cells (e.g. Sf21/Sf9, Trichoplusia ni Bti-Tn5b1-4) or yeast cells (e.g. S. cerevisiae, Pichia, etc). In an alternate embodiment, Fc variants are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the Fc variants may be produced by chemical synthesis methods. Also transgenic expression systems both animal (e.g. cow, sheep or goat milk, embryonated hen's eggs, whole insect larvae, etc.) and plant (e.g. corn, tobacco, duckweed, etc.)

Purification

In a preferred embodiment, Fc variant proteins are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful.

As is well known in the art, a variety of natural proteins bind Fc and antibodies, and these proteins can find use in the present invention for purification of Fc variants. For example, the bacterial proteins A and G bind to the Fc region. Likewise, the bacterial protein L binds to the Fab region of some antibodies, as of course does the antibody's target antigen. Purification can often be enabled by a particular fusion partner. For example, antibody or Fc fusion variant proteins may be purified using glutathione resin if a GST fusion is employed, Ni⁺² affinity chromatography if a His-tag is employed, or immobilized anti-flag antibody if a flag-tag is used.

For general guidance in suitable purification techniques, see Protein Purification: Principles and Practice, 3^{rd} Ed., Scopes, Springer-Verlag, NY, 1994. The degree of purification necessary will vary depending on the screen or use of the antibody or Fc fusion variants. In some instances no purification is necessary. For example in one embodiment, if the antibody or Fc fusion variants are secreted, screening may take place directly from the media. As is well known in the art, some methods of selection do not involve purification of proteins. Thus, for example, if a library of antibody or Fc fusion variants is made into a phage display library, protein purification may not be performed.

Further Modifications

The variant antibodies and Fc fusion proteins of the present invention may be subjected to any of a number of non-covalent modifications. Suitable modifications include PEGylation, glycosylation, and the attachment of chemical (e.g. calichemicin, maytansine, trichothene, aurestatin, etc. and/or various radioactive isotopes) or biological toxins (e.g. diphtheria toxin, ricin, abrin) that enhance the ability of the variant to kill target cells.

Assaying the Activity of the Variants

The variant antibodies and Fc fusion proteins of the invention may be tested for activity using any of a number of methods, including but not limited to those described below. Antibody or Fc fusion protein variants may be screened using a variety of methods, including but not limited to those that use in vitro assays, in vivo and cell-based assays, and selection technologies. Automation and high-throughput screening technologies may be utilized in the screening procedures.

Properties of antibody or Fc fusion protein variants that may be screened include but are not limited to stability, solubility, and antigen binding affinity and specificity. Multiple properties may be screened simultaneously or individually. Proteins may be purified or unpurified, depending on the requirements of the assay.

The biophysical properties of an antibody or Fc fusion variant protein, including but not limited to solubility and overall structural integrity, may be quantitatively or qualitatively determined using a wide range of methods that are known in the art. Methods which may find use in the present invention for characterizing the biophysical properties of an antibody or Fc fusion protein include gel electrophoresis, isoelectric focusing, capillary electrophoresis, chromatography such as size exclusion chromatography, ion-exchange chromatography, and reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use.

Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label of the antibody or Fc fusion protein variant. Assays may employ a variety of detection methods including but not limited to chromogenic, fluorescent, luminescent, or isotopic labels.

In a preferred embodiment, the library is screened using one or more cell-based or in vitro assays. For such assays, cells are treated with one or more antibodies or Fc fusion proteins belonging to a library. Such assays often involve monitoring the response of cells to the antibody or Fc fusion, for example cell survival, cell death, cellular phagocytosis, cell lysis, change in cellular morphology, chemotaxis, or transcriptional activation such as cellular expression of a natural gene or reporter gene.

Methods for monitoring cell death or viability are known in the art, and include the use of dyes, fluorophores, immunochemical, cytochemical, and radioactive reagents. For example, caspase assays or annexin-flourconjugates may enable apoptosis to be measured, and uptake or release of radioactive substrates (e.g. Chromium-51 release assays) or the metabolic reduction of fluorescent dyes such as alamar blue may enable cell growth, proliferationor activation to be monitored. In a preferred embodiment, the DELFIA® EuTDA-based cytotoxicity assay (Perkin Elmer, MA) is used. Alternatively, dead or damaged target cells may be monitoried by measuring the release of one or more natural intracellular proteins, for example lactate dehydrogenase.

Transcriptional activation may also serve as a method for assaying function in cell-based assays. In this case, response may be monitored by assaying for natural genes or proteins which may be upregulated or down-regulated, for example the release of certain interleukins may be measured, or alternatively readout may be via a reporter construct such as luciferase or GFP.

Cell-based assays may also involve the measure of morphological changes of cells as a response to the antibody or Fc fusion protein. Cell types for such assays may be prokaryotic or eukaryotic, and a variety of cell lines that are known in the art may be employed.

Alternatively, cell-based screens are performed using cells that have been transformed or transfected with nucleic acids encoding the Fc variants. In one embodiment, the cell-based screen utilizes a display method, including but are not limited to phage display (Phage display of peptides and proteins: a laboratory manual, Kay et al., 1996, Academic Press, San Diego, Calif., 1996; Lowman et al., 1991, Biochemistry 30:10832-10838; Smith, 1985, Science 228:1315-1317) and its derivatives, display on bacteria (Georgiou et al., 1997, Nat Biotechnol 15:29-34; Georgiou et al., 1993, Trends Biotechnol 11:6-10; Lee et al., 2000, Nat Biotechnol 18:645-648; Jun et al., 1998, Nat Biotechnol 16:576-80), yeast (Boder & Wittrup, 2000, Methods Enzymol 328:430-44; Boder & Wittrup, 1997, Nat Biotechnol 15:553-557), and mammalian cells (Whitehorn et al., 1995, Bio/technology 13:1215-1219). In an alternate embodiment, periplasmic expression and cytometric screening (Chen et al., 2001, Nat Biotechnol 19: 537-542), the protein fragment complementation assay (Johnsson & Varshavsky, 1994, Proc Natl Acad Sci USA 91:10340-10344.; Pelletier et al, 1998, Proc Nat Acad Sci USA 95:12141-12146), or the yeast two hybrid screen (Fields & Song, 1989, Nature 340:245-246) is used. Additionally, if the antibody or Fc fusion protein may be made to impart a selectable growth or survival advantage to a cell, this property may be used to screen or select for desired antibody or Fc fusion protein variants.

Determining the Immunogenicity of the Variants

In a preferred embodiment, the immunogenicity of the antibody and Fc fusion protein variants is determined experimentally to confirm that the variants do have reduced or eliminated immunogenicity relative to the parent protein.

In a preferred embodiment, ex vivo T-cell activation assays are used to experimentally quantitate immunogenicity. In this method, antigen presenting cells and naive T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et al. J. Immunol. Meth., 24: 17-24 (2000)).

Other suitable T-cell assays include those disclosed in Meidenbauer, et al. Prostate 43, 88-100 (2000); Schultes, B. C and Whiteside, T. L., J. Immunol. Methods 279, 1-15 (2003); and Stickler, et al., J. Immunotherapy, 23, 654-660 (2000).

In a preferred embodiment, the PBMC donors used for the above-described T-cell activation assays will comprise class II MHC alleles that are common in patients requiring treatment for antibody and Fc fusion protein responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to antibody and Fc fusion protein responsive disorders.

In a preferred embodiment, the MHC haplotype of PBMC donors or patients that raise an immune response to the wild type or variant antibodies and Fc fusion proteins are compared with the MHC haplotype of patients who do not raise a response. This data may be used to guide preclinical and clinical studies as well as aiding in identification of patients who will be especially likely to respond favorably or unfavorably to the antibody or Fc fusion protein therapeutic.

In an alternate preferred embodiment, immunogenicity is measured in transgenic mouse systems. For example, mice expressing fully or partially human class II MHC molecules may be used.

In an alternate embodiment, immunogenicity is tested by administering the antibody or Fc fusion protein variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see, e.g., Sonderstrup et al. Immunol. Rev. 172: 335-343 (1999) and Forsthuber et al. J. Immunol. 167: 119-125 (2001)).

Formulation and Administration to Patients

Once made, the variant antibodies and Fc fusion proteins and nucleic acids of the invention find use in a number of applications. In a preferred embodiment, the variant antibodies and Fc fusion proteins are administered to a patient to treat an antibody and Fc fusion protein responsive disorder.

In a preferred embodiment, the variant antibody or Fc fusion protein is used to treat an autoimmune disease, cancer, inflammatory disorder, infectious disease, or other responsive condition. Administration may be therapeutic or prophylactic.

The pharmaceutical compositions of the present invention comprise a variant antibody or Fc fusion protein in a form suitable for administration to a patient. Formulations of the proteins of the present invention are prepared for storage by mixing the protein having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. The formulations to be used for in vivo administration are preferably sterile.

The dosing amounts and frequencies of administration are, in a preferred embodiment, selected to be therapeutically or prophylactically effective. As is known in the art, adjustments for antibody or Fc fusion degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the therapeutically active antibody or Fc fusion of the present invention in the formulation may vary from about 0.1 to 100 weight %. In a preferred embodiment, the concentration of the antibody or Fc fusion is in the range of 0.003 to 1.0 molar. In order to treat a patient, a therapeutically effective dose of the antibody or Fc fusion of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.1, 1, 10, or 50 mg/kg of body weight, with 1 to 10 mg/kg being preferred. In some embodiments, only a single dose of the antibody or Fc fusion of the present invention is used. In other embodiments, multiple doses of the antibody or Fc fusion of the present invention are administered. The elapsed time between administrations may be less than 1 hour, about 1 hour, about 1-2 hours, about 2-3 hours, about 3-4 hours, about 6 hours, about 12 hours, about 24 hours, about 48 hours, about 2-4 days, about 4-6 days, about 1 week, about 2 weeks, or more than 2 weeks.

In other embodiments, the antibodies or Fc fusions of the present invention are administered in metronomic dosing regimes, either by continuous infusion or frequent administration without extended rest periods. Such metronomic administration may involve dosing at constant intervals without rest periods. Typically, such regimens encompass chronic low-dose or continuous infusion for an extended period of time, for example 1-2 days, 1-2 weeks, 1-2 months, or up to 6 months or more. The use of lower doses may minimize side effects and the need for rest periods.

In certain embodiments the antibody or Fc fusion of the present invention and one or more other prophylactic or therapeutic agents are cyclically administered to the patient. Cycling therapy involves administration of a first agent at one time, a second agent at a second time, optionally additional agents at additional times, optionally a rest period, and then repeating this sequence of administration one or more times. The number of cycles is typically from 2-10. Cycling therapy may reduce the development of resistance to one or more agents, may minimize side effects, or may improve treatment efficacy.

Methods of Administration

Administration of the pharmaceutical composition comprising an antibody or Fc fusion of the present invention, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances, for example for the treatment of wounds, inflammation, etc., the antibody or Fc fusion may be directly applied as a solution or spray. As is known in the art, the pharmaceutical composition may be formulated accordingly depending upon the manner of introduction.

As is known in the art, antibody therapeutics are often delivered by IV infusion or bolus. The antibodies and Fc fusions of the present invention may also be delivered using such methods. For example, administration may be by intravenous infusion with 0.9% sodium chloride as an infusion vehicle.

Subcutaneous administration may be preferable in some circumstances because the patient may self-administer the pharmaceutical composition. Many antibody therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO 04091658).

Pulmonary delivery may be accomplished using an inhaler or nebulizer and a formulation comprising an aerosolizing agent. For example, AERx® inhalable technology commercially available from Aradigm, or Inhance™ pulmonary delivery system commercially available from Nektar Therapeutics may be used.

In addition, any of a number of delivery systems are known in the art and may be used to administer the antibodies or Fc fusions of the present invention. Examples include, but are not limited to, encapsulation in liposomes, microparticles, microspheres (eg. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol),polylactides, copolymers of L-glutamic acid and ethyl-L-gutamate, ethylene-vinyl acetate, lactic acid-glycolic acid copolymers such as the LUPRON DEPOT®, and poly-D-(−)-3-hydroxyburyric acid.

In an alternate embodiment, variant antibodies and Fc fusion proteins nucleic acids may be administered; i.e., “gene therapy” approaches may be used. In this embodiment, variant antibodies and Fc fusion proteins nucleic acids are introduced into cells in a patient in order to achieve in vivo synthesis of a therapeutically effective amount of variant antibodies and Fc fusion proteins protein. Variant antibodies and Fc fusion proteins nucleic acids may be introduced using a number of techniques, including but not limited to transfection with liposomes, viral (typically retroviral) vectors, and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

In all cases, controlled release systems may be used to release the antibody or Fc fusion at or close to the desired location of action.

Therapeutic Regimen

The antibodies and Fc fusions of the present invention may be administered as a monotherapy or concomitantly with one or more other therapeutic regimens or agents. The additional therapeutic regimes or agents may be used to improve the efficacy or safety of the antibody or Fc fusion. Also, the additional therapeutic regimes or agents may be used to treat the same disease or a comorbidity rather than to alter the action of the antibody or Fc fusion. For example, an antibody or Fc fusion of the present invention may be administered to the patient along with chemotherapy, radiation therapy, surgery, both chemotherapy and radiation therapy, etc. The antibody or Fc fusion of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional antibody or Fc fusion proteins, FcγRIIb or other Fc receptor inhibitors, or other therapeutic agents.

Pharmacogenomics

Class II MHC molecules are polymorphic in the human population. HLA genotype is a major determinant of susceptibility to specific autoimmune diseases (see, e.g., Nepom Clin. Immunol. Immunopathol. 67: S50-S55 (1993)) and infections (see, e.g., Singh et al. Emerg. Infect. Dis. 3: 41-49 (1997)). Furthermore, the set of MHC alleles present in an individual can affect the efficacy of some vaccines (see, e.g., Cailat-Zucman et al. Kidney Int. 53: 1626-1630 (1998) and Poland et al. Vaccine 20: 430-438 (2001)). For a given patient or population of patients, the likelihood of eliciting an immune response to the antibodies and Fc fusions of the present invention may be affected by the presence or absence of specific class II MHC alleles.

In a preferred embodiment, class II MHC alleles that are associated with increased or decreased susceptibility to elicit an immune response to an antibody or Fc fusion protein are identified. For example, patients treated with antibody or Fc fusion protein therapeutics may be tested for the presence of antibodies that recognize the therapeutic antibody or Fc fusion protein and then genotyped for class II MHC. Alternatively, T-cell activation assays such as those described above may be conducted using cells derived from a number of genotyped donors. Alleles that confer susceptibility to immunogenicity may be defined as those alleles that are significantly more common in those who elicit an immune response versus those who do not. Similarly, alleles that confer resistance to immunogenicity may be defined as those that are significantly less common in those who do not elicit an immune response versus those that do. It is also possible to use purely computational techniques to identify which alleles are likely to recognize peptides in an antibody or Fc fusion protein therapeutic.

In a preferred embodiment, the antibodies and Fc fusions of the present invention do not comprise peptides that appreciably bind to any human class II MHC allele. Such therapeutics would be expected to be minimally immunogenic. In an alternate embodiment, the antibodies and Fc fusions of the present invention do not comprise peptides that appreciably bind to any human class II MHC allele that is present in a significant fraction of the relevant patient population. For example, many autoimmune conditions are associated with specific MHC alleles, and therefore the allele frequencies for many autoimmune diseases are different from that of the general population. It is preferred that the antibodies and Fc fusions bind to class II alleles present in less than 10% of patients, with less than 1% or less than 0.1% being especially preferred.

In a preferred embodiment, the HLA haplotype of patients is determined in order to predict the potential immunogenicity of the antibodies and Fc fusions of the present invention. This information may be used, for example, to select patients to include or exclude from clinical trials or, post-approval, to provide guidance to physicians and patients regarding appropriate dosages and treatment options. In one embodiment, patients are selected for inclusion in clinical trials or post-approval treatment with an antibody of the present invention if their genotype indicates that they are less likely to elicit an immune response to an antibody of the present invention as compared to one or more currently used antibody therapeutics. In another embodiment, appropriate dosages, routes of administration, and treatment regimens are determined using such genotype information.

### EXAMPLES

### Example 1

### Identification of MHC-Binding Agretopes in Human Antibody Sequences

Matrix method calculations (Sturniolo, supra) were conducted for the constant domains of human IgG1, IgG2, IgG3, and IgG4 sequences of, respectively, SEQ ID NO:1, SEQ ID NO:2, SEQ ID NO:3, and SEQ ID NO:4.

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1104, DRB1*1301, DRB1*1302, DRB1*1501, and DRB1*1502.

For each nine-mer that is predicted to bind to at least one allele at a 5% threshold, the number of alleles that are hit at 1%, 3% and 5% thresholds were given, as well as the percent of the US population that are predicted to react to the nine-mer. The worst nine-mers are shown in bold. They are predicted to be immunogenic in at least 10% of the US population, using a 1% threshold.

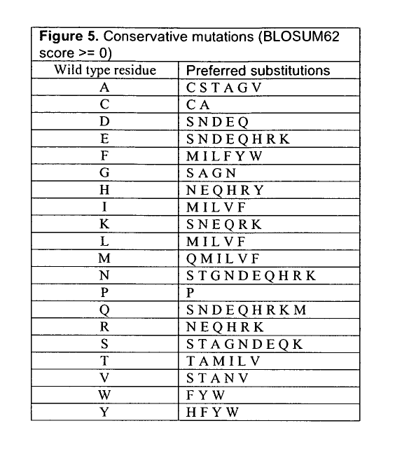

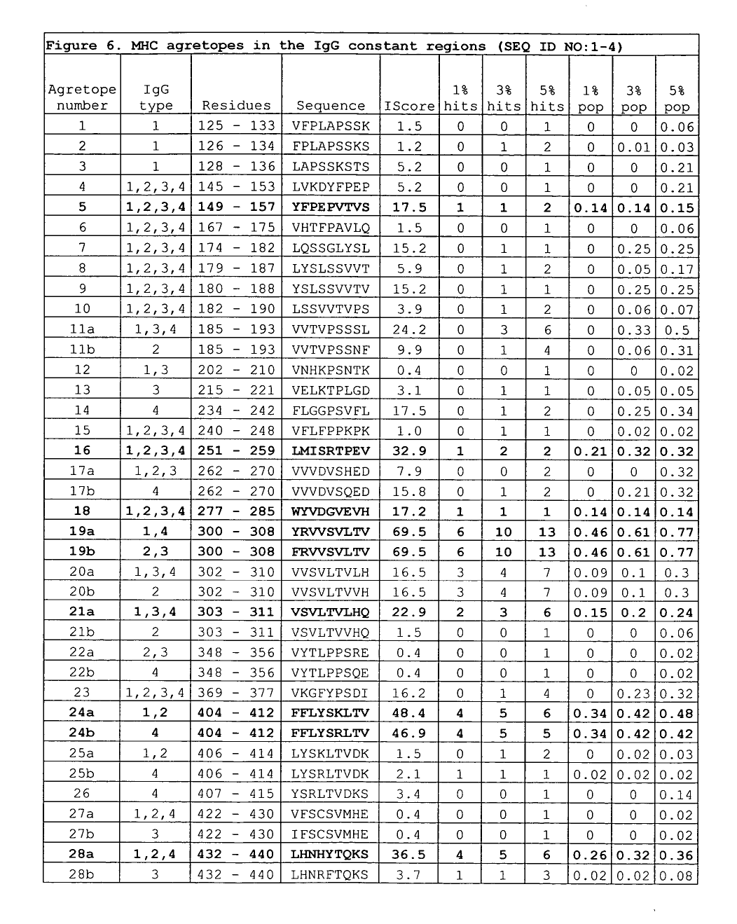

Predicted MHC-binding agretopes in the constant domains of IgG1, IgG2, IgG3, and IgG4 are shown in FIG. 6. IScore, the number of alleles hit at 1%, 3%, and 5% thresholds, and the percent of the population hit at 1%, 3%, and 5% thresholds are shown.

Of special interest are differences among the IgG classes that are predicted to substantially impact immunogenicity. For example, V309 in IgG2 versus L309 in IgG1, IgG3, and IgG4 results in an IScore of 1.5 versus 23 for agretope 21a. Incorporating the L309V substitution into IgG1 substantially decreases MHC binding of one agretope, and does not create any new agretopes or any nine-mers that are not already present in IgG2. Similarly, R435 and F436 in IgG3 versus H435 and Y436 in IgG1, IgG2, and IgG4 results in an IScore of 3.7 for agretope 28b versus an IScore of 36.5 for agretope 28a. Incorporating the H435R/Y436F substitutions into IgG1 substantantially decreases MHC binding for one agretope and does not create any new agretopes or any nine-mers that are not already present in IgG3.

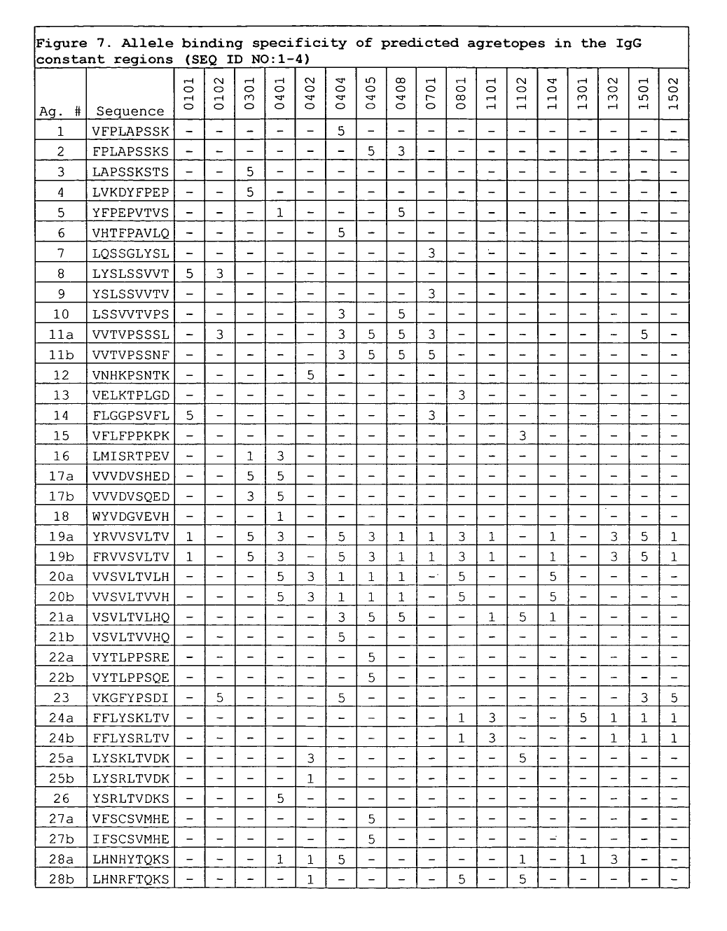

The allele binding specificity of the above agretopes is shown in FIG. 7 below. DRB1 alleles that are predicted to bind to each agretope at 1%, 3%, and 5% cutoffs are marked with “1”, “3”, or “5”, respectively.

Heavy chain variable region (VH), lambda light chain variable region (VLL) and kappa light chain variable region (VLK) sequences from the human germline were analyzed to identify MHC-binding agretopes. Sequences are aligned and numbered according to Kabat. “Start” indicates the first residue in the nine-mer agretope. “-” indicates an IScore of exactly 0, while “0” indicates an IScore greater than 0 and less than 0.5.

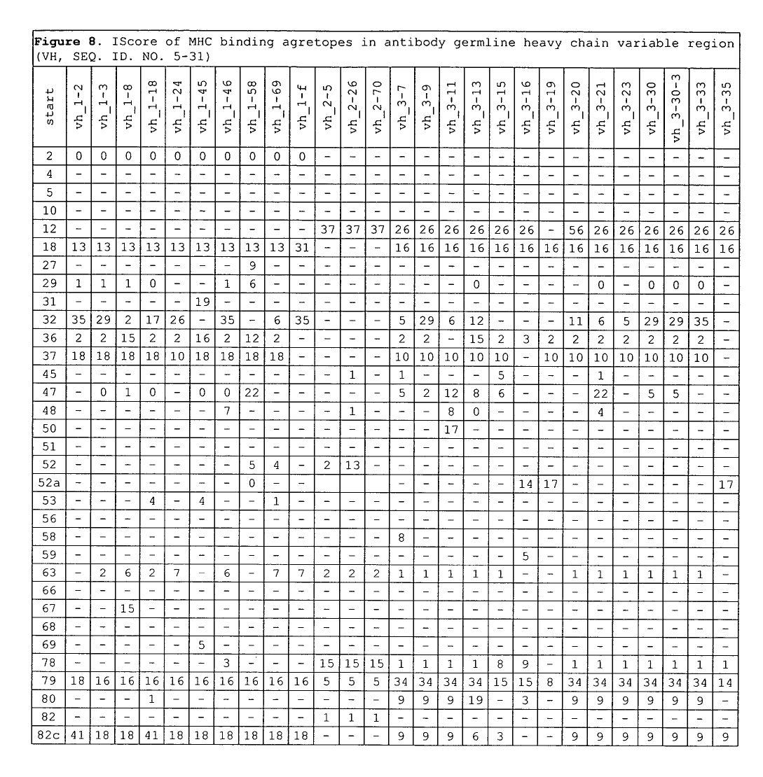

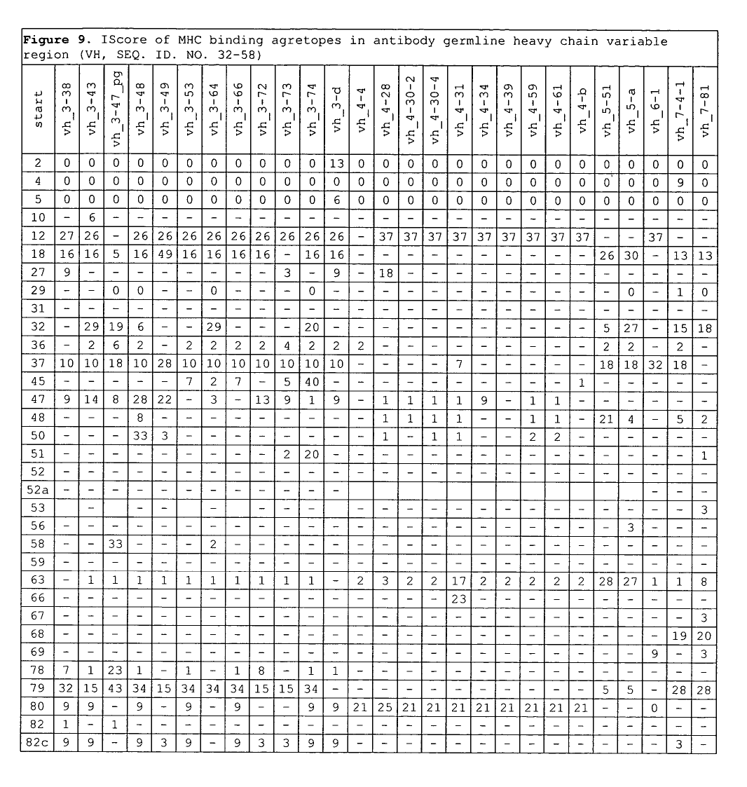

FIGS. 8-13 show the IScore of MHC binding agretopes in antibody germline heavy and light chain chain variable regions.

### Example 2

### Identification of Suitable Less Immunogenic Sequences for MHC-Binding Agretopes in Antibodies and Fc Fusion Proteins

MHC-binding agretopes that were predicted to bind alleles present in at least 10% of the US population, using a 1% threshold, were analyzed to identify suitable less immunogenic variants.

At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 80% of the wild type score.

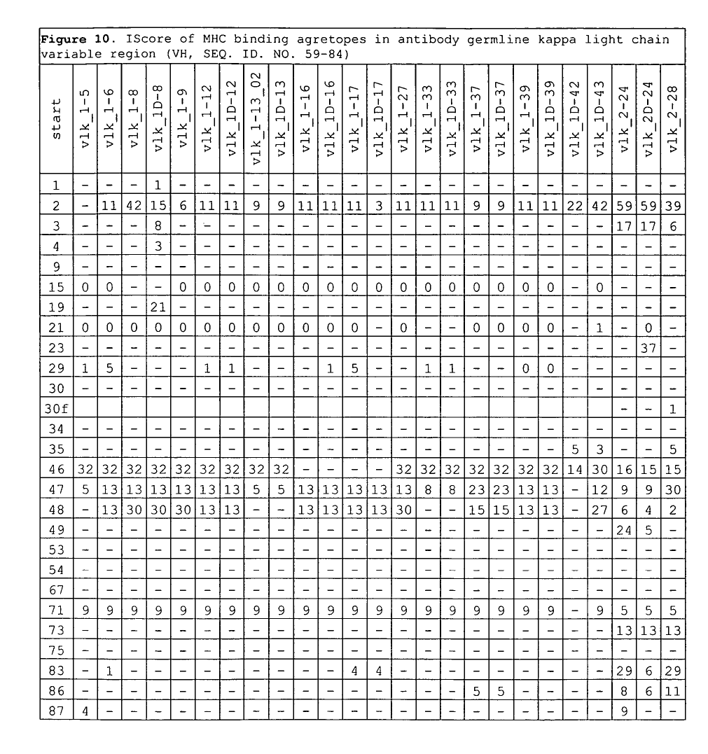

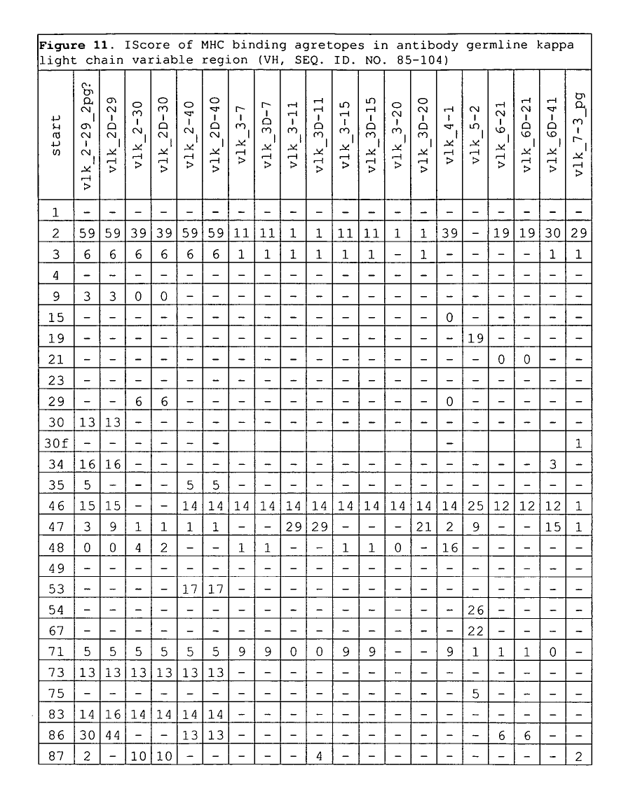

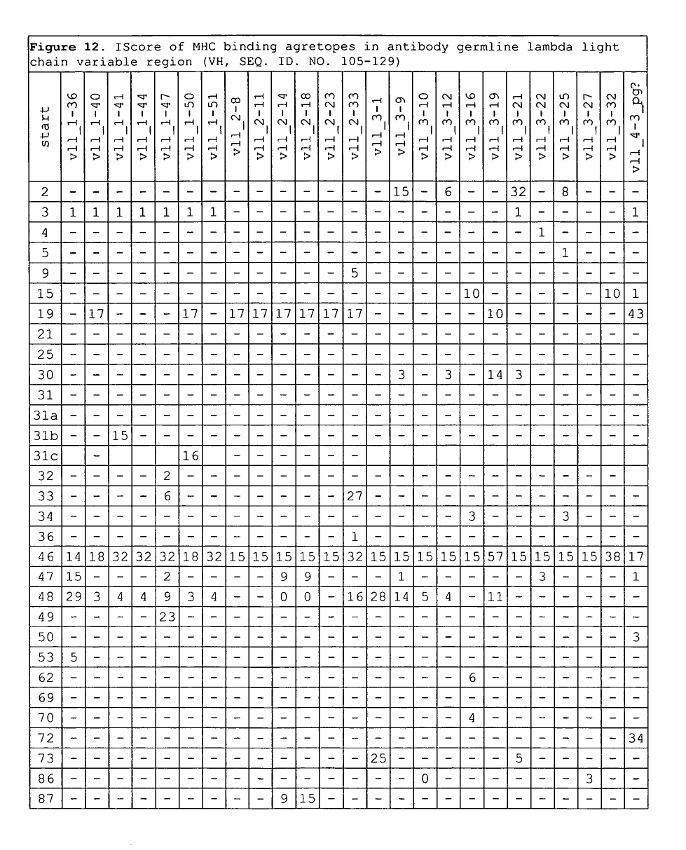

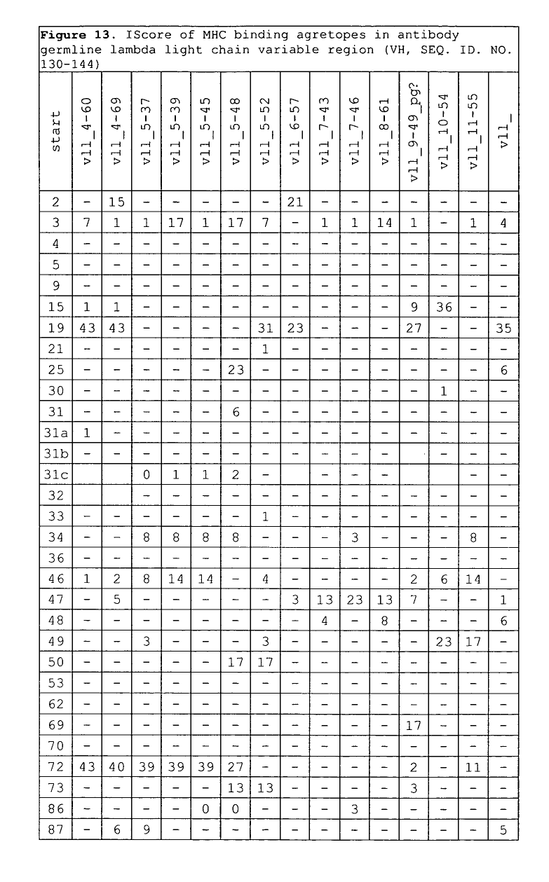

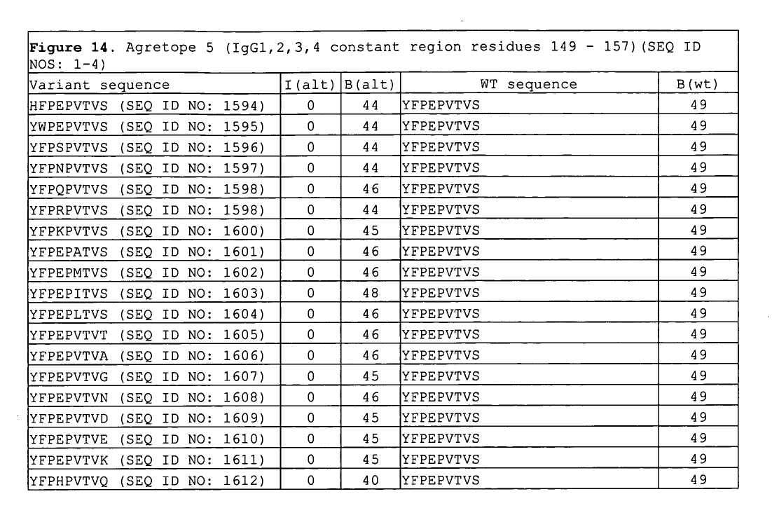

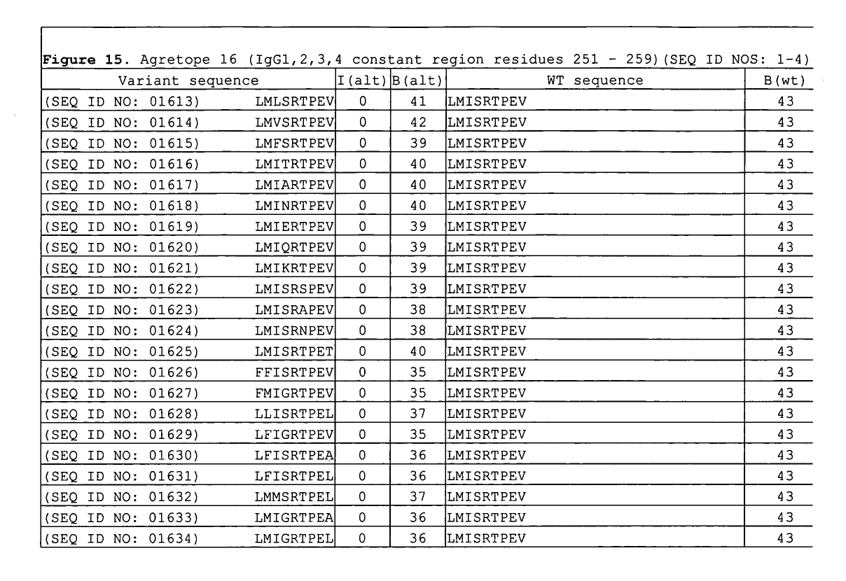

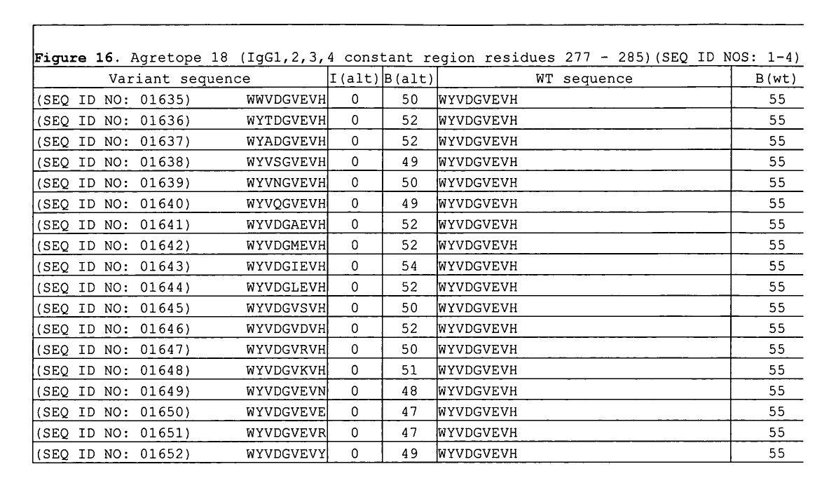

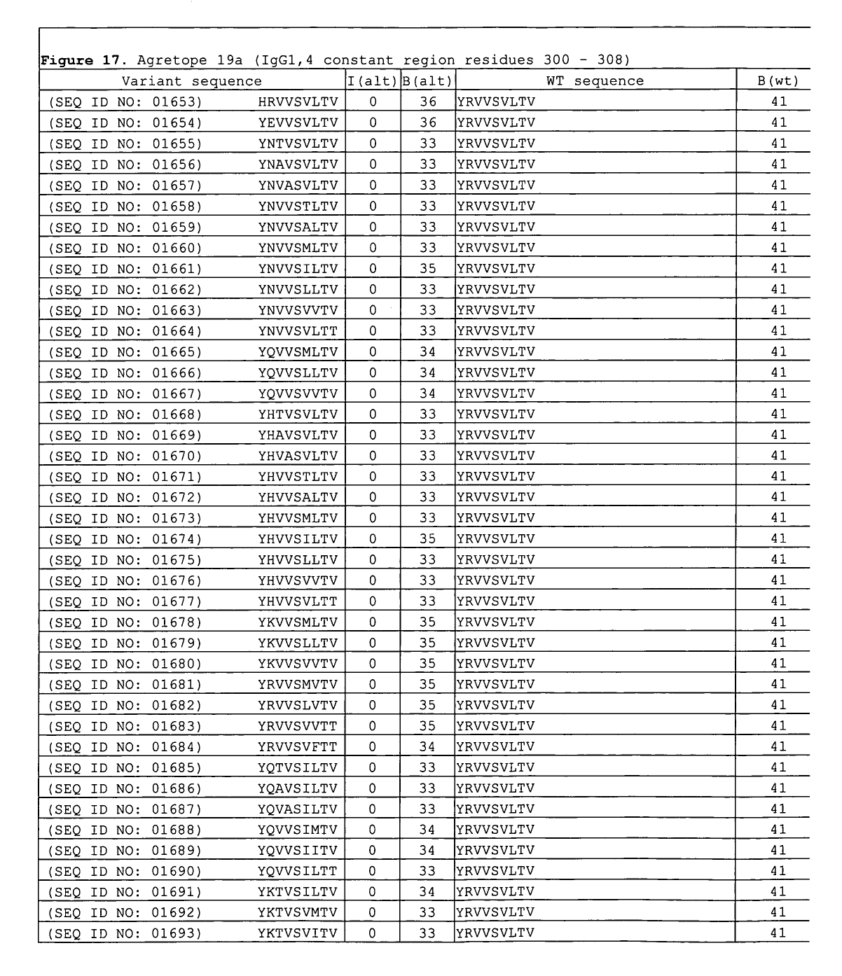

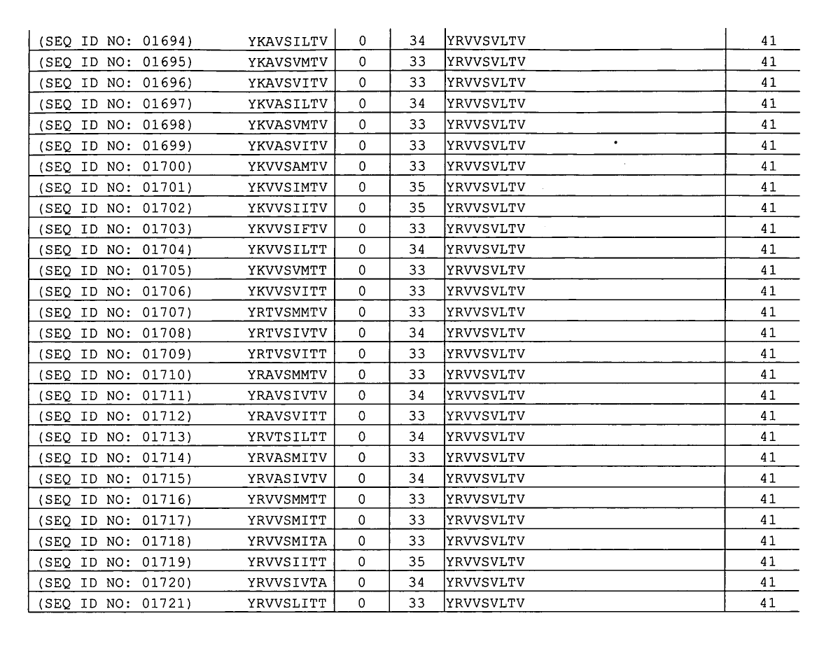

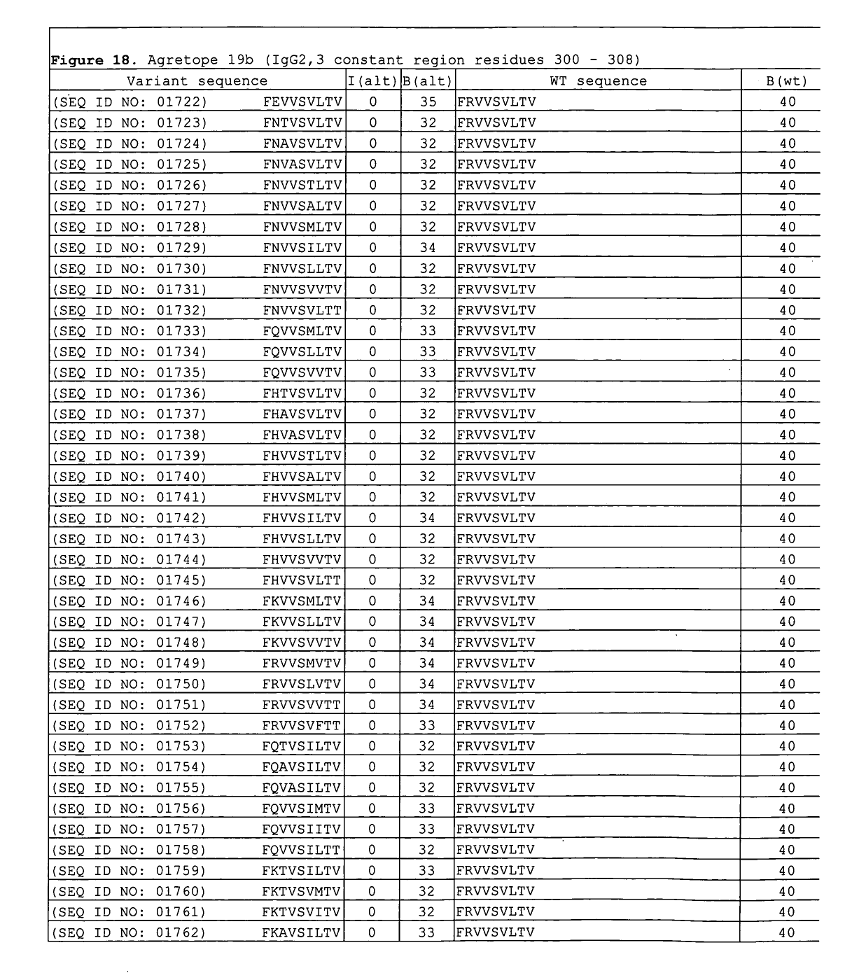

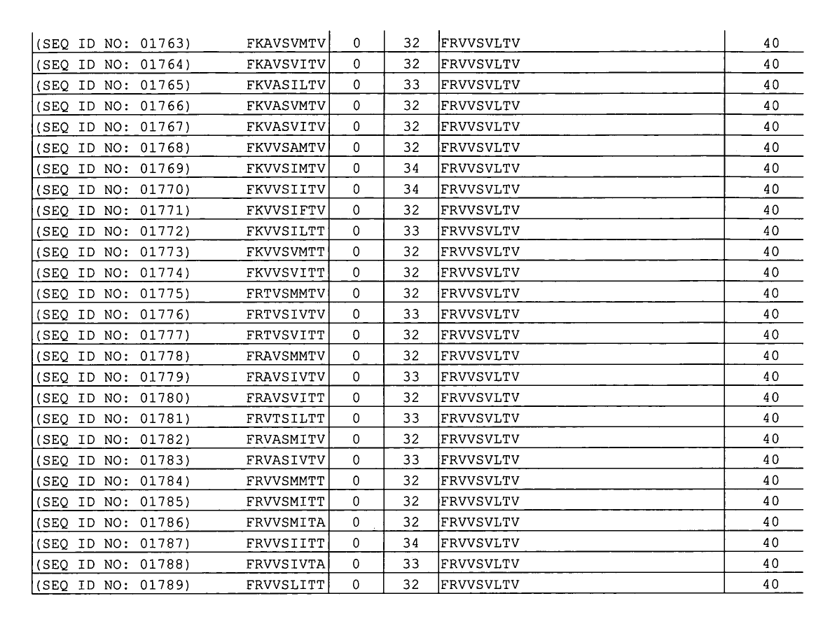

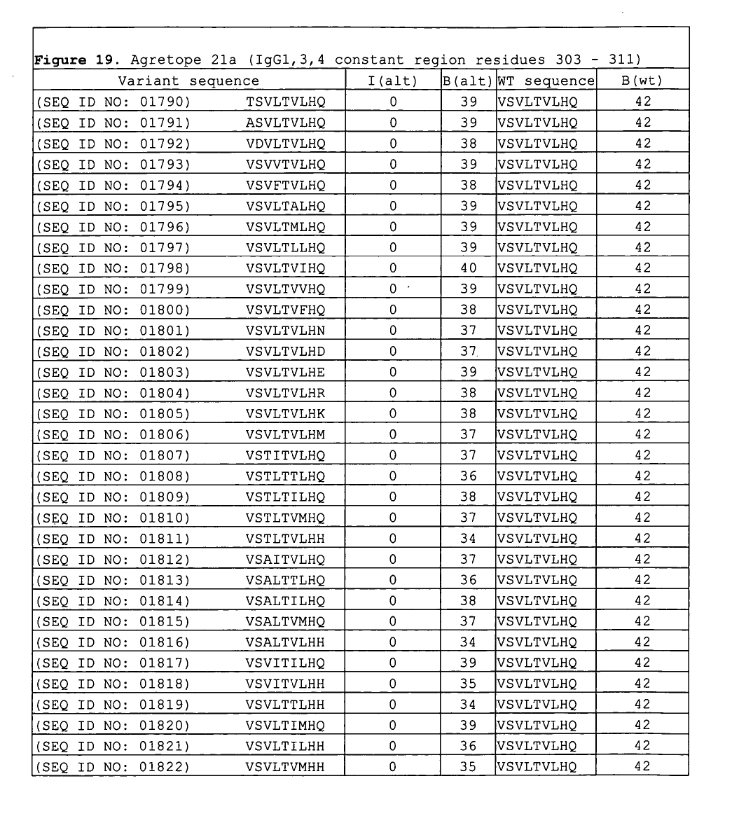

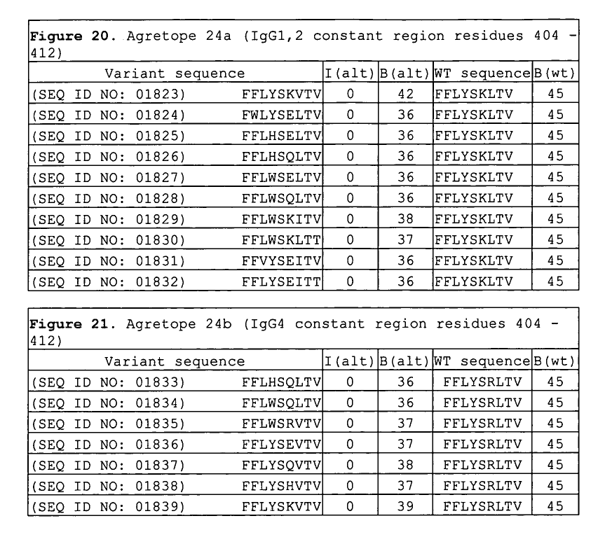

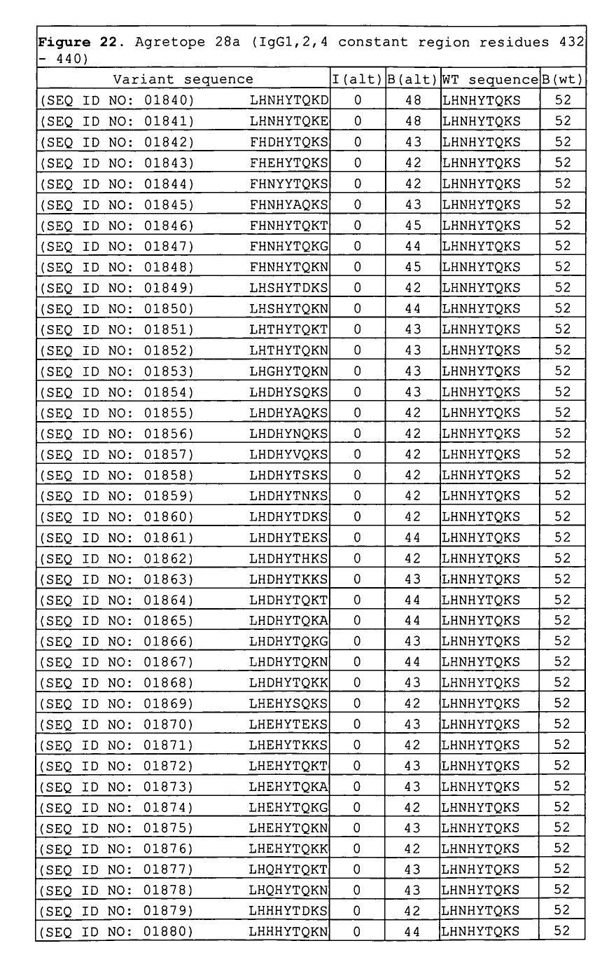

FIGS. 14-20 show suitable less immunogenic variants of agretope 5 (IgG1,2,3,4 constant region residues 149-157), agretope 16 (IgG1,2,3,4 constant region residues 251-259), agretope 18 (IgG1,2,3,4 constant region residues 277-285), agretope 19a (IgG1,4 constant region residues 300-308), agretope 19b (IgG2,3 constant region residues 300-308), agretope 21a (IgG1,3,4 constant region residues 303-311), agretope 24a (IgG1,2 constant region 404-412), agretope 24b (IgG4 constant region 404-412), and agretope 28a (IgG1,2,4 constant region residues 432-440). B(wt) is the BLOSUM62 score of the wild type nine-mer, I(alt) is the percent of the US population containing one or more MHC alleles that are predicted to bind the alternate nine-mer at a 1% threshold, and B(alt) is the BLOSUM62 score of the alternate nine-mer.

### Example 3

### Identification of Suitable Less Immunogenic Sequences for MHC-Binding Agretopes in Antibodies and Fc Fusion Proteins: PDA® technology

MHC-binding agretopes that were predicted to bind alleles present in at least 10% of the US population, using a 1% threshold, using PDA® technology to identify suitable less immunogenic variants.

Each position in the agretopes of interest was analyzed to identify a subset of amino acid substitutions that are potentially compatible with maintaining the structure and function of the protein. PDA® technology calculations were run for each position of each nine-mer agretope and compatible amino acids for each position were saved. In these calculations, side-chains within 5 Angstroms of the position of interest were permitted to change conformation but not amino acid identity. The variant agretopes were then analyzed for immunogenicity. The PDA® energies and IScore values for the wild-type nine-mer agretope were compared to the variants and the subset of variant sequences with lower predicted immunogenicity and PDA® energies within 5.0 kcal/mol of the wild-type (wt) were noted.

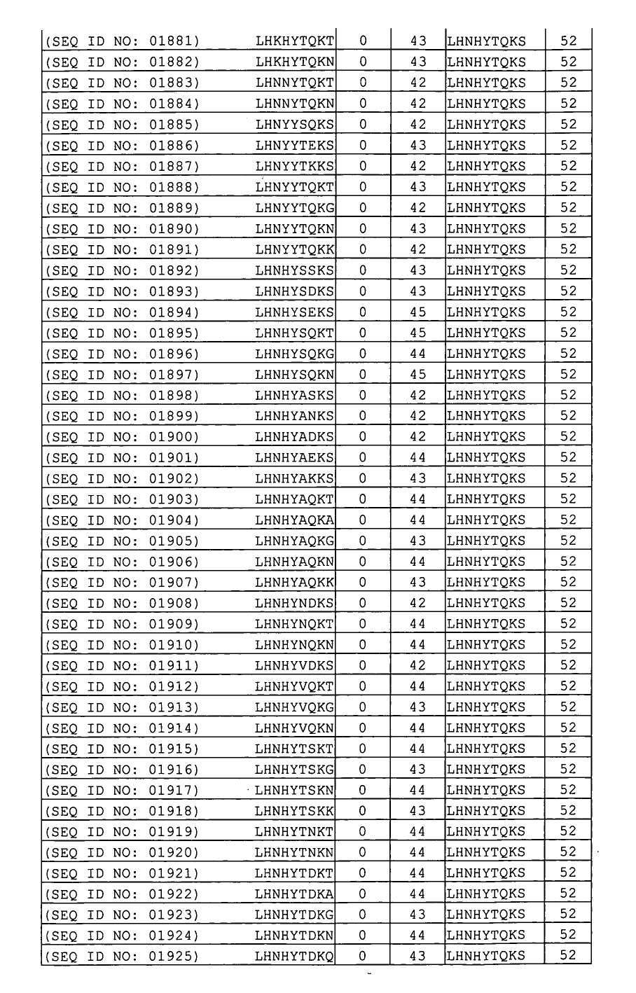

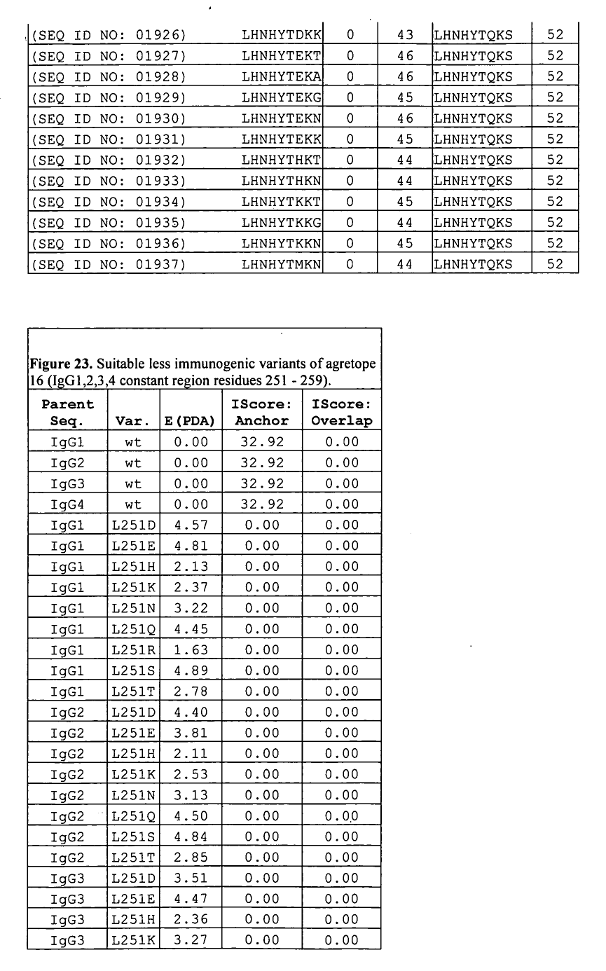

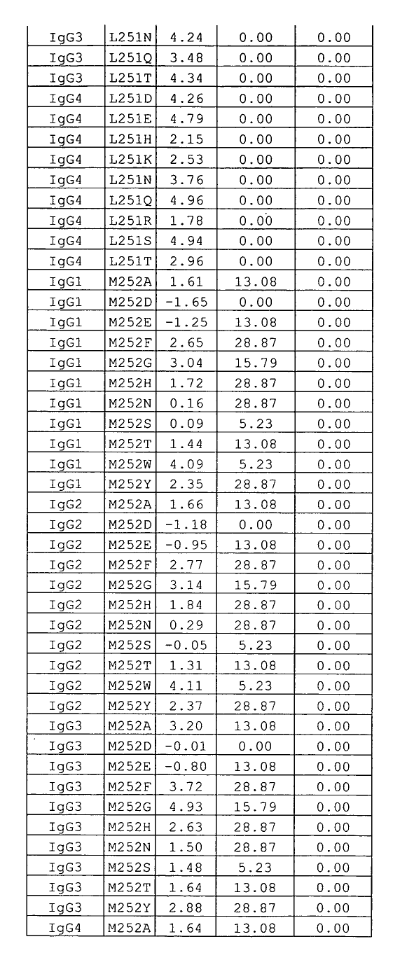

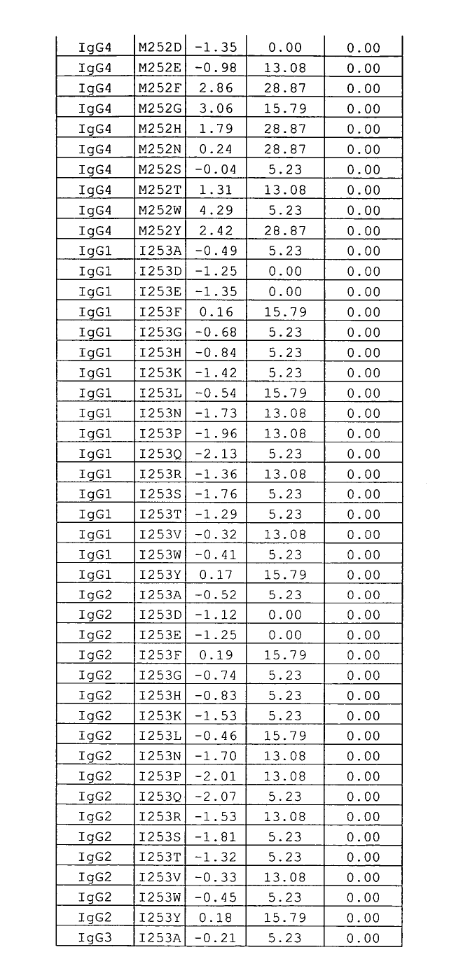

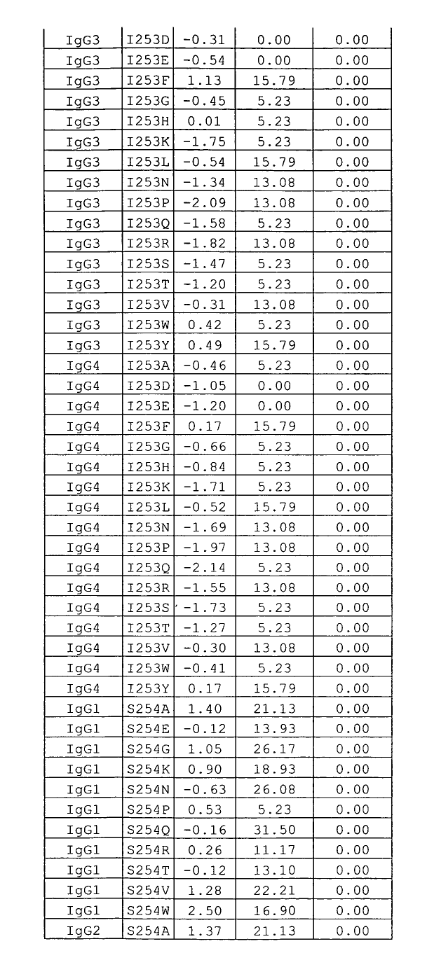

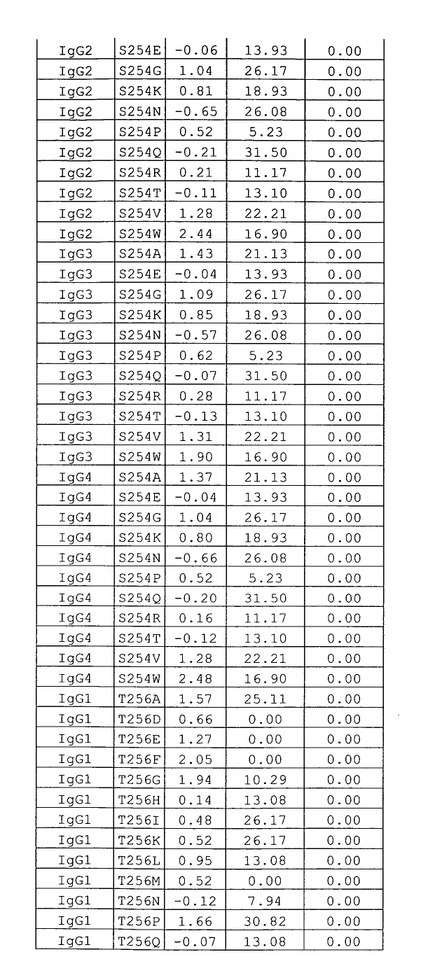

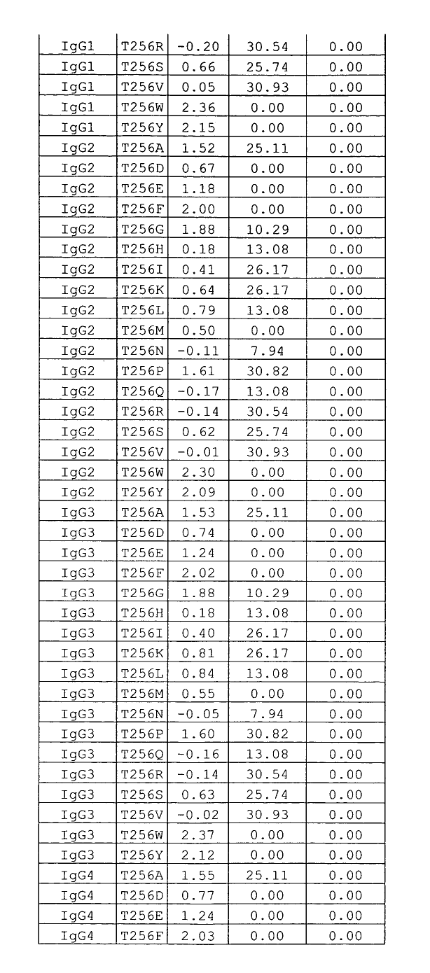

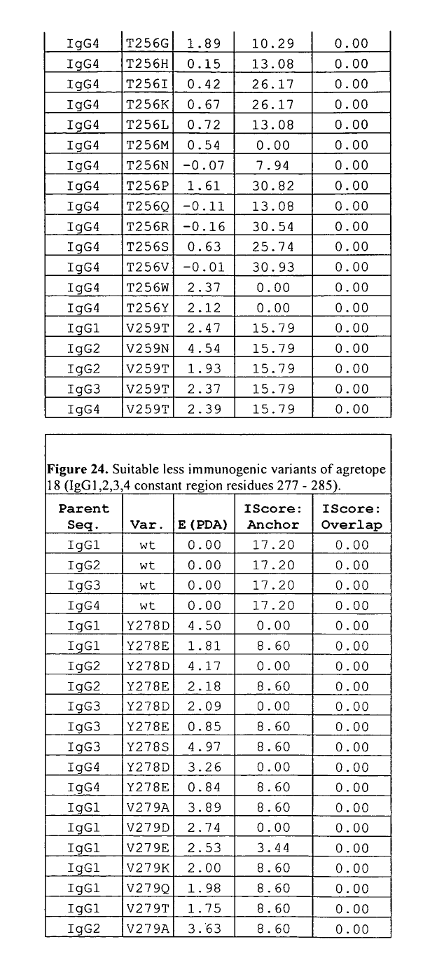

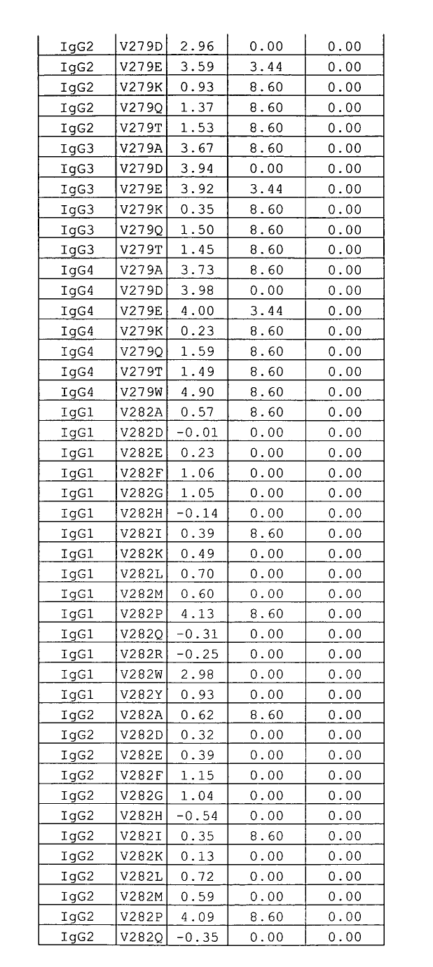

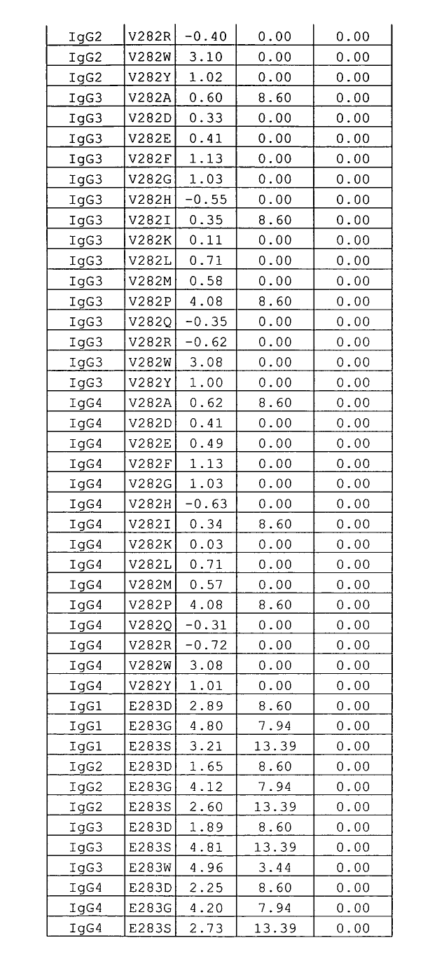

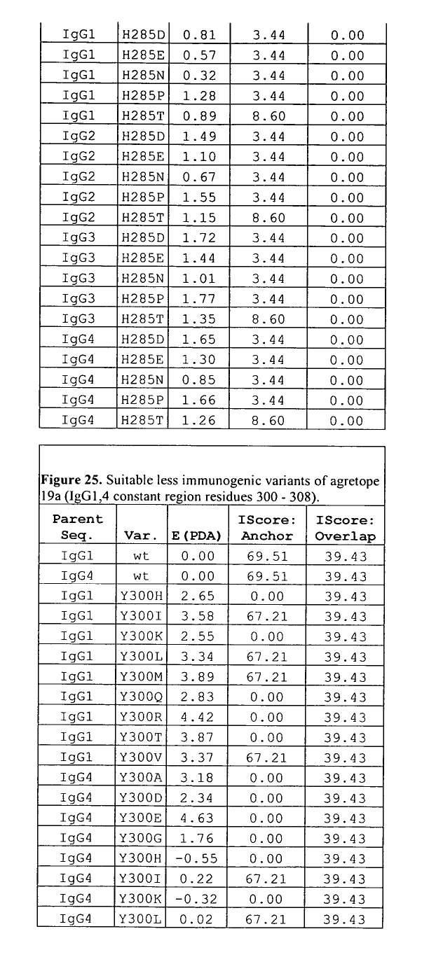

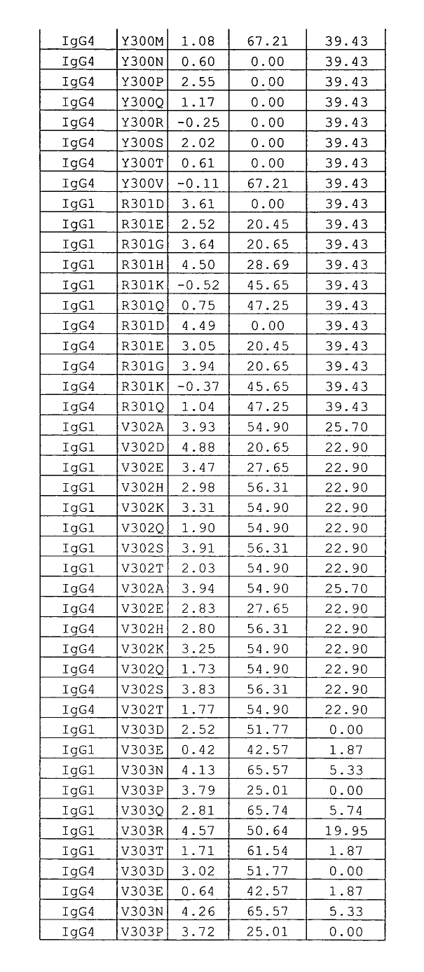

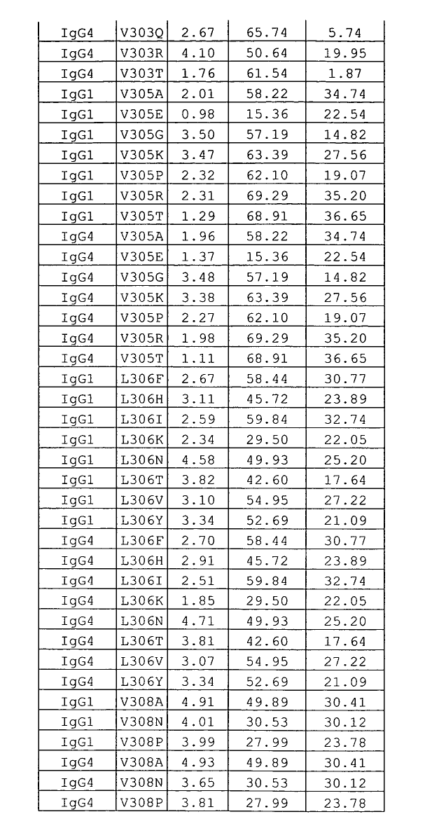

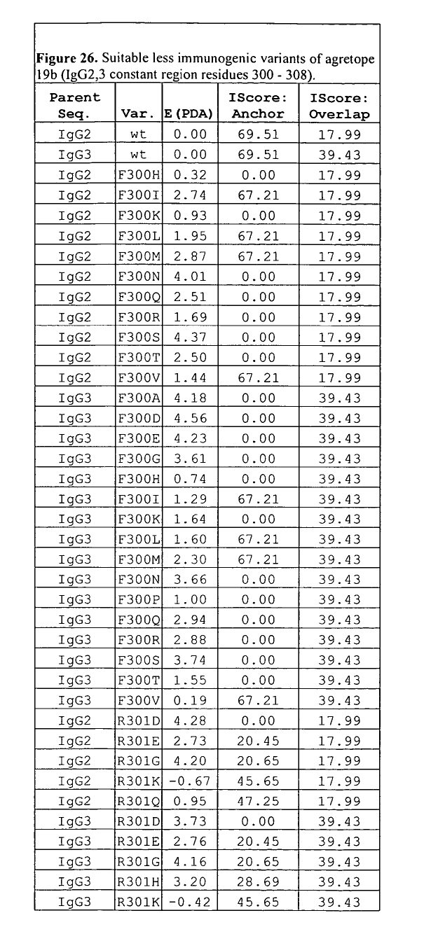

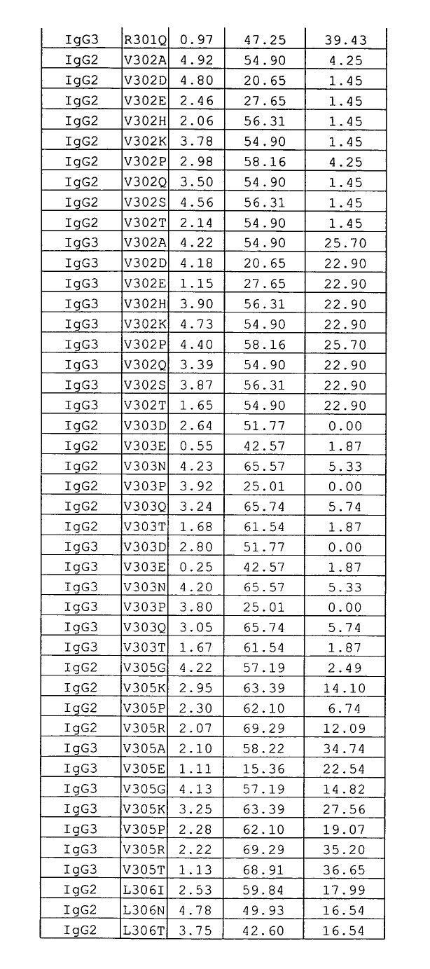

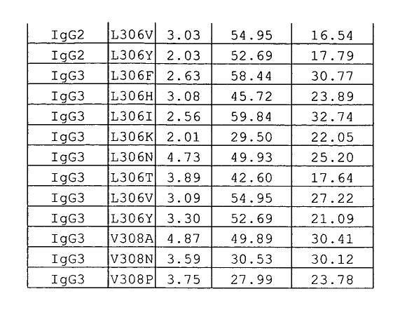

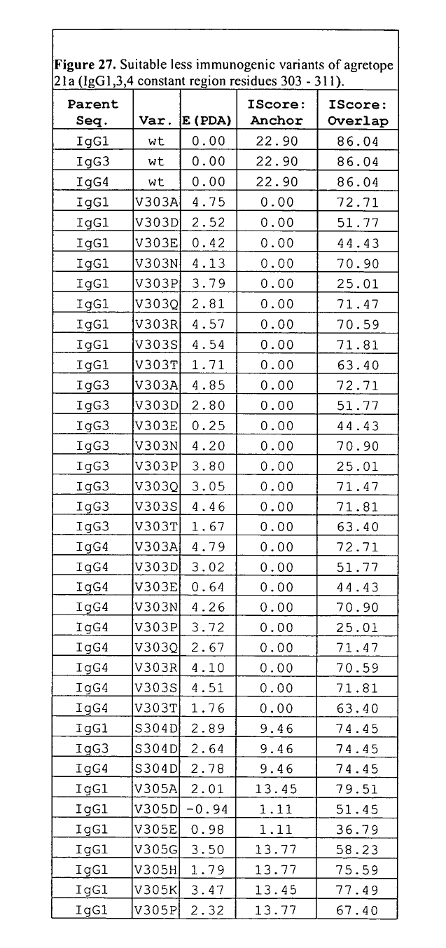

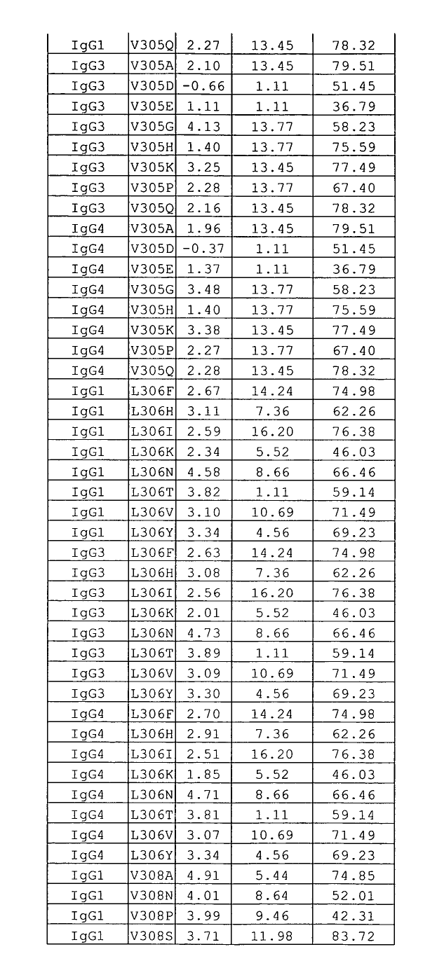

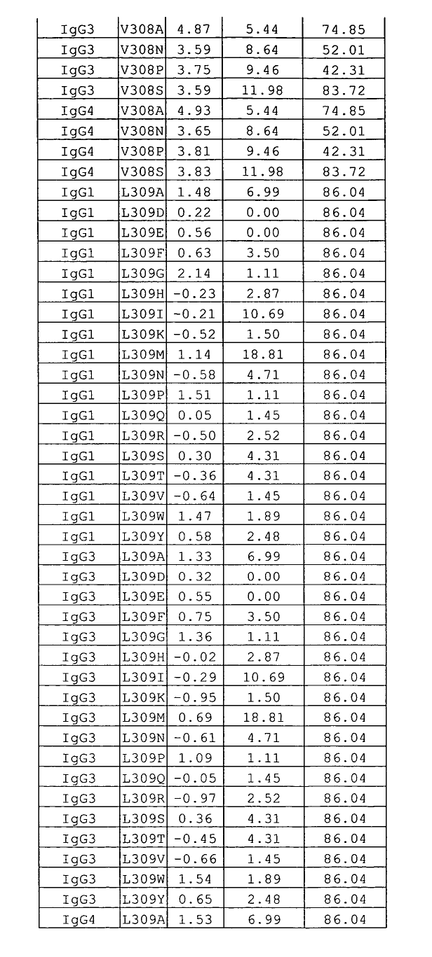

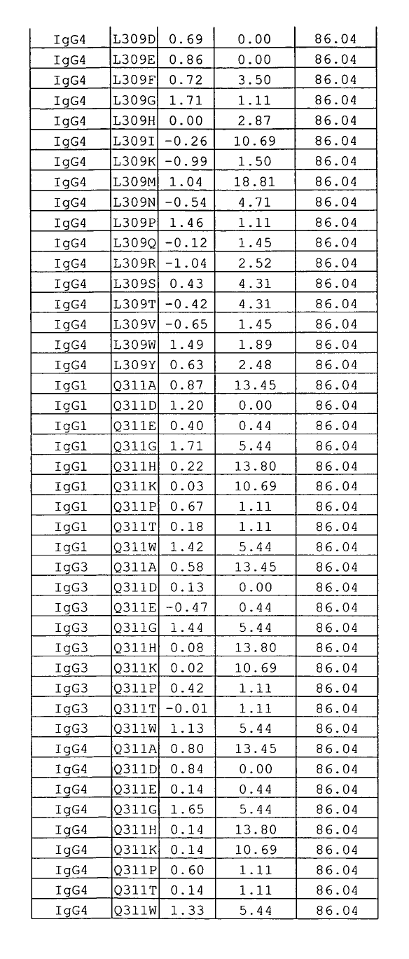

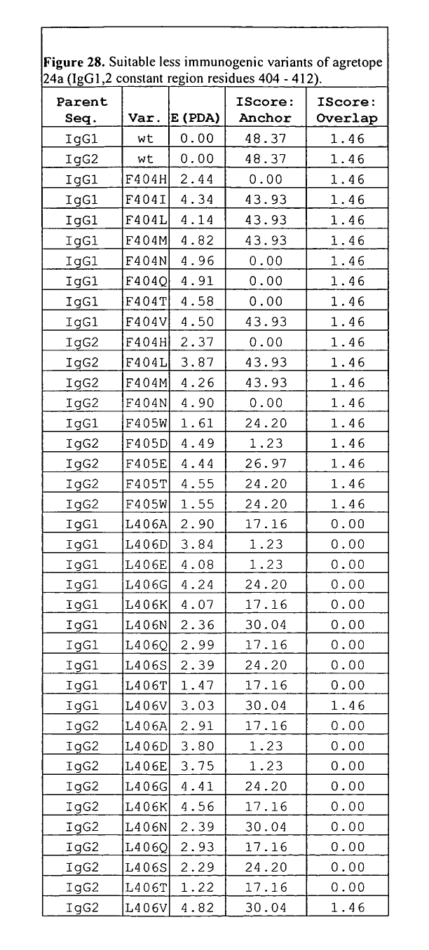

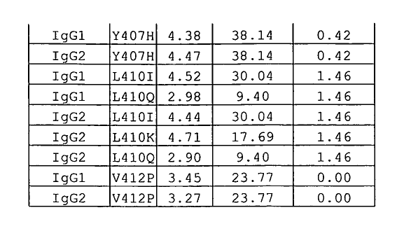

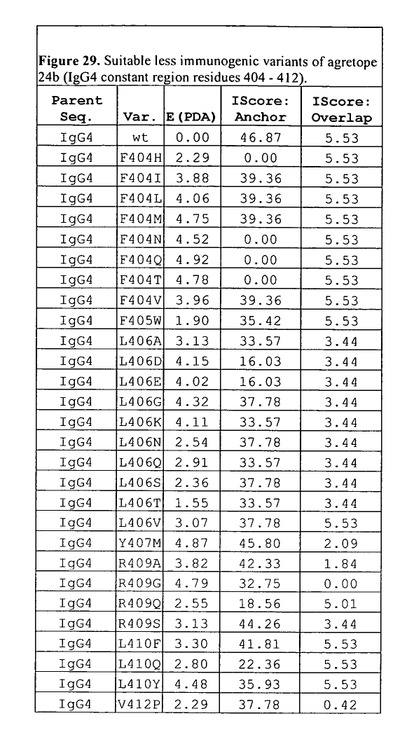

FIGS. 23-30 show suitable less immunogenic variants of agretope 16 (IgG1,2,3,4 constant region residues 251-259), agretope 18 (IgG1,2,3,4 constant region residues 277-285), agretope 19a (IgG1,4 constant region residues 300-308), agretope 19b (IgG2,3 constant region residues 300-308), agretope 21a (IgG1,3,4 constant region residues 303-311), agretope 24a (IgG1,2 constant region residues 404-412), agretope 24b (IgG4 constant region residues 404-412), and agretope 28a (IgG1,2,4 constant region residues 432-440) identified using PDA® technology calculations. In FIGS. 23-30, E(PDA) is the energy determined using PDA® technology calculations compared against the wild-type, IScore: Anchor is the IScore for the agretope, and IScore: Overlap is the sum of the IScores for all of the overlapping agretopes.

### Example 4

### Analysis of Immunogenic Sequences in Fc Variants Engineered for Enhanced Effector Function

A set of antibodies and Fc fusion proteins variants were engineered for a number of properties, including altered binding to Fc gamma receptors, FcRn, and protein A, as well as function in the absence of glycosylation. MHC binding agretopes in the engineered Fc variants were compared with the MHC binding agretopes in the parent sequence of SEQ. ID. NO. 1.

Variants that show a decrease in IScore relative to SEQ. ID. NO. 1 for at least one agretope include SEQ. ID. NO. 420, 423, 428, 429, 432, 433, 434, 435, 436, 437, 444, 447, 450, 451, 452, 453, 460, 461, 462, 463, 464, 472, 473, 491, 494, 522, 550, 551, 553, 554, 555, 601, 602, 603, 607, 608, 642, 643, 644, 667, 668, 670, 671, 712, 717, 722, 723, 724, 725, 726, 727, 730, 731, 732, 747, 748, 750, 751, 755, 757, 758, 759, 760, 762, 765, 766, 773, 799, 800, 801, 802, 803, 804, 805, 806, 807, 808, 809, 810, 812, 813, 845, 846, 848, 849, 851, 852, 853, 854, 855, 856, 857, 858, 859, 860, 862, 863, 864, 868, 869, 927, 956, 957, 958, 959, 960, 1077, 1081, 1087, 1088, 1185, 1186, 1190, 1222, 1223, 1238, 1245, 1246, 1247, 1248, 1249, 1250, 1253, 1255, 1282, 1283, 1284, 1285, 1286, 1287, 1288, 1289, 1291, 1294, 1295, 1296, 1318, 1322, 1333, 1337, 1339, 1340, 1341, 1342, 1343, 1345, 1346, 1347, 1348, 1350, 1351, 1560, 1568, 1569,1571, 1572, 1573, and 1574.

Variants that show an increase in IScore relative to SEQ. ID. NO. 1 for at least one agretope include SEQ. ID. NO. 421, 422, 425, 426, 427, 431, 432, 443, 447, 448, 449, 452, 461, 463, 469, 470, 472, 473, 474, 476, 477, 484, 485, 486, 493, 496, 498, 500, 504, 524, 526, 533, 536, 540, 545, 549, 552, 556, 570, 571, 572, 573, 578, 595, 596, 597, 598, 602, 603, 604, 605, 606, 607, 615, 616, 617, 618, 627, 630, 631, 632, 641, 648, 649, 650, 654, 664, 665, 666, 667, 669, 671, 672, 673, 677, 678, 680, 682, 683, 684, 685, 686, 690, 693, 694, 700, 703, 706, 708, 720, 721, 722, 723, 724, 725, 726, 728, 735, 740, 741, 742, 743, 744, 745, 746, 752, 753, 754, 756, 761, 763, 764, 767, 768, 769, 770, 771, 772, 774, 775, 781, 782, 783, 784, 785, 786, 788, 789, 790, 791, 792, 793, 794, 795, 796, 834, 839, 858, 859, 860, 861, 863, 864, 865, 866, 867, 868, 871, 874, 876, 877, 878, 921, 928, 929, 930, 931, 932, 933, 934, 935, 936, 937, 945, 948, 949, 950, 952, 956, 957, 958, 959, 960, 962, 963, 964, 965, 985, 986, 987, 988, 989, 990, 991, 1016, 1017, 1018, 1037, 1038, 1039, 1041, 1051, 1053, 1067, 1070, 1071, 1072, 1073, 1074, 1079, 1080, 1083, 1085, 1087, 1088, 1092, 1099, 1100, 1101, 1102, 1103, 1104, 1105, 1109, 1112, 1114, 1116, 1127, 1130, 1135, 1136, 1137, 1138, 1139, 1140, 1143, 1144, 1145, 1146, 1148, 1149, 1150, 1151, 1152, 1153, 1154, 1155, 1156, 1157, 1158, 1160, 1162, 1163, 1164, 1165, 1166, 1167, 1168, 1169, 1170, 1171, 1172, 1173, 1174, 1175, 1176, 1187, 1188, 1189, 1190, 1192, 1197, 1208, 1210, 1224, 1225, 1228, 1230, 1233, 1239, 1250, 1251, 1252, 1253, 1254, 1259, 1260, 1270, 1273, 1285, 1288, 1290, 1291, 1292, 1293, 1294, 1295, 1296, 1302, 1309, 1313, 1318, 1322, 1324, 1328, 1333, 1337, 1343, 1344, 1345, 1346, 1347, 1383, 1534, 1535, 1536, 1537, 1538, 1539, 1558, 1559, 1564, 1565, 1568, 1569, and 1570.

Variants that show no change in IScore relative to SEQ. ID. NO.1 for at least one agretope include SEQ. ID. NO. 424, 430, 438, 439, 440, 441, 442, 445, 446, 454, 455, 456, 457, 458, 459, 465, 466, 467, 468, 471, 475, 478, 479, 480, 481, 482, 483, 487, 488, 489, 490, 492, 495, 497, 499, 501, 502, 503, 505, 506, 507, 508, 509, 510, 511, 512, 513, 514, 515, 516, 517, 518, 519, 520, 521, 523, 525, 527, 528, 529, 530, 531, 532, 534, 535, 537, 538, 539, 541, 542, 543, 544, 546, 547, 548, 557, 558, 559, 560, 561, 562, 563, 564, 565, 566, 567, 568, 569, 574, 575, 576, 577, 579, 580, 581, 582, 583, 584, 585, 586, 587, 588, 589, 590, 591, 592, 593, 594, 599, 600, 609, 610, 611, 612, 613, 614, 619, 620, 621, 622, 623, 624, 625, 626, 628, 629, 633, 634, 635, 636, 637, 638, 639, 640, 645, 646, 647, 651, 652, 653, 655, 656, 657, 658, 659, 660, 661, 662, 663, 674, 675, 676, 679, 681, 687, 688, 689, 691, 692, 695, 696, 697, 698, 699, 701, 702, 704, 705, 707, 709, 710, 711, 713, 714, 715, 716, 718, 719, 729, 733, 734, 736, 737, 738, 739, 749, 776, 777, 778, 779, 780, 787, 797, 798, 811, 814, 815, 816, 817, 818, 819, 820, 821, 822, 823, 824, 825, 826, 827, 828, 829, 830, 831, 832, 833, 835, 836, 837, 838, 840, 841, 842, 843, 844, 847, 850, 870, 872, 873, 875, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 922, 923, 924, 925, 926, 938, 939, 940, 941, 942, 943, 944, 946, 947, 951, 953, 954, 955, 961, 966, 967, 968, 969, 970, 971, 972, 973, 974, 975, 976, 977, 978, 979, 980, 981, 982, 983, 984, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1019, 1020, 1021, 1022, 1023, 1024, 1025, 1026, 1027, 1028, 1029, 1030, 1031, 1032, 1033, 1034, 1035, 1036, 1040, 1042, 1043, 1044, 1045, 1046, 1047, 1048, 1049, 1050, 1052, 1054, 1055, 1056, 1057, 1058, 1059, 1060, 1061, 1062, 1063, 1064, 1065, 1066, 1068, 1069, 1075, 1076, 1078, 1082, 1084, 1086, 1089, 1090, 1091, 1093, 1094, 1095, 1096, 1097, 1098, 1106, 1107, 1108, 1110, 1111, 1113, 1115, 1117, 1118, 1119, 1120, 1121, 1122, 1123, 1124, 1125, 1126, 1128, 1129, 1131, 1132, 1133, 1134, 1141, 1142, 1147, 1159, 1161, 1177, 1178, 1179, 1180, 1181, 1182, 1183, 1184, 1191, 1193, 1194, 1195, 1196, 1198, 1199, 1200, 1201, 1202, 1203, 1204, 1205, 1206, 1207, 1209, 1211, 1212, 1213, 1214, 1215, 1216, 1217, 1218, 1219, 1220, 1221, 1226, 1227, 1229, 1231, 1232, 1234, 1235, 1236, 1237, 1240, 1241, 1242, 1243, 1244, 1256, 1257, 1258, 1261, 1262, 1263, 1264, 1265, 1266, 1267, 1268, 1269, 1271, 1272, 1274, 1275, 1276, 1277, 1278, 1279, 1280, 1281, 1297, 1298, 1299, 1300, 1301, 1303, 1304, 1305, 1306, 1307, 1308, 1310, 1311, 1312, 1314, 1315, 1316, 1317, 1319, 1320, 1321, 1323, 1325, 1326, 1327, 1329, 1330, 1331, 1332, 1334, 1335, 1336, 1338, 1349, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1540, 1541, 1542, 1543, 1544, 1545, 1546, 1547, 1548, 1549, 1550, 1551, 1552, 1553, 1554, 1555, 1556, 1557, 1561, 1562, 1563, 1566, 1567, 1575, 1576, 1577, 1578, 1579, 1580, 1581, and 1582.

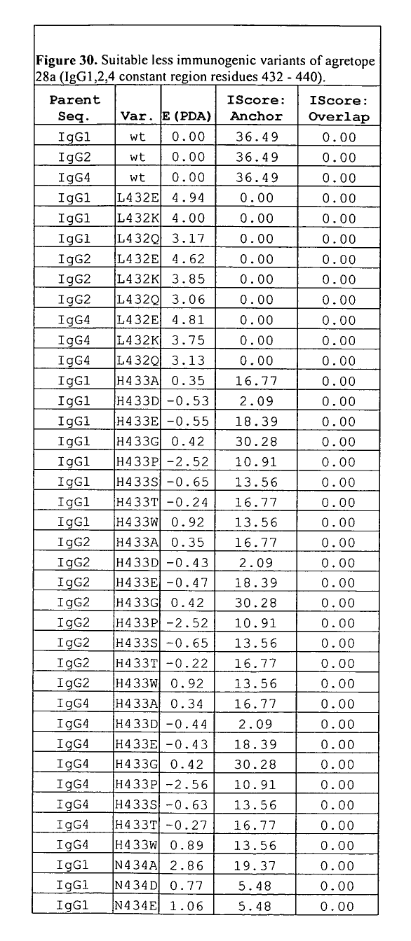

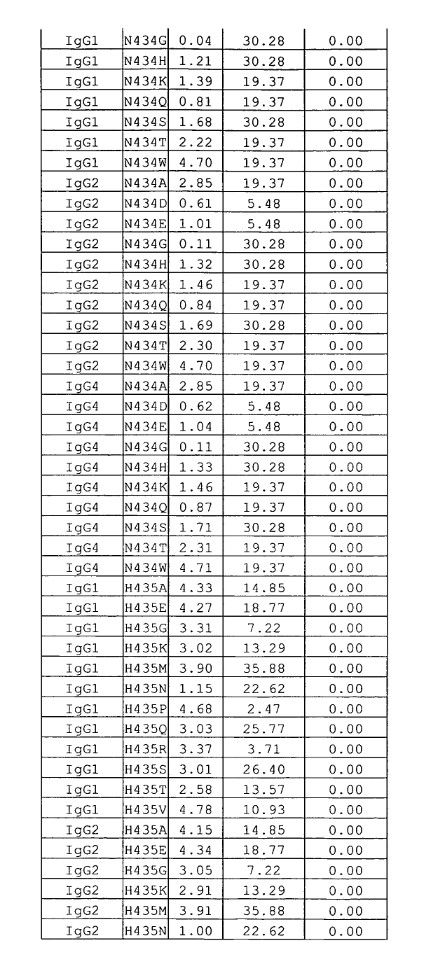

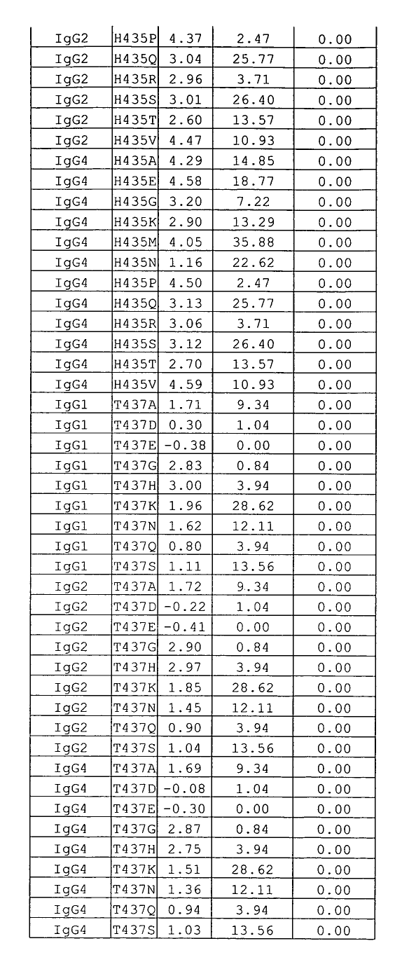

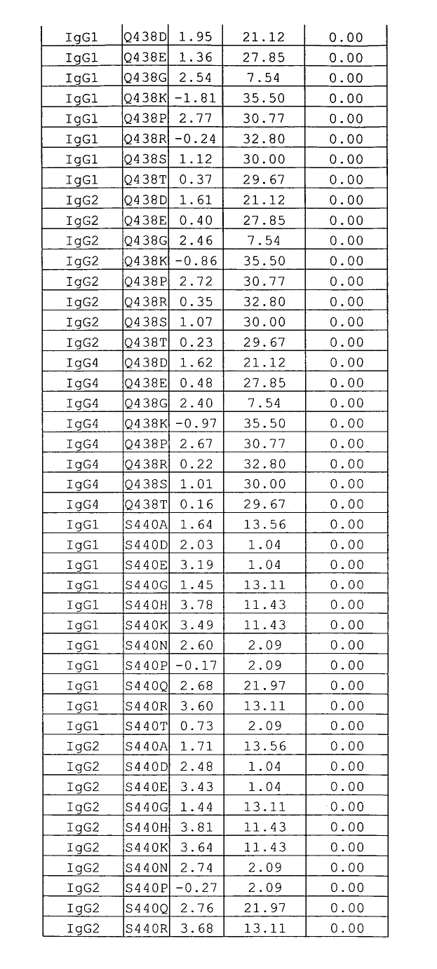

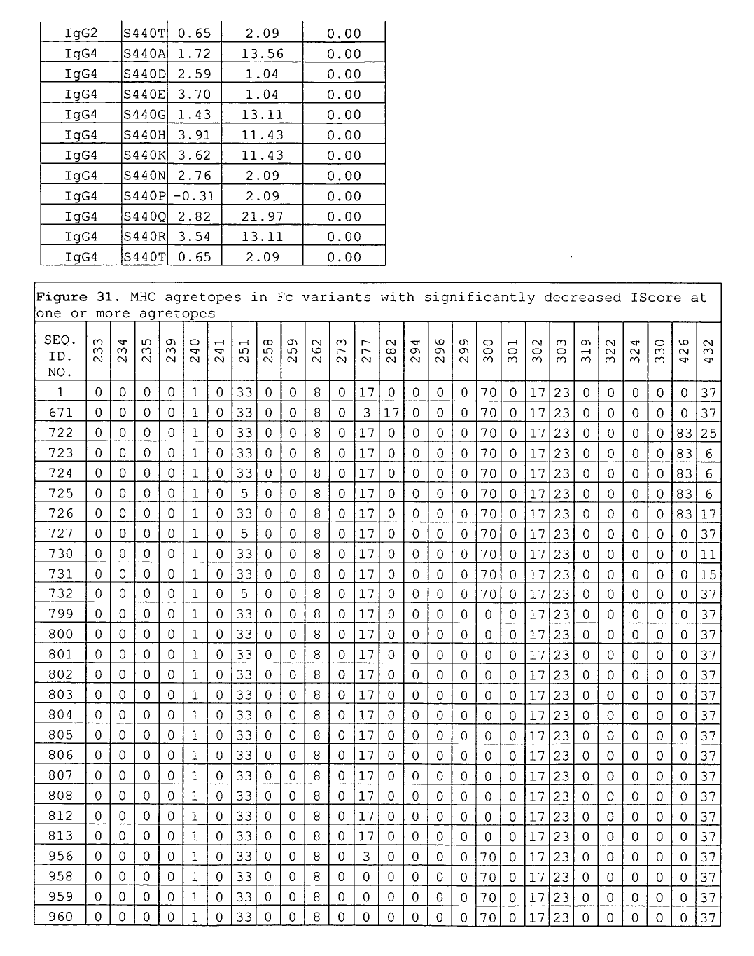

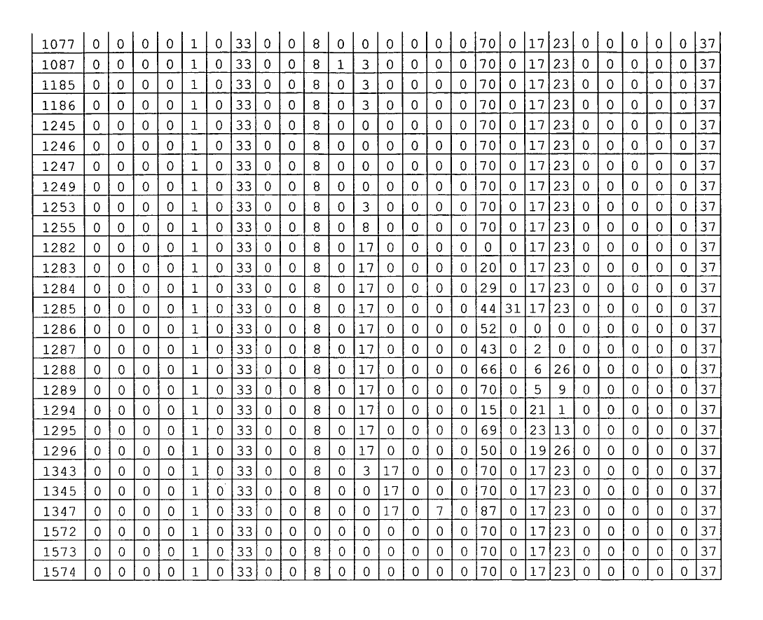

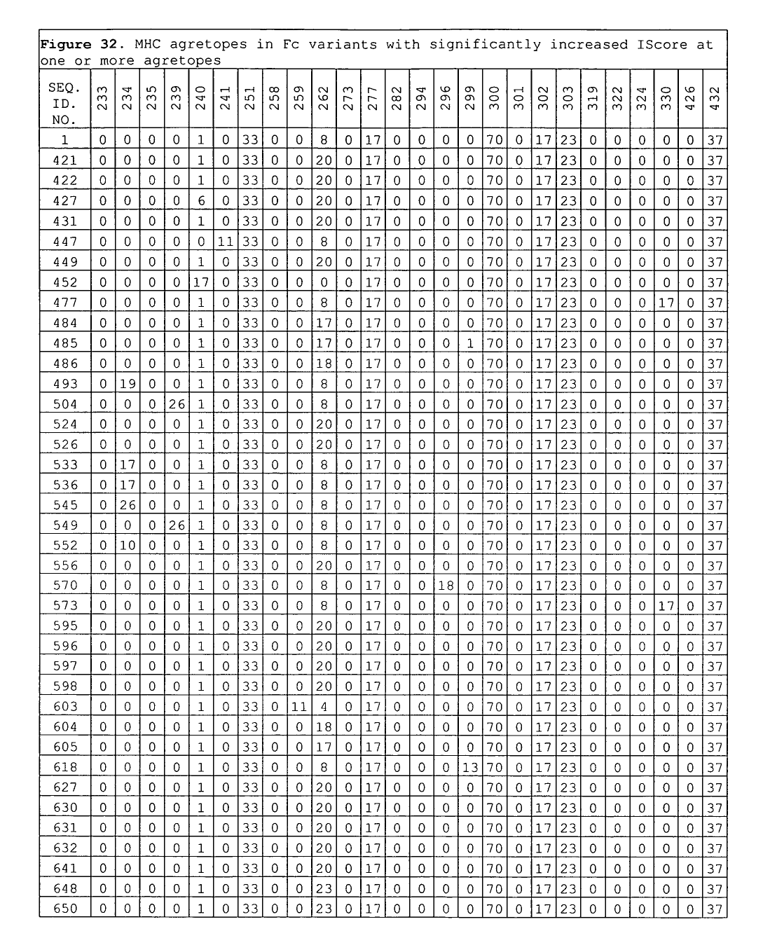

FIGS. 31 and 32 show a comparison of MHC agretopes in the constant domains of human IgG1 (SEQ ID NO:1) versus the engineered Fc variants of SEQ ID NO 420-1582. Columns are included for agretopes with IScore≧10 in at least one variant are shown. The agretopes corresponding to residues 369-377 and 404-412 are the same in all the variants and are therefore not shown below. FIG. 31 shows only those Fc variants that have a decrease in IScore of greater than 9.0 for one or more agretopes, while FIG. 32 shows only those Fc variants that have an increase in IScore of greater than 9.0 for one or more agretopes.

Of special interest are variants with substitutions that lower IScore of at least one agretope from 25 or higher to lower than 10, and that do not exhibit substantial increases in IScore for any nine-mer fragment. Variants that substantially decrease IScore for agretope 16 include, but are not limited to, SEQ_ID_NO: 727 (M252L/I253G) and 732 (I253G). Variants that substantially decrease IScore for agretope 18 include, but are not limited to, SEQ_ID_NO: 956 (D280K), 958 (D280W), 959 (D280P), 960 (D280G), 1077 (Y278D), 1185 (H285D), 1186 (H285E), 1245 (V282E), 1246 (V282K), 1247 (V282Y), 1249 (V282G), 1255 (E283G), 1572 (S267E/V282G), 1573 (G281D/V282G), and 1574 (V282G/P331D). Variants that substantially decrease IScore for agretope 19a include, but are not limited to, SEQ_ID_NO: 799 (Y300D), 800 (Y300E), 801 (Y300N), 802 (Y300Q), 803 (Y300K), 804 (Y300R), 805 (Y300S), 806 (Y300T), 807 (Y300H), 808 (Y300A), 812, (Y300P), 813 (Y300G), and 1282 (R301D). Variants that substantially decrease IScore for agretope 20a include, but are not limited to, SEQ_ID_NO: 1286 (V303D), 1287 (V303E), 1288 (V303Y), and 1289 (S304D). Variants that substantially decrease IScore for agretope 21a include, but are not limited to, SEQ_ID_NO: 1286 (V303D), 1287 (V303E), 1289 (S304D), and 1294 (V305E).

Examples of Fc variants with substantially increased IScore for at least one nine-mer agretope include but are not limited to SEQ ID NO 929-933, which has a significantly higher IScore for agretope 17a (residues 262-270) than the parent protein of SEQ ID NO 1 due to the D270S, D270L, D270I, D270F, and D270M substitutions; SEQ ID NO 1101-1105, which has a new agretope at residues 322-330 with IScore of 31 or 45 that is caused by the K332V, K332I, K332F, K332Y, and K332W substitutions; and SEQ ID NO 1149-1157, which have a new agretope at residues 234-242 with IScore of 15-39 that is caused by the G237S, G237T, G237H, G237H, G237V, G237L, G237I, G237F, G237M, and G237Y substitutions.

Compensatory mutations that are included in the library of Fc variants, reduce IScore for residues 234-242 in SEQ ID NO 1149-1157 to less than 10, and that do not introduce any new agretopes include but are not limited to L234A, L234D, L234E, L234G, L234H, L234K, L234N, L234P, L234S, and L234T; as well as L235D, S239D, and S239E for SEQ ID NO 1149 (G237S) and SEQ ID NO 1150 (G237T); G236D, G236E, L235D, L235E, L235P, L235S, L235T, S239D, S239E, S239H, and V240A for SEQ ID NO 1151 (G237H), SEQ ID NO 1153 (G237L) SEQ ID NO 1156 (G237M); G236D, G236E, L235D, L235P, L235S, S239D, S239E, S239H, and V240A for SEQ ID NO 1152 (G237V) and SEQ ID NO 1155 (G237F); G236D, L235D, L235P, and V240A for SEQ ID NO 1154 (G237I); and G236D, G236E, L235D, L235E, L235P, L235S, L235T, S239D, S239E, and S239H for SEQ ID NO 1157 (G237Y).

Compensatory mutations that are included in the library of Fc variants, reduce IScore for residues 262-270 in SEQ ID NO 929-933 to less than 10, and that do not introduce any new agretopes include but are not limited to V262E, V263A, V263T, V264D, S267D, S267E, S267F, and S267Y; as well as V263I, V263M, V264E, S267M, S267Q, S267W, and H268R for SEQ ID NO 930 (D270L); V263M, V264E, S267M, and S267W for SEQ ID NO 931 (D270I); V264E and S267W for SEQ ID NO 932 (D270F); and V264E and S267W for SEQ ID NO 933 (D270M).

Compensatory mutations that are included in the library of Fc variants, reduce IScore for residues 322-330 in SEQ ID NO 1101-1105 to less than 10, and that do not introduce any new agretopes include but are not limited to S324D, N325P, A327D, A327E; as well as A327W, L328D, L328E, L328G, L328K, L328S, A330Y, A330R, A330W, A330E, A330N, A330P, and A330G for SEQ ID NO 1101 (K332V); L328D and L328G for SEQ ID NO 1104 (K332Y); and L328D and L328G for SEQ ID NO 1105 (K332W).

The MHC agretopes in the most preferred Fc variants are quite similar to the MHC agretopes in the parent human IgG1 sequence of SEQ ID NO:1, as shown in FIG. 33.

### Example 5

### Reducing Variable Domain MHC Binding Without Increasing Non-Human Sequence Content

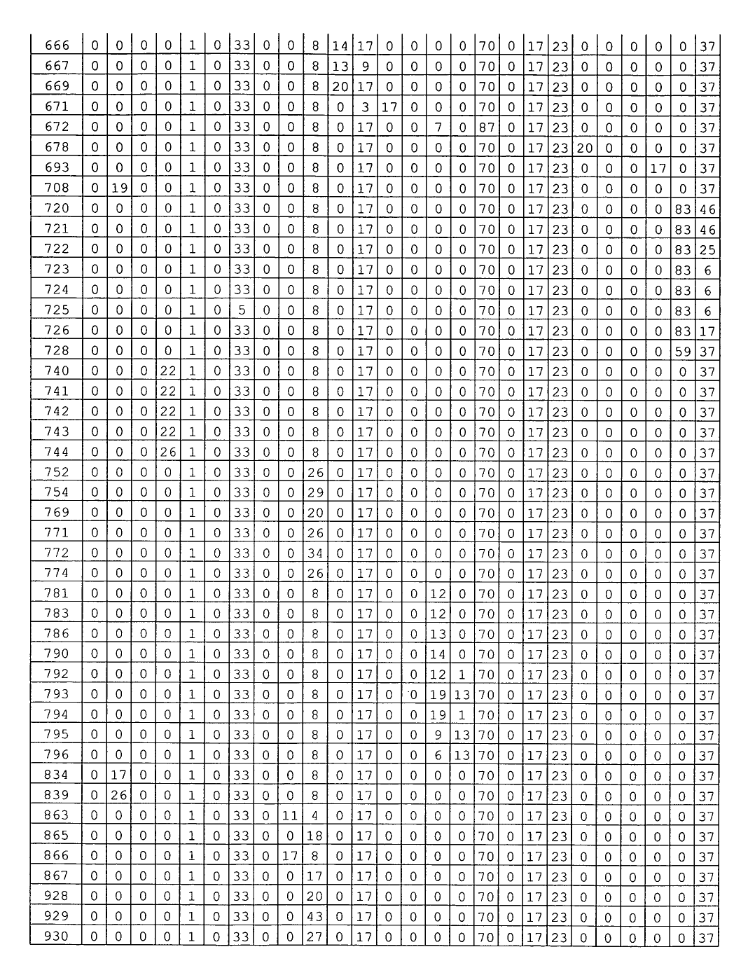

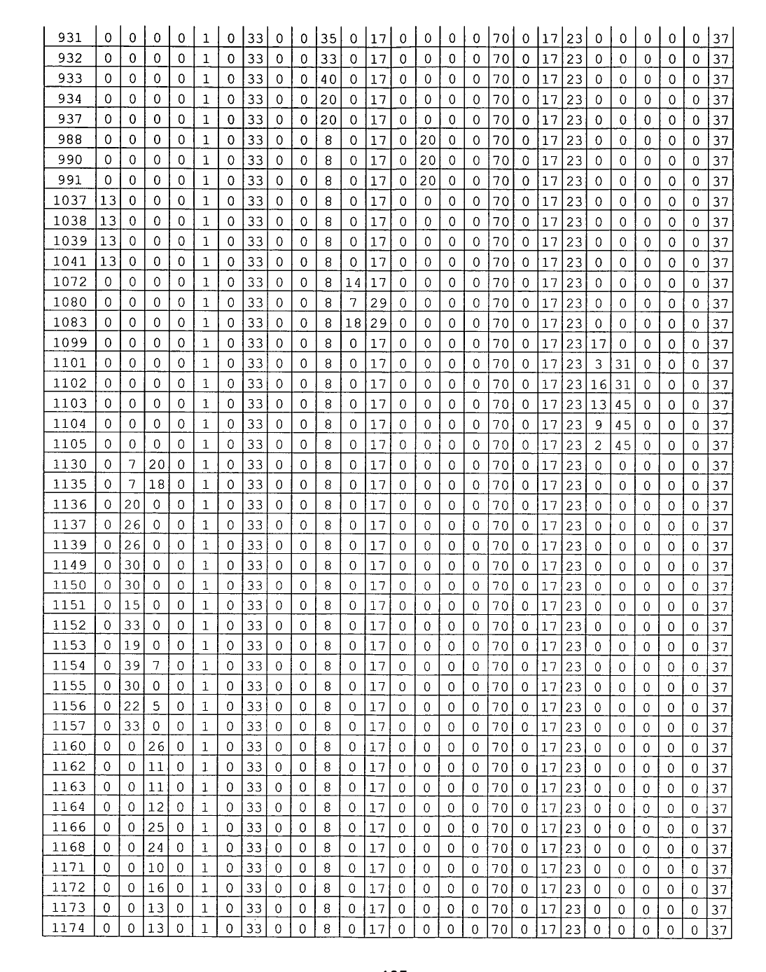

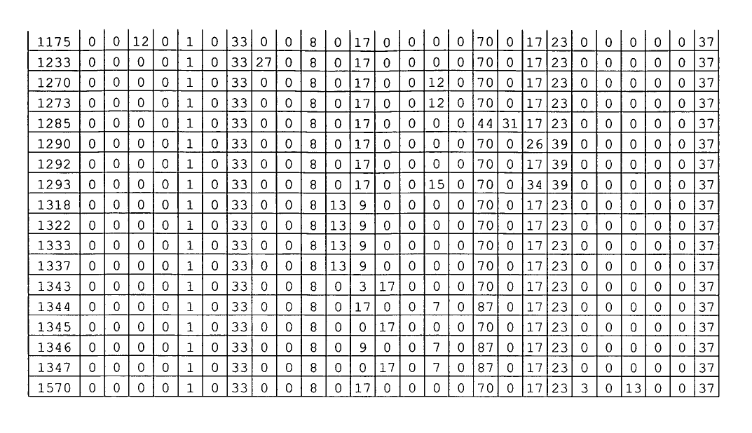

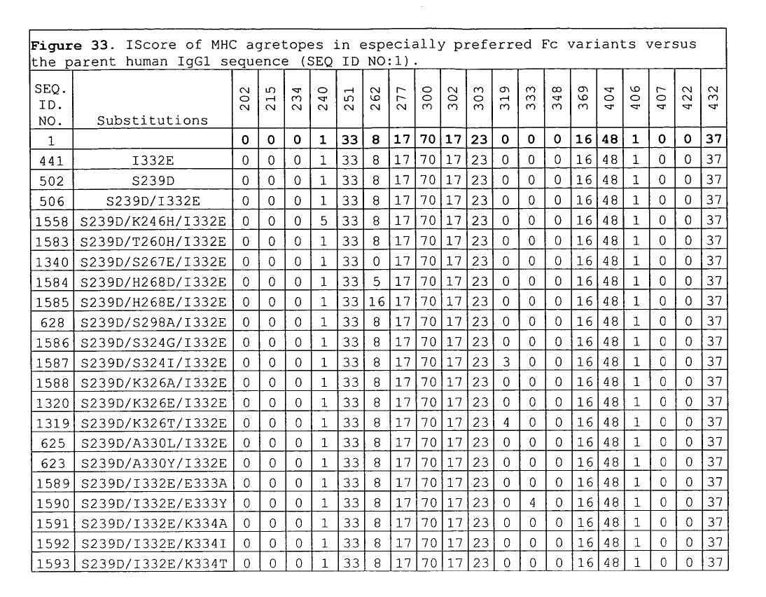

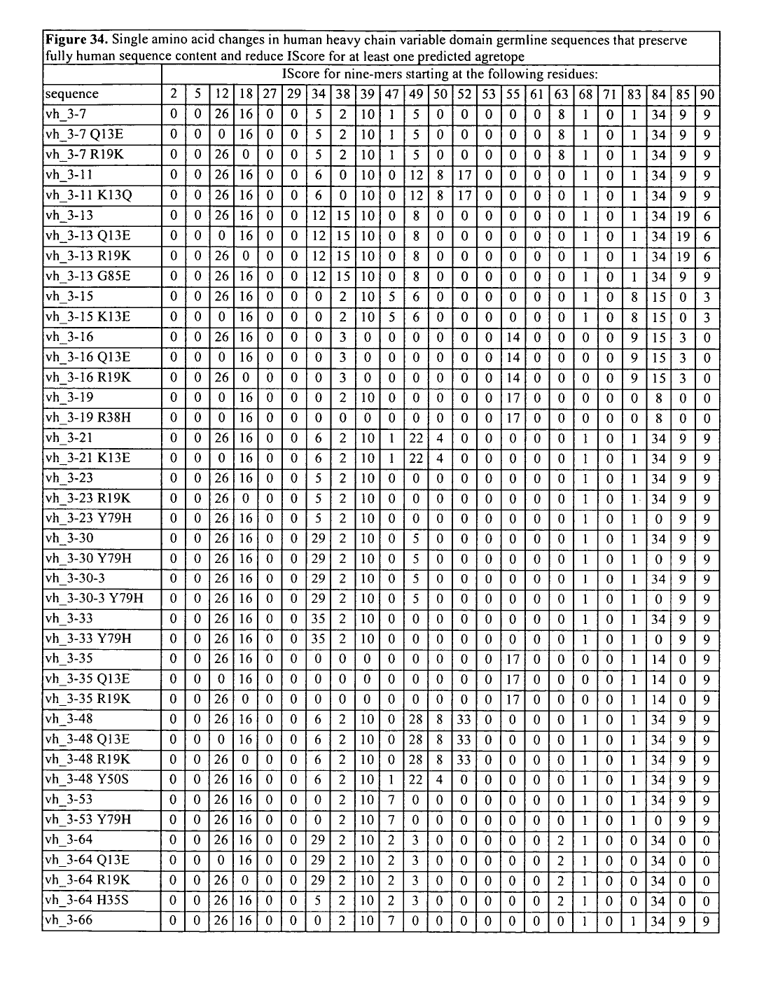

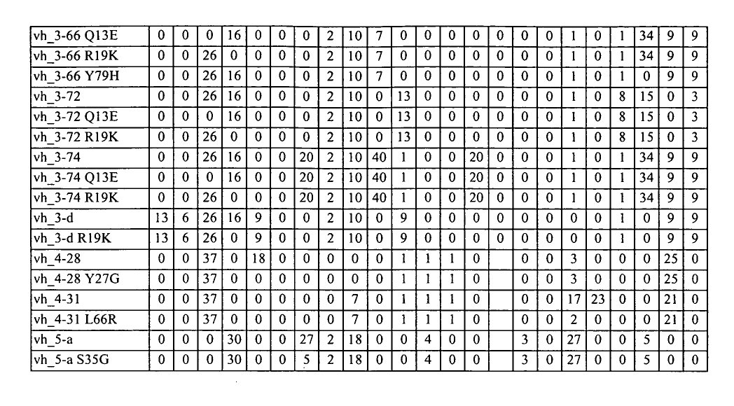

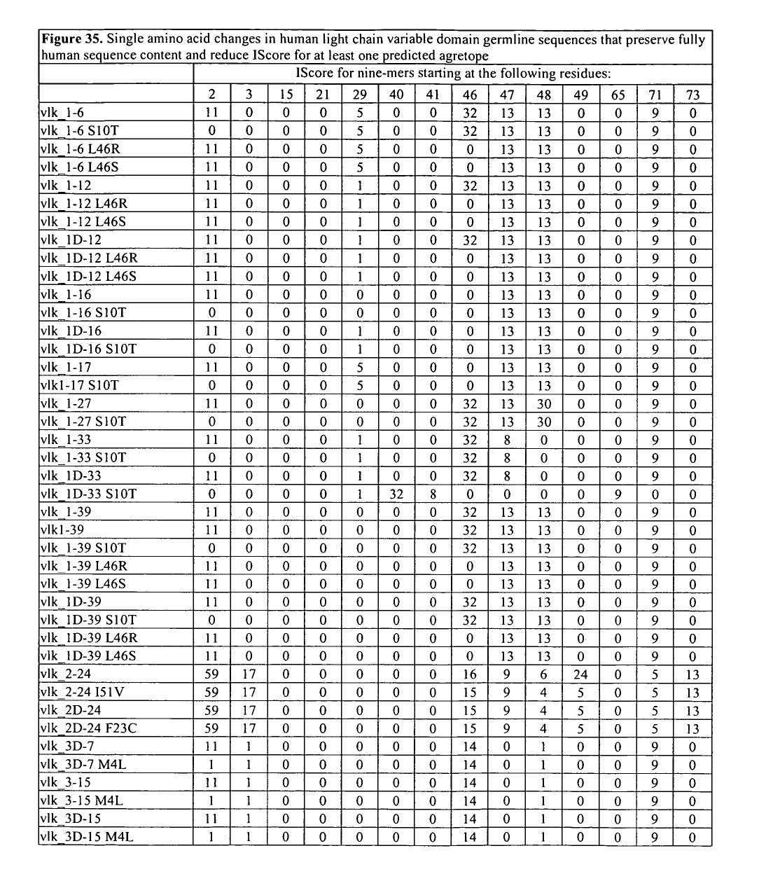

Due to the diversity of human germline variable domains, it is possible to construct additional sequences with fully human sequence content (defined in this example to mean that all nine residue fragments are present in at least one human germline sequence). All of the single amino acid changes that retain fully human sequence content were computationally generated and analyzed for predicted MHC binding. In FIGS. 34 and 35, IScore is shown for each of the nine-mers at which a single amino acid change that preserves fully human sequence content reduces IScore significantly relative to the parent human heavy chain variable domain sequence.

Single amino acid changes in a given variable domain may be combined freely so long as they are separated by nine or more residues. For example, in vlk_{—}1-6 the substitution S10T may be combined with either L46R or L46S to yield a sequence with fully human sequence content and that has low predicted MHC binding for all nine-mer fragments. Furthermore, the approach described here may be extended beyond single substitutions. That is, multiple substitutions (either within a nine-mer fragment or not) may be added while retaining fully human sequence content.

Additional substitutions including but not limited to those described herein may be incorporated into these variants in order to reduce the immunogenicity further. Residue numbering is based on that of IgG1.

While the foregoing invention has been described above, it will be clear to one skilled in the art that various changes and additional embodiments made be made without departing from the scope of the invention. All references cited herein, including patents, patent applications,(provisional, utility and PCT), and publications are incorporated by reference in their entirety.

We claim:

1. A non-naturally occurring protein comprising a variant Fc region having the formula:
-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377j-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-;
wherein
X(118) is A;
X(119) is S;
X(120) is T;
X(121) is K;
X(122) is G;
X(123) is P;
X(124) is S;
X(125) is V;
X(126) is F;
X(127) is P;
X(128) is L;
X(129) is A;
X(130) is P;
X(131) is S;
X(132) is S;
X(133) is K;
X(134) is S;
X(135) is T;
X(136) is S;
X(137) is G;
X(138) is G;
X(139) is T;
X(140) is A;
X(141) is A;
X(142) is L;
X(143) is G;
X(144) is C;
X(145) is L;
X(146) is V;
X(147) is K;
X(148) is D;
X(149) is Y;
X(150) is F;
X(151) is P;
X(152) is E;
X(153) is P;
X(154) is V;
X(155) is T;
X(156) is V;
X(157) is S;
X(158) is W;
X(159) is N;
X(160) is S;
X(161) is G;
X(162) is A;
X(163) is L;
X(164) is T;
X(165) is S;
X(166) is G;
X(167) is V;
X(168) is H;
X(169) is T;
X(170) is F;
X(171) is P;
X(172) is A;
X(173) is V;
X(174) is L;
X(175) is Q;
X(176) is S;
X(177) is S;
X(178) is G;
X(179) is L;
X(180) is Y;
X(181) is S;
X(182) is L;
X(183) is S;
X(184) is S;
X(185) is V;
X(186) is V;
X(187) is T;
X(188) is V;
X(189) is P;
X(190) is S;
X(191) is S;
X(192) is S;
X(193) is L;
X(194) is G;
X(195) is T;
X(196) is Q;
X(197) is T;
X(198) is Y;
X(199) is I;
X(200) is C;
X(201) is N;
X(202) is V;
X(203) is N;
X(204) is H;
X(205) is K;
X(206) is P;
X(207) is S;
X(208) is N;
X(209) is T;
X(210) is K;
X(211) is V;
X(212) is D;
X(213) is K;
X(214) is K;
X(215) is V;
X(216) is E;
X(217) is P;
X(218) is K;
X(219) is S;
X(220) is C;
X(221) is D;
X(222) is K;
X(223) is T;
X(224) is H;
X(225) is T;
X(226) is C;
X(227) is P;
X(228) is P;
X(229) is C;
X(230) is P;
X(231) is A;
X(232) is P;
X(233) is E;
X(234) is L;
X(235) is L;
X(236) is G;
X(237) is G;
X(238) is P;
X(239) is S;
X(240) is V;
X(241) is F;
X(242) is L;
X(243) is F;
X(244) is P;
X(245) is P;
X(246) is K;
X(247) is P;
X(248) is K;
X(249) is D;
X(250) is T;
X(251) is selected from the group consisting of L, D, E, H, and T;
X(252) is selected from the group consisting of M, D, E, and H;
X(253) is selected from the group consisting of I, D, E, F, H, K, L, N, P, Q, R, S, T, V, W, and Y;
X(254) is selected from the group consisting of S, E, K, N, P, Q, R, V, and W;
X(255) is R;
X(256) is selected from the group consisting of T, I, L, M, P, S, V, W, and Y;
X(257) is P;
X(258) is E;
X(259) is selected from the group consisting of V and T;
X(260) is T;
X(261) is C;
X(262) is V;
X(263) is V;
X(264) is V;
X(265) is D;
X(266) is V;
X(267) is S;
X(268) is H;
X(269) is E;
X(270) is D;
X(271) is P;
X(272) is E;
X(273) is V;
X(274) is K;
X(275) is F;
X(276) is N;
X(277) is W;
X(278) is selected from the group consisting of Y, D and E;
X(279) is selected from the group consisting of V, A, Q, and T;
X(280) is D;
X(281) is G;
X(282) is selected from the group consisting of V, F, I, L, P, Q, W, and Y;
X(283) is E;
X(284) is V;
X(285) is selected from the group consisting of H, E, P, and T;
X(286) is N;
X(287) is A;
X(288) is K;
X(289) is T;
X(290) is K;
X(291) is P;
X(292) is R;
X(293) is E;
X(294) is E;
X(295) is Q;
X(296) is Y;
X(297) is N;
X(298) is S;
X(299) is T;
X(300) is Y,
X(301) is selected from the group consisting of R, G, K, and Q;
X(302) is selected from the group consisting of D, E, H, K, Q, S, and T;
X(303) is selected from the group consisting of V, N, P, Q, R, and S;
X(304) is S;
X(305) is selected from the group consisting of V, G, P, and T;
X(306) is selected from the group consisting of L, F, H, I, N, T, V, and Y;
X(307) is T;
X(308) is selected from the group consisting of V, A, N, P, and S;
X(309) is selected from the group consisting of L, F, G, I, M, N, Q, S, T, V, W, and Y;
X(310) is H;
X(311) is selected from the group consisting of Q, D, E, G, P, T, and W;
X(312) is D;
X(313) is W;
X(314) is L;
X(315) is N;
X(316) is G;
X(317) is K;
X(318) is E;
X(319) is Y;
X(320) is K;
X(321) is C;
X(322) is K;
X(323) is V;
X(324) is S;
X(325) is N;
X(326) is K;
X(327) is A;
X(328) is L;
X(329) is P;
X(330) is A;
X(331) is P;
X(332) is l;
X(333) is E;
X(334) is K;
X(335) is T;
X(336) is I;
X(337) is S;
X(338) is K;
X(339) is A;
X(340) is K;
X(341) is G;
X(342) is Q;
X(343) is P;
X(344) is R;
X(345) is E;
X(346) is P;
X(347) is Q;
X(348) is V;
X(349) is Y;
X(350) is T;
X(351) is L;
X(352) is P;
X(353) is P;
X(354) is S;
X(355) is R;
X(356) is D;
X(357) is E;
X(358) is L;
X(359) is T;
X(360) is K;
X(361) is N;
X(362) is Q;
X(363) is V;
X(364) is S;
X(365) is L;
X(366) is T;
X(367) is C;
X(368) is L;
X(369) is V;
X(370) is K;
X(371) is G;
X(372) is F;
X(373) is Y;
X(374) is P;
X(375) is S;
X(376) is D;
X(377) is I;
X(378) is A;
X(379) is V;
X(380) is E;
X(381) is W;
X(382) is E;
X(383) is S;
X(384) is N;
X(385) is G;
X(386) is Q;
X(387) is P;
X(388) is E;
X(389) is N;
X(390) is N;
X(391) is Y;
X(392) is K;
X(393) is T;
X(394) is T;
X(395) is P;
X(396) is P;
X(397) is V;
X(398) is L;
X(399) is D;
X(400) is S;
X(401) is D;
X(402) is G;
X(403) is S;
X(404) is selected from the group consisting of F, H, I, L, M, N, Q, T, and V;
X(405) is selected from the group consisting of F and W;
X(406) is selected from the group consisting of L, A, D, E, G, K, N, Q, S, T, and V;
X(407) is selected from the group consisting of Y and H;
X(408) is S;
X(409) is K;
X(410) is selected from the group consisting of L, I and Q;
X(411) is T;
X(412) is selected from the group consisting of V and P;
X(413) is D;
X(414) is K;
X(415) is S;
X(416) is R;
X(417) is W;
X(418) is Q;
X(419) is Q;
X(420) is G;
X(421) is N;
X(422) is V;
X(423) is F;
X(424) is S;
X(425) is C;
X(426) is S;
X(427) is V;
X(428) is M;
X(429) is H;
X(430) is E;
X(431) is A;
X(432) is selected from the group consisting of L, E and K;
X(433) is selected from the group consisting of H, D, G, P, S, T, and W;
X(434) is selected from the group consisting of N, D, E, G, H, S, T, and W;
X(435) is selected from the group consisting of H, G, K, M, N, P, S, T, and V;
X(436) is Y;
X(437) is selected from the group consisting of T, D, E, G, H, K, N, Q, and S;
X(438) is selected from the group consisting of Q, G, P, S, and T; and,
X(439) is K;
X(440) is selected from the group consisting of S, D, E, G, H, K, N, P, Q, R, and T,
X(441) is L;
X(442) is S;
X(443) is L;
X(444) is S;
X(445) is P;
X(446) is G;
X(447) is K;
said non-naturally occurring protein having reduced immunogenicity as compared with a protein comprising SEQ ID NO:1.

2. A non-naturally occurring protein comprising a variant Fc region comprising at least one amino acid modification of SEQ ID NO:1, said modification at a position selected from the group consisting of positions 251, 252, 253, 254, 256, 259, 278, 279, 282, 285, 301, 302, 303, 305, 306, 308, 309, 311, 404, 405, 406, 407, 410, 412, 432, 433, 434, 435, 437, 438, and 440, wherein said modification at position 251 is selected from the group consisting of D, E, H, and T; wherein said modification at position 252 is selected from the group consisting of D, E, and H; wherein said modification at position 253 is selected from the group consisting of D, E, F, H, K, L, N, P, Q, R, S, T, V, W, and Y; wherein said modification at position 254 is selected from the group consisting of E, K, N, P, Q, R, V, and W; wherein said modification at position 256 is selected from the group consisting of I, L, M, P, S, V, W, and Y; wherein the modification at position 259 is T; wherein said modification at position 278 is selected from the group consisting of D and E; wherein said modification at position 279 is selected from the group consisting of A, Q, and T; wherein said modification at position 282 is selected from the group consisting of F, I, L, P, Q, W, and Y; wherein said modification at position 285 is selected from the group consisting of P and T; wherein said modification at position 301 is selected from the group consisting of G, K, and Q; wherein said modification at position 302 is selected from the group consisting of A, D, E, H, K, Q, S, and T; wherein said modification at position 303 is selected from the group consisting of N, P, Q, R, and S; wherein said modification at position 305 is selected from the group consisting of G, P, and T; wherein said modification at position 306 is selected from the group consisting of F, H, I, N, T, V, and Y; wherein said modification at position 308 is selected from the group consisting of A, N, P, and S; wherein said modification at position 309 is selected from the group consisting of F, G, I, M, N, Q, S, T, V, W, and Y; wherein said modification at position 311 is selected from the group consisting of D, E, G, P, T, and W; wherein said modification at position 404 is selected from the group consisting of H, I, L, M, N, Q, T, and V; wherein the modification at position 405 is W; wherein said modification at position 406 is selected from the group consisting of A, D, E, G, K, N, Q, S, T, and V; wherein the modification at position 407 is H; wherein said modification at position 410 is selected from the group consisting of I and Q; wherein the modification at position 412 is P; wherein said modification at position 432 is selected from the group consisting of E and K; wherein said modification at position 433 is selected from the group consisting of D, G, P, S, T, and W; wherein said modification at position 434 is selected from the group consisting of D, E, G, H, S, T, and W; wherein said modification at position 435 is selected from the group consisting of G, K, M, N, P, S, T, and V; wherein said modification at position 437 is selected from the group consisting of D, E, G, H, K, N, Q, and S; wherein said modification at position 438 is selected from the group consisting of G, P, S, and T; and, wherein said modification at position 440 is selected from the group consisting of D, E, G, H, K, N, P, Q, R, and T, said non-naturally occurring protein having reduced immunogenicity as compared with a protein comprising SEQ ID NO:1.

3. A variant protein of claim 2, wherein said modification at position 251 is selected from the group consisting of D, E, H, and T; wherein the modification at position 252 is D; wherein said modification at position 253 is selected from the group consisting of D and E; wherein said modification at position 256 is selected from the group consisting of M, W, and Y; wherein the modification at position 278 is D; wherein said modification at position 282 is selected from the group consisting of F, L, Q, W, and Y; wherein said modification at position 303 is selected from the group consisting of N, P, Q, R, and S; wherein the modification at position 311 is D; wherein said modification at position 404 is selected from the group consisting of H, N, Q, and T; wherein said modification at position 432 is selected from the group consisting of E and K; and, wherein the modification at position 437 is E.

4. A variant protein of claim 2 wherein at least one modification is made to an amino acid in the group consisting of Agretope 16 (251-259), Agretope 17a (262-270), Agretope 18 (277-285), Agretope 19a (300-308), Agretope 20a (302-310), Agretope 21a (303-311), Agretope 23 (369-377), Agretope 24a (404-412), Agretope 25a (406-414), Agretope 27a (422-430), and Agretope 28a (432-440).

5. A non-naturally occurring protein comprising a variant Fc region having the formula:
-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(l151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-; wherein
X(118) is A;
X(119) is S;
X(120) is T;
X(121) is K;
X(122) is G;
X(123) is P;
X(124) is S;
X(125) is V;
X(126) is F;
X(127) is P;
X(128) is L;
X(129) is A;
X(130) is P;
X(131) is C;
X(132) is S;
X(133) is R;
X(134) is S;
X(135) is T;
X(136) is S;
X(137) is E;
X(138) is S;
X(139) is T;
X(140) is A;
X(141) is A;
X(142) is L;
X(143) is G;
X(144) is C;
X(145) is L;
X(146) is V;
X(147) is K;
X(148) is D;
X(149) is Y;
X(150) is F;
X(151) is P;
X(152) is E;
X(153) is P;
X(154) is V;
X(155) is T;
X(156) is V;
X(157) is S;
X(158) is W;
X(159) is N;
X(160) is S;
X(161) is G;
X(162) is A;
X(163) is L;
X(164) is T;
X(165) is S;
X(166) is G;
X(167) is V;
X(168) is H;
X(169) is T;
X(170) is F;
X(171) is P;
X(172) is A;
X(173) is V;
X(174) is L;
X(175) is Q;
X(176) is S;
X(177) is S;
X(178) is G;
X(179) is L;
X(180) is Y;
X(181) is S;
X(182) is L;
X(183) is S;
X(184) is S;
X(185) is V;
X(186) is V;
X(187) is T;
X(188) is V;
X(189) is P;
X(190) is S;
X(191) is S;
X(192) is N;
X(193) is F;
X(194) is G;
X(195) is T;
X(196) is Q;
X(197) is T;
X(198) is Y;
X(199) is T;
X(200) is C;
X(201) is N;
X(202) is V;
X(203) is D;
X(204) is H;
X(205) is K;
X(206) is P;
X(207) is S;
X(208) is N;
X(209) is T;
X(210) is K;
X(211) is V;
X(212) is D;
X(213) is K;
X(214) is T;
X(215) is V;
X(216) is E;
X(217) is R;
X(218) is K;
X(219) is C;
X(220) is C;
X(221) is a bond;
X(222) is V;
X(223) is a bond;
X(224) is E;
X(225) is a bond;
X(226) is C;
X(227) is P;
X(228) is P;
X(229) is C;
X(230) is P;
X(231) is A;
X(232) is P;
X(233) is P;
X(234) is V;
X(235) is A;
X(236) is a bond;
X(237) is G;
X(238) is P;
X(239) is S;
X(240) is V;
X(241) is F;
X(242) is L;
X(243) is F;
X(244) is P;
X(245) is P;
X(246) is K;
X(247) is P;
X(248) is K;
X(249) is D;
X(250) is T;
X(251) is selected from the group consisting of L, D, E, H, and T;
X(252) is selected from the group consisting of M, D, E, and H;
X(253) is selected from the group consisting of I, D, E, F, H, K, L, N, P, Q, R, S, T, V, W, and Y;
X(254) is selected from the group consisting of S, E, K, N, P, Q, R, V, and W;
X(255) is R;
X(256) is selected from the group consisting of T, I, L, M, P, S, V, W, and Y;
X(257) is P;
X(258) is E;
X(259) is selected from the group consisting of V and T;
X(260) is T;
X(261) is C;
X(262) is V;
X(263) is V;
X(264) is V;
X(265) is D;
X(266) is V;
X(267) is S;
X(268) is H;
X(269) is E;
X(270) is D;
X(271) is P;
X(272) is E;
X(273) is V;
X(274) is Q;
X(275) is F;
X(276) is N;
X(277) is W;
X(278) is selected from the group consisting of Y, D and E;
X(279) is selected from the group consisting of V, A, Q, and T;
X(280) is D;
X(281) is G;
X(282) is selected from the group consisting of V, F, I, L, Q, and W;
X(283) is E;
X(284) is V;
X(285) is selected from the group consisting of H, P, and T;
X(286) is N;
X(287) is A;
X(288) is K;
X(289) is T;
X(290) is K;
X(291) is P;
X(292) is R;
X(293) is E;
X(294) is E;
X(295) is Q;
X(296) is F;
X(297) is N;
X(298) is S;
X(299) is T;
X(300) is F;
X(301) is selected from the group consisting of R, G, K, and Q;
X(302) is selected from the group consisting of V, A, D, E, H, K, P, Q, S, and T;
X(303) is selected from the group consisting of V, N, P, and Q;
X(304) is S;
X(305) is selected from the group consisting of V, G and P;
X(306) is selected from the group consisting of L, I, N, T, V, and Y;
X(307) is T;
X(308) is V;
X(309) is V;
X(310) is H;
X(311) is Q;
X(312) is D;
X(313) is W;
X(314) is L;
X(315) is N;
X(316) is G;
X(317) is K;
X(318) is E;
X(319) is Y;
X(320) is K;
X(321) is C;
X(322) is K;
X(323) is V;
X(324) is S;
X(325) is N;
X(326) is K;
X(327) is G;
X(328) is L;
X(329) is P;
X(330) is A;
X(331) is P;
X(332) is I;
X(333) is E;
X(334) is K;
X(335) is T;
X(336) is I;
X(337) is S;
X(338) is K;
X(339) is T;
X(340) is K;
X(341) is G;
X(342) is Q;
X(343) is P;
X(344) is R;
X(345) is E;
X(346) is P;
X(347) is Q;
X(348) is V;
X(349) is Y;
X(350) is T;
X(351) is L;
X(352) is P;
X(353) is P;
X(354) is S;
X(355) is R;
X(356) is E;
X(357) is E;
X(358) is M;
X(359) is T;
X(360) is K;
X(361) is N;
X(362) is Q;
X(363) is V;
X(364) is S;
X(365) is L;
X(366) is T;
X(367) is C;
X(368) is L;
X(369) is V;
X(370) is K;
X(371) is G;
X(372) is F;
X(373) is Y;
X(374) is P;
X(375) is S;
X(376) is D;
X(377) is I;
X(378) is A;
X(379) is V;
X(380) is E;
X(381) is W;
X(382) is E;
X(383) is S;
X(384) is N;
X(385) is G;
X(386) is Q;
X(387) is P;
X(388) is E;
X(389) is N;
X(390) is N;
X(391) is Y;
X(392) is K;
X(393) is T;
X(394) is T;
X(395) is P;
X(396) is P;
X(397) is M;
X(398) is L;
X(399) is D;
X(400) is S;
X(401) is D;
X(402) is G;
X(403) is S;
X(404) is selected from the group consisting of F, H, L, M, and N;
X(405) is selected from the group consisting of F, D, E, T, and W;
X(406) is selected from the group consisting of L, A, D, E, G, K, N, Q, S, T, and V;
X(407) is selected from the group consisting of Y and H;
X(408) is S;
X(409) is K;
X(410) is selected from the group consisting of L, I, K, and Q;
X(411) is T;
X(412) is selected from the group consisting of V and P;
X(413) is D;
X(414) is K;
X(415) is S;
X(416) is R;
X(417) is W;
X(418) is Q;
X(419) is Q;
X(420) is G;
X(421) is N;
X(422) is V;
X(423) is F;
X(424) is S;
X(425) is C;
X(426) is S;
X(427) is V;
X(428) is M;
X(429) is H;
X(430) is E;
X(431) is A;
X(432) is selected from the group consisting of L, E and K;
X(433) is selected from the group consisting of H, D, G, P, S, T, and W;
X(434) is selected from the group consisting of N, D, E, G, H, S, T, and W;
X(435) is selected from the group consisting of H, G, K, M, N, P, S, T, and V;
X(436) is Y;
X(437) is selected from the group consisting of T, D, E, G, H, K, N, Q, and S;
X(438) is selected from the group consisting of Q, G, P, S, and T; and
X(439) is K;
X(440) is selected from the group consisting of S, D, E, G, H, K, N, P, Q, R, and T;
X(441) is L;
X(442) is S;
X(443) is L;
X(444) is S;
X(445) is P;
X(446) is G; and
X(447) is K;
said non-naturally occurring protein having reduced immunogenicity as compared with a protein comprising SEQ ID NO:2.

6. A non-naturally occurring protein comprising a variant Fc region comprising at least one amino acid modification of SEQ ID NO:2, said modification at a position selected from the group consisting of positions 251, 252, 253, 254, 256, 259, 278, 279, 282, 285, 301, 302, 303, 305, 306, 404, 405, 406, 407, 410, 412, 432, 433, 434, 435, 437, 438, and 440; and, wherein said modification at position 251 is selected from the group consisting of D, E, H, and T; wherein said modification at position 252 is selected from the group consisting of D, E, and H; wherein said modification at position 253 is selected from the group consisting of D, E, F, H, K, L, N, P, Q, R, S, T, V, W, and Y; wherein said modification at position 254 is selected from the group consisting of E, K, N, P, Q, R, V, and W; wherein said modification at position 256 is selected from the group consisting of I, L, M, P, S, V, W, and Y; wherein the modification at position 259 is T; wherein said modification at position 278 is selected from the group consisting of D and E; wherein said modification at position 279 is selected from the group consisting of A, Q, and T; wherein said modification at position 282 is selected from the group consisting of F, I, L, Q, and W; wherein said modification at position 285 is selected from the group consisting of P and T; wherein said modification at position 301 is selected from the group consisting of G, K, and Q; wherein said modification at position 302 is selected from the group consisting of A, D, E, H, K, P, Q, S, and T; wherein said modification at position 303 is selected from the group consisting of N, P, and Q; wherein said modification at position 305 is selected from the group consisting of G and P; wherein said modification at position 306 is selected from the group consisting of I, N, T, V, and Y; wherein said modification at position 404 is selected from the group consisting of H, L, M, and N; wherein said modification at position 405 is selected from the group consisting of D, E, T, and W; wherein said modification at position 406 is selected from the group consisting of A, D, E, G, K, N, Q, S, T, and V; wherein the modification at position 407 is H; wherein said modification at position 410 is selected from the group consisting of I, K, and Q; wherein the modification at position 412 is P; wherein said modification at position 432 is selected from the group consisting of E and K; wherein said modification at position 433 is selected from the group consisting of D, G, P, S, T, and W; wherein said modification at position 434 is selected from the group consisting of D, E, G, H, S, T, and W; wherein said modification at position 435 is selected from the group consisting of G, K, M, N, P, S, T, and V; wherein said modification at position 437 is selected from the group consisting of D, E, G, H, K, N, Q, and S; wherein said modification at position 438 is selected from the group consisting of G, P, S, and T; and, wherein said modification at position 440 is selected from the group consisting of D, E, G, H, K, N, P, Q, R, and T, said non-naturally occurring protein having reduced immunogenicity as compared with a protein comprising SEQ ID NO:2.

7. A variant protein of claim 6, wherein at least one modification is made to the group consisting of positions 251, 252, 253, 254, 256, 259, 278, 279, 282, 285, 302, 303, 305, 306, 404, 405, 406, 407, 410, 412, 432, 433, 434, 435, 437, 438, and 440; and, wherein said modification at position 251 is selected from the group consisting of D, E, H, and T; wherein the modification at position 252 is D; wherein said modification at position 253 is selected from the group consisting of D and E; wherein said modification at position 256 is selected from the group consisting of M, W, and Y; wherein the modification at position 278 is D; wherein said modification at position 282 is selected from the group consisting of F, L, Q, and W; wherein said modification at position 404 is selected from the group consisting of H and N; wherein said modification at position 432 is selected from the group consisting of E and K; and, wherein the modification at position 437 is E.

8. The variant protein of claim 6, wherein at least one modification is made to the group consisting of Agretope 16 (251-259), Agretope 17a (262-270), Agretope 18 (277-285), Agretope 19b (300-308), Agretope 20b (302-310), Agretope 21b (303-311), Agretope 22a (348-356), Agretope 23 (369-377), Agretope 24a (404-412), Agretope 25a (406-414), Agretope 27a (422-430), and Agretope 28a (432-440).

9. A non-naturally occurring protein comprising a variant Fc region having the formula:
-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(l161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-;
wherein
X(118) is A;
X(119) is S;
X(120) is T;
X(121) is K;
X(122) is G;
X(123) is P;
X(124) is S;
X(125) is V;
X(126) is F;
X(127) is P;
X(128) is L;
X(129) is A;
X(130) is P;
X(131) is C;
X(132) is S;
X(133) is R;
X(134) is S;
X(135) is T;
X(136) is S;
X(137) is G;
X(138) is G;
X(139) is T;
X(140) is A;
X(141) is A;
X(142) is L;
X(143) is G;
X(144) is C;
X(145) is L;
X(146) is V;
X(147) is K;
X(148) is D;
X(149) is Y;
X(150) is F;
X(151) is P;
X(152) is E;
X(153) is P;
X(154) is V;
X(155) is T;
X(156) is V;
X(157) is S;
X(158) is W;
X(159) is N;
X(160) is S;
X(161) is G;
X(162) is A;
X(163) is L;
X(164) is T;
X(165) is S;
X(166) is G;
X(167) is V;
X(168) is H;
X(169) is T;
X(170) is F;
X(171) is P;
X(172) is A;
X(173) is V;
X(174) is L;
X(175) is Q;
X(176) is S;
X(177) is S;
X(178) is G;
X(179) is L;
X(180) is Y;
X(181) is S;
X(182) is L;
X(183) is S;
X(184) is S;
X(185) is V;
X(186) is V;
X(187) is T;
X(188) is V;
X(189) is P;
X(190) is S;
X(191) is S;
X(192) is S;
X(193) is L;
X(194) is G;
X(195) is T;
X(196) is Q;
X(197) is T;
X(198) is Y;
X(199) is T;
X(200) is C;
X(201) is N;
X(202) is V;
X(203) is N;
X(204) is H;
X(205) is K;
X(206) is P;
X(207) is S;
X(208) is N;
X(209) is T;
X(210) is K;
X(211) is V;
X(212) is D;
X(213) is K;
X(214) is R;
X(215) is V;
X(216) is E;
X(217) is L;
X(218) is K;
X(219) is T;
X(220) is P;
X(221) is the sequence LGD;
X(222) is T;
X(223) is T;
X(224) is H;
X(225) is T;
X(226) is C;
X(227) is P;
X(228) is the sequence RCPEPKSCDTPPPCPRCPEPKSCDTPPPCPRCPEPKSCDTPPPCPR; (SEQ ID NO: 1939)
X(229) is C;
X(230) is P;
X(231) is A;
X(232) is P;
X(233) is E;
X(234) is L;
X(235) is L;
X(236) is G;
X(237) is G;
X(238) is P;
X(239) is S;
X(240) is V;
X(241) is F;
X(242) is L;
X(243) is F;
X(244) is P;
X(245) is P;
X(246) is K;
X(247) is P;
X(248) is K;
X(249) is D;
X(250) is T;
X(251) is selected from the group consisting of L, D, E, H, and T;
X(252) is selected from the group consisting of M, D, E, and H;
X(253) is selected from the group consisting of I, D, E, F, H, K, L, N, P, Q, R, S, T, V, W, and Y;
X(254) is selected from the group consisting of S, E, K, N, P, Q, R, V, and W;
X(255) is R;
X(256) is selected from the group consisting of T, I, L, M, P, S, V, W, and Y;
X(257) is P;
X(258) is E;
X(259) is selected from the group consisting of V and T;
X(260) is T;
X(261) is C;
X(262) is V;
X(263) is V;
X(264) is V;
X(265) is D;
X(266) is V;
X(267) is S;
X(268) is H;
X(269) is E;
X(270) is D;
X(271) is P;
X(272) is E;
X(273) is V;
X(274) is Q;
X(275) is F;
X(276) is K;
X(277) is W;
X(278) is selected from the group consisting of Y, D, E, and S;
X(279) is selected from the group consisting of V, A, Q, and T;
X(280) is D;
X(281) is G;
X(282) is selected from the group consisting of V, F, G, I, L, P, Q, W, and Y;
X(283) is selected from the group consisting of E and W;
X(284) is V;
X(285) is selected from the group consisting of H, E, P, and T;
X(286) is N;
X(287) is A;
X(288) is K;
X(289) is T;
X(290) is K;
X(291) is P;
X(292) is R;
X(293) is E;
X(294) is E;
X(295) is Q;
X(296) is Y;
X(297) is N;
X(298) is S;
X(299) is T;
X(300) is selected from the group consisting of F, A, D, E, G, H, K, M, N, P, Q, R, S, T, and V;
X(301) is selected from the group consisting of R, D, E, G, H, K, and Q;
X(302) is selected from the group consisting of V, A, D, E, H, K, P, Q, S, and T;
X(303) is selected from the group consisting of V, D, E, N, P, Q, and S;
X(304) is S;
X(305) is selected from the group consisting of V, G, P, and T;
X(306) is selected from the group consisting of L, F, H, I, N, T, V, and Y;
X(307) is T;
X(308) is selected from the group consisting of V, A, N, P, and S;
X(309) is selected from the group consisting of L, F, G, I, M, N, Q, S, T, V, W, and Y;
X(310) is H;
X(311) is selected from the group consisting of Q, D, E, G, P, T, and W;
X(312) is D;
X(313) is W;
X(314) is L;
X(315) is N;
X(316) is G;
X(317) is K;
X(318) is E;
X(319) is Y;
X(320) is K;
X(321) is C;
X(322) is K;
X(323) is V;
X(324) is S;
X(325) is N;
X(326) is K;
X(327) is A;
X(328) is L;
X(329) is P;
X(330) is A;
X(331) is P;
X(332) is I;
X(333) is E;
X(334) is K;
X(335) is T;
X(336) is I;
X(337) is S;
X(338) is K;
X(339) is T;
X(340) is K;
X(341) is G;
X(342) is Q;
X(343) is P;
X(344) is R;
X(345) is E;
X(346) is P;
X(347) is Q;
X(348) is V;
X(349) is Y;
X(350) is T;
X(351) is L;
X(352) is P;
X(353) is P;
X(354) is S;
X(355) is R;
X(356) is E;
X(357) is E;
X(358) is M;
X(359) is T;
X(360) is K;
X(361) is N;
X(362) is Q;
X(363) is V;
X(364) is S;
X(365) is L;
X(366) is T;
X(367) is C;
X(368) is L;
X(369) is V;
X(370) is K;
X(371) is G;
X(372) is F;
X(373) is Y;
X(374) is P;
X(375) is S;
X(376) is D;
X(377) is I;
X(378) is A;
X(379) is V;
X(380) is E;
X(381) is W;
X(382) is E;
X(383) is S;
X(384) is S;
X(385) is G;
X(386) is Q;
X(387) is P;
X(388) is E;
X(389) is N;
X(390) is N;
X(391) is Y;
X(392) is N;
X(393) is T;
X(394) is T;
X(395) is P;
X(396) is P;
X(397) is M;
X(398) is L;
X(399) is D;
X(400) is S;
X(401) is D;
X(402) is G;
X(403) is S;
X(404) is F;
X(405) is F;
X(406) is L;
X(407) is Y;
X(408) is S;
X(409) is K;
X(410) is L;
X(411) is T;
X(412) is V;
X(413) is D;
X(414) is K;
X(415) is S;
X(416) is R;
X(417) is W;
X(418) is Q;
X(419) is Q;
X(420) is G;
X(421) is N;
X(422) is I;
X(423) is F;
X(424) is S;
X(425) is C;
X(426) is S;
X(427) is V;
X(428) is M;
X(429) is H;
X(430) is E;
X(431) is A;
X(432) is L;
X(433) is H;
X(434) is N;
X(435) is R;
X(436) is F;
X(437) is T;
X(438) is Q;
X(439) is K;
X(440) is S;
X(441) is L;
X(442) is S;
X(443) is L;
X(444) is S;
X(445) is P;
X(446) is G; and
X(447) is K; wherein
said non-naturally occurring protein having reduced immunogenicity as compared with a protein comprising SEQ ID NO:3.

10. A non-naturally occurring protein comprising a variant Fc region comprising at least one amino acid modification of a naturally occurring protein sequence selected from the group consisting of SEQ ID NO:3, said modification at a position selected from the group consisting of positions 251, 252, 253, 254, 256, 259, 278, 279, 282, 283, 285, 300, 301, 302, 303, 305, 306, 308, 309, and 311; and, wherein said modification at position 251 is selected from the group consisting of D, E, H, and T; wherein said modification at position 252 is selected from the group consisting of D, E, and H; wherein said modification at position 253 is selected from the group consisting of D, E, F, H, K, L, N, P, Q, R, S, T, V, W, and Y; wherein said modification at position 254 is selected from the group consisting of E, K, N, P, Q, R, V, and W; wherein said modification at position 256 is selected from the group consisting of I, L, M, P, S, V, W, and Y; wherein the modification at position 259 is T; wherein said modification at position 278 is selected from the group consisting of D, E, and S; wherein said modification at position 279 is selected from the group consisting of A, Q, and T; wherein said modification at position 282 is selected from the group consisting of F, G, I, L, P, Q, W, and Y; wherein the modification at position 283 is W; wherein said modification at position 285 is selected from the group consisting of E, P, and T; wherein said modification at position 300 is selected from the group consisting of A, D, E, G, H, K, M, N, P, Q, R, S, T, and V; wherein said modification at position 301 is selected from the group consisting of D, E, G, H, K, and Q; wherein said modification at position 302 is selected from the group consisting of A, D, E, H, K, P, Q, S, and T; wherein said modification at position 303 is selected from the group consisting of D, E, N, P, Q, and S; wherein said modification at position 305 is selected from the group consisting of G, P, and T; wherein said modification at position 306 is selected from the group consisting of F, H, I, N, T, V, and Y; wherein said modification at position 308 is selected from the group consisting of A, N, P, and S; wherein said modification at position 309 is selected from the group consisting of F, G, I, M, N, Q, S, T, V, W, and Y; and, wherein said modification at position 311 is selected from the group consisting of D, E, G, P, T, and W, said non-naturally occurring protein having reduced immunogenicity as compared with a protein comprising SEQ ID NO:3.

11. The non-naturally occurring protein of claim 10, wherein at least one modification is made to the group consisting of positions 251, 252, 253, 254, 256, 259, 278, 279, 282, 283, 285, 302, 303, 305, 306, 308, 309, and 311; and, wherein said modification at position 251 is selected from the group consisting of D, E, H, and T; wherein the modification at position 252 is D; wherein said modification at position 253 is selected from the group consisting of D and E; wherein said modification at position 256 is selected from the group consisting of M, W, and Y; wherein the modification at position 278 is D; wherein said modification at position 282 is selected from the group consisting of F, L, Q, and W; wherein said modification at position 303 is selected from the group consisting of N, P, Q, and S; and, wherein the modification at position 311 is D.

12. The non-naturally occurring protein of claim 10, wherein at least one modification is made to the group consisting of Agretope 4 (145-153), Agretope 5 (149-157), Agretope 6 (167-175), Agretope 7 (174-182), Agretope 8 (179-187), Agretope 9 (180-188), Agretope 10 (182-190), Agretope 11a (185-193), Agretope 12 (202-210), Agretope 13 (215-223), Agretope 15 (240-248), Agretope 16 (251-259), Agretope 17a (262-270), Agretope 18 (277-285), Agretope 19b (300-308), Agretope 20a (302-310), Agretope 21a (303-311), Agretope 22a (348-356), Agretope 23 (369-377), Agretope 27b (422-430), and Agretope 28b (432-440).

13. A non-naturally occurring protein comprising a variant Fc region having the formula:
-X(118)-X(119)-X(120)-X(121)-X(122)-X(123)-X(124)-X(125)-X(126)-X(127)-X(128)-X(129)-X(130)-X(131)-X(132)-X(133)-X(134)-X(135)-X(136)-X(137)-X(138)-X(139)-X(140)-X(141)-X(142)-X(143)-X(144)-X(145)-X(146)-X(147)-X(148)-X(149)-X(150)-X(151)-X(152)-X(153)-X(154)-X(155)-X(156)-X(157)-X(158)-X(159)-X(160)-X(161)-X(162)-X(163)-X(164)-X(165)-X(166)-X(167)-X(168)-X(169)-X(170)-X(171)-X(172)-X(173)-X(174)-X(175)-X(176)-X(177)-X(178)-X(179)-X(180)-X(181)-X(182)-X(183)-X(184)-X(185)-X(186)-X(187)-X(188)-X(189)-X(190)-X(191)-X(192)-X(193)-X(194)-X(195)-X(196)-X(197)-X(198)-X(199)-X(200)-X(201)-X(202)-X(203)-X(204)-X(205)-X(206)-X(207)-X(208)-X(209)-X(210)-X(211)-X(212)-X(213)-X(214)-X(215)-X(216)-X(217)-X(218)-X(219)-X(220)-X(221)-X(222)-X(223)-X(224)-X(225)-X(226)-X(227)-X(228)-X(229)-X(230)-X(231)-X(232)-X(233)-X(234)-X(235)-X(236)-X(237)-X(238)-X(239)-X(240)-X(241)-X(242)-X(243)-X(244)-X(245)-X(246)-X(247)-X(248)-X(249)-X(250)-X(251)-X(252)-X(253)-X(254)-X(255)-X(256)-X(257)-X(258)-X(259)-X(260)-X(261)-X(262)-X(263)-X(264)-X(265)-X(266)-X(267)-X(268)-X(269)-X(270)-X(271)-X(272)-X(273)-X(274)-X(275)-X(276)-X(277)-X(278)-X(279)-X(280)-X(281)-X(282)-X(283)-X(284)-X(285)-X(286)-X(287)-X(288)-X(289)-X(290)-X(291)-X(292)-X(293)-X(294)-X(295)-X(296)-X(297)-X(298)-X(299)-X(300)-X(301)-X(302)-X(303)-X(304)-X(305)-X(306)-X(307)-X(308)-X(309)-X(310)-X(311)-X(312)-X(313)-X(314)-X(315)-X(316)-X(317)-X(318)-X(319)-X(320)-X(321)-X(322)-X(323)-X(324)-X(325)-X(326)-X(327)-X(328)-X(329)-X(330)-X(331)-X(332)-X(333)-X(334)-X(335)-X(336)-X(337)-X(338)-X(339)-X(340)-X(341)-X(342)-X(343)-X(344)-X(345)-X(346)-X(347)-X(348)-X(349)-X(350)-X(351)-X(352)-X(353)-X(354)-X(355)-X(356)-X(357)-X(358)-X(359)-X(360)-X(361)-X(362)-X(363)-X(364)-X(365)-X(366)-X(367)-X(368)-X(369)-X(370)-X(371)-X(372)-X(373)-X(374)-X(375)-X(376)-X(377)-X(378)-X(379)-X(380)-X(381)-X(382)-X(383)-X(384)-X(385)-X(386)-X(387)-X(388)-X(389)-X(390)-X(391)-X(392)-X(393)-X(394)-X(395)-X(396)-X(397)-X(398)-X(399)-X(400)-X(401)-X(402)-X(403)-X(404)-X(405)-X(406)-X(407)-X(408)-X(409)-X(410)-X(411)-X(412)-X(413)-X(414)-X(415)-X(416)-X(417)-X(418)-X(419)-X(420)-X(421)-X(422)-X(423)-X(424)-X(425)-X(426)-X(427)-X(428)-X(429)-X(430)-X(431)-X(432)-X(433)-X(434)-X(435)-X(436)-X(437)-X(438)-X(439)-X(440)-X(441)-X(442)-X(443)-X(444)-X(445)-X(446)-X(447)-;
wherein
X(118) is A;
X(119) is S;
X(120) is T;
X(121) is K;
X(122) is G;
X(123) is P;
X(124) is S;
X(125) is V;
X(126) is F;
X(127) is P;
X(128) is L;
X(129) is A;
X(130) is P;
X(131) is C;
X(132) is S;
X(133) is R;
X(134) is S;
X(135) is T;
X(136) is S;
X(137) is E;
X(138) is S;
X(139) is T;
X(140) is A;
X(141) is A;
X(142) is L;
X(143) is G;
X(144) is C;
X(145) is L;
X(146) is V;
X(147) is K;
X(148) is D;
X(149) is Y;
X(150) is F;
X(151) is P;
X(152) is E;
X(153) is P;
X(154) is V;
X(155) is T;
X(156) is V;
X(157) is S;
X(158) is W;
X(159) is N;
X(160) is S;
X(161) is G;
X(162) is A;
X(163) is L;
X(164) is T;
X(165) is S;
X(166) is G;
X(167) is V;
X(168) is H;
X(169) is T;
X(170) is F;
X(171) is P;
X(172) is A;
X(173) is V;
X(174) is L;
X(175) is Q;
X(176) is S;
X(177) is S;
X(178) is G;
X(179) is L;
X(180) is Y;
X(181) is S;
X(182) is L;
X(183) is S;
X(184) is S;
X(185) is V;
X(186) is V;
X(187) is T;
X(188) is V;
X(189) is P;
X(190) is S;
X(191) is S;
X(192) is S;
X(193) is L;
X(194) is G;
X(195) is T;
X(196) is K;
X(197) is T;
X(198) is Y;
X(199) is T;
X(200) is C;
X(201) is N;
X(202) is V;
X(203) is D;
X(204) is H;
X(205) is K;
X(206) is P;
X(207) is S;
X(208) is N;
X(209) is T;
X(210) is K;
X(211) is V;
X(212) is D;
X(213) is K;
X(214) is R;
X(215) is V;
X(216) is E;
X(217) is S;
X(218) is K;
X(219) is Y;
X(220) is G;
X(221) is a bond;
X(222) is a bond;
X(223) is a bond;
X(224) is P;
X(225) is P;
X(226) is C;
X(227) is P;
X(228) is S;
X(229) is C;
X(230) is P;
X(231) is A;
X(232) is P;
X(233) is E;
X(234) is F;
X(235) is L;
X(236) is G;
X(237) is G;
X(238) is P;
X(239) is S;
X(240) is V;
X(241) is F;
X(242) is L;
X(243) is F;
X(244) is P;
X(245) is P;
X(246) is K;
X(247) is P;
X(248) is K;
X(249) is D;
X(250) is T;
X(251) is selected from the group consisting of L, D, E, H, and T;
X(252) is selected from the group consisting of M, D, E, and H;
X(253) is selected from the group consisting of I, D, E, F, H, K, L, N, P, Q, R, S, T, V, W, and Y;
X(254) is selected from the group consisting of S, E, K, N, P, Q, R, V, and W;
X(255) is R;
X(256) is selected from the group consisting of T, I, L, M, P, S, V, W, and Y;
X(257) is P;
X(258) is E;
X(259) is selected from the group consisting of V and T;
X(260) is T;
X(261) is C;
X(262) is V;
X(263) is V;
X(264) is V;
X(265) is D;
X(266) is V;
X(267) is S;
X(268) is Q;
X(269) is E;
X(270) is D;
X(271) is P;
X(272) is E;
X(273) is V;
X(274) is Q;
X(275) is F;
X(276) is N;
X(277) is W;
X(278) is selected from the group consisting of Y, D and E;
X(279) is selected from the group consisting of V, A, Q, T, and W;
X(280) is D;
X(281) is G;
X(282) is selected from the group consisting of V, F, G, I, L, Q, and W;
X(283) is E;
X(284) is V;
X(285) is selected from the group consisting of H, P, and T;
X(286) is N;
X(287) is A;
X(288) is K;
X(289) is T;
X(290) is K;
X(291) is P;
X(292) is R;
X(293) is E;
X(294) is E;
X(295) is Q;
X(296) is F;
X(297) is N;
X(298) is S;
X(299) is T;
X(300) is Y;
X(301) is selected from the group consisting of R, G, K, and Q;
X(302) is selected from the group consisting of V, A, E, H, K, Q, S, and T;
X(303) is selected from the group consisting of V, N, P, Q, R, and S;
X(304) is S;
X(305) is selected from the group consisting of V, G, P, and T;
X(306) is selected from the group consisting of L, F, H, I, N, T, V, and Y;
X(308) is selected from the group consisting of V, A, N, P, and S;
X(309) is selected from the group consisting of L, F, G, I, M, N, Q, S, T, V, W, and Y;
X(310) is H;
X(311) is selected from the group consisting of Q, D, E, G, P, T, and W;
X(312) is D;
X(313) is W;
X(314) is L;
X(315) is N;
X(316) is G;
X(317) is K;
X(318) is E;
X(319) is Y;
X(320) is K;
X(321) is C;
X(322) is K;
X(323) is V;
X(324) is S;
X(325) is N;
X(326) is K;
X(327) is G;
X(328) is L;
X(329) is P;
X(330) is S;
X(331) is S;
X(332) is I;
X(333) is E;
X(334) is K;
X(335) is T;
X(336) is I;
X(337) is S;
X(338) is K;
X(339) is A;
X(340) is K;
X(341) is G;
X(342) is Q;
X(343) is P;
X(344) is R;
X(345) is E;
X(346) is P;
X(347) is Q;
X(348) is V;
X(349) is Y;
X(350) is T;
X(351) is L;
X(352) is P;
X(353) is P;
X(354) is S;
X(355) is Q;
X(356) is E;
X(357) is E;
X(358) is M;
X(359) is T;
X(360) is K;
X(361) is N;
X(362) is Q;
X(363) is V;
X(364) is S;
X(365) is L;
X(366) is T;
X(367) is C;
X(368) is L;
X(369) is V;
X(370) is K;
X(371) is G;
X(372) is F;
X(373) is Y;
X(374) is P;
X(375) is S;
X(376) is D;
X(377) is I;
X(378) is A;
X(379) is V;
X(380) is E;
X(381) is W;
X(382) is E;
X(383) is S;
X(384) is N;
X(385) is G;
X(386) is Q;
X(387) is P;
X(388) is E;
X(389) is N;
X(390) is N;
X(391) is Y;
X(392) is K;
X(393) is T;
X(394) is T;
X(395) is P;
X(396) is P;
X(397) is V;
X(398) is L;
X(399) is D;
X(400) is S;
X(401) is D;
X(402) is G;
X(403) is S;
X(404) is selected from the group consisting of F, H, I, L, M, N, Q, T, and V;
X(405) is selected from the group consisting of F and W;
X(406) is selected from the group consisting of L, A, D, E, G, K, N, Q, S, T, and V;
X(407) is selected from the group consisting of Y and M;
X(408) is S;
X(409) is selected from the group consisting of R, G, Q, and S;
X(410) is selected from the group consisting of L, F, Q, and Y;
X(411) is T;
X(412) is selected from the group consisting of V and P;
X(413) is D;
X(414) is K;
X(415) is S;
X(416) is R;
X(417) is W;
X(418) is Q;
X(419) is E;
X(420) is G;
X(421) is N;
X(422) is V;
X(423) is F;
X(424) is S;
X(425) is C;
X(426) is S;
X(427) is V;
X(428) is M;
X(429) is H;
X(430) is E;
X(431) is A;
X(432) is selected from the group consisting of L, E and K;
X(433) is selected from the group consisting of H, D, G, P, S, T, and W;
X(434) is selected from the group consisting of N, D, E, G, H, S, T, and W;
X(435) is selected from the group consisting of H, G, K, M, N, P, S, T, and V;
X(436) is Y;
X(437) is selected from the group consisting of T, D, E, G, H, K, N, Q, and S;
X(438) is selected from the group consisting of Q, G, P, S, and T; and
X(439) is K;
X(440) is selected from the group consisting of S, D, E, G, H, K, N, P, Q, R, and T;
X(441) is L;
X(442) is S;
X(443) is L;
X(444) is S;
X(445) is L;
X(446) is G; and
X(447) is K;
said non-naturally occurring protein having reduced immunogenicity as compared with a protein comprising SEQ ID NO:4.

14. A non-naturally occurring protein comprising a variant Fc region comprising at least one amino acid modification of SEQ ID NO:4, said modification at a position selected from the group consisting of positions 251, 252, 253, 254, 256, 259, 278, 279, 282, 283, 285, 300, 301, 302, 303, 305, 306, 308, 309, 311, 404, 405, 406, 407, 409, 410, 412, 432, 433, 434, 435, 437, 438, and 440; and, wherein said modification at position 251 is selected from the group consisting of D, E, H, and T; wherein said modification at position 252 is selected from the group consisting of D, E, and H; wherein said modification at position 253 is selected from the group consisting of D, E, F, H, K, L, N, P, Q, R, S, T, V, W, and Y; wherein said modification at position 254 is selected from the group consisting of E, K, N, P, Q, R, V, and W; wherein said modification at position 256 is selected from the group consisting of I, L, M, P, S, V, W, and Y; wherein the modification at position 259 is T; wherein said modification at position 278 is selected from the group consisting of D and E; wherein said modification at position 279 is selected from the group consisting of A, Q, T, and W; wherein said modification at position 282 is selected from the group consisting of F, G, I, L, P, Q, W, and Y; wherein the modification at position 283 is G; wherein said modification at position 285 is selected from the group consisting of E, P, and T; wherein said modification at position 300 is selected from the group consisting of A, D, E, G, H, K, M, N, P, Q, R, S, T, and V; wherein said modification at position 301 is selected from the group consisting of D, E, G, K, and Q; wherein said modification at position 302 is selected from the group consisting of A, E, H, K, Q, S, and T; wherein said modification at position 303 is selected from the group consisting of D, E, N, P, of R, and S; wherein said modification at position 305 is selected from the group consisting of G, P, and T; wherein said modification at position 306 is selected from the group consisting of F, H, I, N, T, V, and Y; wherein said modification at position 308 is selected from the group consisting of A, N, P, and S; wherein said modification at position 309 is selected from the group consisting of F, G, I, M, N, O, S, T, V, W, and Y; wherein said modification at position 311 is selected from the group consisting of D, E, G, P, T, and W; wherein said modification at position 404 is selected from the group consisting of H, I, L, M, N, Q, T, and V; wherein the modification at position 405 is W; wherein said modification at position 406 is selected from the group consisting of A, D, E, G, K, N, Q, S, T, and V; wherein the modification at position 407 is M; wherein said modification at position 409 is selected from the group consisting of G, Q, and S; wherein said modification at position 410 is selected from the group consisting of F, Q, and Y; wherein the modification at position 412 is P; wherein said modification at position 432 is selected from the group consisting of E and K; wherein said modification at position 433 is selected from the group consisting of D, G, P, S, T, and W; wherein said modification at position 434 is selected from the group consisting of D, E, G, H, S, T, and W; wherein said modification at position 435 is selected from the group consisting of G, K, M, N, P, S, T, and V; wherein said modification at position 437 is selected from the group consisting of D, E, G, H, K, N, Q, and S; wherein said modification at position 438 is selected from the group consisting of G, P, S, and T; and, wherein said modification at position 440 is selected from the group consisting of D, E, G, H, K, N, P, Q, R, and T, said non-naturally occurring protein having reduced immunogenicity as compared with a protein comprising SEQ ID NO:4.

15. The non-naturally occurring protein of claim 14, wherein at least one modification is made to the group consisting of positions 251, 252, 253, 254, 256, 259, 278, 279, 282, 283, 285, 300, 301, 302, 303, 305, 306, 308, 309, 311, 404, 405, 406, 407, 409, 410, 412, 432, 433, 434, 435, 437, 438, and 440; and, wherein said modification at position 251 is selected from the group consisting of D, E, H, and T; wherein the modification at position 252 is D; wherein said modification at position 253 is selected from the group consisting of D and E; wherein said modification at position 256 is selected from the group consisting of M, W, and Y; wherein the modification at position 278 is D; wherein said modification at position 282 is selected from the group consisting of F, G, L, Q, W, and Y; wherein said modification at position 300 is selected from the group consisting of A, D, E, G, H, K, N, P, Q, R, S, and T; wherein the modification at position 301 is D; wherein said modification at position 303 is selected from the group consisting of D, E, N, P, Q, R, and S; wherein the modification at position 311 is D; wherein said modification at position 404 is selected from the group consisting of H, N, Q, and T; wherein said modification at position 432 is selected from the group consisting of E and K; and, wherein the modification at position 437 is E.

16. The non-naturally occurring protein of claim 14 wherein at least one modification is made to the group consisting of Agretope 16 (251-259), Agretope 17b (262-270), Agretope 18 (277-285), Agretope 19a (300-308), Agretope 20a (302-310), Agretope 21a (303-311), Agretope 22b (348-356), Agretope 23 (369-377), Agretope 24b (404-412), Agretope 25b (406-414), Agretope 26 (407-415), Agretope 27a (422-430), and Agretope 28a (432-440).
